(19) United States
(12) Patent Application Publication
(10) Pub. No.: US 2016/0031888 A1
(43) Pub. Date: Feb. 4, 2016

(54) 3-(ARYL OR HETEROARYL) METHYLENEINDOLIN-2-ONE DERIVATIVES AS INHIBITORS OF CANCER STEM CELL PATHWAY KINASES FOR THE TREATMENT OF CANCER

(71) Applicant: BOSTON BIOMEDICAL, INC., Cambridge, MA (US)

(72) Inventors: Chiang Jia Li, Cambridge, MA (US); Ji-feng Liu, Winchester, MA (US); Wei Li, Norwood, MA (US); Amanda Gibeau, Natick, MA (US); Harry Rogoff, Wrentham, MA (US); Katsunori Tsuboi, Osaka (JP); Yosuke Takanashi, Osaka (JP); Shingo Tojo, Osaka (JP); Tomohiro Kodama, Osaka (JP); Katsumi Kubota, Osaka (JP); Toshio Kanai, Osaka (JP)

(73) Assignee: BOSTON BIOMEDICAL, INC., Cambridge, MA (US)

(21) Appl. No.: 14/774,803

(22) PCT Filed: Mar. 13, 2014
(86) PCT No.: PCT/US2014/026498

§ 371 Date: Sep. 11, 2015

Related U.S. Application Data
(60) Provisional application No. 61/780,263, filed on Mar. 13, 2013.
(60) Provisional application No. 61/780,248, filed on Mar. 13, 2013.

(51) Int. Cl. C07D 487/04; C07D 417/14; C07D 471/04; C07D 405/14; C07D 401/14; C07D 409/04; C07D 409/14

(52) U.S. Cl. CPC C07D 487/04; C07D 409/04; C07D 417/14; C07D 409/14; C07D 405/14; C07D 401/14; C07D 471/04

(57) ABSTRACT

The invention provides compounds of Formula I as inhibitors of cancer stem cells as well as cancer stem cell pathway kinase and other related kinases, pharmaceutical compositions and uses thereof in the treatment of cancer or a related disorder in a mammal, wherein i.a. R₂ is an optionally substituted heterocycle or optionally substituted aryl; and one of R₄, R₅, R₆ and R₇ is a substituted heterocycle or substituted aryl.

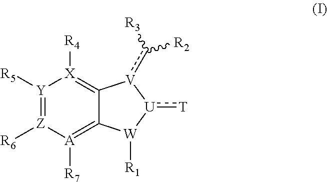

### RELATED APPLICATIONS

This application claims the benefit of U.S. Provisional Application No. 61/780,248, filed Mar. 13, 2013 and U.S. Provisional Application No. 61/780,263, filed Mar. 13, 2013. The contents of each application are hereby incorporated by reference in their entirety.

### TECHNICAL FIELD OF THE INVENTION

The invention generally relates to inhibitors of cancer stem cells. More particularly, the invention relates to novel inhibitors of cancer stem cells as well as cancer stem cell pathway kinase and other related kinases, to pharmaceutical compositions and uses thereof in the treatment of cancer or a related disorder in a mammal, and to methods of making such compounds and compositions.

### BACKGROUND OF THE INVENTION

Despite decades of intensive scientific and clinical research, cancer remains a challenging disease to both the patient and the healthcare provider. In the U.S. alone, it is estimated that there are over 1.5 million new cases of cancer and more than half million of cancer-related deaths in 2011. Worldwide, cancer is the third leading cause of death.

Cancer is characterized by rapidly-proliferating cell growth in the body. Cancer is often able to invade other tissues from its original location and, in a process called metastasis, spread to other parts of the body through blood and lymphatics. There are many types of cancer, which may be classified in pathology and clinical diagnosis into carcinoma, sarcoma, leukemia, lymphoma and myeloma, and malignant tumors of the central nervous system.

At the present time, the leading therapies for cancer include surgery, radiation, and chemotherapy. Surgery and radiotherapy are quite successful in treating primary tumors. However, once a cancer has disseminated to distant sites, chemotherapy is often required to treat the disease. Cytotoxic agents have played a critical role in modern cancer therapy. However, they usually induce substantial toxicity in normal tissues. Targeted therapies that more specifically target cancer cells are more desirable. A relatively new class of agents with selectivity for targets implicated in tumor growth has started to emerge recently, demonstrating impressive efficacy with much less toxicity than cytotoxic agents.

Protein kinases represent potential targets for therapeutic inhibition. (Pyle, et al., 2006 Nat Biotechnol. 24(3): p. 344-50.) Protein kinases are a family of enzymes that regulate a wide variety of cellular processes, including cell growth, cell proliferation, cell differentiation and metabolism. A kinase enzyme that modifies other proteins by chemically adding phosphate groups to them in a phosphorylation process. Protein kinases communicate cell growth signals through sequential chemical modification of pathway partners. Therefore, pharmacologic inhibition of any kinase on a given signal transduction cascade would theoretically block communication along the entire pathway. In addition, it is known that protein kinases play a role in disease states and disorders, for example, kinase mutation and/or overexpression are frequently characterized in cancers, resulting in hyperactivated activity that often correlates with uncontrolled cell growth.

Cancer Stem Cells (CSC) is a subpopulation of cells within a variety of tumor types with a tumorigenic potential that is lacking in the rest of the cells within these tumors. CSC can generate tumors through the stem cell processes of self-renewal and differentiation into multiple cell types. There is mounting evidence that such cells exist in almost all tumor types. CSC give rise to the differentiated cells that form the bulk of the tumor mass and phenotypically characterize the disease. Cancer stem cells have been demonstrated to be fundamentally responsible for carcinogenesis, cancer metastasis, and cancer reoccurrence. In many tumors, CSC and their differentiated progeny appear to have markedly different biologic characteristics.

Therapies specifically targeted at CSCs, therefore, hold unique potential for improvement of survival and quality of life of cancer patients, especially for sufferers of metastatic disease. (PCT/US2008/075418, WO 2009/033033) Conventional therapies that target mature tumor cells may lead to clinical improvement, but are unlikely to be curative unless CSCs are also targeted. Relevant targets unique to the rare cancer stem cells may be missed if clinical activity is judged solely by criteria that reflect the effects of treatment on the bulk of the cancer.

Recent studies have shown that certain compounds inhibit kinases and kill cancer stem cells, demonstrating that kinases are important targets for killing or inhibiting cancer stem cells. These kinases important for CSCs are collectively referred to cancer stem cell pathway kinase (CSCPK) hereinafter. Our results provide a method of targeting cancer stem cells with CSCPK inhibitors.

There are continued unmet needs for novel inhibitors of cancer stem cells as well as cancer stem cell pathway kinase and other related kinases and targets.

### SUMMARY OF THE INVENTION

The invention provides novel inhibitors of cancer stem cells as well as cancer stem cell pathway kinase and other related kinases and targets, as well as pharmaceutical compositions and uses thereof in the treatment of a cancer or a related disorder in a mammal. The invention also provide synthetic and preparation methods of making such compounds and compositions.

In one aspect, the invention generally relates to a compound of Formula I,

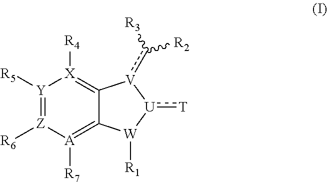

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₂ is monocyclic or bicyclic heterocycle or substituted heterocycle, aryl or substituted aryl;
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
T is O, S or Rₐ;
U, V, and W are each independently a carbon, N, O, or S;
X, Y, Z, and A are each independently a carbon or N, with the proviso that the ring in which X, Y, Z, and A exist is aromatic;
with the provision that
   one of R₄, R₅, R₆, and R₇ is substituted heterocycle or substituted aryl, and
   R₄, R₅, R₆, or R₇ is absent if X, Y, Z, or A, respectively, is a heteroatom;
      wherein
   substituted heterocycle and substituted aryl in R₄, R₅, R₆, and R₇ is the following group:

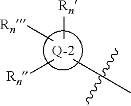

wherein
Q-2 is heterocycle, or aryl;
R_{n′}, R_{n″} and R_{n′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In another aspect, the invention generally relates to a compound of Formula II,

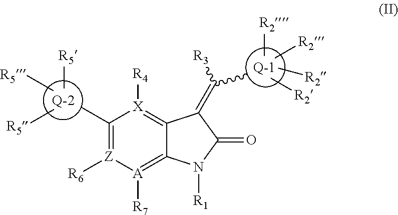

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Z, and A are each independently a carbon or N, with the proviso that the ring in which X, Z, and A exist is aromatic;
Q-1 and Q-2 are independently heterocycle or aryl;
R_{2′}, R_{2″}, R_{2′″} and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{5′}, R_{5″}, and R_{5′″} are each independently hydrogen, halogen, cyano, nitro, CF₃, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In yet another aspect, the invention generally relates to a compound of Formula III,

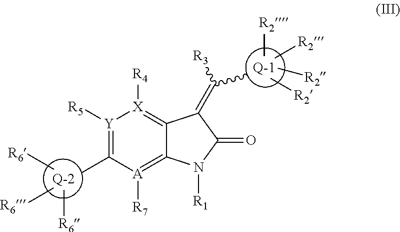

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Y, and A are each independently a carbon or N, with the proviso that the ring in which X, Y, and A exist is aromatic;
Q-1 and Q-2 are each independently heterocycle or aryl;
R_{2′}, R_{2″}, R_{2′″} and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{6′}, R_{6″} and R_{6′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In yet another aspect, the invention generally relates to a compound of Formula IV,

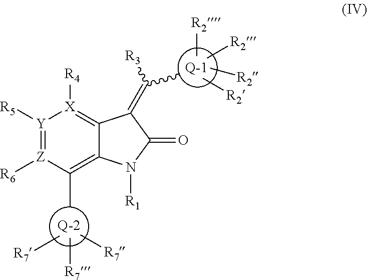

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, and R₆ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Y, and Z are each independently a carbon or N, with the proviso that the ring in which X, Y, and Z exist is aromatic;
Q-1 and Q-2 are each independently heterocycle or aryl;
R_{2′}, R_{2″}, R_{2′″} and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{7′}, R_{7″} and R_{7′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In yet another aspect, the invention generally relates to a compound of Formula V

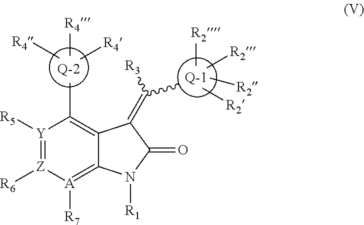

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₅, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
Y, Z and A are each independently a carbon or N, with the proviso that the ring in which Y, Z and A exist is aromatic;
Q-1 and Q-2 are each independently heterocycle or aryl;
R_{2′}, R_{2″}, R_{2′″}, and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{4′}, R_{4″} and R_{4′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In yet another aspect, the invention generally relates to a pharmaceutical composition comprising a compound disclosed herein, or a pharmaceutically acceptable salt, ester or pro-drug thereof, and a pharmaceutically acceptable excipient, carrier, or diluent.

In yet another aspect, the invention generally relates to a method of treating or preventing cancer, or a related disorder or condition thereof in a mammal, including a human, comprising administering to a subject in need thereof a therapeutically effective amount of a pharmaceutical composition comprising a compound disclosed herein, or a pharmaceutically acceptable salt, ester or pro-drug thereof, effective in the treatment or prevention of cancer, or a related disorder or condition thereof in a mammal, including a human, and a pharmaceutically acceptable excipient, carrier, or diluent.

### BRIEF DESCRIPTION OF THE DRAWINGS

FIG. 1 shows the kinase inhibition activity of the compounds.

### DEFINITIONS

Definitions of specific functional groups and chemical terms are described in more detail below. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Given the benefit of this disclosure, one of ordinary skill in the art will appreciate that synthetic methods, as described herein, may utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by preferably readily available, non-toxic reagents that do not attack the other functional groups; the protecting group forms an easily separable derivative (more preferably without the generation of new stereogenic centers); and the protecting group has a minimum of additional functionality to avoid further sites of reaction. Oxygen, sulfur, nitrogen, and carbon protecting groups may be utilized. Examples of a variety of protecting groups can be found in Protective Groups in Organic Synthesis, Third Ed. Greene, T. W. and Wuts, P. G., Eds., John Wiley & Sons, New York: 1999.

It will be appreciated that the compounds, as described herein, may be substituted with any number of substituents or functional moieties. Throughout the specifications, groups and substituents thereof may be chosen to provide stable moieties and compounds.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the term “pharmaceutically acceptable salt” refers to either a pharmaceutical acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

The compounds of the present invention may form salts that are also within the scope of this invention. Reference to a compound of the present invention herein is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps that may be employed during preparation. Salts of the compounds of the present invention may be formed, for example, by reacting a compound I, II or III with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

As used herein, the term “pharmaceutically acceptable ester,” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

As used herein, the term “prodrug” refers to a pharmacological derivative of a parent drug molecule that requires biotransformation, either spontaneous or enzymatic, within the organism to release the active drug. For example, prodrugs are variations or derivatives of the compounds of Formula I that have groups cleavable under certain metabolic conditions, which when cleaved, become the compounds of Formula I. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism (See, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

The term “pharmaceutically-acceptable excipient, carrier, or diluent” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, “Cₓ-C_{y}” refers in general to groups that have from x to y (inclusive) carbon atoms. Therefore, for example, C₁-C₆ refers to groups that have 1, 2, 3, 4, 5, or 6 carbon atoms, which encompass C₁-C₂, C₂-C₃, C₂-C₄, C₂-C₅, C₂-C₆, and all like combinations. “C₁-C₂₀” and the likes similarly encompass the various combinations between 1 and 20 (inclusive) carbon atoms, such as C₁-C₆, C₁-C₁₂ and C₃-C₁₂.

As used herein, the terms “alkyl” refers to a straight or branched chain alkane (hydrocarbon) radical. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing Cl₃), cyano, nitro, CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and R_{d} are independently hydrogen, alkyl, cycloalkyl, heterocycle, aryl, or said R_{b} and R_{c} together with the N to which they are bonded optionally form a heterocycle or substituted heterocycle; and Rₑ is alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle and aryl can themselves be optionally substituted. As used herein, the term “Cₓ-C_{y} alkyl” refers to a saturated linear or branched free radical consisting essentially of x to y carbon atoms, wherein x is an integer from 1 to about 10 and y is an integer from about 2 to about 20. Exemplary Cₓ-C_{y} alkyl groups include “C₁-C₂₀ alkyl,” which refers to a saturated linear or branched free radical consisting essentially of 1 to 20 carbon atoms and a corresponding number of hydrogen atoms. Exemplary C₁-C₂₀ alkyl groups include methyl, ethyl, n-propyl, isopropyl, n-butyl, isobutyl, dodecanyl, etc. Of course, other C₁-C₂₀ alkyl groups will be readily apparent to those of skill in the art given the benefit of the present disclosure. The term “alkyl” is C₁-C₂₀, preferably C₁-C₁₀, more preferably C₁-C₆, further preferably C₁-C₆.

As used herein, the term “alkenyl” refers to a straight or branched chain hydrocarbon radical having at least one carbon-carbon double bond. Exemplary such groups include ethenyl or allyl. “Substituted alkenyl” refers to an alkenyl group substituted with one or more substituents, preferably 1 to 4 substituents, at any available point of attachment. Exemplary substituents include, but are not limited to, alkyl or substituted alkyl, as well as those groups recited above as exemplary alkyl substituents. The exemplary substituents can themselves be optionally substituted. The term “alkenyl” is C₂-C₂₀, preferably C₂-C₁₀, more preferably C₂-C₆.

As used herein, the term “alkynyl” refers to a straight or branched chain hydrocarbon radical having at least one carbon to carbon triple bond. Exemplary such groups include ethynyl. “Substituted alkynyl” refers to an alkynyl group substituted with one or more substituents, preferably 1 to 4 substituents, at any available point of attachment. Exemplary substituents include, but are not limited to, alkyl or substituted alkyl, as well as those groups recited above as exemplary alkyl substituents. The exemplary substituents can themselves be optionally substituted. The term “alkynyl” is C₂-C₂₀, preferably C₂-C₁₀, more preferably C₂-C₆.

As used herein, the term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” or “Substituted phenyl” refers to an aryl or a phenyl group substituted by one or more substituents, preferably 1 to 3 substituents, at any point of attachment. Exemplary substituents include, but are not limited to, nitro, cycloalkyl or substituted cycloalkyl, cycloalkenyl or substituted cycloalkenyl, cyano, alkyl or substituted alkyl, as well as those groups recited above as exemplary alkyl substituents. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

As used herein, the term “cycloalkyl” refers to a fully saturated cyclic hydrocarbon group having from 1 to 4 rings and 3 to 10 carbons per ring. Exemplary such groups include cyclopropyl, cyclobutyl, cyclopentyl, cyclohexyl, cycloheptyl, etc. “Substituted cycloalkyl” refers to a cycloalkyl group substituted with one or more substituents, preferably 1 to 4 substituents, at any available point of attachment. Exemplary substituents include, but are not limited to, nitro, cyano, alkyl or substituted alkyl, as well as those groups recited above as exemplary alkyl substituents. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted. The term “cycloalkyl” is C₃-C₁₀, preferably C₃-C₈, more preferably C₃-C₆.

As used herein, the term “cycloalkenyl” refers to a partially unsaturated cyclic hydrocarbon group containing 1 to 4 rings and 3 to 10 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to nitro, cyano, alkyl or substituted alkyl, as well as those groups recited above as exemplary alkyl substituents. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted. The term “cycloalkenyl” is C₃-C₁₀, preferably C₃-C₈, more preferably C₃-C₆.

As used herein, the terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) that have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

As used herein, “substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, cycloalkyl or substituted cycloalkyl, cycloalkenyl or substituted cycloalkenyl, nitro, oxo (i.e., ═O), cyano, alkyl or substituted alkyl, heterocyclic or substituted heterocyclic, aryl or substituted aryl, as well as those groups recited above as exemplary alkyl substituents. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

As used herein, the term “halogen” refers to fluorine (F), chlorine (Cl), bromine (Br), or iodine (I).

The term “carbocyclic” refers to aromatic or non-aromatic 3 to 7 membered monocyclic and 7 to 11 membered bicyclic groups, in which all atoms of the ring or rings are carbon atoms. “Substituted carbocyclic” refers to a carbocyclic group substituted with one or more substituents, preferably 1 to 4 substituents, at any available point of attachment. Exemplary substituents include, but are not limited to, nitro, cyano, ORₐ, wherein Rₐ is as defined hereinabove, as well as those groups recited above as exemplary cycloalkyl substituents. The exemplary substituents can themselves be optionally substituted.

The term a “protein kinase related disorder” refers to any disease or deleterious condition in which a protein kinase plays a role. Examples include a serine-threonine kinase related disorder, a receptor tyrosine kinase related disorder, a non-receptor tyrosine kinase related disorder, an EGFR related disorder, an IGFR related disorder, a PDGFR related disorder and a flk related disorder.

According to one or more embodiments of the present invention, “cancer stem cell” (“CSC”) or “cancer stem cells” (“CSCs”) refer to a minute population of cancer cells that have self-renewal capability and are tumorigenic. They are also called “Cancer Initiating Cells”, “Tumor Initiating Cells”, “Cancer Stem-Like Cells”, “Stem-Like Cancer Cells”, “aggressive cancer cells”, and “super malignant cancer cells”, etc. The methods of isolating these cells include but not limited to enrichment by their ability of efflux Hoechst 33342, enrichment of surface markers such as CD133, CD44, and others, and enrichment by their tumorigenic property.

The term “CSCPK” or “CSCPKs” refer to protein kinase(s) that are essential for cancer stem cell survival or self-renewal.

Unless otherwise indicated, any heteroatom with unsatisfied valences is assumed to have hydrogen atoms sufficient to satisfy the valences.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. See, WO 2007/005643, WO 2007/005644, WO 2007/016361, and WO 2007/016431.

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

The compounds, salts, esters, prodrugs, hydrates, and solvates presently disclosed can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, all tautomers are within the scope of the present disclosure.

Atropisomers are also within the scope of the present disclosure. Atropisomers refer to compounds that can be separated into rotationally restricted isomers.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates of the compounds of the invention are also contemplated herein. Solvates of the compounds of the present invention include, for example, hydrates.

### DETAILED DESCRIPTION OF THE INVENTION

The invention provides unique novel inhibitors of cancer stem cells as well as cancer stem cell pathway kinase and other related kinases and targets, as well as pharmaceutical compositions and uses thereof in the treatment of a cancer or a related disorder in a mammal.

Specifically, the present invention is as follows.

Item1. A compound of Formula I,

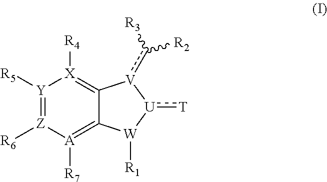

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₂ is monocyclic or bicyclic heterocycle or substituted heterocycle, aryl or substituted aryl;
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, S(O)₂NRₐR_{b};
R₄, R₅, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
T is O, S or Rₐ;
U, V, and W are each independently a carbon, N, O, or S;
X, Y, Z, and A are each independently a carbon or N, with the proviso that the ring in which X, Y, Z, and A exist is aromatic;
with the provision that
   one of R₄, R₅, R₆, and R₇ is substituted heterocycle or substituted aryl, and
   R₄, R₅, R₆, or R₇ is absent if X, Y, Z, or A, respectively, is a heteroatom;
      wherein
   substituted heterocycle and substituted aryl in R₄, R₅, R₆, and R₇ is the following group:

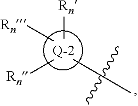

wherein
Q-2 is heterocycle or aryl;
R_{n′}, R_{n″} and R_{n′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

Item2. The compound of Item 1, wherein T is O or S,

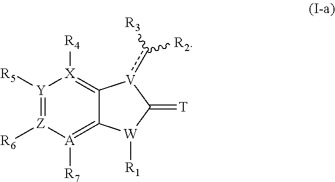

Item3. The compound of Item 2, wherein T is O,

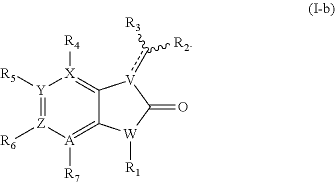

Item4. The compound of Item 2, V is carbon,

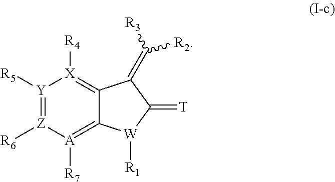

Item5. The compound of Item 2, W is N,

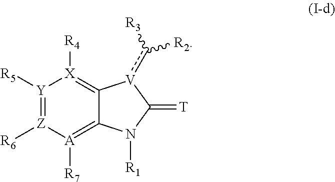

Item6. The compound of Item 5, T is O and W is N,

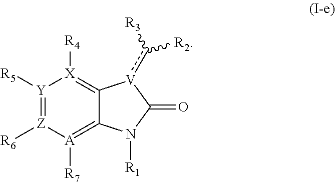

Item7. The compound of Item 4, T is O and V is carbon,

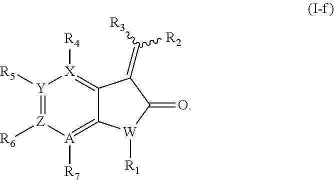

Item8. The compound of Item 1, U is carbon, V is carbon, W is N, and T is O,

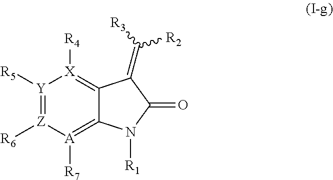

Item9. The compound of any one of Item 1 to Item 8, each of X, Y, Z, and A is carbon.

Item10. The compound of any one of Item 1 to Item 9, R₁ is hydrogen.

Item11. The compound of any one of Item 1 to Item 10, R₂ is

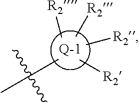

wherein
Q-1 is heterocycle or aryl;
R_{2′}, R_{2″}, R_{2′″}, and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ.

Item12. The compound of Item 10 or Item11, one of X, Y, Z, and A is a heteroatom.

Item13. The compound of any one of Items 10-12, Q-1 is heteroaryl.

Item13′. The compound of any one of Items 10-12, Q-1 is phenyl.

Item14. The compound of any one of Item13, Q-1 is selected from the group consisting of pyrrole, furan, thiophene, pyridine, pyrimidine, pyrazine, pyridazine, imidazole, indole, pyrrolopyridinone, pyridone, pyrrolidine, piridinone, piperidine, and pyrroloazepinone.

Item15. The compound of Item14, Q-1 is selected from the group consisting of pyrrole, furan, thiophene, pyridine, pyrimidine, pyrazine, pyridazine, imidazole, indole, pyrrolopyridinone.

Item16. The compound of Item15, Q-1 is pyrrole.

Item17. The compound of Item 13, Q-1 is pyridone, pyrrolidine, pyridinone, or piperidine.

Item18. The compound of Item 17, Q-1 is pyridone or pyridinone.

Item19. The compound of any one of item11 to Item18, R_{2′}, R_{2″}, R_{2′″}, and R_{2″″} are independently absent, hydrogen, alkyl, substituted alkyl, substituted heterocycle, substituted aryl, C(═O)ORₑ, or C(═O)NR_{b}R_{c},

wherein
R_{b} and R_{c} are independently hydrogen, alkyl, substituted alkyl, substituted heterocycle, or said R_{b} and R_{c} together with the N to which they are bonded optionally form a heterocycle or substituted heterocycle, and
Rₑ is hydrogen.

Item20. The compound of Item19, one of R_{2′}, R_{2″}, R_{2′″}, and R_{2″″} is C(═C)NR_{b}R_{c},

wherein
R_{b} is hydrogen, and
R_{c} is alkyl substituted with NR_{bn}R_{cn} (wherein R_{bn} and R_{cn} are alkyl, or said R_{bn} and R_{cn} together with the N to which they are bonded optionally form a substituted heterocycle (wherein said heterocycle is piperidine, or morpholine)), or R_{b} and R_{c} together with the N to which they are bonded optionally form a substituted heterocycle (wherein said heterocycle is piperidine, or morpholine), and
two of R_{2′}, R_{2″}, R_{2′″}, and R_{2″″} are independently alkyl, and the other is hydrogen.

Item21. The compound of Item20, one of R_{2′}, R_{2″}, R_{2′″}, and R_{2″″} is C(═O)NR_{b}R_{c},

wherein
NR_{b}R_{c} is 2-(di-ethyl amino)ethyl, amino, 2-pyrrolidino ethyl amino, 4-methyl piperazinyl, or morpholino.

Item21′. The compound of Item16, Q-1 is pyrrole, one of R_{2′}, R_{2″}, R_{2′″}, and R_{2″″} is absent, two of R_{2′}, R_{2″}, R_{2′″}, and R_{2″″} are alkyl (e.g., methyl), and one of R_{2′}, R_{2″}, R_{2′″}, and R_{2″″} is C(═O)NR_{b}R_{c}.

Item21″. The compound of Item21′, wherein

R_{b} is hydrogen, and
R_{c} is alkyl substituted with NR_{bn}R_{cn} (wherein R_{bn} and R_{cn} are alkyl, or said R_{bn} and R_{cn} together with the N to which they are bonded optionally form a substituted heterocycle (wherein said heterocycle is piperidine, or morpholine)).

Item21′″. The compound of Item21″, wherein NR_{b}R_{c} is 2-(di-ethyl amino)ethyl, amino, or 2-pyrrolidino ethyl amino.

Item21″″. The compound of Item21′, wherein R_{b} and R_{c} together with the N to which they are bonded optionally form a heterocycle or substituted heterocycle.

Item21′″″. The compound of Item21″″, wherein NR_{b}R_{c} is 4-methyl piperazinyl, or morpholino.

Item22. The compound of any one of Item1 to Item21, R₄, R₅, R₆, and R₇ are each independently hydrogen, halogen, cyano, nitro, alkyl or substituted alkyl, ORₐ, NR_{b}R_{c}, C(═O)ORₑ, C(═O)Rₐ, C(═O)NR_{b}R_{c}, or

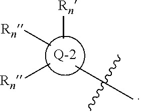

Item23. The compound of any one of Item1 to Item22, R₄, R₅, R₆, and R₇ are each independently hydrogen, halogen, cyano, nitro, alkyl, ORₐ, NR_{b}R_{c}, C(═O)ORₑ, C(═O)Rₐ, C(═O)NR_{b}R_{c} (wherein

Rₐ is hydrogen, or alkyl or substituted alkyl,
R_{b} and R_{c} are independently hydrogen, or alkyl or substituted alkyl, and
Rₑ is alkyl or substituted alkyl (substituted alkyl is optionally substituted with one or more substituent(s) selected from the group consisting of hydroxy, amino, nitro, cyano, halogen, alkoxy, alkylcarbonyl, alkoxycarbonyl, aminocarbonyl, aryl, cycloalkyl, and heterocycle)), and

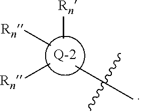

Item24. The compound of any one of Item23, one of R₄, R₅, R₆, and R₇ is

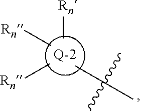

the others of R₄, R₅, R₆, and R₇ are each independently hydrogen.

Item25. The compound of Item24, Q-2 is selected from the group consisting of pyrrole, furan, thiophene, imidazole, pyrazole, oxazole, isoxazole, thiazole, isothiazole, triazole, thiadiazole, oxadiazole, pyrrolidine, piperidine, azepane, tetrahydrofuran, oxane, oxepane, indole, indolinone, indazole, benzothiazole, quinoline, quinazoline, quinoxaline, imidazopyridine, imidazopyridazine, pyrazolopyridine, pyrazolopyrimidine, phthalazinone, and phenyl.

Item26. The compound of Item25, Q-2 is selected from the group consisting of pyrrole, furan, thiophene, imidazole, pyrazole, oxazole, isoxazole, thiazole, isothiazole, triazole, thiadiazole, oxadiazole, pyrrolidine, piperidine, azepane, tetrahydrofuran, oxane, oxepane, indole, indolinone, indazole, benzothiazole, quinoline, quinazoline, quinoxaline, imidazopyridine, imidazopyridazine, pyrazolopyridine, pyrazolopyrimidine, and phthalazinone.

Item27. The compound of Item26, Q-2 is selected from the group consisting of thiophene, imidazole, oxazole, thiazole, thiadiazole, piperidine, and pyrazole.

Item27′. The compound of Item26, Q-2 is selected from the group consisting of indole, indolinone, indazole, benzothiazole, quinoline, quinazoline, quinoxaline, imidazopyridine, imidazopyridazine, pyrazolopyridine, pyrazolopyrimidine, and phthalazinone.

Item28. The compound of Item27, Q-2 is thiazole.

Item29. The compound of Item27, Q-2 is imidazole.

Item30. The compound of Item27, Q-2 is piperidine.

Item31. The compound of Item27, Q-2 is pyrazole.

Item32. The compound of any one of Item22 to 25, R_{n′} is pyrrolidinyl, piperidinyl, azepanyl, tetrahydrofuranyl, oxanyl, oxepanyl, pyranyl, phenyl, thiophenyl, pyrazinyl, pyrimidinyl, pyridazinyl, or pyridyl (said piperidinyl, pyranyl, phenyl, thiophenyl, pyrazinyl, pyrimidinyl, pyridazinyl, and pyridyl are optionally substituted with halogen, cyano, nitro, alkyl or substituted alkyl, ORₐ, NR_{b}R_{c}, C(═O)ORₑ, C(═O)Rₐ, or C(═O)NR_{b}R_{c} (wherein Rₐ is hydrogen, or alkyl or substituted alkyl, R_{b} and R_{c} are independently hydrogen, or alkyl or substituted alkyl, and Rₑ is alkyl or substituted alkyl (substituted alkyl is optionally substituted with one or more substituent(s) selected from the group consisting of hydroxy, amino, nitro, cyano, halogen, alkoxy, alkylcarbonyl, alkoxycarbonyl, aminocarbonyl, aryl, cycloalkyl, and heterocycle)), and R_{n″} and R_{n′″} are independently hydrogen, or alkyl or substituted alkyl (substituted alkyl is optionally substituted with one or more substituent(s) selected from the group consisting of hydroxy, amino, nitro, cyano, halogen, alkoxy, alkylcarbonyl, alkoxycarbonyl, aminocarbonyl, aryl, cycloalkyl, and heterocycle).

Item32′. The compound of any one of Item22 to 25, R_{n′}, R_{n″} and R_{n′″} are independently hydrogen, alkyl, or methoxy.

Item32″. The compound of any one of Item22 to 25, R_{n′}, R_{n″} and R_{n′″} are each hydrogen.

Item33. The compound of Item32, R_{n′} is pyrrolidinyl, piperidinyl, tetrahydrofuranyl, pyranyl, phenyl, pyrazinyl, pyrimidinyl, or pyridyl (said piperidinyl, pyranyl, phenyl, pyrazinyl, pyrimidinyl, and pyridyl are optionally substituted with halogen, cyano, alkyl or substituted alkyl, ORₐ, or C(═O)ORₑ (wherein Rₐ is hydrogen, or alkyl or substituted alkyl, and Rₑ is alkyl or substituted alkyl (substituted alkyl is optionally substituted with one or more substituent(s) selected from the group consisting of hydroxy, amino, nitro, cyano, halogen, alkoxy, alkylcarbonyl, alkoxycarbonyl, aminocarbonyl, aryl, cycloalkyl, and heterocycle)), and

R_{n″} and R_{n′″} are independently hydrogen, alkyl, or amino.

Item 33′. The compound of Item 33, R_{n′} is phenyl or substituted phenyl, and R_{n″} and R_{n′″} are independently hydrogen, or alkyl, or amino.

Item34. The compound of Item33, R_{n″} and R_{n′″} are independently hydrogen or alkyl.

Item35. The compound of Item 32 or 33, Q-2 is selected from the group consisting of the following group:

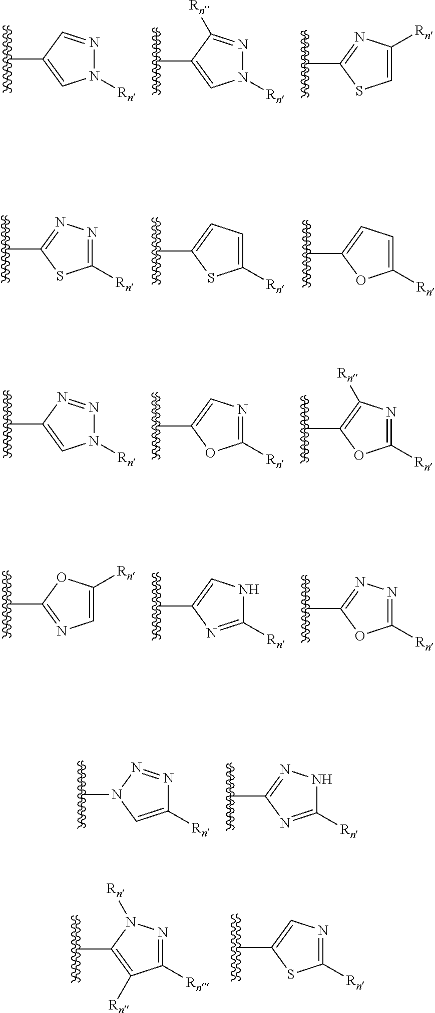

Item36. The compound of Item 32 or 33, Q-2 is selected from the group consisting of the following group:

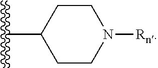

Item37. The compound of any one of Item1, selected from the group consisting of:

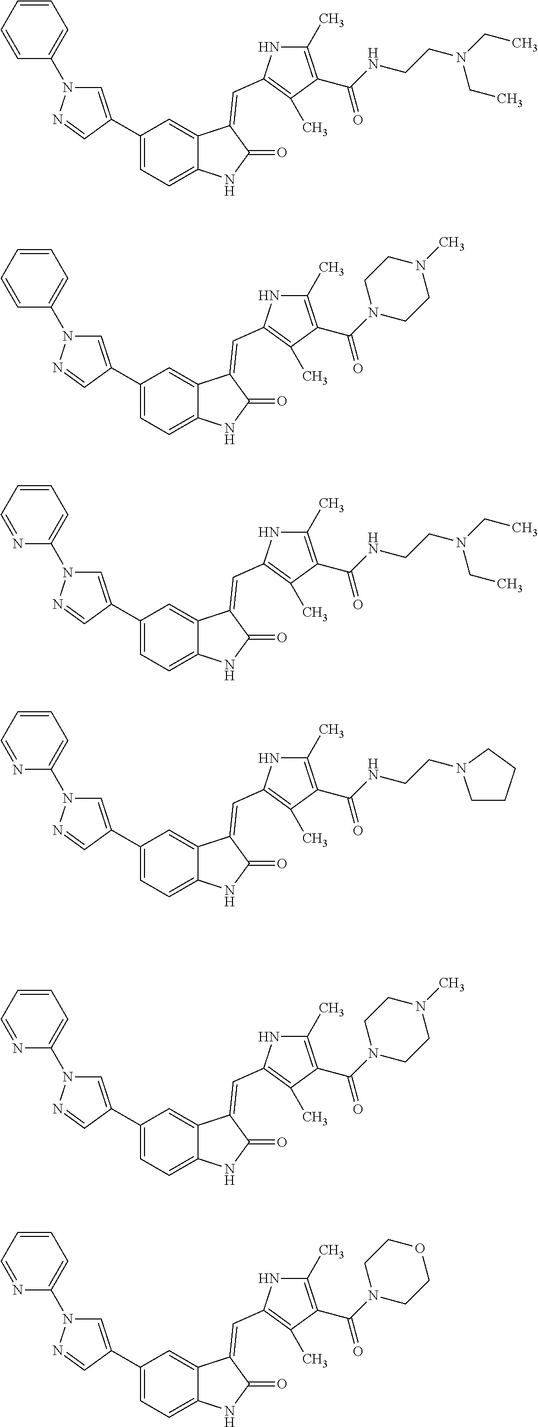 [SMILES: CCN(CC)CCNC(=O)c1c(C)[nH]c(/C=C2\C(=O)Nc3ccc(-c4cnn(-c5ccccc5)c4)cc32)c1C ; Cc1[nH]c(/C=C2\C(=O)Nc3ccc(-c4cnn(-c5ccccc5)c4)cc32)c(C)c1C(=O)N1CCN(C)CC1 ; CCN(CC)CCNC(=O)c1c(C)[nH]c(/C=C2\C(=O)Nc3ccc(-c4cnn(-c5ccccn5)c4)cc32)c1C ; Cc1[nH]c(/C=C2\C(=O)Nc3ccc(-c4cnn(-c5ccccn5)c4)cc32)c(C)c1C(=O)NCCN1CCCC1 ; Cc1[nH]c(/C=C2\C(=O)Nc3ccc(-c4cnn(-c5ccccn5)c4)cc32)c(C)c1C(=O)N1CCN(C)CC1 ; Cc1[nH]c(/C=C2\C(=O)Nc3ccc(-c4cnn(-c5ccccn5)c4)cc32)c(C)c1C(=O)N1CCOCC1]

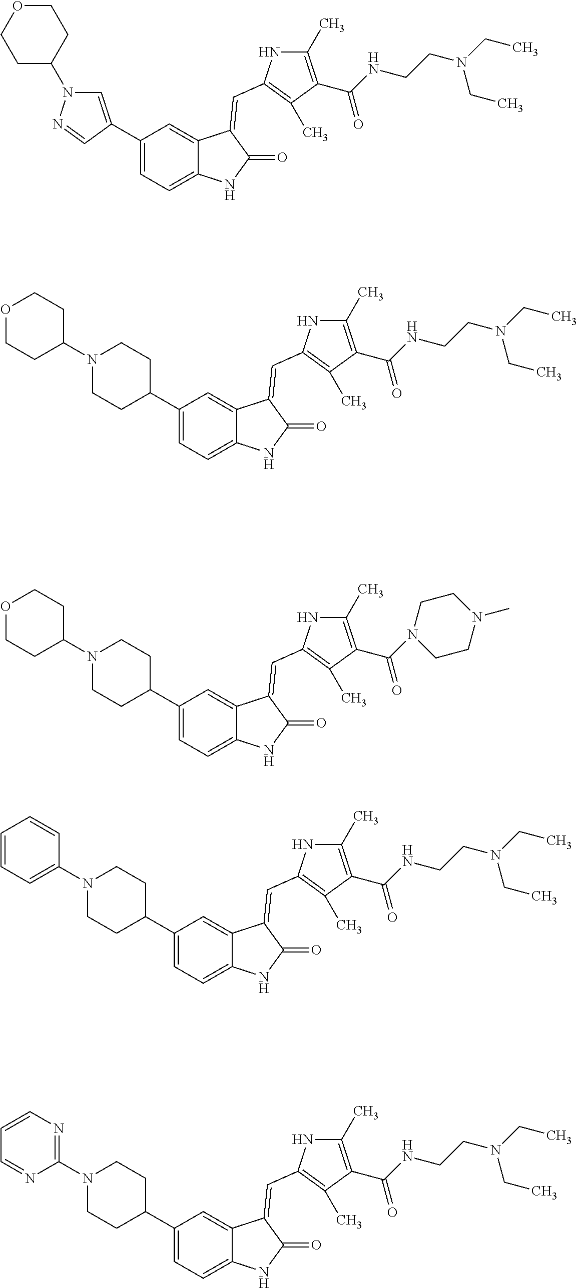 [SMILES: CCN(CC)CCNC(=O)c1c(C)[nH]c(/C=C2\C(=O)Nc3ccc(-c4cnn(C5CCOCC5)c4)cc32)c1C ; CCN(CC)CCNC(=O)c1c(C)[nH]c(/C=C2\C(=O)Nc3ccc(C4CCN(C5CCOCC5)CC4)cc32)c1C ; Cc1[nH]c(/C=C2\C(=O)Nc3ccc(C4CCN(C5CCOCC5)CC4)cc32)c(C)c1C(=O)N1CCN(C)CC1 ; CCN(CC)CCNC(=O)c1c(C)[nH]c(/C=C2\C(=O)Nc3ccc(C4CCN(c5ccccc5)CC4)cc32)c1C ; CCN(CC)CCNC(=O)c1c(C)[nH]c(/C=C2\C(=O)Nc3ccc(C4CCN(c5ncccn5)CC4)cc32)c1C]

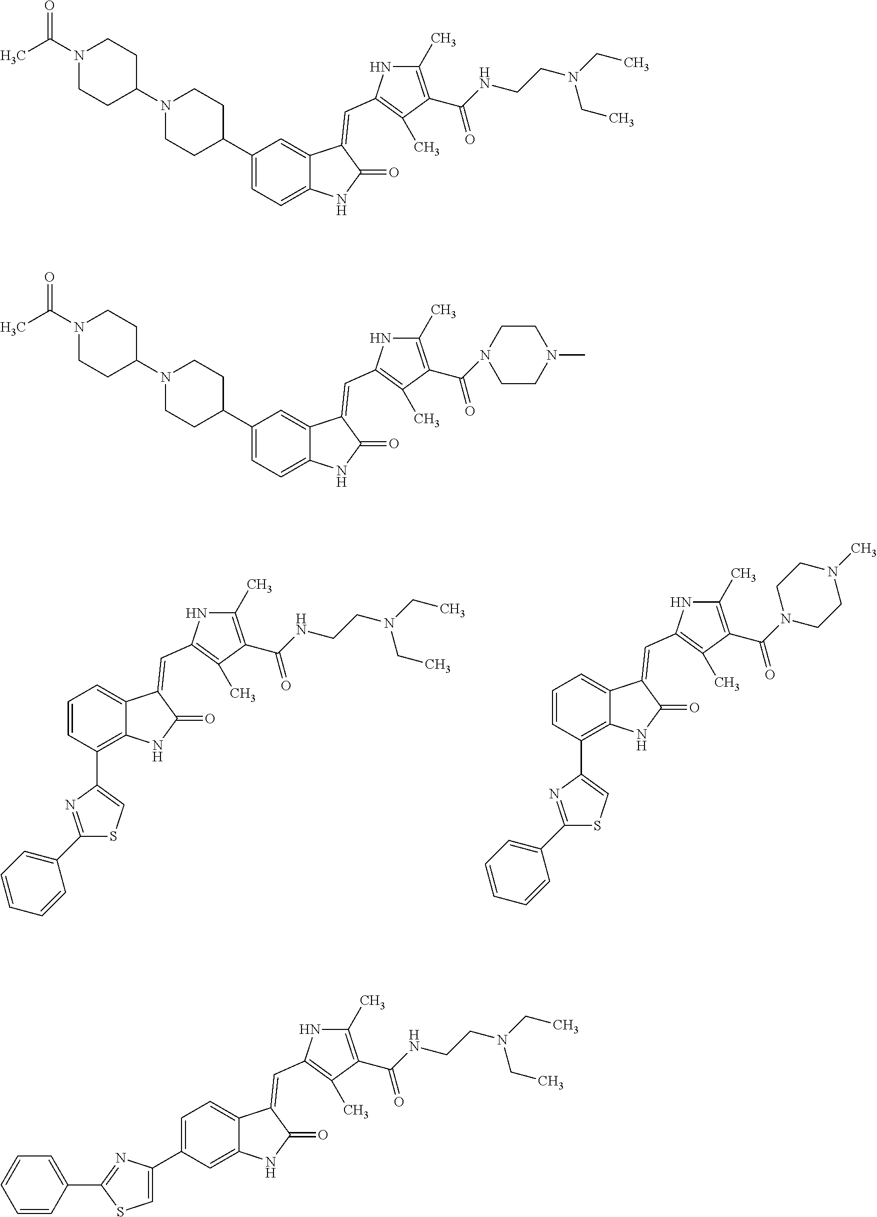

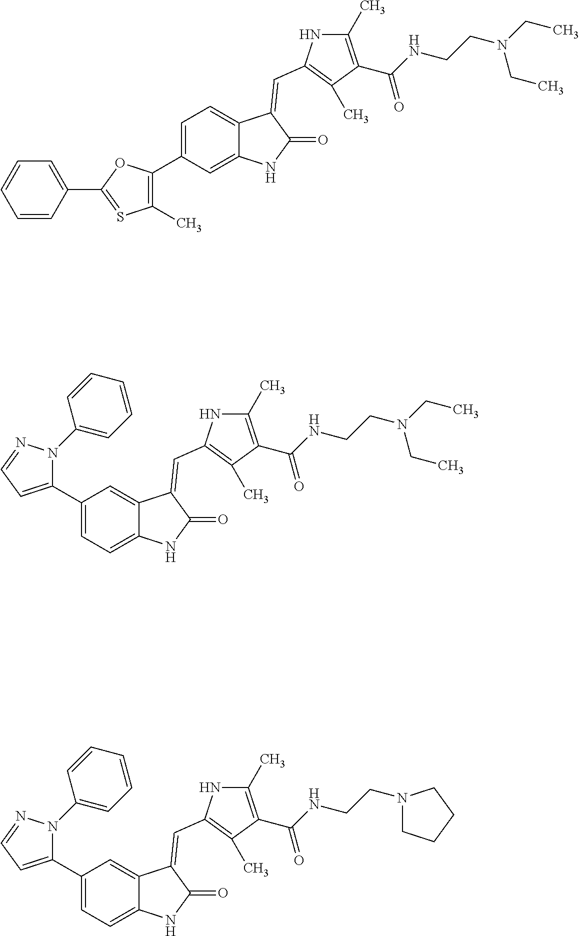

Item38. A compound of Formula II:

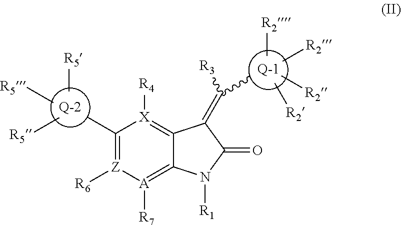

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, S(O)₂NRₐR_{b};
R₄, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Z, and A are each independently a carbon or N, with the proviso that the ring in which X, Z, and A exist is aromatic;
Q-1 and Q-2 is independently is heterocycle, or aryl;
R_{2′}, R_{2″}, R_{2′″}, and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{5′}, R_{5″} and R_{5′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

Item39. The compound of Item38, wherein each of X, Z, and A is carbon.

Item40. The compound of Item38, wherein one of X, Z, and A is a heteroatom.

Item41. The compound of Item 38, the compound has the formula

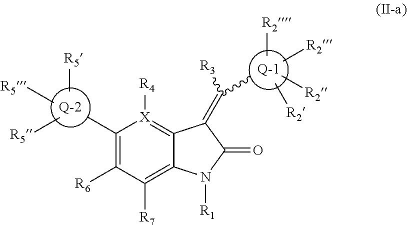

wherein R₁, R_{2′}, R_{2″}, R_{2′″}, R_{2″″}, R₃, R₄, R_{5′}, R_{5″}, R_{5′″}, R₆, R₇, X, Q-1, and Q-2 are the same as the above definitions.

Item42. The compound of Item 38, the compound has the formula,

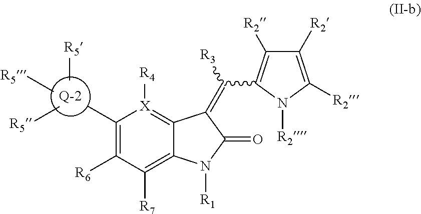

wherein

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R_{5′}, R_{5″}, R_{5′″}, R₆, R₇, X, and Q-2 are the same as the above definitions.

Item43. The compound of Item 42, wherein X is C.

Item44. The compound of Item 42, wherein X is N.

Item45. The compound of Item 42 to 44, wherein R_{2″″} is H.

Item46. The compound of Item 42 to 45, wherein each of R_{2″} and R_{2′″} is H.

Item46′. The compound of Item 42 to 45, wherein each of R_{2″} and R_{2′″} is methyl.

Item47. The compound of the Item 38, the compound has the formula

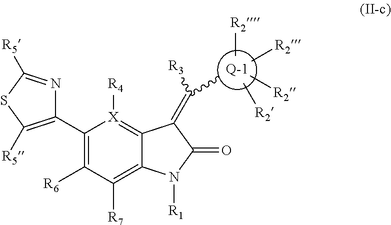

wherein

R₁, R_{2′}, R_{2″}, R_{2′″}, R_{2′″}, R₃, R₄, R_{5′}, R_{5″}, R₆, R₇, X, and Q-1 are the same as the above definitions.

Item48. The compound of Item 38, the compound has the formula of

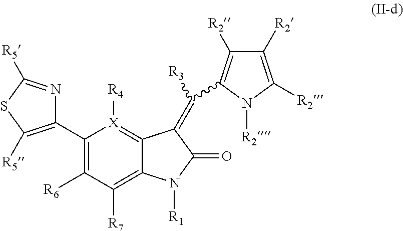

wherein

X is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R₃, R₄, R_{5′}, R_{5″}, R₆, and R₇ are the same as the above definitions.

Item49. The compound of Item 38, the compound of formula (II-e),

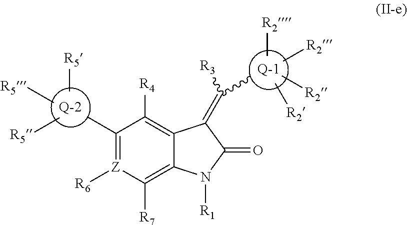

wherein Z is C or N,

R₁, R_{2′}, R_{2″}, R_{2′″}, R₂, R₃, R₄, R_{5′}, R_{5″}, R_{5′″}, R₆, R₇, Z, Q-1, and Q-2 are the same as the above definitions.

Item50. The compound of Item 38, the compound has of the formula of

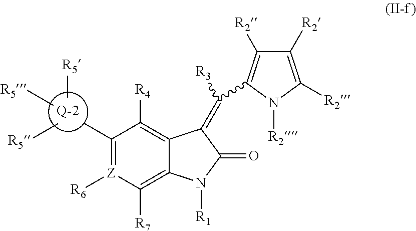

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R_{2′}, R_{2″}, R_{2′″}, R_{2′″}, R₃, R₄, R_{5′}, R_{5″}, R_{5′″}, R₆, R₇, and Q-2 are the same as the above definitions.

Item51. The compound of Item 50, wherein Z is C.

Item52. The compound of Item 51, wherein Z is N.

Item53. The compound of Item 52, wherein R_{2″″} is H.

Item54. The compound of Item 50 to 53, wherein each of R_{2″} and R_{2′″} is H.

Item54′. The compound of Item 50 to 53, wherein each of R_{2″} and R_{2′″} is methyl.

Item55. The compound of Item 38, the compound formula

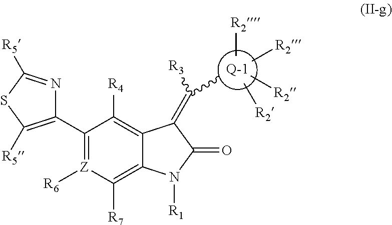

wherein Z is C or N,

R₁, R_{2′}, R_{2″}, R_{2′″}, R_{2′″}, R₃, R₄, R_{5′}, R_{5″}, R₆, R₇, and Q-1 are the same as the above definitions.

Item56. The compound of Item 38, the compound has the formula of

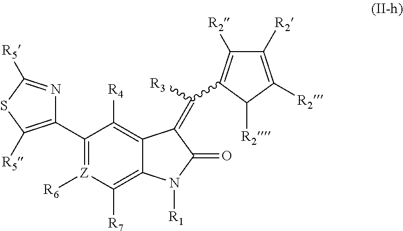

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R_{5′}, R_{5″}, R₆, and R₇ are the same as the above definitions.

Item57. The compound of Item 38, the compound has the formula of

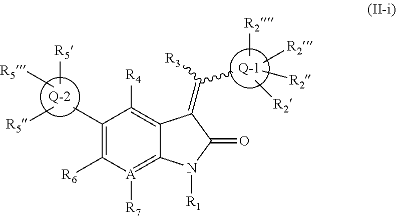

wherein A is C or N,

R₁, R_{2′}, R_{2″}, R_{2′″}, R_{2′″}, R₃, R₄, R_{5′}, R_{5″}, R_{5′″}, R₆, R₇, Q-1 and Q-2 are the same as the above definitions.

Item58. The compound of Item 38, the compound has the formula of

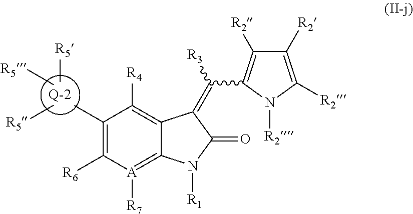

wherein

A is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R_{5′}, R_{5″}, R_{5′″}, R₆, R₇, and Q-2 are the same as the above definitions.

Item59. The compound of Item 58, whrein A is C.

Item60. The compound of Item 58, wherein, A is N.

Item61. The compound of Item 58 to 60, wherein, R_{2″″} is H. In certain embodiments, each of R_{2″} and R_{2′″} is H. In other embodiments, each of R_{2″} and R_{2′″} is methyl.

Item62. The compound of Item 38, the compound has the formula of

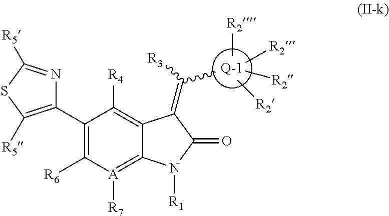

wherein A is C or N,

R₁, R_{2′}, R_{2″}, R_{2′″}, R_{2′″}, R₃, R₄, R_{5′}, R_{5″}, R₆, R₇, and Q-1 are the same as the above definitions.

Item63. The compound of Item 38, the compound has the formula of

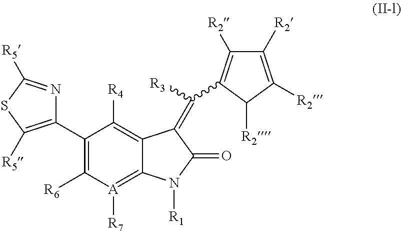

wherein

A is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R₃, R₄, R_{5′}, R_{5″}, R₆, and R₇ are the same as the above definitions.

Item64. The compound of Item 38, the compound has the formula of

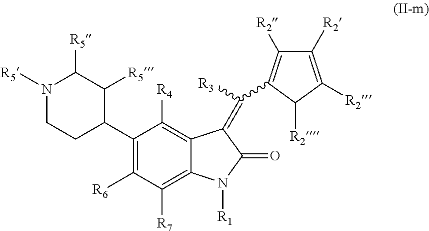

wherein

R₁, R_{2′}, R_{2″}, R_{2′″}, R_{2′″}, R₃, R₄, R_{5′}, R_{5″}, R_{5′″}, R₆, and R₇ are the same as the above definitions.

Item65. The compound of Item 38, the compound has the formula of

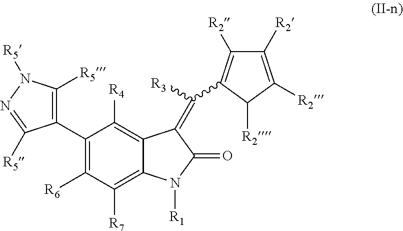

wherein

R₁, R_{2′}, R_{2″}, R_{2′″}, R_{2′″}, R₃, R₄, R_{5′}, R_{5″}, R_{5′″}, R₆, and R₇ are the same as the above definitions.

Item66. The compound of Item 38, the compound has the formula of

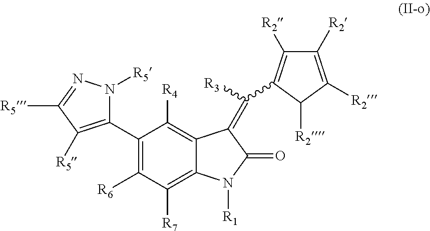

wherein

R₁, R_{2′}, R_{2″}, R_{2′″}, R_{2″″}, R₃, R₄, R_{5′}, R_{5″}, R_{5′″}, R₆, and R₇ are the same as the above definitions.

Item66′. The compound of Item38, wherein each of R₁, R_{2′}, R_{2″}, R_{2′″}, R_{2″″}, R₃, R₄, R_{5′}, R_{5″}, R_{5′″}, R₆, R₇, X, Z, A, Q-1, and Q-2 can be selected from any of the groups illustrated hereinabove.

Item67. A compound of Formula III,

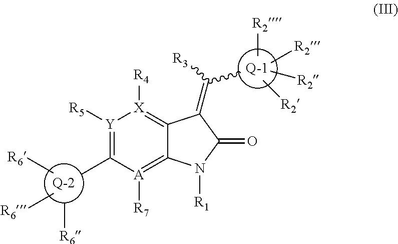

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Y, and A are each independently a carbon or N, with the proviso that the ring in which X, Y, and A exist is aromatic;
Q-1 and Q-2 are each independently is heterocycle or aryl;
R_{2′}, R_{2″}, R_{2′″} and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{6′}, R_{6″} and R_{6′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

Item68. The compound of Item 67, wherein each of X, Y, and A is carbon.

Item69. The compound of Item 67, wherein one of X, Y, and A is a heteroatom.

Item70. The compound of Item 67, the compound has the formula of

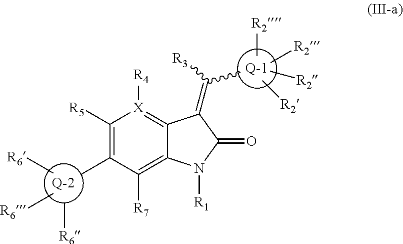

wherein X is C or N,

R₁, R_{2′}, R_{2″}, R_{2′″}, R_{2″″}, R₃, R₄, R₅, R_{6′}, R_{6″}, R_{6′″}, R₇, Q-1, and Q-2 are the same as the above definitions.

Item71. The compound of Item 67, the compound has the formula of

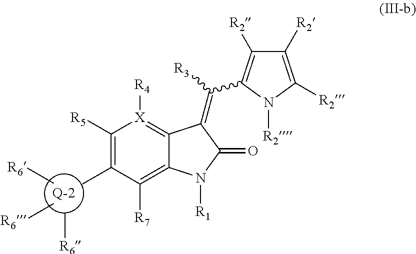

wherein

X is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R₅, R_{6′}, R_{6″}, R_{6′″}, R₇, and Q-2 are the same as the above definitions.

Item72. The compound of Item 71, wherein X is C.

Item73. The compound of Item 71, wherein X is N.

Item74. The compound of Item 71 to 73, wherein R_{2″″} is H.

Item75. The compound of Item 71 to 74, wherein each of R_{2″} and R_{2′″} is H.

Item75′. The compound of Item 71 to 74, wherein each of R_{2″} and R_{2′″} is methyl.

Item76. The compound of Item 67, the compound has the formula of

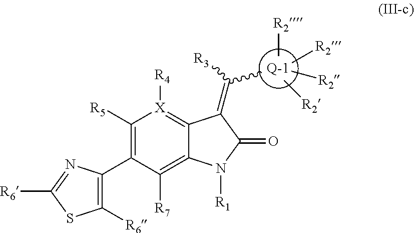

wherein X is C or N,

R₁, R_{2′}, R_{2″}, R_{2′″}, R_{2″″}, R₃, R₄, R₅, R_{6′}, R_{6″}, R₇, and Q-1 are the same as the above definitions.

Item77. The compound of Item 67, the compound has the formula of

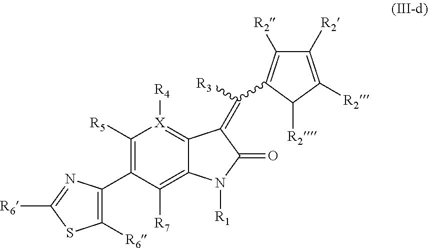

wherein

X is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R₃, R₄, R₅, R_{6′}, R_{6″}, and R₇ are the same as the above definitions.

Item78. The compound of Item 67, the compound has the formula of

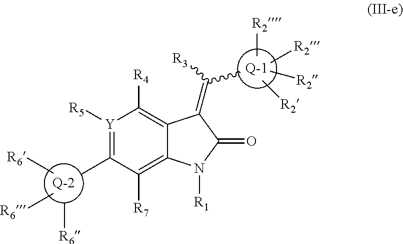

wherein Y is C or N,

R₁, R_{2′}, R_{2″}, R_{2′″}, R_{2″″}, R₃, R₄, R₅, R_{6′}, R_{6″}, R_{6′″}, R₇, Q-1, and Q-2 are the same as the above definitions.

Item79. The compound of Item 67, the compound has the formula of

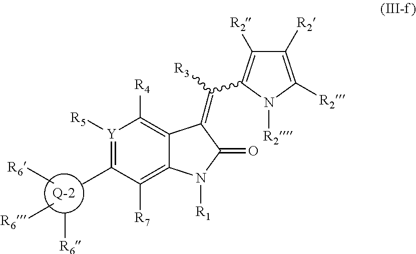

wherein

Y is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R₃, R₄, R₅, R_{6′}, R_{6″}, R_{6′″}, R₇, and Q-2 are the same as the above definitions.

Item80. The compound of Item 79, wherein Y is C.

Item81. The compound of Item 79, wherein Y is N.

Item82. The compound of Item 79 to 81, wherein R_{2″″} is H.

Item83. The compound of Item 79 to 82, wherein each of R_{2″} and R_{2′″} is H.

Item84. The compound of Item 79 to 82, wherein each of R_{2″} and R_{2′″} is methyl.

Item85. The compound of Item 67, the compound has the formula of

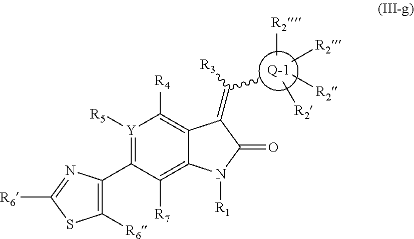

wherein Y is C or N,

R₁, R_{2′}, R_{2″}, R_{2′″}, R_{2″″}, R₃, R₄, R₅, R_{6′}, R_{6″}, R₇, and Q-1 are the same as the above definitions.

Item86. The compound of Item 67, the compound has the formula of

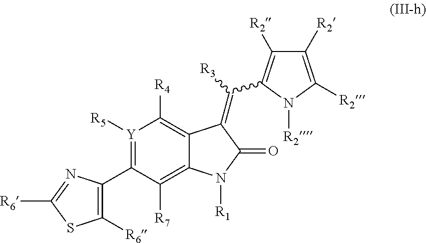

wherein

Y is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R₃, R₄, R₅, R_{6′}, R_{6″}, and R₇ are the same as the above definitions.

Item87. The compound of Item 67, the compound has the formula of

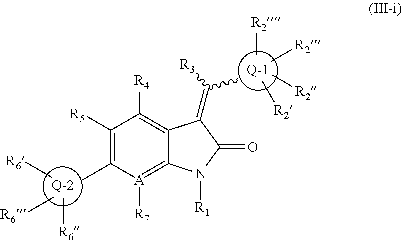

wherein A is C or N,

R₁, R_{2′}, R_{2″}, R_{2′″}, R_{2″″}, R₃, R₄, R₅, R_{6′}, R_{6″}, R_{6′″}, R₇, Q-1, and Q-2 are the same as the above definitions.

Item88. The compound of Item 67, the compound has the formula of

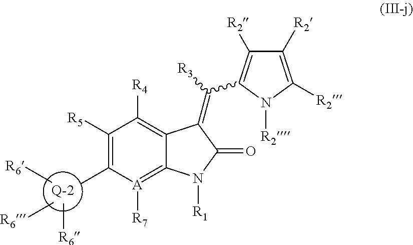

wherein

A is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c}, and

R₁, R₃, R₄, R₅, R_{6′}, R_{6″}, R_{6′″}, R₇, and Q-2 are the same as the above definitions.

Item89. The compound of Item 88, wherein A is C.

Item90. The compound of Item 88, wherein A is N.

Item91. The compound of Item 88 to 90, wherein R_{2″″} is H.

Item92. The compound of item 88 to 91, wherein each of R_{2″} and R_{2′″} is H.

Item93. The compound of item 88 to 91, wherein each of R_{2″} and R_{2′″} is methyl.

Item94. The compound of Item 67, the compound has the formula of

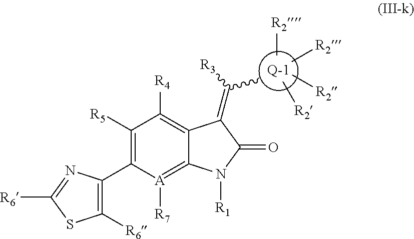

wherein A is C or N,

R₁, R_{2′}, R_{2″}, R_{2′″}, R_{2′″}, R₃, R₄, R₅, R_{6′}, R_{6″}, R₇, and Q-1 are the same as the above definitions.

Item95. The compound of Item 67, the compound has the formula of

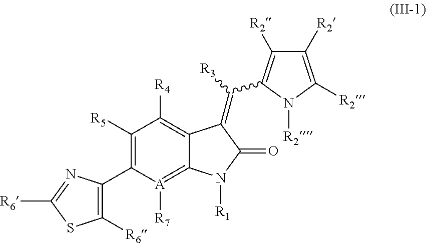

wherein

A is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R₅, R_{6′}, R_{6″}, and R₇ are the same as the above definitions.

Item95′. The compound of Item67, wherein each of R₁, R_{2′}, R_{2″}, R_{2′″}, R_{2″″}, R₃, R₄, R₅, R_{6′}, R_{6″}, R_{6′″}, R₇, X, Y, A, Q-1, and Q-2 can be selected from any of the groups illustrated hereinabove.

Item96. A compound of Formula IV,

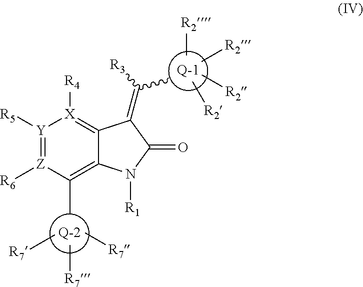

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, and R₆ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Y, and Z are each independently a carbon or N, with the proviso that the ring in which X, Y, and Z exist is aromatic;
Q-1 and Q-2 are each independently is heterocycle or aryl;
R_{2′}, R_{2″}, R_{2′″} and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{7′}, R_{7″} and R_{7′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

Item97. The compound of Item 96, wherein each of X, Y, and Z is carbon.

Item98. The compound of Item 96, wherein one of X, Y, and Z is a heteroatom.

Item99. The compound of Item 96, the compound has the formula of

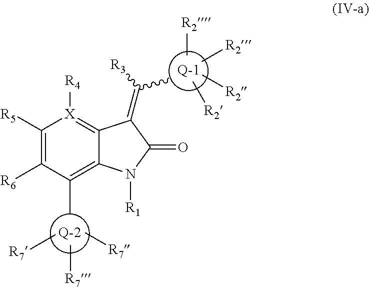

wherein X is C or N,

R₁, R_{2′}, R_{2″}, R_{2′″}, R_{2″″}, R₃, R₄, R₅, R₆, R_{7′}, R_{7″}, R_{7′″}, R_{7″″}, Q-1, and Q-2 are the same as the above definitions.

Item100. The compound of Item 96, the compound has the formula:

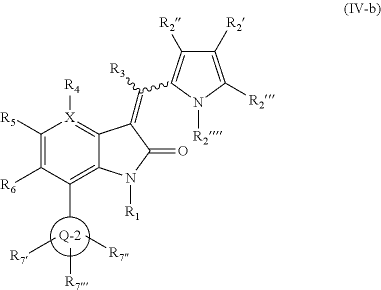

wherein

X is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═C)₂ORₑ, C(═O)OR_{d}, C(═O)Rₐ, or C(═O)NR_{b}R_{c},

R₁, R₃, R₄, R₅, R₆, R_{7′}, R_{7″}, R_{7′″}, R_{7″″}, and Q-2 are the same as the above definitions.

Item101. The compound of Item 100, wherein X is C.

Item102. The compound of Item 100, wherein X is N.

Item103. The compound of Item 100 to 102, wherein R_{2″″} is H.

Item104. The compound of Item 100 to 103, each of R_{2″} and R_{2′″} is H.

Item105. The compound of Item 100 to 103, each of R_{2″} and R_{2′″} is methyl.

Item106. The compound of Item 100, the compound has the formula of

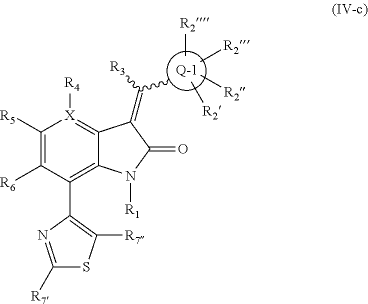

wherein X is C or N,

R₁, R_{2′}, R_{2″}, R_{2′″}, R_{2″″}, R₃, R₄, R₅, R₆, R_{7′}, R_{7″}, and Q-1 are the same as the above definitions.

Item107. The compound of Item 96, the compound has the formula of

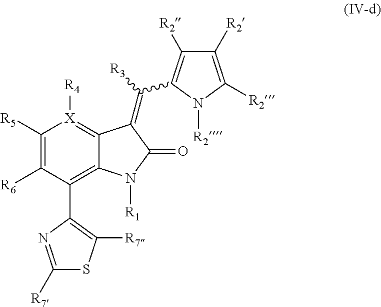

wherein

X is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R₅, R₆, R_{7′}, and R_{7″} are the same as the above definitions.

Item108. The compound of Item 96, the compound has the formula of

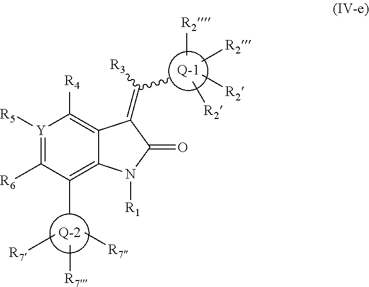

wherein Y is C or N,

R₁, R_{2′}, R_{2″}, R_{2′″}, R_{2″″}, R₃, R₄, R₅, R₆, R_{7′}, R_{7″}, R_{7′″}, Q-1, and Q-2 are the same as the above definitions.

Item109. The compound of Item 96, the compound has the formula of

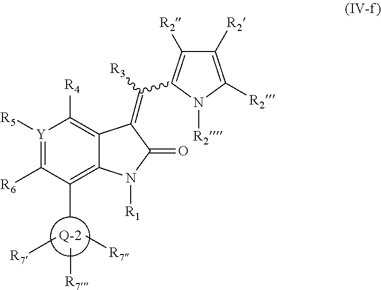

wherein

Y is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═C)₂ORₑ, C(═O)OR_{d}, C(═O)Rₐ, or C(═O)NR_{b}R_{c},

R₁, R₃, R₄, R₅, R₆, R_{7′}, R_{7″}, R_{7′″}, and Q-2 are the same as the above definitions.

Item110. The compound of Item 109, wherein Y is C.

Item111. The compound of Item 109, wherein Y is N.

Item112. The compound of Item 109 to 111, R_{2″″} is H.

Item113. The compound of Item 109 to 112, each of R_{2″} and R_{2′″} is H.

Item114. The compound of Item 109 to 112, each of R_{2″} and R_{2′″} is methyl.

Item115. The compound of Item 96, the compound has the formula of

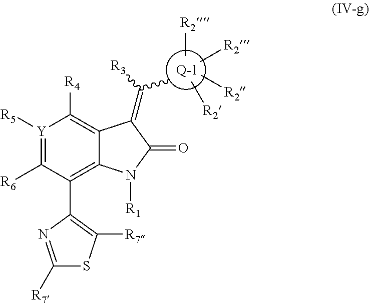

wherein Y is C or N,

R₁, R_{2′}, R_{2″}, R_{2′″}, R_{2″″}, R₃, R₄, R₅, R₆, R_{7′}, R_{7″}, and Q-1 are the same as the above definitions.

Item116. The compound of Item 96, the compound has the formula of

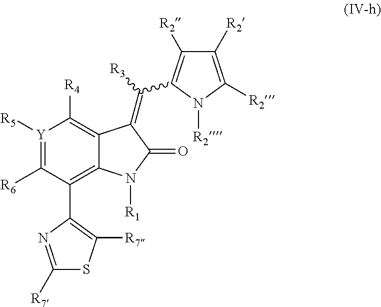

wherein

Y is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R₅, R₆, R_{7′}, and R_{7″} are the same as the above definitions.

Item117. The compound of Item 96, the compound has the formula of

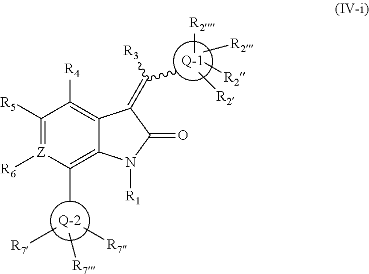

wherein Z is C or N,

R₁, R_{2′}, R_{2″}, R_{2′″}, R_{2″″}, R₃, R₄, R₅, R₆, R_{7′}, R_{7″}, R_{7′″}, Q-1, and Q-2 are the same as the above definitions.

Item118. The compound of Item 96. The compound has the formula:

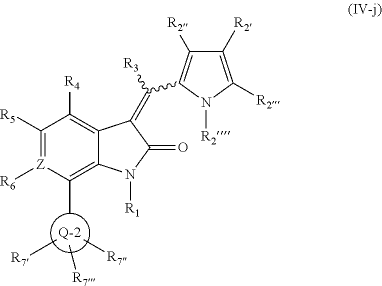

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═C)₂ORₑ, C(═O)OR_{d}, C(═O)Rₐ, or C(═O)NR_{b}R_{c},

R₁, R₃, R₄, R₅, R₆, R_{7′}, R_{7″}, R_{7′″}, and Q-2 are the same as the above definitions.

Item119. The compound of Item 118, wherein Z is C.

Item120. The compound of Item 118, wherein Z is N.

Item121. The compound of Item 118 to 120, wherein R_{2″″} is H.

Item122. The compound of Item 118 to 121, wherein each of R_{2″} and R_{2′″} is H.

Item123. The compound of Item 118 to 121, wherein each of R_{2″} and R_{2′″} is methyl.

Item124. The compound of Item 96, the compound has the formula:

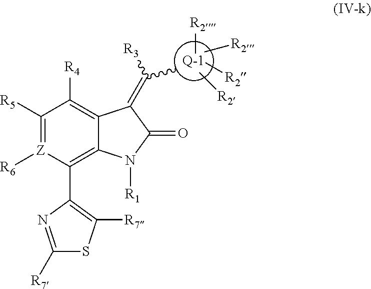

wherein Z is C or N,

R₁, R_{2′}, R_{2″}, R_{2′″}, R_{2″″}, R₃, R₄, R₅, R₆, R_{7′}, R_{7″}, and Q-1 are the same as the above definitions.

Item125. The compound of Item 96, the compound has the formula:

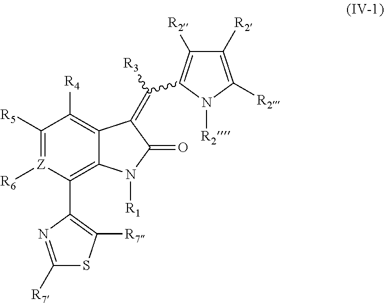

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R₄, R₅, R₆, R_{7′}, and R_{7″} are the same as the above definitions.

Item125′. The compound of Item96, wherein each of R₁, R_{2′}, R_{2″}, R_{2′″}, R_{2″″}, R₃, R₄, R₅, R₆, R_{7′}, R_{7″}, R_{7′″}, X, Y, Z, Q-1, and Q-2 can be selected from any of the groups illustrated hereinabove.

Item126. A compound of Formula V,

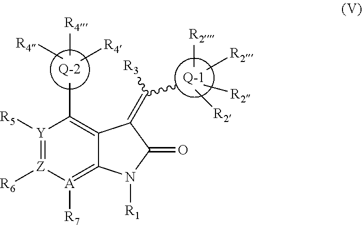

or an enantiomer, diastereomer, tautomer, or pharmaceutically acceptable salt or solvate thereof,
wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₅, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
Y, Z and A are each independently a carbon or N, with the proviso that the ring in which Y, Z and A exist is aromatic;
Q-1 and Q-2 are each independently is heterocycle or aryl;
R_{2′}, R_{2″}, R_{2′″} and R_{2″″} are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R_{4′}, R_{4″} and R_{4′″} are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
   wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

Item127. The compound of Item 126, wherein each of Y, Z and A is carbon.

Item128. The compound of Item 126, wherein one of Y, Z and A is a heteroatom.

Item129. The compound of Item 126, the compound has the formula of

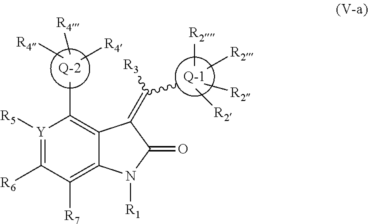

wherein Y is C or N,

R₁, R_{2′}, R_{2″}, R_{2′″}, R_{2″″}, R₃, R_{4′}, R_{4″}, R_{4′″}, R₅, R₆, R₇, Q-1, and Q-2 are the same as the above definitions.

Item130. The compound of Item 126, the compound has the formula of

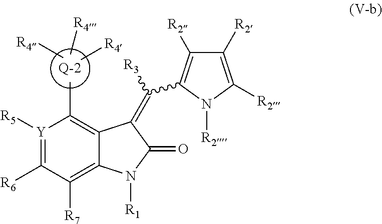

wherein

Y is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2′″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)ORₐ, C(═O)Rₐ, or C(═O)NR_{b}R_{c},

R₁, R₃, R_{4′}, R_{4″}, R_{4′″}, R₅, R₆, R₇, and Q-2 are the same as the above definitions.

Item131. The compound of Item 130, wherein Y is C.

Item132. The compound of Item 130, wherein Y is N.

Item133. The compound of Item 130 to 132, wherein R_{2″″} is H.

Item134. The compound of Item 130 to 133, wherein each of R_{2″} and R_{2′″} is H.

Item135. The compound of Item 130 to 133, wherein each of R_{2″} and R_{2′″} is methyl.

Item136. The compound of Item 126, the compound has the formula of

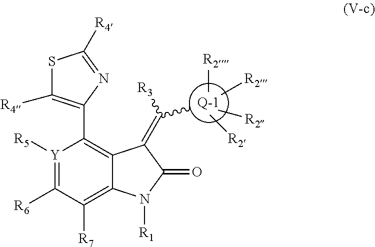

wherein Y is C or N,

R₁, R_{2′}, R_{2″}, R_{2′″}, R_{2″″}, R₃, R_{4′}, R_{4″}, R₅, R₆, R₇, and Q-1 are the same as the above definitions.

Item137. The compound of Item 126, the compound has the formula of

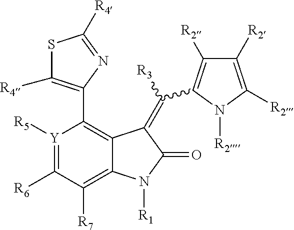

wherein

Y is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R_{4′}, R_{4″}, R₅, R₆, and R₇ are the same as the above definitions.

Item138. The compound of Item 126, the compound has the formula of

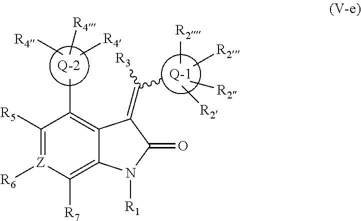

wherein Z is C or N,

R₁, R_{2′}, R_{2″}, R_{2′″}, R_{2″″}, R₃, R_{4′}, R_{4″}, R_{4′″}, R₅, R₆, R₇, Q-1, and Q-2 are the same as the above definitions.

Item139. The compound of Item 126, the compound has the formula of

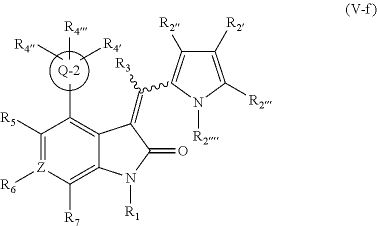

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R_{4′}, R_{4″}, R_{4′″}, R₅, R₆, R₇, and Q-2 are the same as the above definitions.

Item140. The compound of Item 139, wherein Z is C.

Item141. The compound of item 139, wherein Z is N.

Item142. The compound of Item 139 to 141, wherein R_{2′″} is H.

Item143. The compound of Item 139 to 142, wherein each of R_{2″} and R_{2′″} is H.

Item144. The compound of Item 126, the compound has the formula of

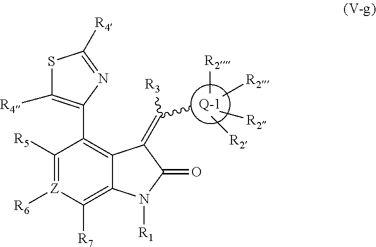

wherein Z is C or N,

R₁, R_{2′}, R_{2″}, R_{2′″}, R_{2″″}, R₃, R_{4′}, R_{4″}, R₅, R₆, R₇, and Q-1 are the same as the above definitions.

Item145. The compound of Item 126, the compound has the formula of

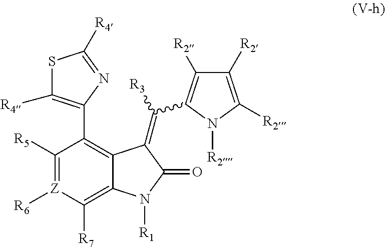

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R_{4′}, R_{4″}, R₅, R₆, and R₇ are the same as the above definitions.

Item146. The compound of Item 126, the compound has the formula of

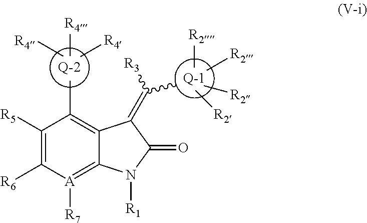

wherein Z is C or N,

R₁, R_{2′}, R_{2″}, R_{2′″}, R_{2″″}, R₃, R_{4′}, R_{4″}, R_{4′″}, R₅, R₆, R₇, Q-1, and Q-2 are the same as the above definitions.

Item147. The compound of Item 126, the compound has the formula of

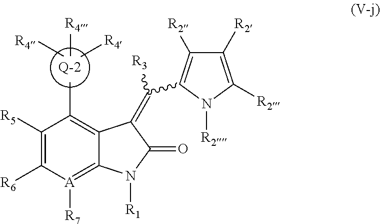

wherein

Z is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R_{4′}, R_{4″}, R_{4′″}, R₅, R₆, R₇, and Q-2 are the same as the above definitions.

Item148. The compound of Item 147, wherein Z is C.

Item149. The compound of Item 147, wherein Z is N.

Item150. The compound of any one of Item 147 to 149, wherein R_{2″″} is H.

Item151. The compound of any one of Item 147 to 150, wherein each of R_{2″} and R_{2′″} is H.

Item152. The compound of any one of Item 147 to 150, wherein each of R_{2″} and R_{2′″} is methyl.

Item153. The compound of Item 126, the compound has the formula of

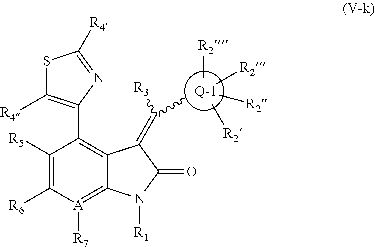

wherein A is C or N,

R₁, R_{2′}, R_{2″}, R_{2′″}, R_{2″″}, R₃, R_{4′}, R_{4″}, R₅, R₆, R₇, and Q-1 are the same as the above definitions.

Item154. The compound of Item 126, the compound has the formula of

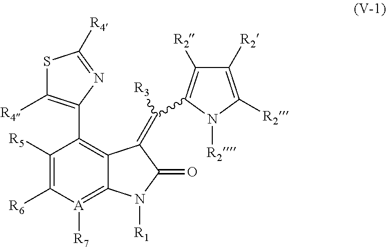

wherein

A is C or N,

R_{2′}, R_{2″}, and R_{2′″} are each independently hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, and

R_{2″″} is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c},

R₁, R₃, R_{4′}, R_{4″}, R₅, R₆, and R₇ are the same as the above definitions.

Item154′. The compound of Item126, wherein each of R₁, R_{2′}, R_{2″}, R_{2′″}, R_{2″″}, R₃, R_{4′}, R_{4″}, R_{4′″}, R₅, R₆, R₇, Y, Z, A, Q-1, and Q-2 can be selected from any of the groups illustrated hereinabove.

Item155. A pharmaceutical composition comprising a compound of any one of Item1 to 147, or a pharmaceutically acceptable salt, ester or pro-drug thereof, and a pharmaceutically acceptable excipient, carrier, or diluent.

Item156. A method of treating or preventing cancer, or a related disorder or condition thereof in a mammal, including a human, comprising administering to a subject in need thereof a therapeutically effective amount of a pharmaceutical composition comprising a compound of any one of Item1 to 154, or a pharmaceutically acceptable salt, ester or pro-drug thereof, effective in the treatment or prevention of cancer, or a related disorder or condition thereof in a mammal, including a human, and a pharmaceutically acceptable excipient, carrier, or diluent.

Item157. A method of treating, preventing or ameliorating a protein kinase related disorder in a mammal, comprising administering to the mammal in need thereof a therapeutically effective amount of a pharmaceutical composition comprising a compound of any one of Item1 to 154.

Item158. The method of Item157, wherein the protein kinase related disorder is a cancer such as lung cancer, bladder cancer, head and neck cancer, melanoma, ovarian cancer, prostate cancer, breast cancer, small-cell lung cancer, glioma, colorectal cancer, non-small cell lung cancer, genitourinary cancer, pancreatic cancer, thyroid cancer, Hodgkin's lymphoma, non-Hodgkin's lymphoma, gastrointestinal cancer, gastric cancer, hepatoma, gastrointestinal stromal tumor, squamous cell carcinoma, renal cell carcinoma, astrocytoma, Kaposi's sarcoma, chronic myelogenous leukemia, acute myelogenous leukemia, myeloproliferative disorders, and glioblastoma.

Item159. The method of any one of Item156 or 157, wherein the protein kinase is CSCPK.

Item160. The method of any one of Item156 or 157, wherein the protein kinase includes serine-threonine kinases, receptor tyrosine kinases and non-receptor tyrosine kinases.

Item161. The method of any one of Item 156 to 160, wherein the protein kinase related disorder includes diabetes, an autoimmune disorder, a hyperproliferation disorder, angiogenesis, an inflammatory disorder, an immunological disorder, a cardiovascular disorder, restenosis, fibrosis, psoriasis, von Heppel-Lindau disease, osteoarthritis, neurodegeneration, infection, and rheumatoid arthritis.

Item162. A method of inhibiting, reducing, and/or diminishing cancer stem cell survival and/or proliferation, self-renewal in a mammal by inhibiting or decreasing unwanted activity of CSCPKs.

Item163. A method of inhibiting cancer stem cell niche, or stromal cell signaling by targeting CSCPKs.

Item164. A method of treating cancer, inhibiting/reducing/diminishing cancer stem cell survival and/or proliferation.

Item165. A method of modulating the catalytic activity of a protein kinase.

Item13. The method of Item 162 to 165, comprises contacting said protein kinase with a compound of any one of Item 1 to 154, or a pharmaceutically-acceptable salt, ester or pro-drug thereof. In certain embodiments, the protein kinase includes a serine-threonine kinase, a receptor tyrosine kinase and a non-receptor tyrosine kinase. In the above Item 1 to 36, the definition of R_{n′} can replace the definition of R_{4′}, R_{5′}, R_{6′}, or R_{7′}, the definition of R_{n″} can replace the definition of R_{4″}, R_{5″}, R_{6″}, or R_{7″}, and the definition of R_{n′″} can replace the definition of R_{4′″}, R_{5′″}, R_{6′″}, or R_{7′″}.

Preparation methods for a compound of Formula I are explained. A compound Formula I or a pharmaceutically acceptable salt thereof is illustrated, but the present invention is not intended to be limited thereto.

In the following method, the starting materials and the intermediates of the reaction may be isolated and purified if desired using conventional techniques, including but not limited to filtration, distillation, crystallization, chromatography and the like.

The materials of invention can be characterized by using conventional means including but not limited to physical constants and spectral data. The reactions are performed in solvents appropriate to the reagents and materials employed and are suitable for transformations being effected. The representative examples include, but are not limited to, tetrahyrdofuran, dimethylforamide, methanol, ethanol, water, dimethylforamide, chloroform, dichloromethane, hexane, toluene, 1,4-dioxane or ethyl acetate.

Unless specified, the reactions described herein were performed at atmospheric pressure over a temperature range from about −78° C. to about 150° C.

For heating, any methods can be used which depends on reagent and target material. The representative examples include, but are not limited to, water bath, oil bath, water bath, or microwave reactor.

The compound of Formula I in the present invention may be prepared from known compounds by optionally combining the method of the following Preparation methods I to II, similar methods to the following Preparation methods, or synthetic known to a skilled person.

Preparation of Method

A compound of Formula I may be synthesized by the following method.

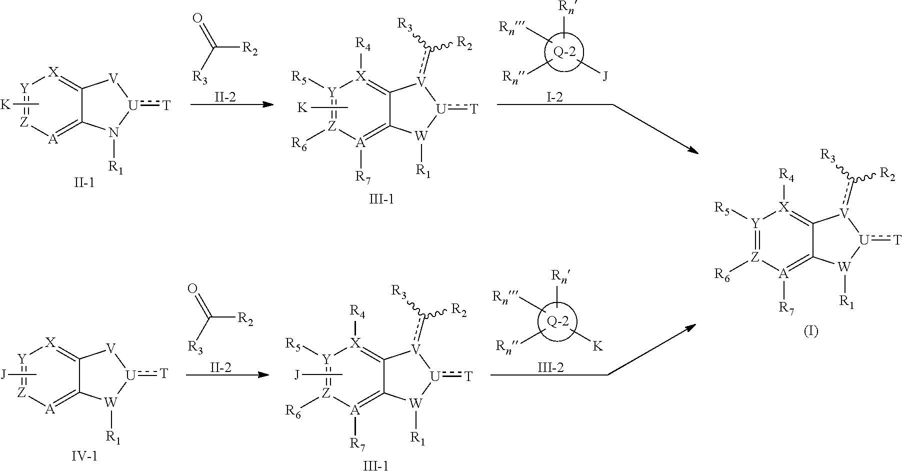

In the scheme, R₁, R₂, R₃, R₄, R₅, R₆, R₇, T, U, V, X, Y, Z, A, R_{n′}, R_{n″}, R_{n′″} and Q-2 are as defined in the above Item 1, except that in I-1 and III-1, R₄, R₅, R₆, and R₇ are not

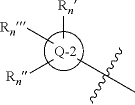

J is metal containing group such as boronic acid, boronic acid pinacol ester, trifluoro boran, organic tin, zinc halide, magnesium halide, organic silicon, and organic lithium. K is leaving group such as Cl, Br, I, and OTf.

Preparation of Method I

A compound of Preparation of method may be synthesized by the following method.

Among a compound of Formula I, Compound I-3 or a pharmaceutically acceptable salt thereof is prepared by the following method.

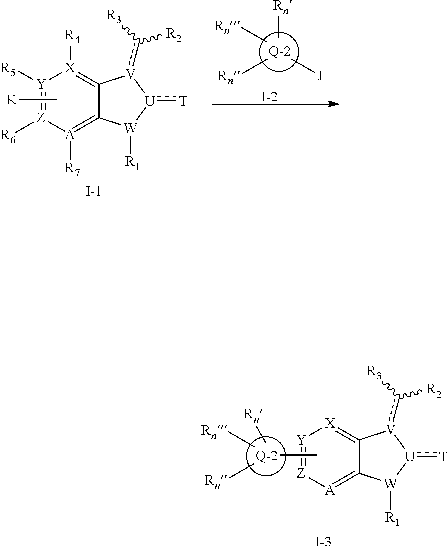

In the scheme, the symbols have the same meaning as defined above.

A compound of formula I-1 can react with a compound of formula I-2 in the presence of transition metal catalyst (representative examples include, but are not limited to tetrakis(triphenylphosphine)palladium(0), [1,1′-bis(diphenylphosphino)ferrocene]palladium(II) dichloride, palladium carbon, dichlorobis(triphenylphosphine)nickel(II), or bis(triphenylphosphine)palladium(II) dichloride), alkali metal carbonate (representative examples include, but are not limited to potassium carbonate, sodium carbonate, or cesium carbonate) or other alkali metal salt (sodium hydroxide, potassium hydroxide, sodium ethoxide, sodium methoxide, sodium tert-butoxide, potassium tert-butoxide, sodium hydride, sodium phosphate, potassium phosphate) and appropriate solvent or without solvent to give a compound of formula 1-3.

Preparation Method II

A compound I-1 may be prepared from a compound II-2.

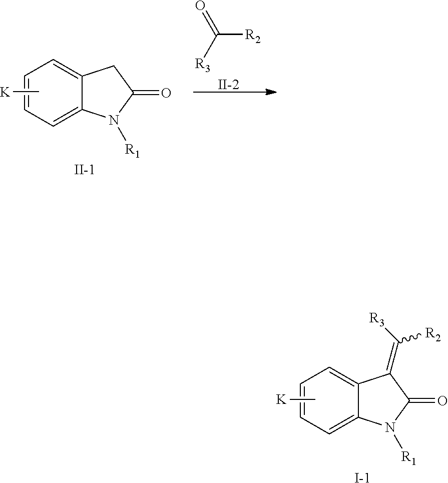

In the scheme, the symbols have the same meaning as defined above.

A compound of formula II-1 can react with a compound of formula II-2 in the presence of a base (representative examples include, but are not limited to pyrrolidine and piperidine) or an acid (representative examples include, but are not limited to hydrochloric acid, acetic acid, trifluoroacetic acid), and appropriate solvent or without solvent to give a compound of formula I-1.

Preparation Method III

A compound I-3 may be prepared from a compound III-1.

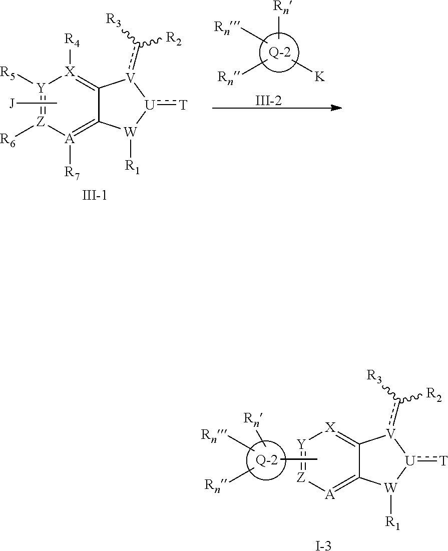

In the scheme, the symbols have the same meaning as defined above.

A compound of formula III-1 can react with a compound of formula III-2 in the presence of transition metal catalyst (representative examples include, but are not limited to, tetrakis(triphenylphosphine)palladium(0), [1,1′-bis(diphenylphosphino)ferrocene]palladium(II) dichloride, palladium carbon, dichlorobis(triphenylphosphine)nickel(II), or bis(triphenylphosphine)palladium(II) dichloride), alkali metal carbonate (representative examples include, but are not limited to, potassium carbonate, sodium carbonate, or cesium carbonate) or other alkali metal salt (sodium hydroxide, potassium hydroxide, dodium ethoxide, sodium methoxide, sodium tert-butoxide, potassium tert-butoxide, sodium hydride, sodium phosphate, potassium phosphate), and appropriate solvent or without solvent to give a compound of formula I-3.

Preparation Method IV

A compound III-1 may be prepared from a compound IV-1.

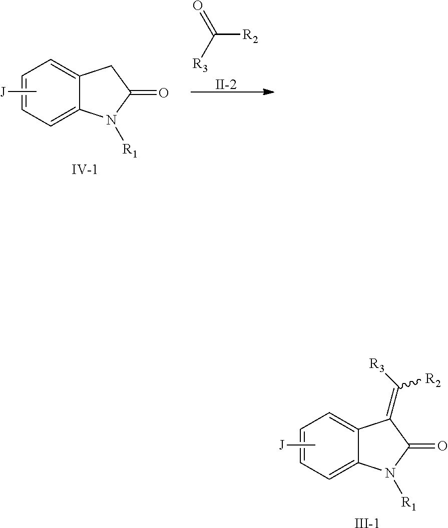

In the scheme, the symbols have the same meaning as defined above.

A compound of formula IV-1 can react with a compound of formula II-2 in the presence of a base (representative examples include, but are not limited to pyrrolidine and piperidine) or an acid (representative examples include, but are not limited to hydrochloric acid, acetic acid, trifluoroacetic acid), and appropriate solvent or without solvent to give a compound of formula III-1.

Preparation Method V

A compound of formula III-1 may be prepared from a compound I-1.

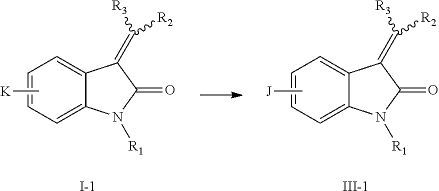

In the scheme, the symbols have the same meaning as defined above.

A compound of formula I-1 can react with a compound of boron reagent (representative examples include, but are not limited to, bis(pinacolato)diboron, bis(neopentyl Glycolato)diboron, or bis(catecholato)diboron) in the presence of transition metal catalyst (representative examples include, but are not limited to, dichloro[1,1′-bis(diphenylphosphino)ferrocene]palladium, [1,1′-bis(diphenylphosphino)ferrocene]palladium(II) dichloride, or bis(triphenylphosphine)palladium(II) dichloride), alkali metal carbonate or alkali metal acetate (representative examples include, but are not limited to, potassium carbonate, sodium carbonate, cesium carbonate, or potassium acetate), and appropriate solvent or without solvent to give a compound of formula III-1.

Preparation Method VI

A compound of formula I-3 may be prepared from a compound VI-1.

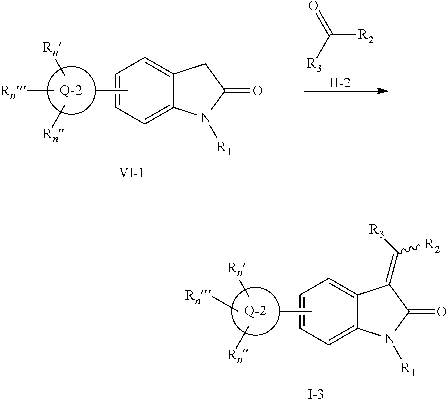

In the scheme, the symbols have the same meaning as defined above.

A compound of formula VI-1 can react with a compound of formula II-2 in the presence of a base (representative examples include, but are not limited to pyrrolidine and piperidine) or an acid (representative examples include, but are not limited to hydrochloric acid, acetic acid, trifluoroacetic acid), and appropriate solvent or without solvent to give a compound of formula I-3.

Preparation Method VII

A compound of formula VI-1 may be prepared from a compound of formula II-1.

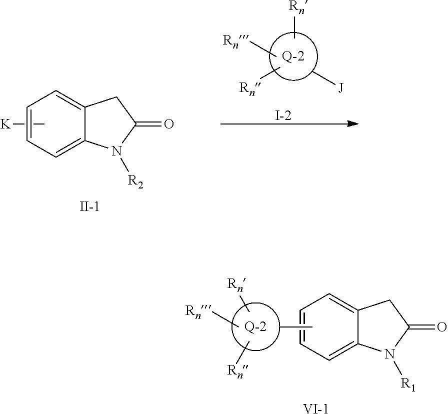

In the scheme, the symbols have the same meaning as defined above.

A compound of formula II-1 can react with a compound of formula I-2 in the presence of transition metal catalyst (representative examples include, but are not limited to, tetrakis(triphenylphosphine)palladium(0), [1,1′-bis(diphenylphosphino)ferrocene]palladium(II) dichloride, palladium carbon, dichlorobis(triphenylphosphine)nickel(II), or bis(triphenylphosphine)palladium(II) dichloride), alkali metal carbonate (representative examples include, but are not limited to, potassium carbonate, sodium carbonate, or cesium carbonate) or other alkali metal salt (sodium hydroxide, potassium hydroxide, dodium ethoxide, sodium methoxide, sodium tert-butoxide, potassium tert-butoxide, sodium hydride, sodium phosphate, potassium phosphate), and appropriate solvent or without solvent to give a compound of formula VI-1.

Preparation Method VIII

A compound of formula VI-1 may be prepared from a compound of formula IV-1.

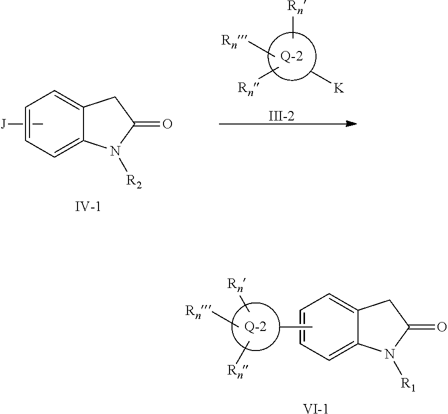

In the scheme, the symbols have the same meaning as defined above.

A compound of formula IV-1 can react with a compound of formula III-2 in the presence of transition metal catalyst (representative examples include, but are not limited to, tetrakis(triphenylphosphine)palladium(0), [1,1′-bis(diphenylphosphino)ferrocene]palladium(II) dichloride, palladium carbon, dichlorobis(triphenylphosphine)nickel(II), or bis(triphenylphosphine)palladium(II) dichloride), alkali metal carbonate (representative examples include, but are not limited to, potassium carbonate, sodium carbonate, or cesium carbonate) or other alkali metal salt (sodium hydroxide, potassium hydroxide, dodium ethoxide, sodium methoxide, sodium tert-butoxide, potassium tert-butoxide, sodium hydride, sodium phosphate, potassium phosphate) and appropriate solvent or without solvent to give a compound of formula VI-1.

Preparation Method IX

A compound of formula IX-3 may be prepared from a compound of formula IX-1.

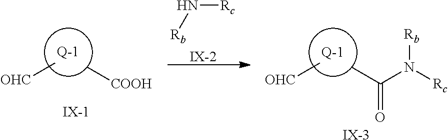

In the scheme, the symbols have the same meaning as defined above.

A compound of formula IX-1 can react with a compound of formula IX-2 (representative examples include, but are not limited to, N¹,N¹-diethylethane-1,2-diamine, N¹,N¹-dimethylethane-1,2-diamine, 2-(pyrrolidin-1-yl)ethanamine, N-methyl-piperazine, N-methyl-homopiperazine, 2-morpholinoethanamine, or morpholine) in the presence of coupling reagent (representative examples include, but are not limited to, N,N′-dicyclohexylcarbodiimide, N,N′-diisopropylcarbodiimide, or 1-ethyl-3-(3-dimethylaminopropyl)carbodiimide hydrochloride), and appropriate solvent or without solvent to give a compound of formula IX-3. This amide formation reaction can be performed in the presence of appropriate additives (representative examples include, but are not limited to, 1-hydroxybenzotriazole, or N-hydroxysuccinimide).

Preparation Method X

A compound of formula X-5 and X-6 may be prepared from a compound of formula IV-1.

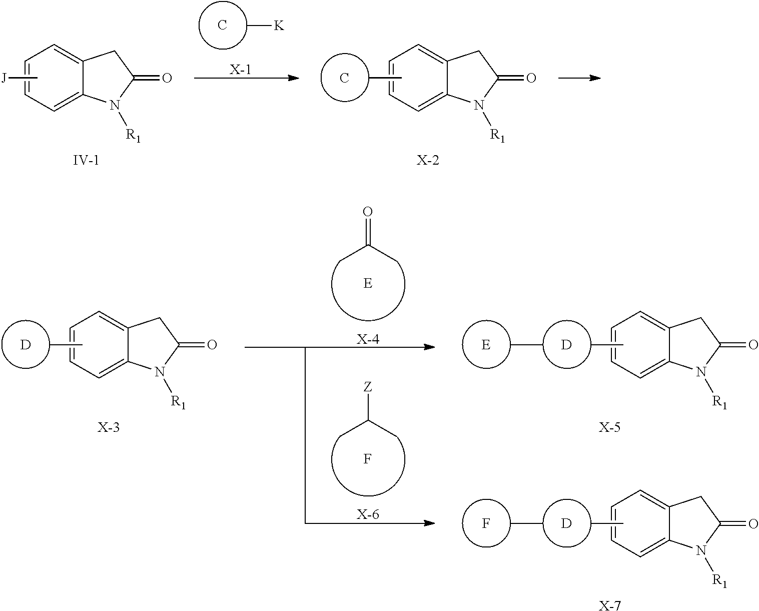

In the scheme, R₁, J and K are same as the above definition. C is optionally substituted heterocycle group (said heterocycle group is unsaturated, and one of double bond is attached to J or K). D is optionally substituted heterocycle group (wherein heterocycle group is saturated). E is optionally substituted heterocycle (wherein heterocycle group is saturated). F is optionally substituted heterocycle group.

A compound of formula IV-1 can react with a compound of formula X-1 in the presence of transition metal catalyst (representative examples include, but are not limited to, tetrakis(triphenylphosphine)palladium(0), [1,1′-bis(diphenylphosphino)ferrocene]palladium(II) dichloride, palladium carbon, dichlorobis(triphenylphosphine)nickel(II), or bis(triphenylphosphine)palladium(II) dichloride), alkali metal carbonate (representative examples include, but are not limited to, potassium carbonate, sodium carbonate, or cesium carbonate) or other alkali metal salt (sodium hydroxide, potassium hydroxide, dodium ethoxide, sodium methoxide, sodium tert-butoxide, potassium tert-butoxide, sodium hydride, sodium phosphate, potassium phosphate), and appropriate solvent or without solvent to give a compound of formula X-2.

A compound of formula X-2 can further react in the presence of transition metal catalyst (representative examples include, but are not limited to, palladium carbon, platinum carbon or rhodium carbon), and appropriate solvent or without solvent under hydrogen atmosphere to give a compound of formula X-3. The reaction can be performed in any hydrogen pressure which depends on reagent and target material. However, preferable pressure is between 1 to 10 atm, and even more preferably between 1 to 5 atm.

A compound of formula X-3 can react with a compound of formula X-4 in the presence of reducing reagent (representative examples include, but are not limited to, sodium triacetoxyborohydride, tetramethyl triacetoxyborohydride, picolyl borane, or sodium cyanoborohydride), acid (representative examples include, but are not limited to acetic acid, or trifluoroacetic acid), and appropriate solvent or without solvent to give a compound of formula X-5.

A compound of formula X-3 can react with a compound of formula X-6 (wherein Z is leaving group representative examples include, but are not limited to, chloro, bromo, iodo, trifluoromethanesulfonyl, or p-tosyl) in the presence of tertiary amine (representative examples include, but are not limited to, diisopyropylethylamine, triethylamine, or pyridine), and appropriate solvent or without solvent to give a compound of formula X-7.

Preparation Method XI

A compound of formula X-2 may be prepared from a compound of formula II-2.

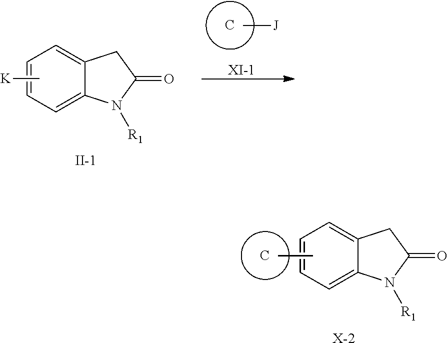

In the scheme, the symbols have the same meaning as defined above.

A compound of formula II-1 can react with a compound of formula XI-1 in the presence of transition metal catalyst (representative examples include, but are not limited to, tetrakis(triphenylphosphine)palladium(0), [1,1′-bis(diphenylphosphino)ferrocene]palladium(II) dichloride, palladium carbon, dichlorobis(triphenylphosphine)nickel(II) or bis(triphenylphosphine)palladium(II) dichloride), alkali metal carbonate (potassium carbonate, sodium carbonate, or cesium carbonate) or other alkali metal salt (sodium hydroxide, potassium hydroxide, dodium ethoxide, sodium methoxide, sodium tert-butoxide, potassium tert-butoxide, sodium hydride, sodium phosphate, potassium phosphate), and appropriate solvent or without solvent to give a compound of formula X-2.

Preparation Method XII

A compound of formula XII-4 may be prepared from a compound of formula XII-1.

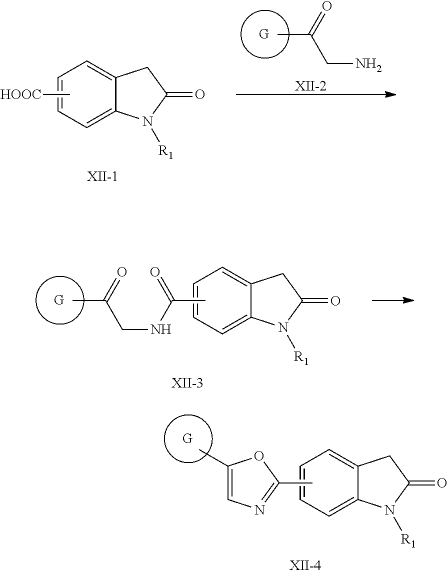

In the scheme, R₁ is same as the above definition. G is aryl or substituted aryl, or heterocycle or substituted heterocycle.

A compound of formula XII-1 can react with a compound of formula XII-2 in the presence of coupling reagent (representative examples include, but are not limited to, N,N′-dicyclohexylcarbodiimide, N,N′-diisopropylcarbodiimide, or 1-ethyl-3-(3-dimethylaminopropyl), primary or secondary amine (representative examples include, but are not limited to, 2-amino-1-phenylethanone, 2-amino-1-p-tolylethanone, 2-amino-1-(4-chlorophenyl)ethanone, 2-amino-1-(4-methoxyphenyl)ethanone, or 2-amino-1-(pyridin-4-yl)ethanone), and appropriate solvent or without solvent to give a compound of formula XII-3. This amide formation reaction can be performed in the presence of appropriate additives (representative examples include, but are not limited to, 1-hydroxybenzotriazole, or N-hydroxysuccinimide).

A compound of formula XII-3 can further react in the presence of acid (representative examples include, but are not limited to, trifluoroacetic acid, methanesulfonic acid, p-toluenesulfonic acid, benzenesulfonic acid, or sulfuric acid) to give compound of formula XII-4.

Preparation Method XIII

A compound of formula XIII-6 may be prepared from a compound of formula XIII-1.

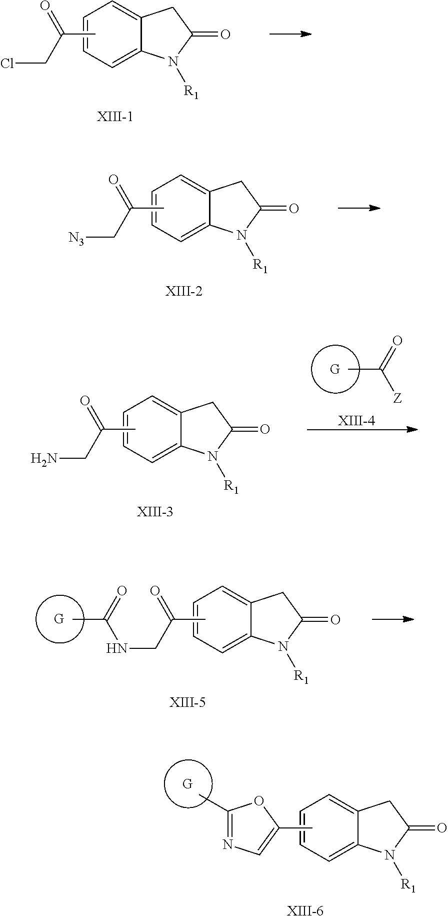

In the scheme, the symbols have the same meaning as defined above.

A compound of formula XIII-1 can react with azide salt (representative examples include, but are not limited to, sodium azide), and appropriate solvent or without solvent to give a compound of formula XIII-2. This reaction can be performed in the presence of additive (representative examples include, but are not limited to, potassium iodide, or tetrabutylammonium iodide).

A compound of formula XIII-2 can further react in the presence of metal catalyst (representative examples include, but are not limited to, palladium carbon, or platinum carbon), and appropriate solvent or without solvent under hydrogen atmosphere to give a compound of formula XIII-3. The reaction can be performed in any hydrogen pressure which depends on reagent and target material. However, preferable pressure is between 1 to 10 atm, and even more preferably between 1 to 5 atm.

A compound of formula XIII-3 can further react with a compound of formula XIII-4 (wherein “Z” is defined as leaving group such as Cl, Br and the likes. Representative examples include, but are not limited to, benzoyl chloride, benzoyl bromide, 4-chlorobenzoyl chloride, 4-methoxybenzoyl chloride, 4-methylbenzoyl chloride, isonicotinoyl chloride, nicotinoyl chloride, picolinoyl chloride, or tetrahydro-2H-pyran-4-carbonyl chloride), and appropriate solvent or without solvent to give a compound of formula XIII-5. This reaction can be performed in the presence of additive (representative examples include, but are not limited to, diisopropylethylamine, pyridine, or triethylamine).

A compound of formula XIII-5 can further react in the presence of acids (representative examples include, but are not limited to, trifluoroacetic acid, methanesulfonic acid, p-toluenesulfonic acid, benzenesulfonic acid, or sulfuric acid), and appropriate solvent or without solvent to give a compound of formula XIII-6.

Preparation Method XIV

A compound of formula XIV-4 may be prepared from a compound of formula XIV-1.

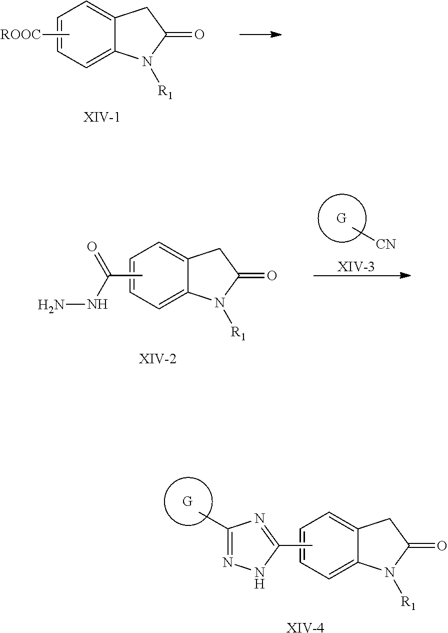

In the scheme, R is alkyl. The symbols have the same meaning as defined above.

A compound of formula XIV-1 can react with hydrazine (representative examples include, but are not limited to, hydrazine hydrate, or hydrazine) in the presence of solvent or without solvent to give a compound of formula XIV-2.

A compound of formula XIV-2 can react with aryl nitrile (representative examples include, but are not limited to, benzonitrile, 4-methylbenzonitrile, 4-chlorobenzonitrile, 4-methoxybenzonitrile, 3-methylbenzonitrile, isonicotinonitrile, or tetrahydro-2H-pyran-4-carbonitrile) in the presence of alkali metal carbonate (representative examples include, but are not limited to, potassium carbonate, sodium carbonate, or cesium carbonate) and appropriate solvent or without solvent to give a compound of formula XIV-4.

Preparation Method XV

A compound of formula XV-2 may be prepared from a compound of formula XIII-1.

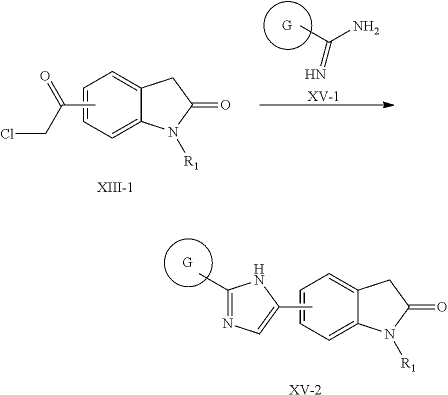

In the scheme, the symbols have the same meaning as defined above.

A compound of formula XIII-1 can react with a compound of formula XV-1 (representative examples include, but are not limited to, benzimidamide, substituted benzimidamide, or isonicotinimidamide), and appropriate solvent or without solvent to give a compound of formula XV-2. This reaction can be performed in the presence of additive (representative examples include, but are not limited to, sodium iodide or potassium iodide).

Preparation Method XVI

A compound of formula XVI-2 may be prepared from a compound of formula XIII-1.

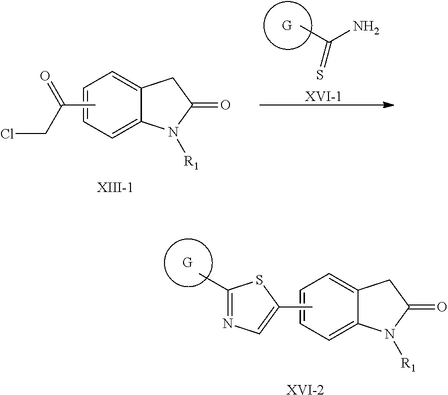

In the scheme, the symbols have the same meaning as defined above.

A compound of formula XIII-1 can react with a compound of formula XVI-1 (representative examples include, but are not limited to, benzothioamide, 4-methylbenzothioamide, 4-chlorobenzothioamide, 4-methoxybenzothioamide, 3-methylbenzothioamide, pyridine-4-carbothioamide, pyridine-3-carbothioamide, pyridine-2-carbothioamide or tert-butyl 4-carbamothioylpiperidine-1-carboxylate), and appropriate solvent or without solvent to give a compound of formula XVI-2.

Preparation Method XVII

A compound of formula XVII-3 may be prepared from a compound of formula IV-1.

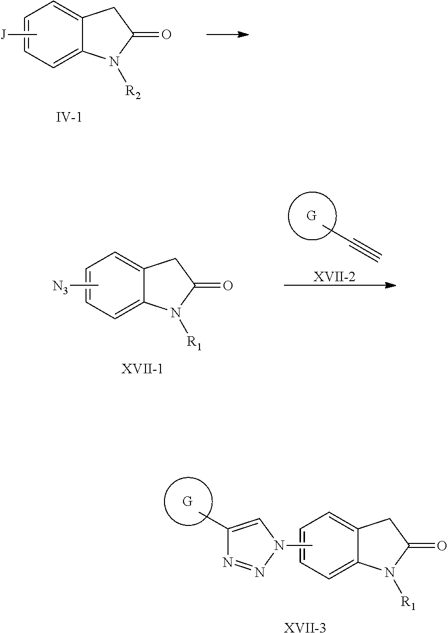

In the scheme, the symbols have the same meaning as defined above.

A compound of formula IV-1 can react with azide salt (representative examples include, but are not limited to, sodium azide, or hydrogen azide) and a compound of formula XVII-2 (representative examples include, but are not limited to, phenyl acetylene, 1-ethynyl-4-methylbenzene, 4-chloro-1-ethynyl-benzene, or 4-ethynylpyridine) in the presence of alkali base carbonate (representative examples include, but are not limited to, sodium carbonate, potassium carbonate, or cesium carbonate), copper salt (representative examples include, but are not limited to, copper chloride (I), copper bromide (I), or copper iodide (I)), ascorbate (representative examples include, but are not limited to, sodium ascorbate, or potassium ascorbate), amine (representative examples include, but are not limited to, N,N′-dimethylethylenediamine) and appropriate solvent or without solvent to give a compound of formula XVII-3.

Preparation Method XVIII

A compound of formula XVIII-4 and XVIII-6 may be prepared from a compound of formula IV-1.

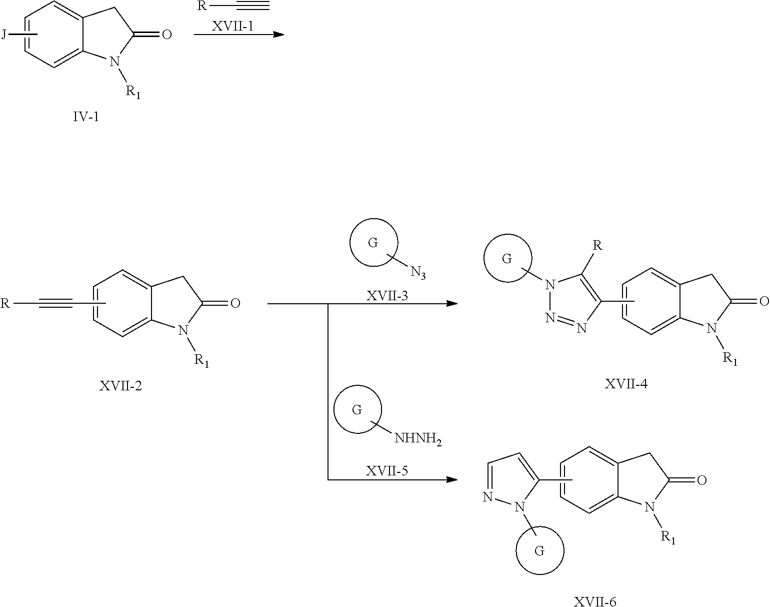

In the scheme, the symbols have the same meaning as defined above.

A compound of formula IV-1 (wherein R is alkyl, or trialkyl silyl) can react with a compound of formula XVIII-1 (representative examples include, but are not limited to, phenylacetylene, prop-1-yne, or 3,3-diethoxyprop-1-yne) in the presence of transition metal catalyst (representative examples include, but are not limited to, tetrakis(triphenylphosphine)palladium(0), [1,1′-bis(diphenylphosphino)ferrocene]palladium(II) dichloride, or bis(triphenylphosphine)palladium(II) dichloride), copper catalyst (representative examples include, but are not limited to, copper chloride (I), copper bromide (I), or copper iodide (I)), organic base (representative examples include, but are not limited to, diisopropylethyamine, or triethylamine), and appropriate solvent or without solvent to give a compound of formula XVIII-2.

A compound of formula XVIII-2 can further react with a compound of formula XVIII-3 (representative examples include, but are not limited to, phenylazide, 1-azido-4-methylbenzene, 1-azido-4-chlorobenzene, or 4-azidopyridine) in the presence of copper catalyst (representative examples include, but are not limited to, copper chloride (I), copper bromide (I), or copper iodide (I)), alkali metal carbonate (representative examples include, but are not limited to, sodium carbonate, potassium carbonate, or cesium carbonate), amine (representative examples include, but are not limited to, N,N′-dimethylethylenediamine) and appropriate solvent or without solvent to give a compound of formula XVIII-4.

A compound of formula XVIII-2 (wherein R contains ketone, aldehyde or their equivalent (representative examples include, but are not limited to, 5-(3,3-diethoxyprop-1-ynyl)indolin-2-one, or 5-(3,3-diethoxybut-1-ynyl)indolin-2-one) next to triple bond) can react with a compound of formula XVIII-5 (representative examples include, but are not limited to, phenylhydrazine, p-tolylhydrazine, or p-cyanophenylhydrazine) in the presence of appropriate solvent or without solvent to give a compound of formula XVIII-6. This reaction can be performed in the presence of acid (representative examples include, but are not limited to, sulfuric acid, p-toluenesulfonyl acid, or methanesulfonyl acid).

Presently disclosed pharmaceutical compositions can be used in an animal or human. A presently disclosed compound can be formulated as a pharmaceutical composition for oral, buccal, parenteral (e.g., intravenous, intramuscular or subcutaneous), topical, rectal or intranasal administration or in a form suitable for administration by inhalation or insufflation. The compounds presently disclosed may also be formulated for sustained delivery according to methods well known to those of ordinary skill in the art. Examples of such formulations can be found in U.S. Pat. Nos. 3,119,742; 3,492,397; 3,538,214; 4,060,598; and 4,173,626.

The formulations may conveniently be presented in unit dosage form and may be prepared by any methods well known in the art of pharmacy. The amount of active ingredient that can be combined with a carrier material to produce a single dosage form will vary depending upon the mammal being treated and the particular mode of administration. The amount of active ingredient, which can be combined with a carrier material to produce a single dosage form will generally be that amount of the compound which produces a therapeutic effect. Generally, out of 100%, this amount will range, for example, from about 0.1% to about 25% (e.g., 1%, 2%, 5%, 10%, 15%, 20%) of active ingredient.

Therapeutic compositions or formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the alcohol or inhibitor according to the invention is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-.beta.-cyclodextrin, may be used to solubilize compounds.

Besides inert diluents, the oral compositions can also include adjuvants such as wetting agents, emulsifying and suspending agents, sweetening, flavoring, coloring, perfuming and preservative agents. Suspensions, in addition to the alcohols or inhibitors according to the invention, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more alcohols or inhibitors according to the invention, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention. Formulations of the present invention which are suitable for vaginal administration also include pessaries, tampons, creams, gels, pastes, foams or spray formulations containing such carriers as are known in the art to be appropriate.

Dosage forms for the topical or transdermal administration of an alcohol or other inhibitor according to the invention include powders, sprays, ointments, pastes, creams, lotions, gels, solutions, patches and inhalants. The active compound may be mixed under sterile conditions with a pharmaceutically-acceptable excipient, carrier, or diluent, including any preservatives, buffers, or propellants which may be required.

For intranasal administration or administration by inhalation, presently disclosed compounds may be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the presently disclosed compound. Capsules and cartridges (made, for example, from gelatin) for use in an inhaler or insufflator may be formulated containing a powder mix of a presently disclosed compound and a suitable powder base such as lactose or starch.

The ointments, pastes, creams and gels may contain, in addition to an alcohol or other inhibitor according to the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of this invention.

Pharmaceutical compositions of this invention suitable for parenteral administration comprise one or more alcohols or inhibitors according to the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of the alcohol or inhibitor according to the invention, it is desirable to slow the absorption of the alcohol or inhibitor from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered composition is accomplished by dissolving or suspending the alcohol or inhibitor in an oil vehicle. One strategy for depot injections includes the use of polyethylene oxide-polypropylene oxide copolymers wherein the vehicle is fluid at room temperature and solidifies at body temperature.

The pharmaceutical compounds of this invention may be administered alone, or simultaneously, subsequently or sequentially with one or more active agents, other pharmaceutical agents, or with other anti-cancer or cytotoxic agent as described hereinabove, as well as in combination with a pharmaceutically-acceptable excipient, carrier, or diluent as described above.

The amount of pharmacological agent in the oral unit dosage form, with as a single or multiple dosage, is an amount that is effective for treating a neurological disorder. As one of skill in the art will recognize, the precise dose to be employed will depend on a variety of factors, examples of which include the condition itself, the seriousness of the condition being treated, the particular composition used, as well as various physical factors related to the individual being treated. In vitro or in vivo assays can optionally be employed to help identify optimal dosage ranges.

A proposed dose of a presently disclosed compound for oral, parenteral or buccal administration to the average adult human for the treatment or prevention of a disease state herein relevant is about 0.1 mg to about 2000 mg. In certain embodiments, the proposed dose is from about 0.1 mg to about 200 mg (e.g., 1 mg, 5 mg, 10 mg, 20 mg, 50 mg, 75 mg, 100 mg, 150 mg) of the active ingredient per unit dose. Irrespective of the amount of the proposed dose, administration of the compound can occur, for example, 1, 2, 3, or 4 times per day, or 1, 2, 3, 4 or 5 times a week.

Aerosol formulations for the treatment or prevention of the conditions referred to herein the average adult human are preferably arranged so that each metered dose or “puff’ of aerosol contains about 20 μg to about 10,000 μg, preferably, about 20 μg to about 1000 μg (e.g., 25 μg, 50 μg, 100 μg, 200 μg, 500 μg, 750 μg) of a presently disclosed compound. The overall daily dose with an aerosol will be within the range from about 100 μg to about 100 mg (e.g., 200 μg, 500 μg, 1 mg, 2 mg, 5 mg, 10 mg, 25 mg, 50 mg, 75 mg). In certain embodiments, the overall daily dose with an aerosol generally will be within the range from about 100 μg to about 10 mg (e.g., 200 μg, 500 μg, 1 mg, 2 mg, 5 mg, 7.5 mg). Administration may be several times daily, for example 1, 2, 3, 4, 5 or 8 times, giving for example, 1, 2 or 3 doses each time.

The compounds of the present invention can be prepared using the methods described below, together with synthetic methods known to one skilled in the art of organic synthesis, medicinal chemistry and related fields, or variations thereon. The reactions are performed in solvents where appropriate to the reagents and materials employed and are suitable for transformations being effected. The starting materials for the examples contained herein are either commercially available or are readily prepared by standard methods from known materials. For example, the following reactions are illustrations but not limitations of the preparation of some of the starting materials and examples used herein.

### EXAMPLES

### Chemical Synthesis

### Reference Example 1

### Production of 5-(5-phenylthiophen-2-yl)indolin-2-one

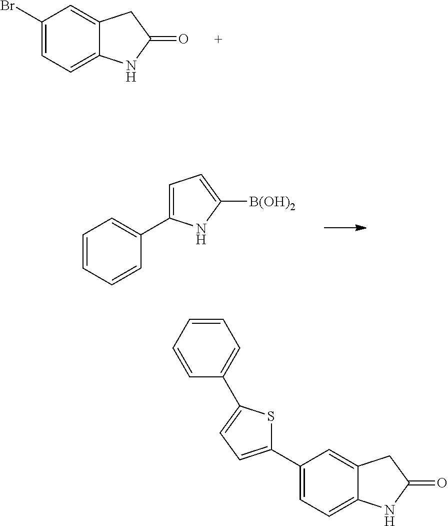 [SMILES: O=C1Cc2cc(Br)ccc2N1 ; OB(O)c1ccc(-c2ccccc2)[nH]1 ; O=C1Cc2cc(-c3ccc(-c4ccccc4)s3)ccc2N1]

To a solution of 5-bromooxindole (100 mg, 0.572 mmol) in dioxane/H₂O (3 ml/1 ml) was added Pd(PPh₃)₄ (55 mg, 0.047 mmol), 5-phenylthiophene-2-boronic acid (106 mg, 0.519 mmol) and potassium carbonate (196 mg, 1.42 mmol). The mixture was stirred at 120° C. for 1 hour under microwave irradiation. The residue was extracted with CHCl₃, and the organic layer was washed with H₂O and brine, dried over Na₂SO₄ and concentrated in vacuo. The residue was purified by column chromatography (CHCl₃/MeOH) to give 5-(5-phenylthiophen-2-yl)indolin-2-one (44 mg) as a pale yellow solid.

MS m/z 292.4 (M+H).

### Reference Examples 2 to 8

Reactions and treatments were carried out in the same manner as Reference example 1 using the corresponding starting material compounds, thereby giving the compounds of Reference example 2 to 8 shown in Table 1.

TABLE 1

| Reference | | |
|---|---|---|
| Example | Structure | Spectral data |
| 2 | 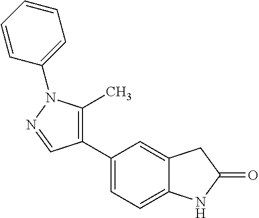 [SMILES: Cc1c(-c2ccc3c(c2)CC(=O)N3)cnn1-c1ccccc1] | LCMS m/z 290.3 (M + H) |
| 3 | 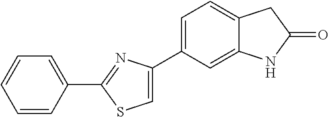 [SMILES: O=C1Cc2ccc(-c3csc(-c4ccccc4)n3)cc2N1] | 300 MHz ¹H-NMR (DMSO-d₆, δ) 10.48 (s, 1H), 8.12 (s, 1H), 8.03-7.98 (m, 2H), 7.65-7.49 (m, 5H), 7.29 (d, 1H, J = 7.7 Hz), 3.52 (s, 2H) |
| 4 | 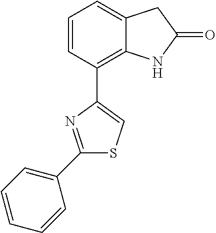 [SMILES: O=C1Cc2cccc(-c3csc(-c4ccccc4)n3)c2N1] | 300 MHz ¹H-NMR (DMSO-d₆, δ) 9.97 (s, 1H), 8.20 (s, 1H), 8.06-8.00 (m, 2H), 7.75 (d, 1H, J = 7.5 Hz), 7.61-7.50 (m, 3H), 7.25 (d, 1H, J = 7.2 Hz), 7.06 (dd, 1H, J = 7.2, 7.5 Hz), 3.60 (s, 2H) |
| 5 | 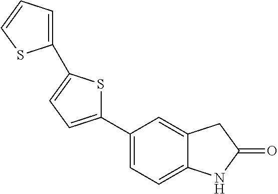 [SMILES: O=C1Cc2cc(-c3ccc(-c4cccs4)s3)ccc2N1] | LCMS m/z 298.2 (M + H) |
| 6 | 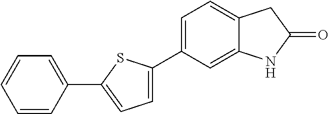 [SMILES: O=C1Cc2ccc(-c3ccc(-c4ccccc4)s3)cc2N1] | LCMS m/z 292.4 (M + H) |
| 7 | 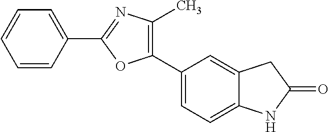 [SMILES: Cc1nc(-c2ccccc2)oc1-c1ccc2c(c1)CC(=O)N2] | LCMS m/z 291.3 (M + H) |
| 8 | 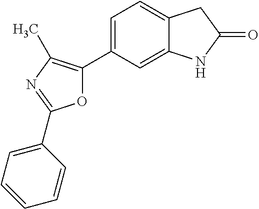 [SMILES: Cc1nc(-c2ccccc2)oc1-c1ccc2c(c1)NC(=O)C2] | LCMS m/z 291.3 (M + H) |

### Reference Example 9

### Production of 5-(5-phenyl-1,3,4-thiadiazol-2-yl)indolin-2-one

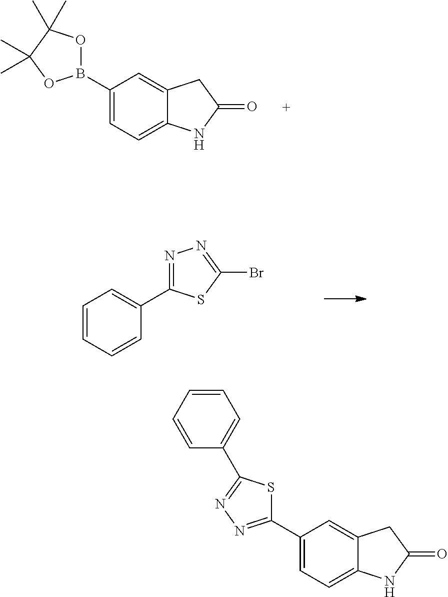 [SMILES: CC1(C)OB(c2ccc3c(c2)CC(=O)N3)OC1(C)C ; Brc1nnc(-c2ccccc2)s1 ; O=C1Cc2cc(-c3nnc(-c4ccccc4)s3)ccc2N1]

To a solution of 5-(4,4,5,5-tetramethyl-1,3,2-dioxaborolan-2-yl)indolin-2-one (98 mg, 0.38 mmol) in dioxane (0.76 ml) was added PdCl₂(dppf) CH₂Cl₂ (28 mg, 0.039 mmol), 2-bromo-5-phenyl-1,3,4-thiadiazole (138 mg, 0.57 mmol) and 2 M potassium carbonate (aq, 568 μL). The mixture was stirred at 90° C. for 4 hour. The residue was extracted with EtOAc, and the organic layer was washed with H₂O and brine, dried over Na₂SO₄, and concentrated in vacuo. The residue was purified by column chromatography (n-hexane/EtOAc) to give 5-(5-phenyl-1,3,4-thiadiazol-2-yl)indolin-2-one (28 mg) as brown oil.

LCMS m/z 294.3 (M+H)

### Reference Examples 10 to 14

Reactions and treatments were carried out in the same manner as Reference example 9 using the corresponding starting material compounds, thereby giving the compounds of Reference example 10 to 14 shown in Table 2.

TABLE 2

| Reference | | |
|---|---|---|
| Example | Structure | Spectral data |
| 10 | 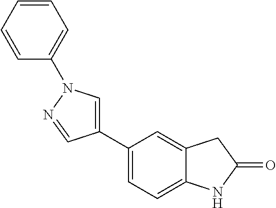 [SMILES: O=C1Cc2cc(-c3cnn(-c4ccccc4)c3)ccc2N1] | LCMS m/z 276.3 (M + H) |
| 11 | 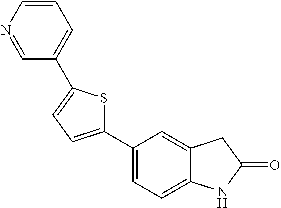 [SMILES: O=C1Cc2cc(-c3ccc(-c4cccnc4)s3)ccc2N1] | LCMS m/z 293.2 (M + H) |
| 12 | 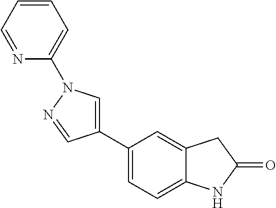 [SMILES: O=C1Cc2cc(-c3cnn(-c4ccccn4)c3)ccc2N1] | LCMS m/z 277.3 (M + H) |
| 13 | 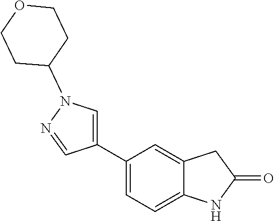 [SMILES: O=C1Cc2cc(-c3cnn(C4CCOCC4)c3)ccc2N1] | 300 MHz ¹H-NMR (CDCl₃, δ) 7.61 (s, 1H), 7.46 (s, 1H), 4.40-4.30 (m, 1H), 4.33-4.09 (m, 2H), 3.50 (d, 2H, J = 2.4, 8.8 Hz), 2.11-1.93 (m, 2H). |
| 14 | 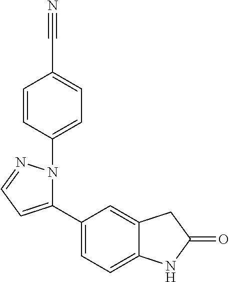 [SMILES: N#Cc1ccc(-n2nccc2-c2ccc3c(c2)CC(=O)N3)cc1] | 400 MHz ¹H-NMR (CDCl₃, δ) 8.58 (brs, 1H), 7.78 (d, 1H, J = 1.8 Hz), 7.65 (dd, 2H, J = 1.9, 6.8 Hz), 7.47 (dd, 2H, J = 1.9, 6.8 Hz), 7.16 (s, 1H), 7.09 (dd, 1H, J = 1.6, 8.1 Hz), 6.88 (d, 1H, J = 1.6, 8.1 Hz), 6.50 (d, 1H, J = 1.8 Hz), 3.56 (s, 2H). |

### Reference Example 15

### Production of 5-(5-phenyloxazol-2-yl)indolin-2-one

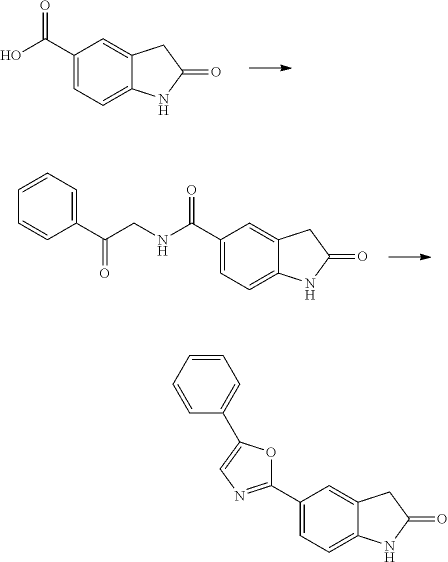 [SMILES: O=C1Cc2cc(C(=O)O)ccc2N1 ; O=C1Cc2cc(C(=O)NCC(=O)c3ccccc3)ccc2N1 ; O=C1Cc2cc(-c3ncc(-c4ccccc4)o3)ccc2N1]

To a solution of 2-oxoindoline-5-carboxylic acid (1.3 g, 7.3 mmol) in DMF (50 ml) was added iPr₂NEt (3.8 ml, 22 mmol), HOBt (1.2 g, 8.8 mmol), WSCI (1.7 g, 8.8 mmol) and 2-amino-1-phenylethanone hydrochloride (1.3 g, 7.3 mmol). The reaction mixture was stirred for 2 h at room temperature. The mixture was poured into H₂O and EtOAc. The resulting precipitate was removed by filtration, and the filtrate was separated. The organic layer was washed with sat. NaHCO₃ solution, sat. NH₄Cl solution and brine, and then dried over Na₂SO₄. The solvent was evaporated and the residue (0.73 g) was used for the next reaction without further purification.

Sulfuric acid (5 ml) was added to the residue, and the mixture was heated for 2 h at 100° C. Ice was added, and the mixture was extracted with EtOAc. The organic layer was washed with H₂O and brine, dried over Na₂SO₄ and evaporated. The residue was crystallized from EtOH to afford 5-(5-phenyloxazol-2-yl)indolin-2-one (0.27 g, 13%).

¹H NMR (300 MHz, DMSO-d₆) δ 10.68 (s, 1H), 7.96-7.91 (m, 2H), 7.84-7.80 (m, 2H), 7.77 (s, 1H), 7.52-7.47 (m, 2H), 7.37 (m, 1H), 6.97 (d, 1H, J=8.0 Hz), 3.60 (s, 2H).

### Reference Example 16

### Production of 5-(2-phenyloxazol-5-yl)indolin-2-one

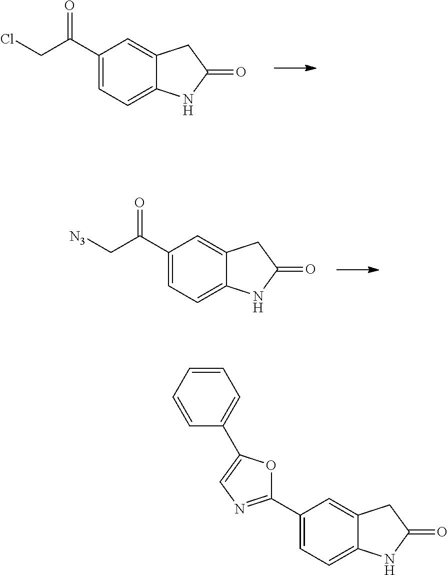 [SMILES: O=C1Cc2cc(C(=O)CCl)ccc2N1 ; [N-]=[N+]=NCC(=O)c1ccc2c(c1)CC(=O)N2 ; O=C1Cc2cc(-c3ncc(-c4ccccc4)o3)ccc2N1]

To a solution of 5-(2-chloroacetyl)indolin-2-one (1.0 g, 4.8 mmol) in DMF (20 ml) was added NaI (0.14 g, 0.96 mmol) and NaN₃ (0.37 g, 5.7 mmol), and the mixture was stirred for 2 h at room temperature. H₂O and EtOAc were added to the mixture, and the resulting precipitate was filtered and dried to afford 5-(2-azidoacetyl)indolin-2-one (0.38 g, 37%).

¹H NMR (300 MHz, DMSO-d₆) δ 10.77 (s, 1H), 7.84 (dd, 1H, J=8.2, 1.6 Hz), 7.79 (d, 1H, J=1.6 Hz), 6.93 (d, 1H, J=8.2 Hz), 4.80 (s, 2H), 3.57 (s, 2H).

To a solution of 5-(2-azidoacetyl)indolin-2-one (0.20 g, 1.1 mmol) in DMF (5 ml) was added 10% Pd—C (0.20 g), and the mixture was stirred for 3.5 h at room temperature under H₂ atmosphere. The mixture was passed through Celite. To the filtrate was added benzoyl chloride (0.12 ml, 1.1 mmol) and iPr₂NEt (0.36 ml, 2.2 mmol), and the reaction mixture was stirred for 1 h at 0° C. H₂O and EtOAc were added to the mixture, and insoluble solid was removed by filtration. The filtrate was separated and the organic layer was washed with H₂O and brine, dried over Na₂SO₄ and evaporated. The residue was dissolved in sulfuric acid (2.0 ml) and the mixture was heated for 2 h at 90° C. The mixture was cooled to room temperature, and H₂O was added. The mixture was extracted with EtOAc, washed with H₂O and brine, dried over Na₂SO₄ and evaporated. Purification by column chromatography (EtOAc/hex) gave 5-(2-phenyloxazol-5-yl)indolin-2-one (0.07 g, 24%).

¹H NMR (400 MHz, DMSO-d₆) δ 10.58 (s, 1H), 8.08-8.05 (m, 2H), 7.72-7.65 (m, 3H), 7.58-7.50 (m, 3H), 6.92 (d, 1H, J=8.1 Hz), 3.57 (s, 2H).

### Reference Example 17

### Production of 5-(3-phenyl-1H-1,2,4-triazol-5-yl)indolin-2-one

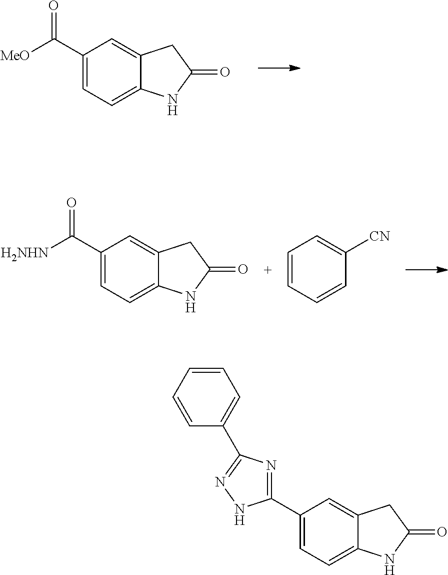

To a solution of methyl 2-oxoindoline-5-carboxylate (0.40 g, 4.8 mmol) in EtOH (8 ml) was added hydrazine monohydrate (2 ml), and the mixture was stirred for 6 h at 80° C. The mixture was cooled to room temperature, and the resulting precipitate was filtered and dried to afford 2-oxoindoline-5-carbohydrazide (0.25 g, 63%).

¹H NMR (400 MHz, DMSO-d6) δ 9.58 (s, 1H), 7.71-7.67 (m, 2H), 6.83 (d, 1H, J=8.0 Hz), 4.41 (br, 2H), 3.51 (s, 2H).

To a solution of 2-oxoindoline-5-carbohydrazide (200 mg, 1.05 mmol) in n-BuOH/DMF (6 ml/2 ml) was added benzonitrile (324 mg, 3.14 mmol) and potassium carbonate (29 mg, 0.21 mmol). The mixture was heated at 150° C. for 3 hours under microwave irradiation. CHCl₃/MeOH (20 ml/1 ml) was added to the mixture and insoluble solid was removed by filtration. The filtrate was concentrated. H₂O was added to the residue and extracted with CHCl₃. The organic layer was dried over Na₂SO₄ and concentrated in vacuo. Purification by column chromatography (CHCl₃/MeOH) gave 5-(3-phenyl-1H-1,2,4-triazol-5-yl)indolin-2-one (15 mg).

LCMS m/z 277.3 (M+H)

### Reference Example 18

### Production of 5-(2-phenyl-1H-imidazol-5-yl)indolin-2-one

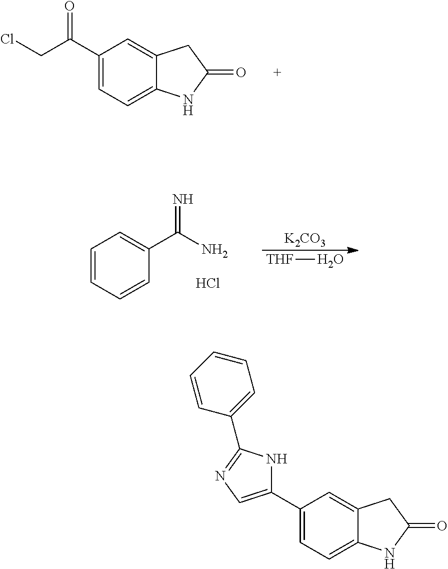

To a solution of 5-(2-chloroacetyl)indolin-2-one (100 mg, 0.477 mmol) in THF/H₂O (3 ml/1 ml) was added benzimidamide hydrochloride (75 mg, 0.477 mmol) and potassium carbonate (198 mg, 1.43 mmol). The mixture was stirred for 7 hours under reflux. The mixture was extracted with CHCl₃, and the organic layer washed with H₂O, dried over Na₂SO₄ and concentrated in vacuo. The residue purified by column chromatography (CHCl₃/MeOH) to give 5-(3-phenyl-1H-imidazol-5-yl)indolin-2-one (19 mg).

LCMS m/z 276.30 (M+H)

### Reference Example 19

### Production of 5-(4-phenyl-1H-1,2,3-triazol-1-yl)indolin-2-one

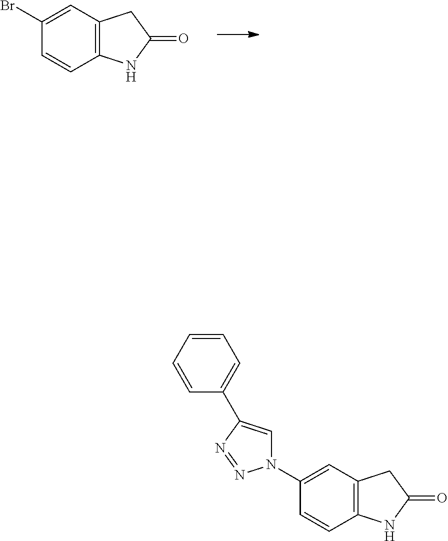 [SMILES: O=C1Cc2cc(Br)ccc2N1 ; O=C1Cc2cc(-n3cc(-c4ccccc4)nn3)ccc2N1]

The mixture of 5-bromoindolin-2-one (530 mg, 2.5 mmol), N,N′-dimethylethylenediamine (44 mg, 0.5 mmol), ethynylbenzene (274 μl, 2.5 mmol), CuI (48 mg, 0.25 mmol), sodium azide (325 mg, 5 mmol) and sodium ascorbate (99 mg, 0.5 mmol) in EtOH (7 ml), H₂O (3 ml) was heated to 80° C. for 18 h. All reagents were re-added and heated to 80° C. for 10 h. After confirming the reaction complete, reaction mixture was cooled to room temperature and EtOH was removed under reduced pressure. 20 ml of water was added and filtered. The filtrate was washed with water and hexane and dried under vacuo to give 5-(4-phenyl-1H-1,2,3-triazol-1-yl)indolin-2-one (450 mg).

### Reference Example 20

### Production of 5-(1-phenyl-1H-1,2,3-triazol-4-yl)indolin-2-one

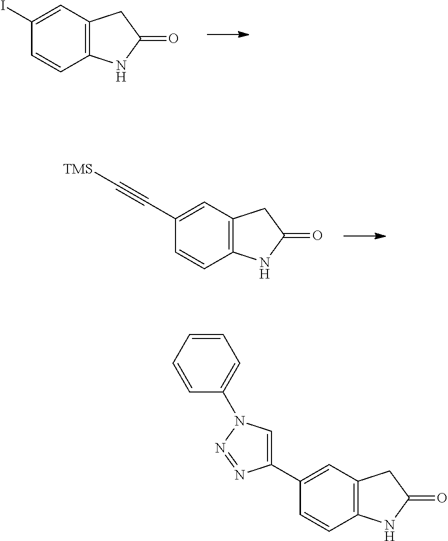 [SMILES: O=C1Cc2cc(I)ccc2N1 ; C[Si](C)(C)C#Cc1ccc2c(c1)CC(=O)N2 ; O=C1Cc2cc(-c3cn(-c4ccccc4)nn3)ccc2N1]

To a solution of 5-iodoindolin-2-one (518 mg, 2 mmol), TEA (3 ml) and CuI (38 mg) in DMF (3 ml) was added to PdCl₂(PPh₃)₂ (70 mg). The mixture was cooled to 0° C. and a solution of TMS-acetylene (1 ml). The mixture was maintained same temperature for 3 h, then warmed to rt. After stirring for overnight, the reaction mixture was concentrated in vacuo. The residue was purified by silica gel column chromatography to give 5-((trimethylsilyl)ethynyl)indolin-2-one (451 mg).

7.37-7.34 (2H, m), 6.80 (1H, d, J=9.0 Hz), 3.51 (2H, s), and 0.24 (9H, s).

To a mixture of iodobenzene (204 mg, 1 mmol), sodium azide (130 mg, 2 mmol), sodium carbonate (53 mg, 0.5 mmol), CuI (19 mg, 0.1 mmol), sodium ascorbate (20 mg) and N,N′-dimethylethylenediamine (18 ul, 0.2 mmol) in EtOH (1.5 ml) and water (0.5 ml) were added 5-((trimethylsilyl)ethynyl)indolin-2-one (115 mg, 0.5 mmol), and stirred at 80° C. for 2 h. After cooling to ambient temp, EtOH was removed under reduced pressure. The residue was suspended in EtOH and stirred for 1 h at rt and filtered. The filtrate was washed with water and hexane and dried under vacuo to give 5-(1-phenyl-1H-1,2,3-triazol-4-yl)indolin-2-one (106 mg).

### Reference Example 21

### Production of 5-(1-phenyl-1H-pyrazol-5-yl)indolin-2-one

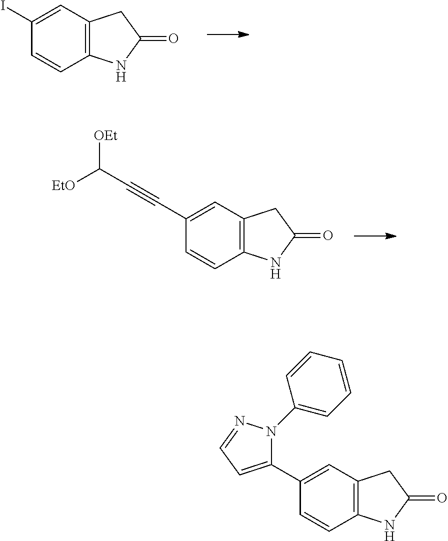 [SMILES: O=C1Cc2cc(I)ccc2N1 ; CCOC(C#Cc1ccc2c(c1)CC(=O)N2)OCC ; O=C1Cc2cc(-c3ccnn3-c3ccccc3)ccc2N1]

To a solution of 5-iodo-2-oxoindoline (497 mg, 1.9 mmol) in THF (20 ml) were added triethylamine (0.80 ml, 5.7 mmol), 3,3-diethoxyprop-1-yne (738 mg, 5.7 mmol), CuI (73 mg, 0.38 mmol) and Pd(PPh₃)₄ (222 mg, 0.19 mmol). The reaction mixture was stirred for 4 h at 50° C. The mixture was poured into H₂O and EtOAc. The mixture was separated into an aqueous layer and an organic layer. The aqueous layer was extracted with ethyl acetate 3 times. The combined organic layer was washed with sat. NaHCO₃ solution, and brine, and then dried over Na₂SO₄. The solvent was evaporated and the residue purified by column chromatography (EtOAc then CHCl₃/MeOH) to give 5-(3,3-diethoxyprop-1-ynyl)indolin-2-one as a brown solid (292 mg, 59%).

¹H NMR (400 MHz, DMSO-d₆) δ 7.73 (s, 1H), 7.19 (d, 1H, J=8.0 Hz), 7.16 (s, 1H), 6.63 (d, 1H, J=8.0 Hz), 5.31 (s, 1H), 3.64 (dq, 2H, J=9.4, 7.1 Hz), 3.48 (dq, 2H, J=9.4, 7.1 Hz), 3.34 (s, 2H), 1.10 (t, 6H, J=7.1 Hz).

To a solution of 5-(3,3-diethoxyprop-1-ynyl)indolin-2-one (100 mg, 0.39 mmol) in acetonitrile (5 ml) were added phenyl hydrazine (38 μL, 0.38 mmol) and sulfuric acid (52 μL, 0.98 mmol), and the mixture was stirred for 3 h at room temperature, then the mixture was stirred for 2 h at 50° C. The reaction mixture was poured into water (50 mL), and the resulting precipitate was filtered and dried. The precipitate was dissolved in acetonitrile (5 mL), then water (52 μL, 3.9 mmol) and sulfuric acid (93 μL, 1.75 mmol) were added. The mixture was heated at 80 C for 4 h. The mixture was cooled to room temperature, and then neutralized with sat. NaHCO₃. The mixture was extracted with CHCl₃/EtOAc 3 times. The combined organic extracts were washed with sat. NaCl, dried over Na₂SO₄, and evaporated in vacuo. The residue purified by column chromatography (EtOAc/n-hexane) to give the title compound as a brown solid (44 mg, 41%).

¹H NMR (400 MHz, CDCl₃) δ 8.28 (brs, 1H), 7.73 (d, 1H, J=1.8 Hz), 7.39-7.28 (m, 5H), 7.13-7.08 (m, 2H), 6.81 (d, 1H, J=8.0 Hz), 6.48 (d, 1H, J=1.8 Hz), 3.51 (s, 2H).

MS m/z 276.3 (M+H)

### Reference Example 22

### Production of 5-(1-(tetrahydro-2H-pyran-4-yl)piperidin-4-yl)indolin-2-one

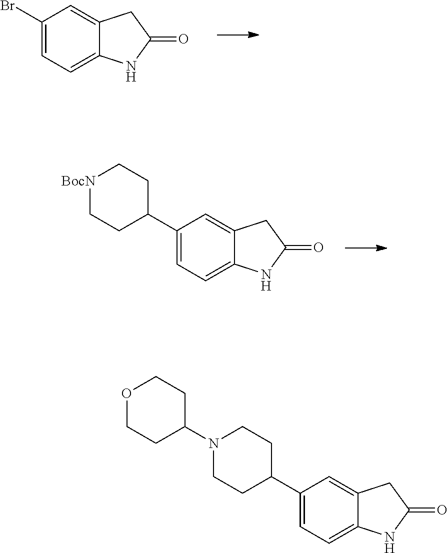 [SMILES: O=C1Cc2cc(Br)ccc2N1 ; CC(C)(C)OC(=O)N1CCC(c2ccc3c(c2)CC(=O)N3)CC1 ; O=C1Cc2cc(C3CCN(C4CCOCC4)CC3)ccc2N1]

To a suspension of 5-bromoindolin-2-one (600 mg, 2.83 mmol) in 1,4-Dioxane (9 ml) and H₂O (3 ml) were added tert-butyl 4-(4,4,5,5-tetramethyl-1,3,2-dioxaborolan-2-yl)-5,6-dihydropyridine-1(2H)-carboxylate (1.05 g, 3.40 mmol), Pd(PPh₃)₄ (164 mg, 0.142 mmol) and K₂CO₃ (1.17 g, 8.50 mmol). After stirring at 120° C. in microwave reactor for 1 h, the reaction mixture was diluted with sat. NaHCO₃ aq. and extracted with CHCl₃. The organic layer was dried over Na₂SO₄ and concentrated. The residue was purified by column chromatography (CHCl₃/MeOH) to give tert-butyl 4-(2-oxoindolin-5-yl)-5,6-dihydropyridine-1(2H)-carboxylate (912 mg) as mixture with triphenylphosphin oxide.

LCMS m/z 315 (M+H)

To a solution of tert-butyl 4-(2-oxoindolin-5-yl)-5,6-dihydropyridine-1(2H)-carboxylate (912 mg, 2.90 mmol) in THF (10 ml) and MeOH (10 ml) was added 10% Pd/C (453 mg) and stirred at room temperature under H₂ (1 atom) atmosphere for 7 h. The reaction mixture was filtered through a Celite pad and concentrated. The residue was purified by column chromatography (CHCl₃/MeOH) to afford tert-butyl 4-(2-oxoindolin-5-yl)piperidine-1-carboxylate (846 mg, 92%).

¹H NMR (300 MHz, DMSO-d₆) δ 10.25 (s, 1H), 7.65-7.49 (m, 1H), 7.07 (s, 1H), 7.00 (d, 1H, J=7.8 Hz), 6.71 (d, 1H, J=7.8 Hz), 4.10-3.96 (m, 2H), 3.41 (s, 2H), 2.86-2.66 (m, 2H), 2.66-2.50 (m, 1H), 1.75-1.62 (m, 2H), 1.51-1.30 (m, 2H), 1.40 (s, 9H).

To a solution of TFA (10 ml) was added tert-butyl 4-(2-oxoindolin-5-yl)piperidine-1-carboxylate (789 mg, 2.49 mmol) and stirred at room temperature for 30 min. The reaction mixture was concentrated. The residue was diluted with 1N HCl and extracted with CHCl₃. The aqueous layer was added with 28% NH₃ aq until pH 8 and extracted with CHCl₃/EtOH (3/1). The organic layer was dried over Na₂SO₄ and concentrated to give 5-(piperidin-4-yl)indolin-2-one (409 mg, 76%).

LCMS m/z 217 (M+H)

To a solution of 5-(piperidin-4-yl)indolin-2-one (64.6 mg, 0.299 mmol) in THF (1.5 ml) and MeOH (3 ml) were added dihydro-2H-pyran-4(3H)-one (0.132 ml, 1.34 mmol), acetic acid (0.170 ml, 29.5 mmol) and NaBH₃(CN) (61.6 mg, 0.931 mmol). After stirring at room temperature for 4 days, the reaction mixture was concentrated. The residue was diluted with sat. NaHCO₃ aq. and extracted with CHCl₃. The organic layer was dried over Na₂SO₄ and concentrated. The residue was purified by column chromatography (CHCl₃/MeOH) to give 5-(1-(tetrahydro-2H-pyran-4-yl)piperidin-4-yl)indolin-2-one (84.9 mg, 95%).

¹H NMR (300 MHz, DMSO-d₆) δ 10.23 (s, 1H), 7.06 (s, 1H), 7.00 (d, 1H, J=7.9 Hz), 6.70 (d, 1H, J=7.9 Hz), 3.89-3.85 (m, 2H), 3.40 (s, 2H), 3.30-3.20 (m, 2H), 2.99-2.92 (m, 2H), 2.50-2.31 (m, 2H), 2.21-2.13 (m, 2H), 1.74-1.62 (m, 4H), 1.61-1.36 (m, 4H).

### Reference Example 23

Reactions and treatments were carried out in the same manner as Reference example 22 using the corresponding starting material compounds, thereby giving the compounds of Reference example 23 shown in Table 3.

TABLE 3

| Reference | | |
|---|---|---|
| Example | Structure | Spectral data |
| 23 | 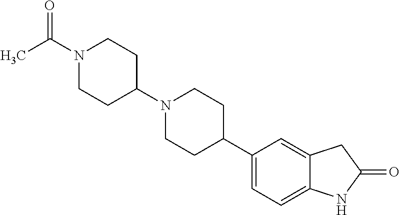 [SMILES: CC(=O)N1CCC(N2CCC(c3ccc4c(c3)CC(=O)N4)CC2)CC1] | 300 MHz ¹H-NMR (DMSO-d₆, δ) 10.24 (s, 1H), 7.06 (s, 1H), 7.00 (d, 1H, J = 8.1 Hz), 6.69 (d, 1H, J = 8.1 Hz), 4.42-4.34 (m, 1H), 3.91-3.77 (m, 2H), 3.72-3.56 (m, 2H), 3.40 (s, 2H), 3.16-3.07 (m, 1H), 3.01-2.86 (m, 4H), 2.45-2.30 (m, 1H), 2.27-2.16 (m, 2H), 1.96 (s, 3H), 1.79-1.45 (m, 3H), 1.45-1.13 (m, 2H). |

### Reference Example 24

### Production of 5-(1-(pyrimidin-2-yl)piperidin-4-yl)indolin-2-one

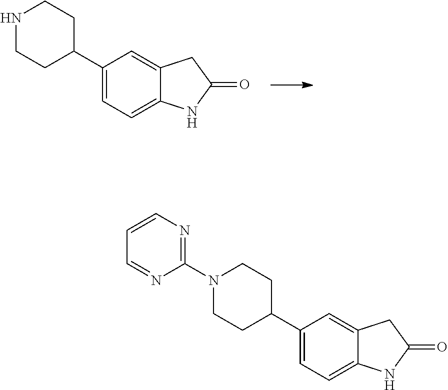 [SMILES: O=C1Cc2cc(C3CCNCC3)ccc2N1 ; O=C1Cc2cc(C3CCN(c4ncccn4)CC3)ccc2N1]

To a solution of 5-(piperidin-4-yl)indolin-2-one (39.8 mg, 0.184 mmol) in EtOH 3 ml) were added 2-chloropyrimidine (33.7 mg, 0.294 mmol) and iPr₂NEt (0.095 ml, 0.551 mmol). After stirring at 80° C. for 6 h, the reaction mixture was concentrated. The residue was purified by column chromatography (CHCl₃/MeOH) to give 5-(1-(pyrimidin-2-yl)piperidin-4-yl)indolin-2-one

(47.9 mg, 88%).

¹H NMR (300 MHz, CDCl₃) δ 8.35 (s, 1H), 8.33 (s, 1H), 7.54 (brs, 1H), 7.08 (s, 1H), 7.04 (d, 1H, J=7.9 Hz), 6.77 (d, 1H, J=7.9 Hz), 6.58-6.47 (m, 1H), 4.99-4.90 (m, 2H), 3.49 (s, 2H), 3.03-2.91 (m, 2H), 2.81-2.69 (m, 1H), 1.98-1.88 (m, 2H), 1.73-1.50 (m, 2H).

### Reference Example 25

### Production of 5-(1-phenylpiperidin-4-yl)indolin-2-one

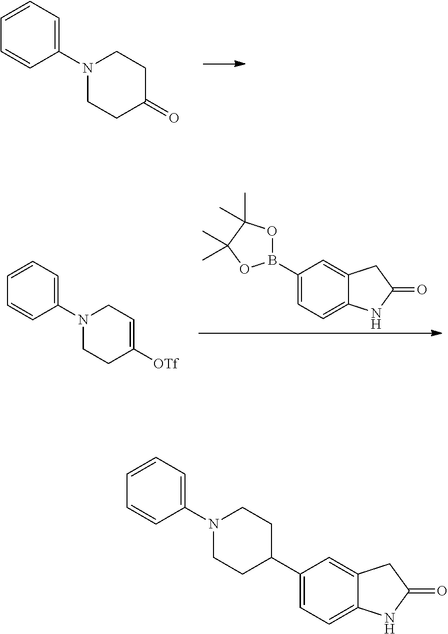 [SMILES: O=C1CCN(c2ccccc2)CC1 ; O=S(=O)(OC1=CCN(c2ccccc2)CC1)C(F)(F)F ; CC1(C)OB(c2ccc3c(c2)CC(=O)N3)OC1(C)C ; O=C1Cc2cc(C3CCN(c4ccccc4)CC3)ccc2N1]

To a solution of LHMDS (3.2 ml, 1.10 M in hexane, 3.52 mmol) in THF (30 ml) was added a solution of 1-phenylpiperidin-4-one (559 mg, 3.19 mmol) in THF (7 ml) at −78° C. over 3 min. After stirring at the same temperature for 30 min, PhNTf₂ (1.48 g, 4.15 mmol) was added. After stirring at −78° C. for 20 min, then the reaction mixture was stirred at 0° C. for 20 min. The reaction mixture was quenched by sat. NH₄Cl aq. and extracted with CHCl₃. The organic layer was dried over Na₂SO₄ and concentrated. The residue was purified by column chromatography (hexane/EtOAc) to give 1-phenyl-1,2,3,6-tetrahydropyridin-4-yl trifluoromethanesulfonate (563 mg, 58%).

¹H NMR (300 MHz, CDCl₃) δ 7.32-7.24 (m, 2H), 6.97-6.87 (m, 3H), 5.90-5.86 (m, 1H), 3.87-3.82 (m, 2H), 3.50 (t, 2H, J=5.6 Hz), 2.62-2.56 (m, 2H).

To a solution of 1-phenyl-1,2,3,6-tetrahydropyridin-4-yl trifluoromethanesulfonate (104 mg, 0.339 mmol) in 1,4-Dioxane (3 ml) and H₂O (1 ml) were added 5-(4,4,5,5-tetramethyl-1,3,2-dioxaborolan-2-yl)indolin-2-one (97.6 mg, 0.377 mmol), Pd(PPh₃)₄ (38.8 mg, 0.00336 mmol), LiCl (47.1 mg, 1.11 mmol) and K₂CO₃ (140 mg, 1.01 mmol). After stirring at 120° C. in microwave reactor for 1 h, the reaction mixture was quenched by sat. NaHCO₃ aq. The resulting mixture was extracted with CHCl₃, the organic layer was washed with brine, dried over Na₂SO₄ and concentrated. The residue was purified by column chromatography (CHCl₃/MeOH) to give 5-(1-phenyl-1,2,3,6-tetrahydropyridin-4-yl)indolin-2-one (75.8 mg) as mixture of triphenylphosphine oxide.

MS m/z 291 (M+H)

To a solution of 5-(1-phenyl-1,2,3,6-tetrahydropyridin-4-yl)indolin-2-one (75.8 mg, 0.261 mmol) in THF (3 ml) and MeOH (3 ml) was added 10% Pd/C (210 mg) and stirred at room temperature under H₂ (1 atom) atmosphere for 2 h. The reaction mixture was filtered through a Celite pad and concentrated. The residue was purified by column chromatography (CHCl₃/MeOH) to afford 5-(1-phenylpiperidin-4-yl)indolin-2-one (51.2 mg) as mixture of triphenylphosphine oxide.

MS m/z 293 (M+H)

### Reference Example 26

### Production of (Z)—N-(2-(diethylamino)ethyl)-2,4-dimethyl-5-((2-oxo-5-(4,4,5,5-tetra methyl-1,3,2-dioxaborolan-2-yl)indolin-3-ylidene)methyl)-1H-pyrrole-3-carboxamide

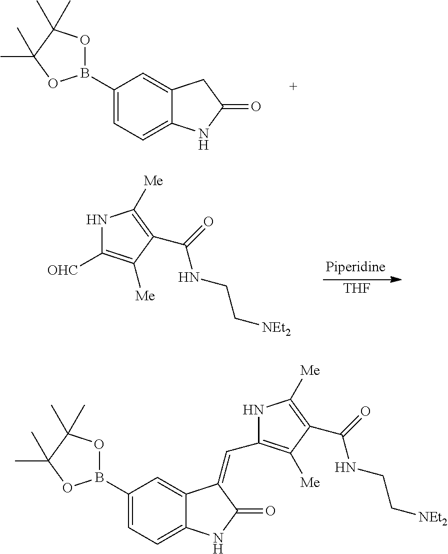

To a solution of 5-(4,4,5,5-tetramethyl-1,3,2-dioxaborolan-2-yl)indolin-2-one (195 mg, 0.75 mmol) in EtOH (3 ml) was added N-(2-(diethylamino)ethyl)-5-formyl-2,4-dimethyl-1H-pyrrole-3-carboxamide (200 mg, 0.76 mmol) and piperidine (82 μL, 0.83 mmol). The mixture was stirred at 80° C. for 1 hour. After cooled down to room temperature, the reaction mixture was concentrated, filtrated, and washed with EtOH to give (Z)—N-(2-(diethylamino)ethyl)-2,4-dimethyl-5-((2-oxo-5-(4,4,5,5-tetramethyl-1,3,2-dioxaborolan-2-yl)indolin-3-ylidene)methyl)-1H-pyrrole-3-carboxamide (218 mg) as yellow solid.

MS m/z 507.6 (M+H)

### Reference Example 27

### Production of (Z)-5-((5-bromo-2-oxoindolin-3-ylidene)methyl)-N-(2-(diethylamino)ethyl)-2,4-dimethyl-1H-pyrrole-3-carboxamide

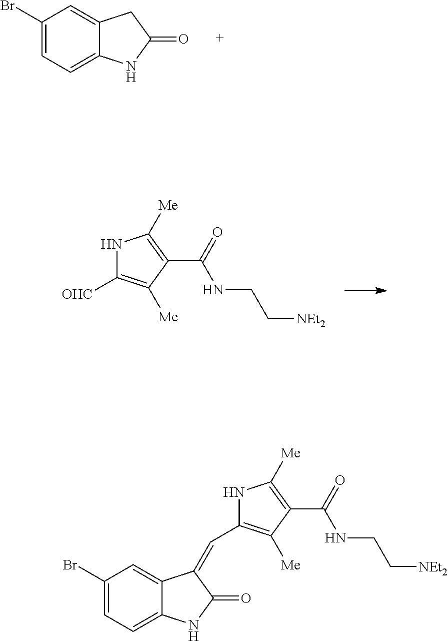

To the solution of 5-bromoindolin-2-one (262 mg, 1.24 mmol) in EtOH (5 ml) was added N-(2-(diethylamino)ethyl)-5-formyl-2,4-dimethyl-1H-pyrrole-3-carboxamide (298 mg, 1.12 mmol) and piperidine (112 μL, 1.13 mmol). The mixture was stirred at 80° C. for 1 hour. After cooled down to room temperature, the reaction mixture was concentrated, filtrated, and washed with EtOH to give (Z)-5-((5-bromo-2-oxoindolin-3-ylidene)methyl)-N-(2-(diethylamino)ethyl)-2,4-dimethyl-1H-pyrrole-3-carboxamide (368 mg) as orange solid.

MS m/z 459.4/461.4 (M+H)

Reactions and treatments were carried out in the same manner as Reference example 21 using the corresponding starting material compounds, thereby giving the compounds of Reference example 28 to 38 shown in Table 4.

TABLE 4

| Reference | | |
|---|---|---|
| Example | Structure | Spectral data |
| 28 | 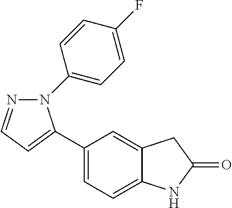 [SMILES: O=C1Cc2cc(-c3ccnn3-c3ccc(F)cc3)ccc2N1] | LCMS m/z 294.09 (M + H) |
| 29 | 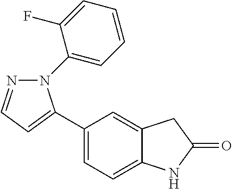 [SMILES: O=C1Cc2cc(-c3ccnn3-c3ccccc3F)ccc2N1] | LCMS m/z 294.04 (M + H) |
| 30 | 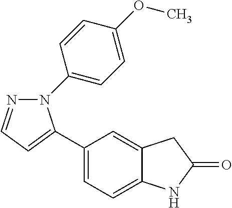 [SMILES: COc1ccc(-n2nccc2-c2ccc3c(c2)CC(=O)N3)cc1] | LCMS m/z 306.14 (M + H) |
| 31 | 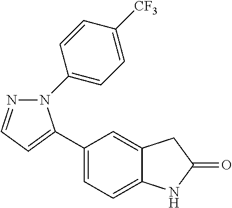 [SMILES: O=C1Cc2cc(-c3ccnn3-c3ccc(C(F)(F)F)cc3)ccc2N1] | LCMS m/z 344.09 (M + H) |
| 32 | 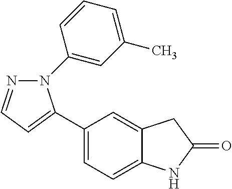 [SMILES: Cc1cccc(-n2nccc2-c2ccc3c(c2)CC(=O)N3)c1] | LCMS m/z 290.18 (M + H) |
| 33 | 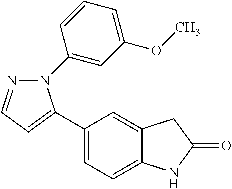 [SMILES: COc1cccc(-n2nccc2-c2ccc3c(c2)CC(=O)N3)c1] | LCMS m/z 306.14 (M + H) |
| 34 | 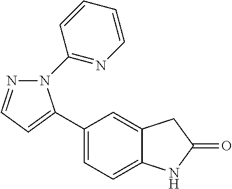 [SMILES: O=C1Cc2cc(-c3ccnn3-c3ccccn3)ccc2N1] | LCMS m/z 277.13 (M + H) |
| 35 | 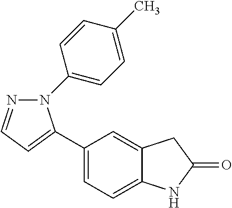 | LCMS m/z 310.09 (M + H) |
| 36 | 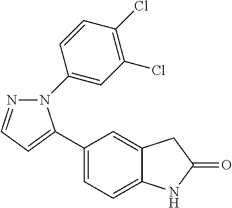 [SMILES: O=C1Cc2cc(-c3ccnn3-c3ccc(Cl)c(Cl)c3)ccc2N1] | LCMS m/z 344.04 (M + H) |
| 37 | 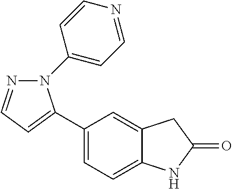 [SMILES: O=C1Cc2cc(-c3ccnn3-c3ccncc3)ccc2N1] | LCMS m/z 277.13 (M + H) |
| 38 | 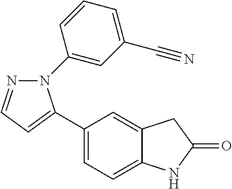 [SMILES: N#Cc1cccc(-n2nccc2-c2ccc3c(c2)CC(=O)N3)c1] | LCMS m/z 301.09 (M + H) |

Reactions and treatments were carried out in the same manner as Reference example 9 using the corresponding starting material compounds, thereby giving the compounds of Reference example 39 to 45 shown in Table 5.

TABLE 5

| Reference | | |
|---|---|---|
| Example | Structure | Spectral data |
| 39 | 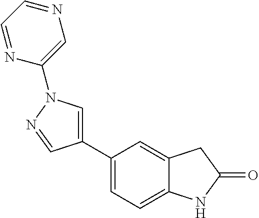 [SMILES: O=C1Cc2cc(-c3cnn(-c4cnccn4)c3)ccc2N1] | LCMS m/z 278.13 (M + H) |
| 40 | 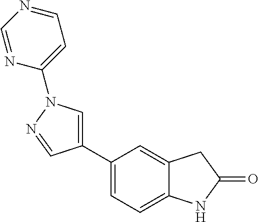 [SMILES: O=C1Cc2cc(-c3cnn(-c4ccncn4)c3)ccc2N1] | LCMS m/z 278.13 (M + H) |
| 41 | 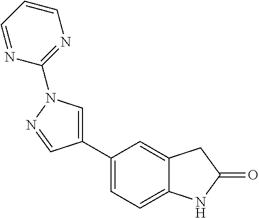 [SMILES: O=C1Cc2cc(-c3cnn(-c4ncccn4)c3)ccc2N1] | LCMS m/z 278.13 (M + H) |
| 42 | 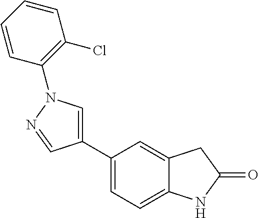 [SMILES: O=C1Cc2cc(-c3cnn(-c4ccccc4Cl)c3)ccc2N1] | 400 MHz ¹H-NMR (CDCl₃, δ) 8.13 (brs, 1H), 8.07 (s, 1H), 7.96 (s, 1H), 7.63 (m, 1H), 7.55 (m, 1H), 7.45- 7.32 (m, 4H), 6.91 (d, 1H, J = 8.0 Hz). |
| 43 | 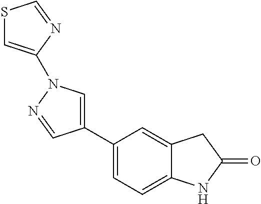 [SMILES: O=C1Cc2cc(-c3cnn(-c4cscn4)c3)ccc2N1] | LCMS m/z 283.08 (M + H) |
| 44 | 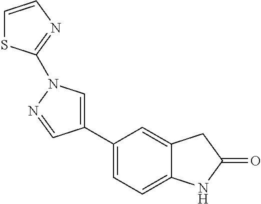 [SMILES: O=C1Cc2cc(-c3cnn(-c4nccs4)c3)ccc2N1] | LCMS m/z 283.08 (M + H) |
| 45 | 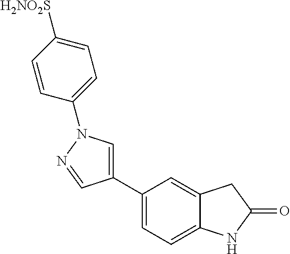 [SMILES: NS(=O)(=O)c1ccc(-n2cc(-c3ccc4c(c3)CC(=O)N4)cn2)cc1] | LCMS m/z 355.10 (M + H) |

### Reference Example 46

### Production of 5-(2-phenylthiazol-4-yl)indolin-2-one

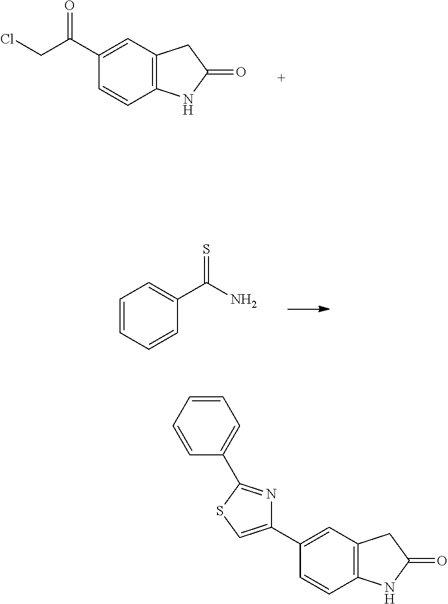 [SMILES: O=C1Cc2cc(C(=O)CCl)ccc2N1 ; NC(=S)c1ccccc1 ; O=C1Cc2cc(-c3csc(-c4ccccc4)n3)ccc2N1]

A suspension of 5-chloroacetyloxindole (838 mg, 4 mmol) and thiobenzamide (550 mg, 4 mmol) in DMF (8 mL) was heated at 70° C. for 16 h and then cooled down to room temperature. At 0° C., while stirring, Na2CO3 aq (1N, 8 mL) was added drop wise to the reaction mixture. The mixture was stirred at room temperature for 20 min, filtrated, and washed with H2O (5 mL×2). The cake was put into a flask and EtOH (5 mL) was added. The mixture was stirred at room temperature for 30 min, filtrated, and washed with EtOH (2 mL×2). The collected solid was dried down under vacuum to yield a light brown solid (1.0 g, 85%).

Reactions and treatments were carried out in the same manner as Reference example 46 using the corresponding starting material compounds, thereby giving the compounds of Reference example 47 to 49 shown in Table 6.

TABLE 6

| Reference | | |
|---|---|---|
| Example | Structure | Spectral data |
| 47 | 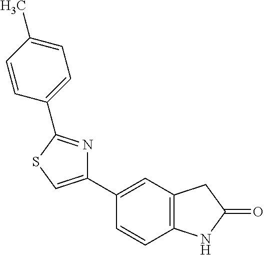 [SMILES: Cc1ccc(-c2nc(-c3ccc4c(c3)CC(=O)N4)cs2)cc1] | LCMS m/z 307.2 (M + H) |
| 48 | 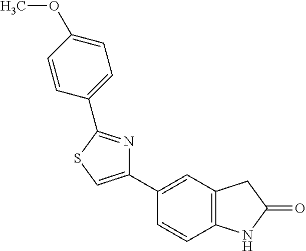 [SMILES: COc1ccc(-c2nc(-c3ccc4c(c3)CC(=O)N4)cs2)cc1] | LCMS m/z 323.2 (M + H) |
| 49 | 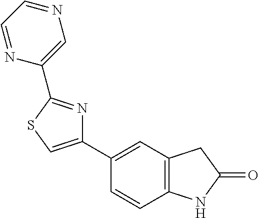 [SMILES: O=C1Cc2cc(-c3csc(-c4cnccn4)n3)ccc2N1] | LCMS m/z 295.2 (M + H) |

### Example 1

### Production of (Z)—N-(2-(diethylamino)ethyl)-2,4-dimethyl-5-((2-oxo-5-(5-phenylthiophen-2-yl)indolin-3-ylidene)methyl)-1H-pyrrole-3-carboxamide 1

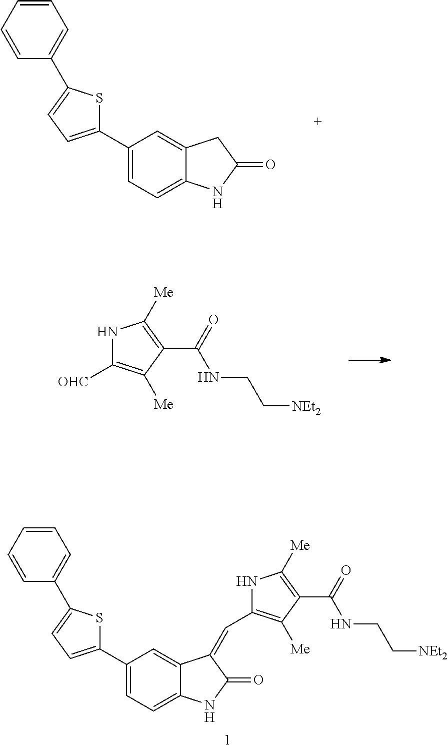

To a solution of 5-(5-phenylthiophen-2-yl)indolin-2-one (23 mg, 0.079 mmol) in THF/EtOH (1 ml/1 ml) was added N-(2-(diethylamino)ethyl)-5-formyl-2,4-dimethyl-1H-pyrrole-3-carboxamide (25.2 mg, 0.095 mmol) and piperidine (0.7 mg, 0.008 mmol). The mixture was stirred at 80° C. for 10 hours. After cooled down to the room temperature, the reaction mixture was concentrated, filtrated, and washed with EtOH to give (Z)—N-(2-(diethylamino)ethyl)-2,4-dimethyl-5-((2-oxo-5-(5-phenylthiophen-2-yl)indolin-3-ylidene)methyl)-1H-pyrrole-3-carboxamide 1 (18 mg) as an orange solid.

¹H NMR (300 MHz, DMSO-d₆) δ 13.67 (s, 1H), 11.02 (s, 1H), 8.31 (s, 1H), 7.81 (s, 1H), 7.70-7.76 (m, 2H), 7.51 (s, 2H), 7.42-7.45 (m, 4H), 7.32-7.39 (m, 1H), 6.90-6.93 (m, 1H), 3.25-3.34 (m, 4H), 2.4-2.6 (m, 10H), 0.94-0.99 (m, 6H); MS m/z 539.70 (M+H).

### Examples 2 to 50

Reactions and treatments were carried out in the same manner as in Example 1 using the corresponding starting material compounds, thereby giving the compounds of Examples 2 to 50 shown in Table 7.

TABLE 7

| Example | Structure | Spectral data |
|---|---|---|
| 2 | 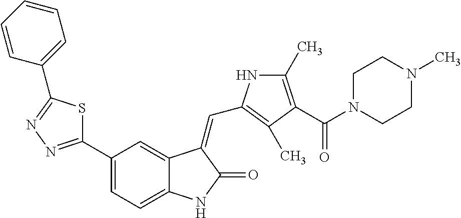 [SMILES: Cc1[nH]c(/C=C2\C(=O)Nc3ccc(-c4nnc(-c5ccccc5)s4)cc32)c(C)c1C(=O)N1CCN(C)CC1] | 300 MHz ¹H-NMR (CDCl₃, δ) 13.29 (s, 1H), 8.48 (s, 1H), 8.19 (d, 1H), 7.99-7.96 (m, 1H), 7.66 (dd, 1H, J = 8.4, 1.8 Hz), 7.48-7.45 (m, 5H), 6.95 (d, 1H, J = 8.1 Hz), 3.74- 3.47 (brs, 4H), 2.38-2.24 (m, 13H) |
| 3 | 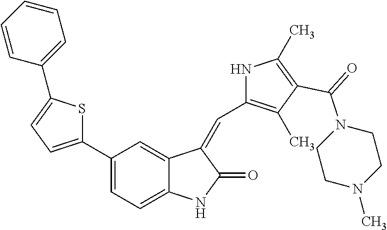 [SMILES: Cc1[nH]c(/C=C2\C(=O)Nc3ccc(-c4ccc(-c5ccccc5)s4)cc32)c(C)c1C(=O)N1CCN(C)CC1] | 300 MHz ¹H-NMR (DMSO-d₆, δ) 13.62 (s, 1H), 11.02 (s, 1H), 8.17 (s, 1H), 7.80 (s, 1H), 7.70-7.68 (m, 2H), 7.55-7.51 (m, 2H), 7.45-7.41 (m, 3H), 7.32-7.29 (m, 1H), 6.94- 6.92 (m, 1H), 3.47-3.42 (m, 4H), 2.49 (s, 3H), 2.32 (s, 3H), 2.30- 2.27 (m, 4H), 2.19 (s, 3H) |
| 4 | 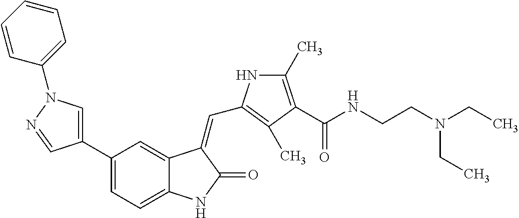 [SMILES: CCN(CC)CCNC(=O)c1c(C)[nH]c(/C=C2\C(=O)Nc3ccc(-c4cnn(-c5ccccc5)c4)cc32)c1C] | 300 MHz ¹H-NMR (CDCl₃, δ) 13.29 (s, 1H), 8.53 (s, 1H), 7.67 (d, 2H, J = 8.4 Hz), 7.50 (s, 1H), 7.40 (t, 2H, J = 7.8 Hz), 7.30-7.23 (m, 3H), 6.83 (d, 1H, J = 7.8 Hz), 6.70 (brs, 1H), 3.48 (d, 2H, J = 5.4 Hz), 2.68- 2.57 (m, 6H), 2.49 (s, 3H), 2.39 (s, 3H), 1.02 (t, 6H, J = 6.9 Hz) |
| 5 | 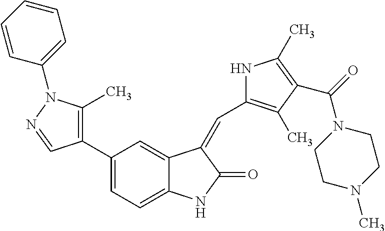 [SMILES: Cc1[nH]c(/C=C2\C(=O)Nc3ccc(-c4cnn(-c5ccccc5)c4C)cc32)c(C)c1C(=O)N1CCN(C)CC1] | 300 MHz ¹H-NMR (CDCl₃, δ) 13.63 (s, 1H), 8.14 (s, 1H), 7.78 (s, 1H), 7.45-7.42 (m, 4H), 7.40- 7.36 (m, 2H), 7.24-7.17 (m, 2H), 6.95-6.92 (m, 1H), 3.74-3.72 (m, 4H), 2.63-2.56 (m, 4H), 2.42 (s, 3H), 2.38 (s, 3H), 2.32 (s, 3H), 2.30 (s, 3H) |
| 6 | 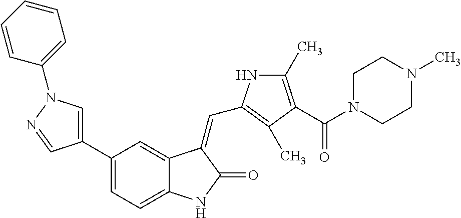 [SMILES: Cc1[nH]c(/C=C2\C(=O)Nc3ccc(-c4cnn(-c5ccccc5)c4)cc32)c(C)c1C(=O)N1CCN(C)CC1] | 300 MHz ¹H-NMR (CDCl₃, δ) 13.34 (s, 1H), 8.15 (s, 1H), 7.98 (s, 1H), 7.73 (d, 2H, J = 7.5 Hz), 7.60 (s, 1H), 7.46-7.41 (m, 3H), 7.32 (t, 2H, J = 7.5 Hz), 6.90 (d, 1H, J = 7.8 Hz), 3.69 (brs, 4H), 2.42-2.33 (m, 14H) |
| 7 | 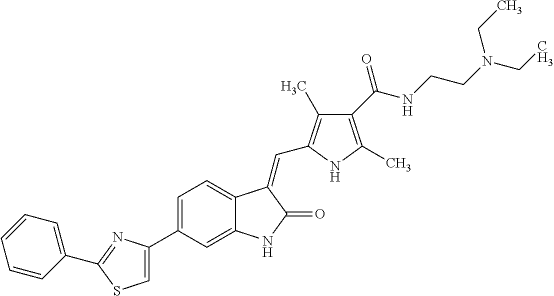 [SMILES: CCN(CC)CCNC(=O)c1c(C)[nH]c(/C=C2\C(=O)Nc3cc(-c4csc(-c5ccccc5)n4)ccc32)c1C] | 300 MHz ¹H-NMR (DMSO-d₆, δ) 13.63 (s, 1H), 11.01 (s, 1H), 8.15 (s, 1H), 8.05-8.00 (m, 2H), 7.87 (d, 1H, J = 8.1 Hz), 7.72-7.68 (m, 2H), 7.58- 7.50 (m, 2H), 7.46-7.41 (m, 1H), 3.38-3.25 (m, 4H), 2.60-2.47 (m, 4H), 2.45 (s, 3H), 2.43 (s, 3H), 0.98 (t, 6H, J = 7.0 Hz) |
| 8 | 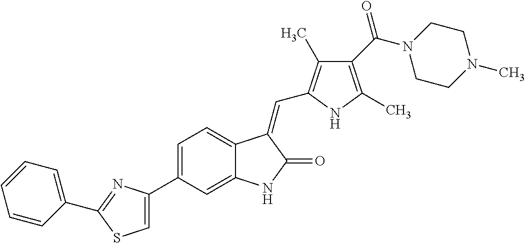 [SMILES: Cc1[nH]c(/C=C2\C(=O)Nc3cc(-c4csc(-c5ccccc5)n4)ccc32)c(C)c1C(=O)N1CCN(C)CC1] | 300 MHz ¹H-NMR (DMSO-d₆, δ) 13.57 (s, 1H), 11.01 (s, 1H), 8.15 (s, 1H), 8.04-8.00 (m, 2H), 7.86 (d, 1H, J = 8.1 Hz), 7.70 (dd, 1H, J = 1.5, 6.6 Hz), 7.66 (s, 1H), 7.58-7.51 (m, 5H), 3.55-3.35 (m, 4H), 2.37- 2.23 (m, 4H), 2.29 (s, 3H), 2.27 (s, 3H), 2.18 (s, 3H) |
| 9 | 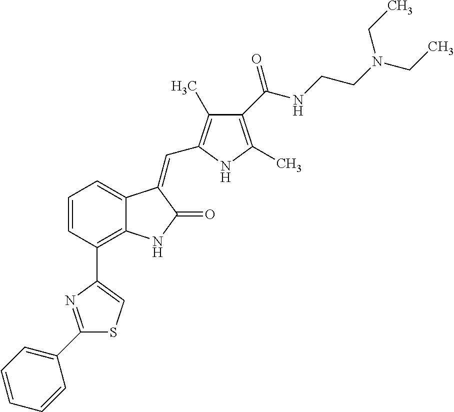 [SMILES: CCN(CC)CCNC(=O)c1c(C)[nH]c(/C=C2\C(=O)Nc3c2cccc3-c2csc(-c3ccccc3)n2)c1C] | 300 MHz ¹H-NMR (DMSO-d₆, δ) 13.65 (s, 1H), 10.41 (s, 1H), 8.24 (s, 1H), 8.08-8.04 (m, 2H), 7.87 (d, 1H, J = 7.5 Hz), 7.75 (s, 1H), 7.72 (d, 1H, J = 8.3 Hz), 7.62-7.54 (m, 3H), 7.47- 7.43 (m, 1H), 7.14 (dd, 1H, J = 7.7, 7.9 Hz), 3.36-3.23 (m, 4H), 2.56- 2.45 (m, 4H), 2.46 (s, 3H), 2.44 (s, 3H), 0.97 (t, 6H, J = 7.1 Hz) |
| 10 | 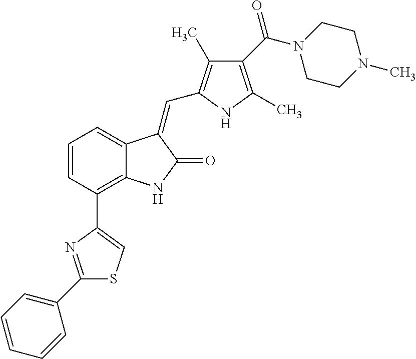 [SMILES: Cc1[nH]c(/C=C2\C(=O)Nc3c2cccc3-c2csc(-c3ccccc3)n2)c(C)c1C(=O)N1CCN(C)CC1] | 300 MHz ¹H-NMR (DMSO-d₆, δ) 13.60 (s, 1H), 10.41 (s, 1H), 8.25 (s, 1H), 8.08-8.05 (m, 2H), 7.86 (d, 1H, J = 7.7 Hz), 7.73 (s. 1H), 7.71 (d, 1H, J = 8.8 Hz), 7.62-7.54 (m, 3H), 7.14 (t, 1H, J = 7.6 Hz), 3.57- 3.37 (m, 4H), 2.32-2.25 (m, 4H), 2.30 (s, 3H), 2.28 (s, 3H), 2.18 (s, 3H) |
| 11 | 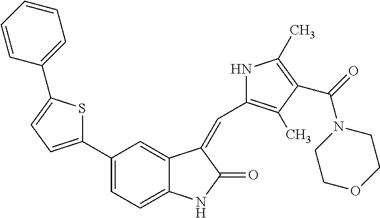 [SMILES: Cc1[nH]c(/C=C2\C(=O)Nc3ccc(-c4ccc(-c5ccccc5)s4)cc32)c(C)c1C(=O)N1CCOCC1] | 300 MHz ¹H-NMR (DMSO-d₆, δ) 13.62 (s, 1H), 11.02 (s, 1H), 8.17 (s, 1H), 7.80 (s, 1H), 7.70-7.67 (m, 2H), 7.53-7.49 (m, 2H), 7.49-7.42 (m, 3H), 7.32-7.30 (m, 1H), 6.94- 6.91 (m, 1H), 3.60-3.30 (m, 8H), 2.30 (s, 3H), 2.28 (s, 3H) |
| 12 | 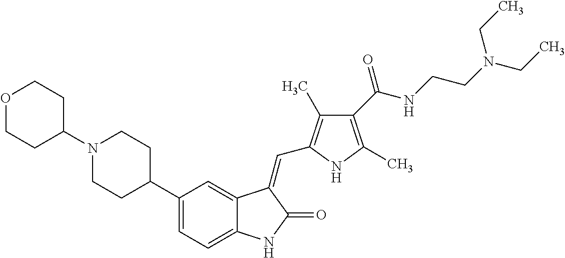 [SMILES: CCN(CC)CCNC(=O)c1c(C)[nH]c(/C=C2\C(=O)Nc3ccc(C4CCN(C5CCOCC5)CC4)cc32)c1C] | 300 MHz ¹H-NMR (DMSO-d₆, δ) 13.66 (s, 1H), 10.77 (s, 1H), 7.75 (s, 1H), 7.67 (s, 1H), 7.38 (t, 1H, J = 5.5 Hz), 6.96 (brd, 1H, J = 7.9 Hz), 6.75 (d, 1H, J = 7.9 Hz), 3.92- 3.85 (m, 2H), 3.34-3.22 (m, 6H), 3.03-2.97 (m, 2H), 2.57-2.39 (m, 6H), 2.42 (s, 3H), 2.42 (s, 3H), 2.23-2.12 (m 2H), 1.76-1.65 (m, 6H), 1.52-1.36 (m, 2H), 0.97 (t, 6H, J = 7.2 Hz) |
| 13 | 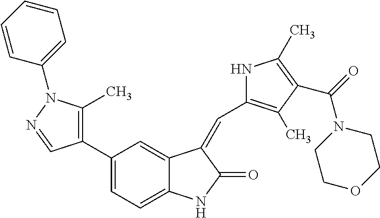 [SMILES: Cc1[nH]c(/C=C2\C(=O)Nc3ccc(-c4cnn(-c5ccccc5)c4C)cc32)c(C)c1C(=O)N1CCOCC1] | 300 MHz ¹H-NMR (CDCl₃, δ) 13.35 (s, 1H), 8.14-8.11 (m, 1H), 7.74 (s, 1H), 7.51-7.45 (m, 4H), 7.42-7.38 (m, 2H), 7.29-7.21 (m, 2H), 6.96-6.90 (m, 1H), 3.80- 3.40 (m, 8H), 2.43 (s, 3H), 2.40 (s, 3H), 2.24 (s, 3H) |
| 14 | 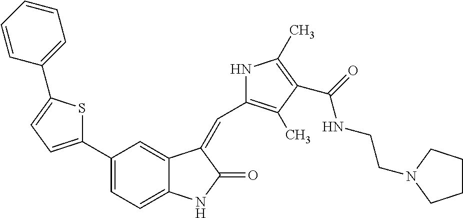 [SMILES: Cc1[nH]c(/C=C2\C(=O)Nc3ccc(-c4ccc(-c5ccccc5)s4)cc32)c(C)c1C(=O)NCCN1CCCC1] | 300 MHz ¹H-NMR (DMSO-d₆, δ) 13.66 (s, 1H), 11.02 (s, 1H), 8.19 (s, 1H), 7.81 (s, 1H), 7.70-7.67 (m, 2H), 7.45-7.42 (m, 2H), 7.40- 7.32 (m, 3H), 7.30-7.27 (m, 1H), 6.93-6.91 (m, 1H), 3.60-3.30 (m, 4H), 2.71-2.65 (m, 4H), 2.50 (s, 3H), 2.49 (s, 3H), 1.70-1.60 (m, 4H) |
| 15 | 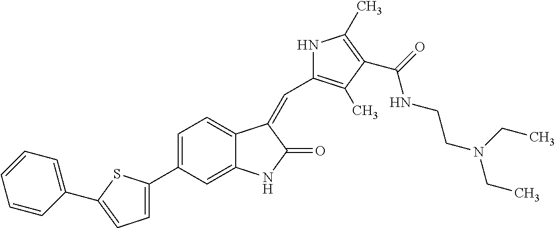 [SMILES: CCN(CC)CCNC(=O)c1c(C)[nH]c(/C=C2\C(=O)Nc3cc(-c4ccc(-c5ccccc5)s4)ccc32)c1C] | LCMS m/z 539.70 (M + H) |
| 16 | 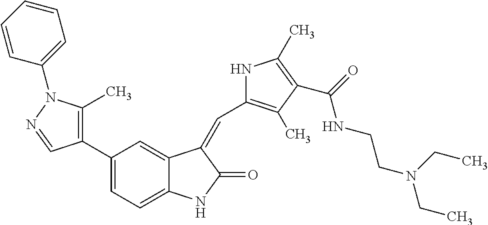 [SMILES: CCN(CC)CCNC(=O)c1c(C)[nH]c(/C=C2\C(=O)Nc3ccc(-c4cnn(-c5ccccc5)c4C)cc32)c1C] | 300 MHz ¹H-NMR (CDCl₃, δ) 13.37 (s, 1H), 8.70 (s, 1H), 7.77 (s, 1H), 7.49-7.47 (m, 4H), 7.40- 7.36 (m, 2H), 7.20-7.17 (m, 1H), 6.94-6.85 (m, 2H), 3.58-3.56 (m, 2H), 2.78-2.71 (m, 6H), 2.56 (s, 3H), 2.42 (s, 3H), 2.41 (s, 3H), 1.12-1.08 (m, 6H) |
| 17 | 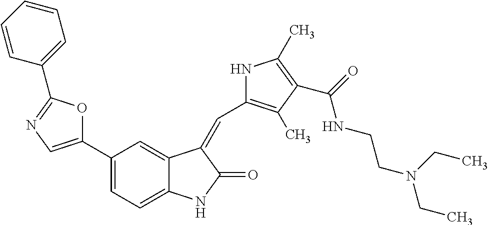 [SMILES: CCN(CC)CCNC(=O)c1c(C)[nH]c(/C=C2\C(=O)Nc3ccc(-c4cnc(-c5ccccc5)o4)cc32)c1C] | 400 MHz ¹H-NMR (DMSO-d₆, δ) 13.67 (s, 1H), 11.11 (br, 1H), 8.29 (d, 1H, J = 1.5 Hz), 8.15- 8.10 (m, 2H), 7.83 (s, 1H), 7.73 (s, 1H), 7.65-7.43 (m, 5H), 7.00 (d, 1H, J = 8.1 Hz), 3.35-3.26 (m, 4H), 2.58- 2.45 (m, 10H), 0.98 (t, 6H, J = 7.1 Hz) |
| 18 | 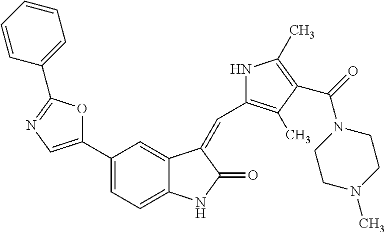 [SMILES: Cc1[nH]c(/C=C2\C(=O)Nc3ccc(-c4cnc(-c5ccccc5)o4)cc32)c(C)c1C(=O)N1CCN(C)CC1] | 400 MHz ¹H-NMR (DMSO-d₆, δ) 13.61 (s, 1H), 11.10 (br, 1H), 7.81 (s, 1H), 7.73 (s, 1H), 7.65-7.51 (m, 4H), 7.00 (d, 1H, J = 8.1 Hz), 3.7-3.3 (m, 4H), 2.35-2.22 (m, 4H), 2.34 (s, 3H), 2.31 (s, 3H), 2.19 (s, 3H) |
| 19 | 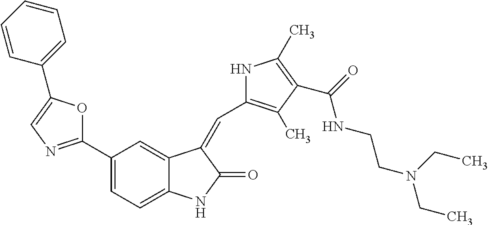 [SMILES: CCN(CC)CCNC(=O)c1c(C)[nH]c(/C=C2\C(=O)Nc3ccc(-c4ncc(-c5ccccc5)o4)cc32)c1C] | 400 MHz ¹H-NMR (CDCl₃, δ) 13.35 (s, 1H), 8.36 (br, 1H), 8.20 (d, 1H, J = 1.4 Hz), 7.93 (dd, 1H, J = 8.1, 1.4 Hz), 7.75-7.70 (m, 2H), 7.52 (s, 1H), 7.48-7.30 (m, 4H), 6.99 (d, 1H, J = 8.1 Hz), 6.62 (br, 1H), 3.52 (m, 2H), 2.70 (t, 2H, J = 5.8 Hz), 2.62 (q, 4H, J = 7.1 Hz), 2.59 (s, 3H), 2.48 (s, 3H), 1.06 (t, 6H, J = 7.1 Hz) |
| 20 | 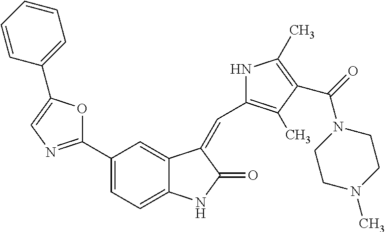 [SMILES: Cc1[nH]c(/C=C2\C(=O)Nc3ccc(-c4ncc(-c5ccccc5)o4)cc32)c(C)c1C(=O)N1CCN(C)CC1] | 400 MHz ¹H-NMR (CDCl₃, δ) 13.32 (s, 1H), 8.22 (d, 1H, J = 1.6 Hz), 8.14 (br, 1H), 7.93 (dd, 1H, J = 8.1, 1.6 Hz), 7.76-7.72 (m, 2H), 7.52 (s, 1H), 7.49-7.32 (m, 4H), 7.00 (d, 1H, J = 8.1 Hz), 4.0-3.3 (m, 4H), 2.6-2.3 (m, 4H), 2.40 (s, 3H), 2.34 (s, 3H), 2.33 (s, 3H) |
| 21 | 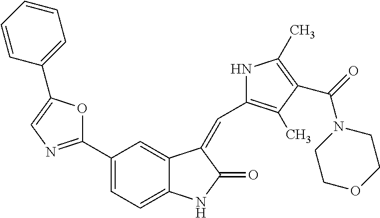 [SMILES: Cc1[nH]c(/C=C2\C(=O)Nc3ccc(-c4ncc(-c5ccccc5)o4)cc32)c(C)c1C(=O)N1CCOCC1] | 400 MHz ¹H-NMR (CDCl₃, δ) 13.34 (s, 1H), 8.24 (br, 1H), 7.96- 7.91 (m, 2H), 7.77-7.72 (m, 2H), 7.53 (s, 1H), 7.49-7.32 (m, 4H), 7.00 (d, 1H, J = 8.1 Hz), 4.0-3.3 (m, 8H), 2.41 (s, 3H), 2.36 (s, 3H) |
| 22 | 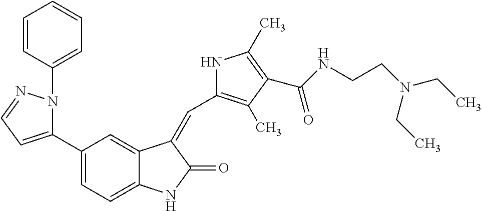 [SMILES: CCN(CC)CCNC(=O)c1c(C)[nH]c(/C=C2\C(=O)Nc3ccc(-c4ccnn4-c4ccccc4)cc32)c1C] | 400 MHz ¹H-NMR (CDCl₃, δ) 13.26 (s, 1H), 7.69 (s, 1H), 7.67 (d, 1H, J = 1.8 Hz), 7.30-7.23 (m, 6H), 7.17 (s, 1H), 6.90 (dd, 1H, J = 1.6, 8.1 Hz), 6.73 (d, 1H, J = 8.1 Hz), 6.47 (d, 1H, J = 1.8 Hz), 3.55- 3.40 (m, 2H), 2.73-2.47 (m, 6H), 2.53 (s, 3H), 2.39 (s, 3H), 1.09- 0.91 (m, 6H). |
| 23 | 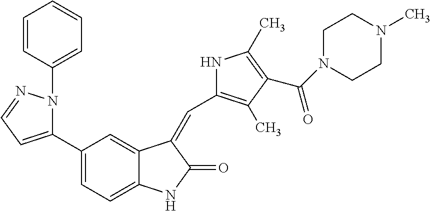 [SMILES: Cc1[nH]c(/C=C2\C(=O)Nc3ccc(-c4ccnn4-c4ccccc4)cc32)c(C)c1C(=O)N1CCN(C)CC1] | 400 MHz ¹H-NMR (CDCl₃, δ) 13.27 (s, 1H), 8.05 (s, 1H), 7.76 (d, 1H, J = 1.8 Hz), 7.39-7.31 (m, 6H), 7.21 (s, 1H), 6.99 (dd, 1H, J = 1.6, 8.1 Hz), 6.81 (d, 1H, J = 8.1 Hz), 6.55 (d, 1H, J = 1.8 Hz), 3.95- 3.37 (br, 4H), 2.56-2.30 (m, 4H), 2.41 (s, 3H), 2.35 (s, 3H), 2.27 (s, 3H). |
| 24 | 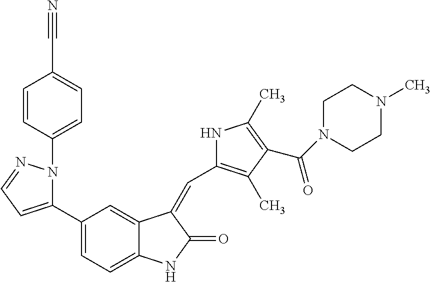 [SMILES: Cc1[nH]c(/C=C2\C(=O)Nc3ccc(-c4ccnn4-c4ccc(C#N)cc4)cc32)c(C)c1C(=O)N1CCN(C)CC1] | 400 MHz ¹H-NMR (CDCl₃, δ) 13.22 (s, 1H), 7.84 (s, 1H), 7.71 (d, 1H, J = 1.8 Hz), 7.55 (dt, 2H, J = 1.9, 8.7 Hz), 7.41 (dt, 2H, J = 1.9, 8.7 Hz), 7.35 (1H, d, J = 1.5 Hz), 7.22 (s, 1H), 6.84 (dd, 1H, J = 1.5, 8.0 Hz), 6.78 (d, 1H, J = 8.0 Hz), 6.48 (d, 1H, J = 1.8 Hz), 3.90- 3.30 (br, 4H), 2.47-2.20 (m, 4H), 2.33 (s, 3H), 2.27 (s, 3H), 2.17 (s, 3H). |
| 25 | 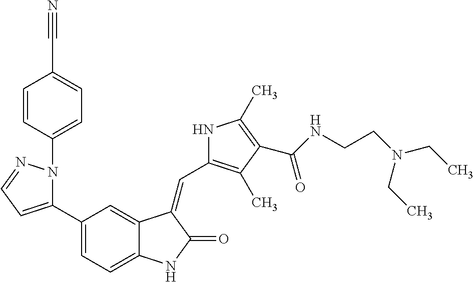 [SMILES: CCN(CC)CCNC(=O)c1c(C)[nH]c(/C=C2\C(=O)Nc3ccc(-c4ccnn4-c4ccc(C#N)cc4)cc32)c1C] | 400 MHz ¹H-NMR (CDCl₃, δ) 13.30 (s, 1H), 7.96 (s, 1H), 7.71 (d, 1H, J = 1.8 Hz), 7.55 (dt, 2H, J = 2.1, 8.7 Hz), 7.41 (dt, 2H, J = 2.1, 8.7 Hz), 7.36 (1H, d, J = 1.5 Hz), 7.26 (s, 1H), 6.83 (dd, 1H, J = 1.5, 8.0 Hz), 6.78 (d, 1H, J = 8.0 Hz), 6.48 (d, 1H, J = 1.8 Hz), 3.47- 3.40 (m, 2H), 2.65-2.49 (m, 6H), 2.53 (s, 3H), 2.38 (s, 3H), 0.98 (t, 6H, J = 6.9 Hz). |
| 26 | 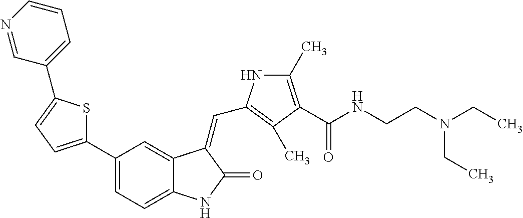 [SMILES: CCN(CC)CCNC(=O)c1c(C)[nH]c(/C=C2\C(=O)Nc3ccc(-c4ccc(-c5cccnc5)s4)cc32)c1C] | 300 MHz ¹H-NMR (CDCl₃, δ) 13.38 (s, 1H), 8.86 (s, 1H), 8.48 (d, 1H, J = 4.5 Hz), 8.21 (s, 1H), 7.84 (d, 1H, J = 7.5 Hz), 7.63 (s, 1H), 7.43- 7.34 (m, 2H), 7.30-7.25 (m, 2H), 6.92 (d, 1H, J = 8.1 Hz), 3.72 (brs, 2H), 3.03-2.92 (m, 6H), 2.59 (s, 3H), 2.52 (s, 3H), 1.26 (d, 6H, J = 5.1 Hz) |
| 27 | 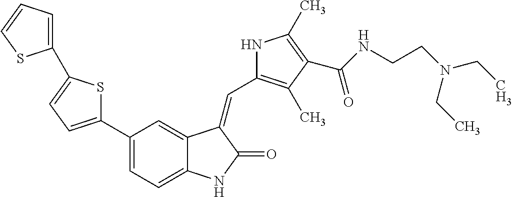 [SMILES: CCN(CC)CCNC(=O)c1c(C)[nH]c(/C=C2\C(=O)Nc3ccc(-c4ccc(-c5cccs5)s4)cc32)c1C] | 400 MHz ¹H-NMR (CDCl₃, δ) 13.33 (s, 1H), 7.80 (s, 1H), 7.60 (d, 1H, J = 1.6 Hz), 7.40 (s, 1H), 7.35 (dd, 1H, J = 1.7, 8.1 Hz), 7.16- 7.12 (m, 3H), 7.08 (d, 1H, J = 3.7 Hz), 6.97 (dd, 1H, J = 3.7, 5.1 Hz), 6.84 (d, 1H, J = 8.1 Hz), 3.69- 3.63 (m, 2H), 3.54-3.40 (m, 2H), 2.72-2.49 (m, 4H), 2.54 (s, 3H), 2.47 (s, 3H), 1.08-0.95 (m, 6H). |
| 28 | 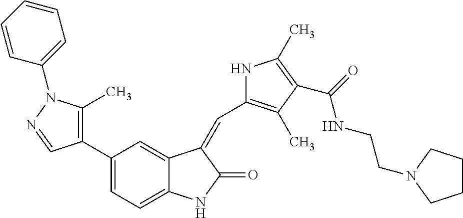 [SMILES: Cc1[nH]c(/C=C2\C(=O)Nc3ccc(-c4cnn(-c5ccccc5)c4C)cc32)c(C)c1C(=O)NCCN1CCCC1] | 300 MHz ¹H-NMR (CDCl₃, δ) 13.22 (s, 1H), 9.63 (s, 1H), 7.76 (s, 1H), 7.46-7.37 (m, 5H), 7.19- 7.11 (m, 3H), 6.90-6.88 (m, 1H), 3.69-3.60 (m, 2H), 3.05-2.80 (m, 6H), 2.51 (s, 3H), 2.41 (s, 3H), 2.21 (s, 3H), 2.02-1.91 (m, 4H) |
| 29 | 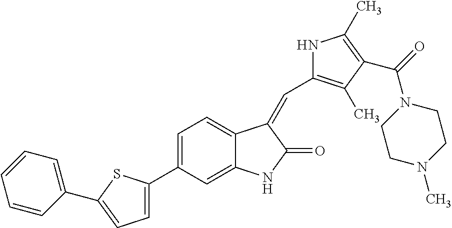 [SMILES: Cc1[nH]c(/C=C2\C(=O)Nc3cc(-c4ccc(-c5ccccc5)s4)ccc32)c(C)c1C(=O)N1CCN(C)CC1] | LCMS m/z 523.66 (M + H) |
| 30 | 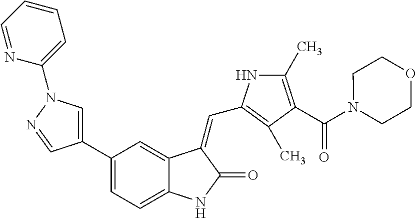 [SMILES: Cc1[nH]c(/C=C2\C(=O)Nc3ccc(-c4cnn(-c5ccccn5)c4)cc32)c(C)c1C(=O)N1CCOCC1] | 300 MHz ¹H-NMR (CDCl₃, δ) 13.34 (s, 1H), 8.82 (s, 1H), 8.43 (d, 1H, J = 4.2 Hz), 8.01 (t, 2H, J = 3.6 Hz), 7.86-7.81 (m, 2H), 7.67 (s, 1H), 7.42-7.37 (m, 2H), 7.20 (t, 1H, J = 6.3 Hz), 6.91 (d, 1H, J = 7.8 Hz), 3.69 (brs, 8H), 2.89 (s, 3H), 2.36 (s, 3H) |
| 31 | 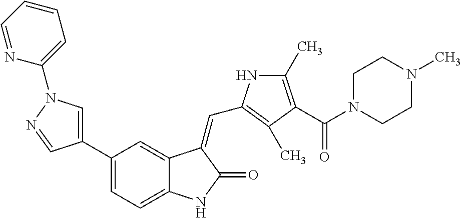 [SMILES: Cc1[nH]c(/C=C2\C(=O)Nc3ccc(-c4cnn(-c5ccccn5)c4)cc32)c(C)c1C(=O)N1CCN(C)CC1] | 300 MHz ¹H-NMR (DMSO₆, δ) 13.61 (s, 1H), 10.93 (s, 1H), 9.09 (s, 1H), 8.50 (d, 1H, J = 4.8 Hz), 8.32 (s, 1H), 8.25 (s, 1H), 8.00-7.95 (m, 2H), 7.78 (s, 1H), 7.54 (d, 1H, J = 8.4 Hz), 7.37 (t, 1H, J = 5.4 Hz), 6.89 (d, 1H, J = 7.8 Hz), 3.47 (brs, 4H), 2.32- 2.26 (m, 13H) |
| 32 | 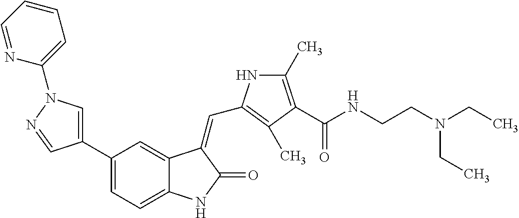 [SMILES: CCN(CC)CCNC(=O)c1c(C)[nH]c(/C=C2\C(=O)Nc3ccc(-c4cnn(-c5ccccn5)c4)cc32)c1C] | 300 MHz ¹H-NMR (CDCl₃, δ) 13.38 (s, 1H), 8.79 (s, 1H), 8.42 (d, 1H, J = 3.6 Hz), 8.11 (s, 1H), 8.00- 7.97 (m, 2H), 7.81 (t, 1H, J = 6.9 Hz), 7.64 (s, 1H), 7.42 (s, 1H), 7.19 (d, 1H, J = 6.6 Hz), 6.91 (d, 1H, J = 7.8 Hz), 3.56 (brs, 4H), 2.79-2.70 (m, 6H), 2.58 (s, 3H), 2.51 (s, 3H), |
| 33 | 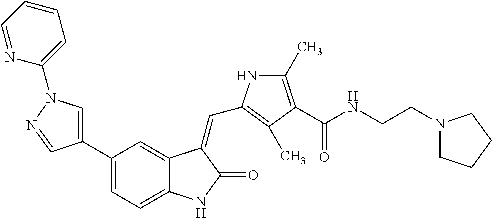 [SMILES: Cc1[nH]c(/C=C2\C(=O)Nc3ccc(-c4cnn(-c5ccccn5)c4)cc32)c(C)c1C(=O)NCCN1CCCC1] | 300 MHz ¹H-NMR (CDCl₃, δ) 13.34 (s, 1H), 8.78 (s, 1H), 8.40 (d, 1H, J = 3.9 Hz), 8.30 (s, 1H), 7.99- 7.95 (m, 2H), 7.80 (t, 1H, J = 7.5 Hz), 7.62 (s, 1H), 7.38-7.35 (m, 2H), 7.18 (d, 1H, J = 7.2 Hz), 6.89 (d, 1H, J = 7.8 Hz), 3.70 (brs, 2H), 3.02 (bs, 6H), 2.56 (s, 3H), 2.47 (s, 3H), 1.96 (brs, 4H) |
| 34 | 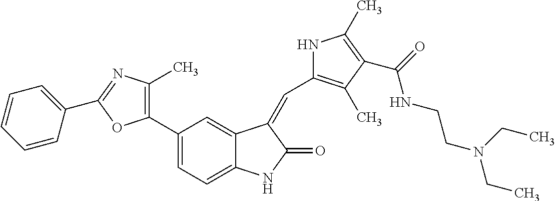 [SMILES: CCN(CC)CCNC(=O)c1c(C)[nH]c(/C=C2\C(=O)Nc3ccc(-c4oc(-c5ccccc5)nc4C)cc32)c1C] | 300 MHz ¹H-NMR (DMSO-d₆, δ) 13.71 (s, 1H), 11.09 (s, 1H), 8.10- 8.07 (m, 3H), 7.82 (s, 1H), 7.53- 7.45 (m, 5H), 7.03-7.00 (m, 1H), 3.34-3.27 (m, 7H), 2.45-2.39 (m, 10H), 0.99-0.94 (m, 6H) |
| 35 | 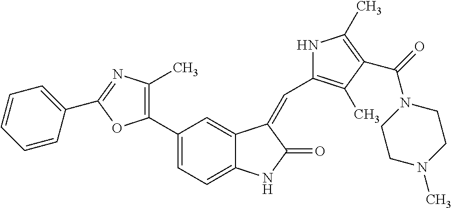 [SMILES: Cc1nc(-c2ccccc2)oc1-c1ccc2c(c1)/C(=C/c1[nH]c(C)c(C(=O)N3CCN(C)CC3)c1C)C(=O)N2] | 300 MHz ¹H-NMR (DMSO-d₆, δ) 13.64 (s, 1H), 11.86 (s, 1H), 8.12- 8.07 (m, 2H), 7.81 (s, 1H), 7.58- 7.48 (m, 5H), 7.09-7.03 (m, 1H), 3.96-3.46 (m, 7H), 2.50-2.47 (m, 4H), 2.34 (s, 3H), 2.27 (s, 3H), 2.14 (s, 3H) |
| 36 | 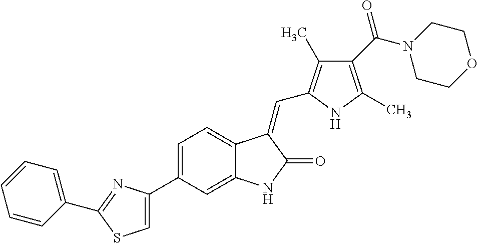 [SMILES: Cc1[nH]c(/C=C2\C(=O)Nc3cc(-c4csc(-c5ccccc5)n4)ccc32)c(C)c1C(=O)N1CCOCC1] | 300 MHz ¹H-NMR (DMSO-d₆, δ) 13.59 (s, 1H), 11.02 (s, 1H), 8.15 (s, 1H), 8.05-8.00 (m, 2H), 7.87 (d, 1H, J = 8.3 Hz), 7.72-7.67 (m, 2H), 7.58- 7.53 (m, 4H), 3.65-3.53 (m, 4H), 2.31 (s, 3H), 2.29 (s, 3H) |
| 37 | 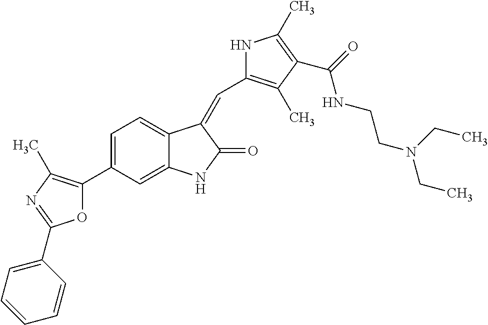 [SMILES: CCN(CC)CCNC(=O)c1c(C)[nH]c(/C=C2\C(=O)Nc3cc(-c4oc(-c5ccccc5)nc4C)ccc32)c1C] | LCMS m/z 538.65 (M + H) |
| 38 | 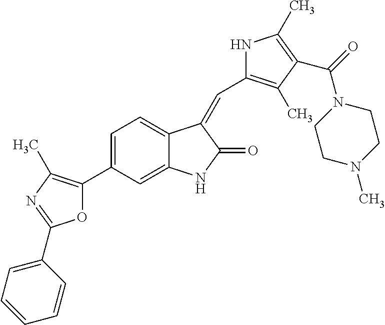 [SMILES: Cc1nc(-c2ccccc2)oc1-c1ccc2c(c1)NC(=O)/C2=C\c1[nH]c(C)c(C(=O)N2CCN(C)CC2)c1C] | LCMS m/z 522.61 (M + H) |
| 39 | 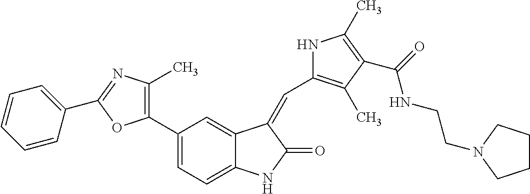 [SMILES: Cc1nc(-c2ccccc2)oc1-c1ccc2c(c1)/C(=C/c1[nH]c(C)c(C(=O)NCCN3CCCC3)c1C)C(=O)N2] | LCMS m/z 536.64 (M + H) |
| 40 | 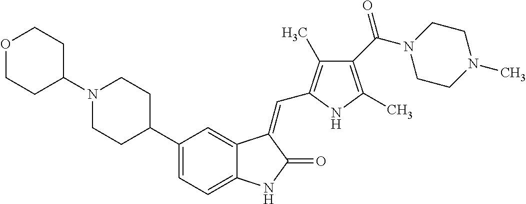 [SMILES: Cc1[nH]c(/C=C2\C(=O)Nc3ccc(C4CCN(C5CCOCC5)CC4)cc32)c(C)c1C(=O)N1CCN(C)CC1] | 300 MHz ¹H-NMR (DMSO-d₆, δ) 13.60 (s, 1H), 10.76 (s, 1H), 7.73 (s, 1H), 7.65 (s, 1H), 6.96 (dd, 1H, J = 7.9, 8.4 Hz), 6.75 (d, 1H, J = 7.9 Hz), 3.92-3.84 (m, 2H), 3.57- 3.35 (m, 4H), 3.04-2.96 (m, 2H), 2.52-2.35 (m, 3H), 2.32-2.15 (m, 7H), 2.26 (s, 3H), 2.26 (s, 3H), 2.18 (s, 3H), 1.78-1.64 (m, 6H), 1.52-1.37 (m, 2H) |
| 41 | 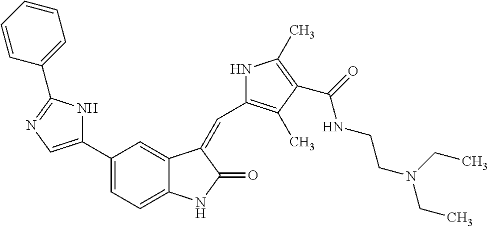 [SMILES: CCN(CC)CCNC(=O)c1c(C)[nH]c(/C=C2\C(=O)Nc3ccc(-c4cnc(-c5ccccc5)[nH]4)cc32)c1C] | 300 MHz ¹H-NMR (DMSO-d₆, δ) 13.68 (s, 1H), 12.61 (s, 1H), 10.93 (s, 1H), 8.18 (s, 1H), 8.03- 8.01 (m, 2H), 7.78-7.70 (m, 2H), 7.51-7.44 (m, 2H), 7.32-7.37 (m, 1H), 6.91-6.89 (m, 1H), 3.34- 3.30 (m, 4H), 2.64-2.60 (m, 4H), 2.50-2.48 (m, 6H), 0.99-0.96 (m, 6H) |
| 42 | 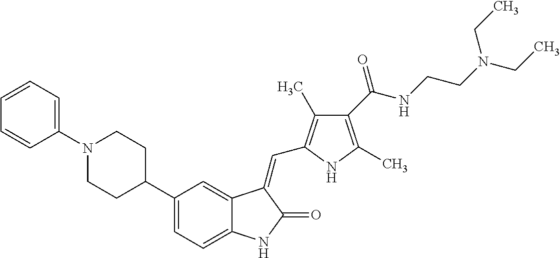 [SMILES: CCN(CC)CCNC(=O)c1c(C)[nH]c(/C=C2\C(=O)Nc3ccc(C4CCN(c5ccccc5)CC4)cc32)c1C] | 300 MHz ¹H-NMR (DMSO-d₆, δ) 13.68 (s, 1H), 10.80 (s, 1H), 7.78 (s, 1H), 7.68 (s, 1H), 7.24-7.19 (m, 2H), 7.04-6.95 (m, 3H), 6.80- 6.74 (m, 2H), 3.84-3.76 (m, 2H), 3.35-3.22 (m, 3H), 2.78-2.53 (m, 6H), 2.50-2.36 (m, 2H), 2.43 (s, 3H), 2.42 (s, 3H), 1.91-1.81 (m, 4H), 1.01 (brs, 6H) |
| 43 | 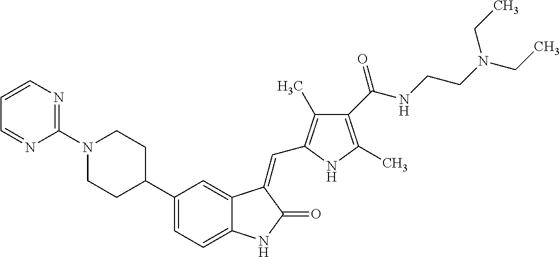 [SMILES: CCN(CC)CCNC(=O)c1c(C)[nH]c(/C=C2\C(=O)Nc3ccc(C4CCN(c5ncccn5)CC4)cc32)c1C] | 300 MHz ¹H-NMR (DMSO-d₆, δ) 13.66 (s, 1H), 10.79 (s, 1H), 8.36(s, 1H), 8.34 (s, 1H), 7.73 (s, 1H), 7.65 (s, 1H), 7.40-7.36 (m, 1H), 6.99 (brd, 1H, J = 7.9 Hz), 6.77 (d, 1H, J = 7.9 Hz), 6.58 (t, 1H, J = 4.6 Hz), 4.90-4.80 (m, 2H), 3.30- 3.20 (m, 2H), 2.98-2.86 (m, 2H), 2.86-2.69 (m, 1H), 2.56- 2.44 (m, 6H), 2.42 (s, 3H), 2.40 (s, 3H), 1.89-1.78 (m, 3H), 1.72- 1.55 (m, 2H), 0.96 (t, 6H, J = 7.0 Hz) |
| 44 | 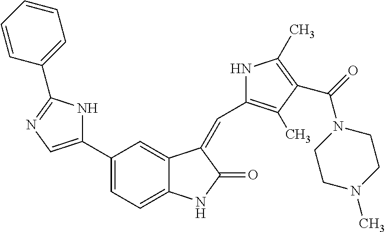 [SMILES: Cc1[nH]c(/C=C2\C(=O)Nc3ccc(-c4cnc(-c5ccccc5)[nH]4)cc32)c(C)c1C(=O)N1CCN(C)CC1] | 300 MHz ¹H-NMR (DMSO-d₆, δ) 13.62 (s, 1H), 12.60 (s, 1H), 10.90 (s, 1H), 8.14 (s, 1H), 8.02- 7.99 (m, 2H), 7.71-7.67 (m, 2H), 7.51-7.44 (m, 2H), 7.37-7.32 (m, 1H), 6.90-6.87 (m, 1H), 3.45- 3.33 (m, 4H), 2.50-2.48 (m, 4H), 2.29 (s, 3H), 2.19 (s, 3H), 2.14 (s, 3H) |
| 45 | 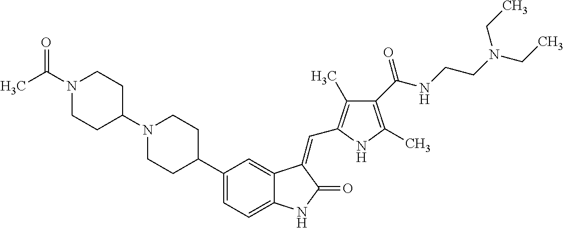 [SMILES: CCN(CC)CCNC(=O)c1c(C)[nH]c(/C=C2\C(=O)Nc3ccc(C4CCN(C5CCN(C(C)=O)CC5)CC4)cc32)c1C] | 300 MHz ¹H-NMR (DMSO-d₆, δ) 13.66 (s, 1H), 10.77 (s, 1H), 7.75 (s, 1H), 7.67 (s, 1H), 7.38 (t, 1H, J = 5.6 Hz), 6.96 (brd, 1H, J = 7.9 Hz), 6.75 (d, 1H, J = 7.9 Hz), 4.43- 4.36 (m, 1H), 3.86-3.80 (m, 1H), 3.35-3.23 (m, 4H), 2.98-2.93 (m, 2H), 2.55-2.38 (m, 8H), 2.42 (s, 3H), 2.42 (s, 3H), 2.30-2.15 (m, 2H), 1.98 (s, 3H), 1.70-1.64 (m, 6H), 1.48-1.15 (m, 2H), 0.96 (t, 6H, J = 7.2 Hz) |
| 46 | 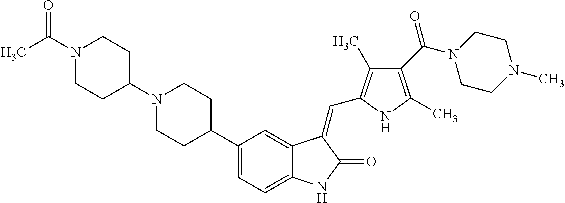 [SMILES: CC(=O)N1CCC(N2CCC(c3ccc4c(c3)/C(=C/c3[nH]c(C)c(C(=O)N5CCN(C)CC5)c3C)C(=O)N4)CC2)CC1] | 300 MHz ¹H-NMR (DMSO-d₆, δ) 13.60 (s, 1H), 10.77 (s, 1H), 7.73 (s, 1H), 7.65 (s, 1H), 6.96 (d, 1H, J = 7.9 Hz), 6.75 (brd, 1H, J = 7.9 Hz), 4.43-4.35 (m, 1H), 3.88-3.78 (m, 1H), 3.60-3.38 (m, 4H), 3.04- 2.90 (m, 2H), 2.56-2.20 (m, 8H), 2.26 (s, 3H), 2.26(s, 3H), 2.19 (s, 3H), 2.00-1.91 (m, 2H), 1.98 (s, 3H), 1.70-1.65 (m, 6H), 1.48- 1.18 (m, 2H) |
| 47 | 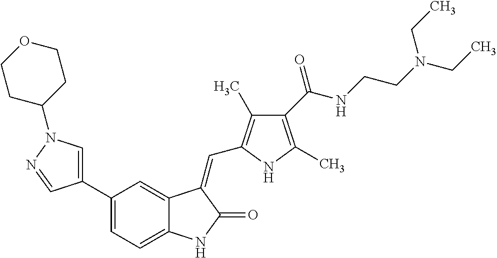 [SMILES: CCN(CC)CCNC(=O)c1c(C)[nH]c(/C=C2\C(=O)Nc3ccc(-c4cnn(C5CCOCC5)c4)cc32)c1C] | 300 MHz ¹H-NMR (DMSO-d₆, δ) 13.64 (s, 1H), 10.88 (s, 1H), 8.19 (s, 1H), 8.00 (s, 1H), 7.90 (s, 1H), 7.68 (s, 1H), 7.45-7.38 (m, 1H), 7.35 (d, 1H, J = 7.9 Hz), 6.85 (d, 1H, J = 7.9 Hz), 4.44-4.33 (m, 1H), 4.02-3.91 (m, 2H), 3.52-3.43 (m, 2H), 3.32-3.24 (m, 2H), 2.58-2.48 (m, 6H), 2.44 (s, 3H), 2.44 (s, 3H), 2.06- 1.89 (m, 4H), 0.97 (t, 6H, J = 7.0 Hz) |
| 48 | 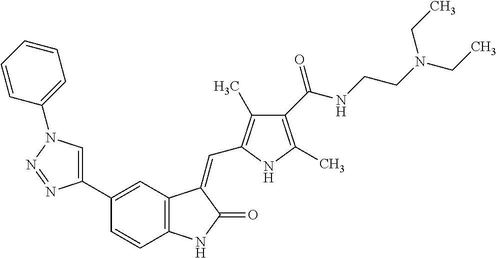 [SMILES: CCN(CC)CCNC(=O)c1c(C)[nH]c(/C=C2\C(=O)Nc3ccc(-c4cn(-c5ccccc5)nn4)cc32)c1C] | 300 MHz ¹H-NMR (DMSO-d₆, δ) 13.64 (s, 1H), 11.06 (s, 1H), 9.18 (s, 1H), 8.30 (s, 1H), 7.96 (d, 2H, J = 9 Hz), 7.55-7.45 (m, 6H), 7.00 (d, 1H, J = 9 Hz), 3.39-3.26 (m, 4H), 2.56-2.45 (m, 10H), and 0.97 (t, J = 7.5 Hz). |
| 49 | 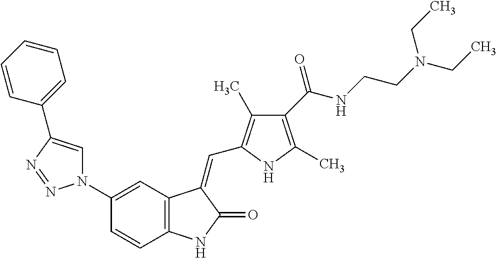 [SMILES: CCN(CC)CCNC(=O)c1c(C)[nH]c(/C=C2\C(=O)Nc3ccc(-n4cc(-c5ccccc5)nn4)cc32)c1C] | LCMS m/z 524.5 (M + H) |
| 50 | 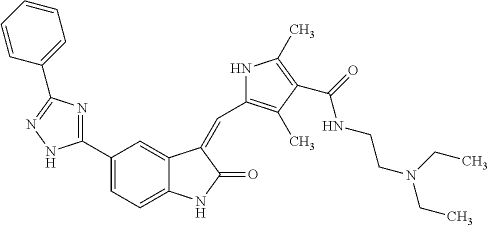 [SMILES: CCN(CC)CCNC(=O)c1c(C)[nH]c(/C=C2\C(=O)Nc3ccc(-c4nc(-c5ccccc5)n[nH]4)cc32)c1C] | 300 MHz ¹H-NMR (DMSO-d₆, δ) 13.64 (s, 1H), 11.14 (s, 1H), 8.41 (s, 1H), 8.12-8.11 (m, 2H), 7.87-7.85 (m, 1H), 7.84 (s, 1H), 7.55-7.48 (m, 4H), 7.03-7.01 (m, 1H), 3.34-3.25 (m, 4H), 2.54-2.45 (m, 10H), 0.99- 0.95 (m, 6H) |

### Example 51

### Production of (Z)—N-(2-(diethylamino)ethyl)-2,4-dimethyl-5-((2-oxo-5-(4-phenylthiazol-2-yl)indolin-3-ylidene)methyl)-1H-pyrrole-3-carboxamide 51

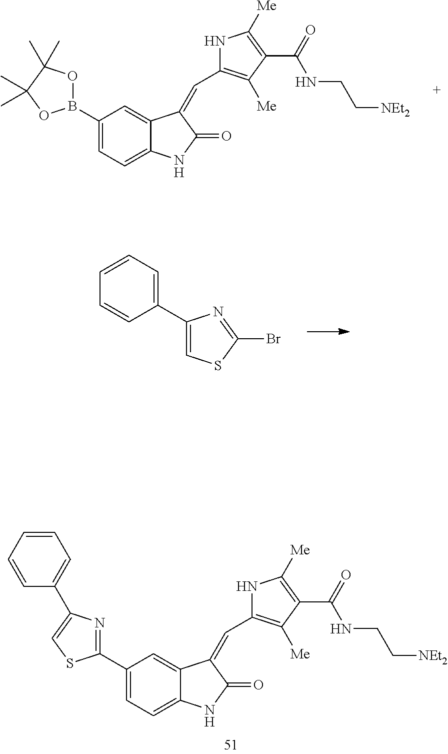

To a solution of (Z)—N-(2-(diethylamino)ethyl)-2,4-dimethyl-5-((2-oxo-5-(4,4,5,5-tetramethyl-1,3,2-dioxaborolan-2-yl)indolin-3-ylidene)methyl)-1H-pyrrole-3-carboxamide (40 mg, 0.079 mmol) in DMF/H₂O (3 ml/1 ml) was added Pd(PPh₃)₄ (9.1 mg, 0.008 mmol), 2-bromo-4-phenylthiazole (23 mg, 0.095 mmol) and potassium carbonate (33 mg, 0.237 mmol). The mixture was stirred at 110° C. for 1 hour under microwave irradiation. The mixture was extracted with CHCl₃, and the organic layer was washed with H₂O, dried over Na₂SO₄ and concentrated in vacuo. The residue was purified by column chromatography (CHCl₃/MeOH) to give (Z)—N-(2-(diethylamino)ethyl)-2,4-dimethyl-5-((2-oxo-5-(4-phenylthiazol-2-yl)indolin-3-ylidene)methyl)-1H-pyrrole-3-carboxamide 51 (10 mg) as yellow solid.

¹H NMR (300 MHz, DMSO-d₆) δ 13.70 (s, 1H), 11.20 (s, 1H), 8.43 (s, 1H), 8.07-8.11 (m, 3H), 7.79-7.90 (m. 2H), 7.39-7.50 (m, 4H), 7.02 (m, 1H), 3.25-3.35 (m, 4H), 2.4-2.6 (m, 10H), 0.95-0.99 (m, 6H); MS m/z 540.69 (M+H).

### Examples 52 to 55

Reactions and treatments were carried out in the same manner as in Example 1 using the corresponding starting material compounds, thereby giving the compounds of Examples 52 to 55 shown in Table 8.

TABLE 8

| Example | Structure | Spectral data |
|---|---|---|
| 52 | 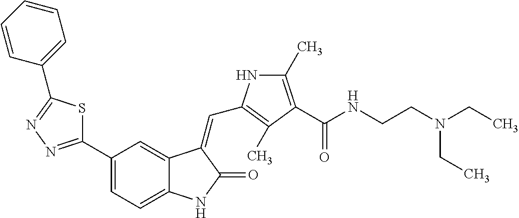 [SMILES: CCN(CC)CCNC(=O)c1c(C)[nH]c(/C=C2\C(=O)Nc3ccc(-c4nnc(-c5ccccc5)s4)cc32)c1C] | 300 MHz ¹H-NMR (CDCl₃, δ) 13.24 (s, 1H), 8.03 (s, 1H), 7.89 (t, 1H, J = 3.3 Hz), 7.83 (s, 1H), 7.58 (t, 1H, J = 90 Hz), 7.41-7.27 (m, 4H), 6.91- 6.82 (m, 1H), 3.62 (brs, 2H), 2.93- 2.82 (m, 6H), 2.62-2.36 (m, 6H), 1.18-1.15 (m, 6H) |
| 53 | 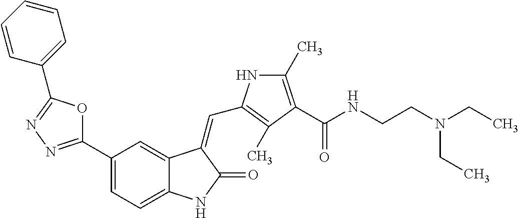 [SMILES: CCN(CC)CCNC(=O)c1c(C)[nH]c(/C=C2\C(=O)Nc3ccc(-c4nnc(-c5ccccc5)o4)cc32)c1C] | 300 MHz ¹H-NMR (CD₃OD, δ) 8.34 (s, 1H), 8.16 (d, 2H, J = 7.8 Hz), 7.92 (d, 1H, J = 8.4 Hz), 7.65-7.60 (m, 5H), 7.08 (d, 1H, J = 8.1 Hz), 3.59 (t, 2H, J = 6.6 Hz), 2.99-2.88 (m, 6H), 2.52 (s, 3H), 2.50 (s, 3H), 1.22 (t, 6H, J = 7.2 Hz) |
| 54 | 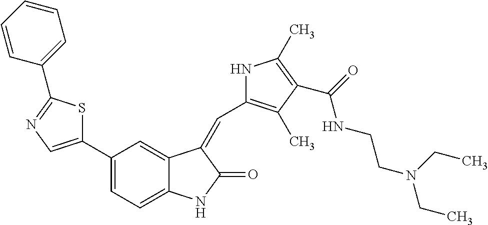 [SMILES: CCN(CC)CCNC(=O)c1c(C)[nH]c(/C=C2\C(=O)Nc3ccc(-c4cnc(-c5ccccc5)s4)cc32)c1C] | 300 MHz ¹H-NMR (DMSO-d₆, δ) 13.64 (s, 1H), 11.07 (s, 1H), 8.28 (s, 1H), 8.23 (s, 1H), 7.96-7.94 (m, 2H), 7.83 (s, 1H), 7.55-7.42 (m, 4H), 6.97-6.94 (m, 1H), 3.38-3.25 (m, 4H), 2.56-2.40 (m, 10H), 0.99-0.95 (m, 6H) |
| 55 | 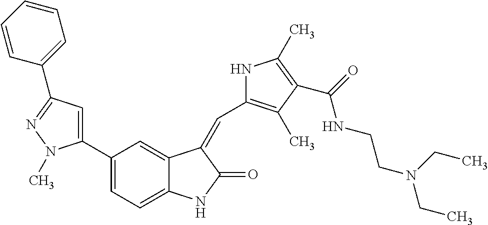 [SMILES: CCN(CC)CCNC(=O)c1c(C)[nH]c(/C=C2\C(=O)Nc3ccc(-c4cc(-c5ccccc5)nn4C)cc32)c1C] | 300 MHz ¹H-NMR (DMSO₆, δ) 13.65 (s, 1H), 11.25 (s, 1H), 7.86 (s, 1H), 7.75 (s, 1H), 7.55 (s, 1H), 7.44- 7.41 (m, 3H), 7.27-7.21 (m, 3H), 6.91- 6.89 (m, 1H), 6.78-6.75 (m, 1H), 3.96 (s, 3H), 3.34-3.28 (m, 4H), 2.55- 2.50 (m, 4H), 2.45 (s, 3H), 2.34 (s, 3H), 0.99-0.97 (m, 6H) |

### Example 56

### Production of (Z)—N-(2-(diethylamino)ethyl)-2,4-dimethyl-5-((2-oxo-5-(5-phenylfuran-2-yl)indolin-3-ylidene)methyl)-1H-pyrrole-3-carboxamide 56

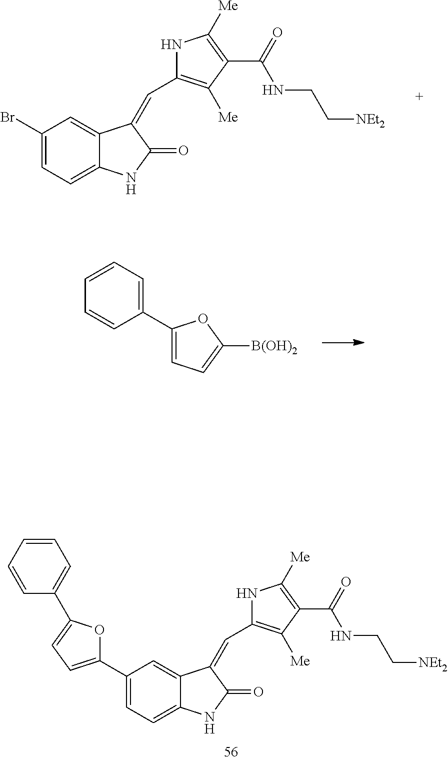

To a solution of (Z)-5-((5-bromo-2-oxoindolin-3-ylidene)methyl)-N-(2-(diethylamino)ethyl)-2,4-dimethyl-1H-pyrrole-3-carboxamide (31 mg, 0.068 mmol) in DMF/H₂O (0.75 ml/0.25 ml) was added Pd(PPh₃)₄ (15.9 mg, 0.014 mmol), 5-phenylfuran-2-boronic acid (17 mg, 0.090 mmol) and potassium carbonate (14 mg, 0.100 mmol). The mixture was stirred at 120° C. for 1 hour under microwave irradiation. The mixture was concentrated in vacuo. The residue was purified by reverse phase column chromatography (H₂O/CH₃CN) to give (Z)—N-(2-(diethylamino)ethyl)-2,4-dimethyl-5-((2-oxo-5-(5-phenylfuran-2-yl)indolin-3-ylidene)methyl)-1H-pyrrole-3-carboxamide 56 (12 mg) as an orange solid.

¹H NMR (300 MHz, CDCl₃) δ 7.76-7.72 (m, 3H), 7.55 (d, 1H, J=9.9 Hz), 7.45-7.37 (m, 3H), 7.27-7.24 (m, 3H), 6.91 (d, 1H, J=8.4 Hz), 6.72 (d, 1H, J=3.3 Hz), 6.67 (d, 1H, J=3.6 Hz), 3.57 (brs, 2H), 2.71 (brs, 4H), 2.58 (s, 3H), 2.52 (s, 3H), 1.11 (brs, 6H).

### Examples 57 to 75

Reactions and treatments were carried out in the same manner as in Example 1 using the corresponding starting material compounds, thereby giving the compounds of Examples 57 to 75 shown in Table 9.

TABLE 9

| Example | Structure | Spectral data |
|---|---|---|
| 57 | 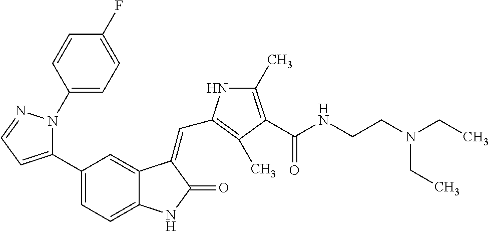 [SMILES: CCN(CC)CCNC(=O)c1c(C)[nH]c(/C=C2\C(=O)Nc3ccc(-c4ccnn4-c4ccc(F)cc4)cc32)c1C] | LCMS m/z 541.38 (M + H) |
| 58 | 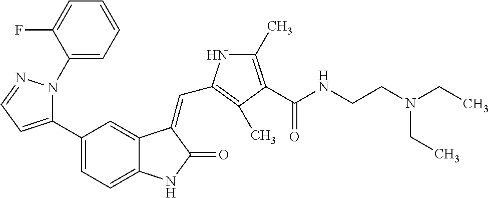 [SMILES: CCN(CC)CCNC(=O)c1c(C)[nH]c(/C=C2\C(=O)Nc3ccc(-c4ccnn4-c4ccccc4F)cc32)c1C] | LCMS m/z 541.47 (M + H) |
| 59 | 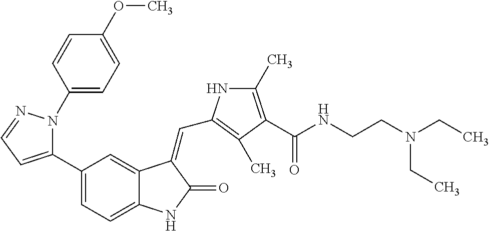 [SMILES: CCN(CC)CCNC(=O)c1c(C)[nH]c(/C=C2\C(=O)Nc3ccc(-c4ccnn4-c4ccc(OC)cc4)cc32)c1C] | LCMS m/z 553.39 (M + H) |
| 60 | 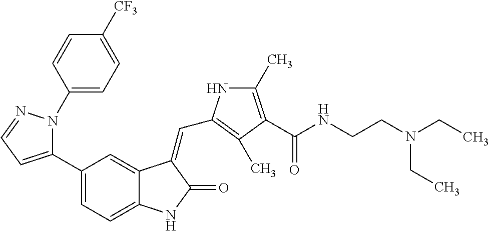 [SMILES: CCN(CC)CCNC(=O)c1c(C)[nH]c(/C=C2\C(=O)Nc3ccc(-c4ccnn4-c4ccc(C(F)(F)F)cc4)cc32)c1C] | LCMS m/z 591.24 (M + H) |
| 61 | 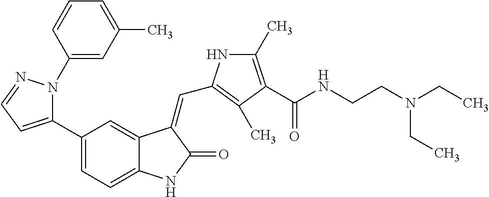 [SMILES: CCN(CC)CCNC(=O)c1c(C)[nH]c(/C=C2\C(=O)Nc3ccc(-c4ccnn4-c4cccc(C)c4)cc32)c1C] | LCMS m/z 269.33 (M + 2H) |
| 62 | 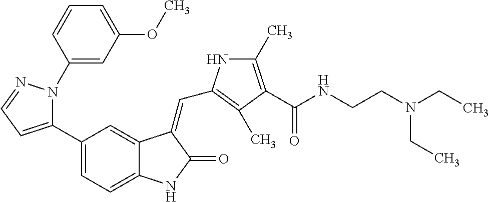 [SMILES: CCN(CC)CCNC(=O)c1c(C)[nH]c(/C=C2\C(=O)Nc3ccc(-c4ccnn4-c4cccc(OC)c4)cc32)c1C] | LCMS m/z 553.39 (M + H) |
| 63 | 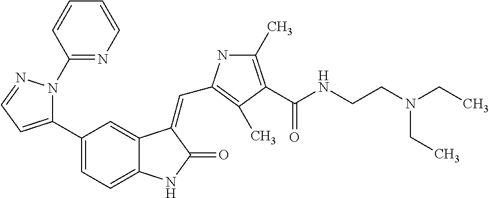 [SMILES: CCN(CC)CCNC(=O)c1c(C)[nH]c(/C=C2\C(=O)Nc3ccc(-c4ccnn4-c4ccccn4)cc32)c1C] | LCMS m/z 524.33 (M + H) |
| 64 | 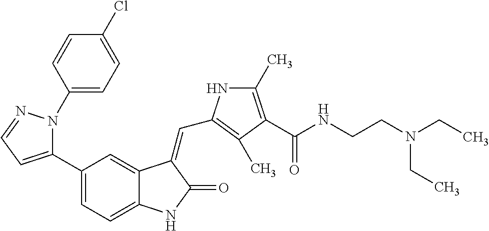 [SMILES: CCN(CC)CCNC(=O)c1c(C)[nH]c(/C=C2\C(=O)Nc3ccc(-c4ccnn4-c4ccc(Cl)cc4)cc32)c1C] | LCMS m/z 557.29 (M + H) |
| 65 | 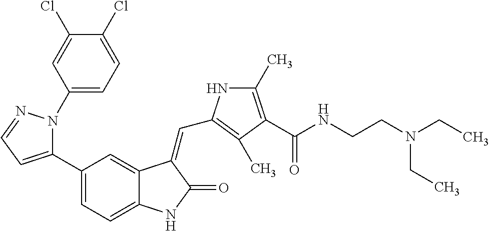 [SMILES: CCN(CC)CCNC(=O)c1c(C)[nH]c(/C=C2\C(=O)Nc3ccc(-c4ccnn4-c4ccc(Cl)c(Cl)c4)cc32)c1C] | LCMS m/z 591.35 (M + H) |
| 66 | 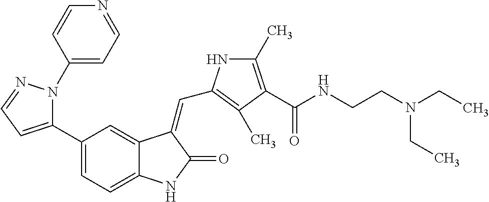 [SMILES: CCN(CC)CCNC(=O)c1c(C)[nH]c(/C=C2\C(=O)Nc3ccc(-c4ccnn4-c4ccncc4)cc32)c1C] | LCMS m/z 524.33 (M + H) |
| 67 | 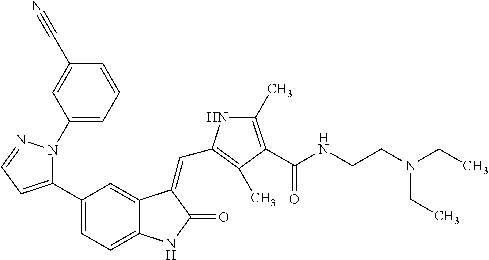 [SMILES: CCN(CC)CCNC(=O)c1c(C)[nH]c(/C=C2\C(=O)Nc3ccc(-c4ccnn4-c4cccc(C#N)c4)cc32)c1C] | LCMS m/z 548.33 (M + H) |
| 68 | 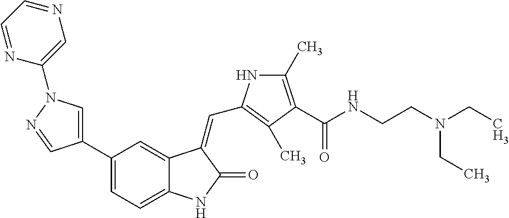 [SMILES: CCN(CC)CCNC(=O)c1c(C)[nH]c(/C=C2\C(=O)Nc3ccc(-c4cnn(-c5cnccn5)c4)cc32)c1C] | LCMS m/z 525.33 (M + H) |
| 69 | 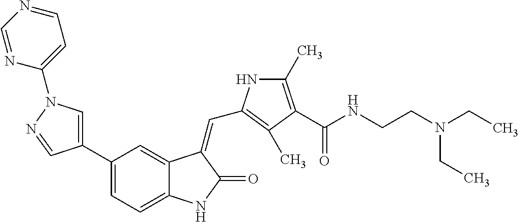 [SMILES: CCN(CC)CCNC(=O)c1c(C)[nH]c(/C=C2\C(=O)Nc3ccc(-c4cnn(-c5ccncn5)c4)cc32)c1C] | LCMS m/z 525.28 (M + H) |
| 70 | 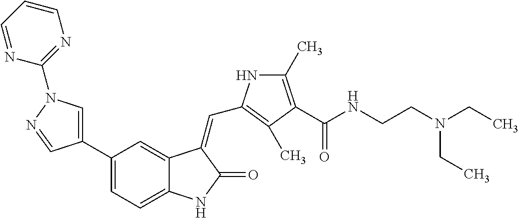 [SMILES: CCN(CC)CCNC(=O)c1c(C)[nH]c(/C=C2\C(=O)Nc3ccc(-c4cnn(-c5ncccn5)c4)cc32)c1C] | LCMS m/z 525.33 (M + H) |
| 71 | 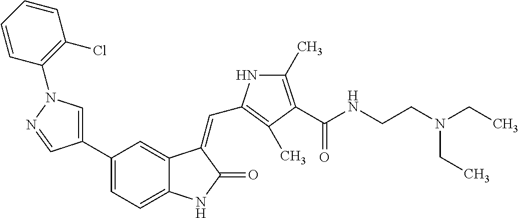 [SMILES: CCN(CC)CCNC(=O)c1c(C)[nH]c(/C=C2\C(=O)Nc3ccc(-c4cnn(-c5ccccc5Cl)c4)cc32)c1C] | LCMS m/z 557.34 (M + H) |
| 72 | 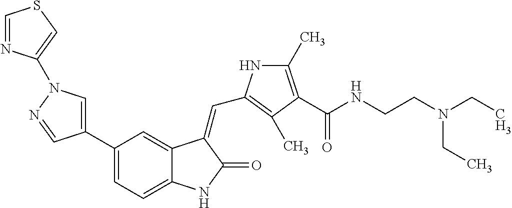 [SMILES: CCN(CC)CCNC(=O)c1c(C)[nH]c(/C=C2\C(=O)Nc3ccc(-c4cnn(-c5cscn5)c4)cc32)c1C] | LCMS m/z 530.28 (M + H) |
| 73 | 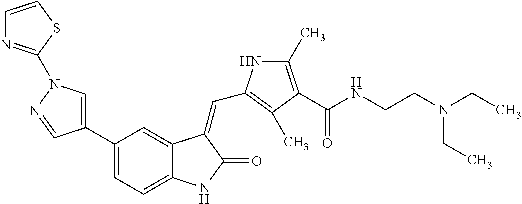 [SMILES: CCN(CC)CCNC(=O)c1c(C)[nH]c(/C=C2\C(=O)Nc3ccc(-c4cnn(-c5nccs5)c4)cc32)c1C] | LCMS m/z 530.28 (M + H) |
| 74 | 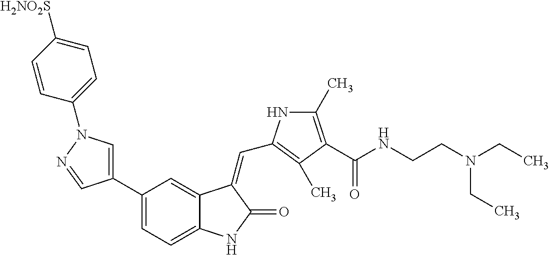 [SMILES: CCN(CC)CCNC(=O)c1c(C)[nH]c(/C=C2\C(=O)Nc3ccc(-c4cnn(-c5ccc(S(N)(=O)=O)cc5)c4)cc32)c1C] | LCMS m/z 602.30 (M + H) |
| 75 | 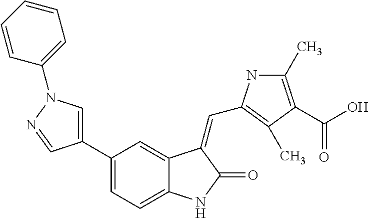 [SMILES: Cc1[nH]c(/C=C2\C(=O)Nc3ccc(-c4cnn(-c5ccccc5)c4)cc32)c(C)c1C(=O)O] | LCMS m/z 425.31 (M + H) |

### Examples 76 to 87

Reactions and treatments were carried out in the same manner as in Example 51 using the corresponding starting material compounds, thereby giving the compounds of Examples 76 to 87 shown in Table 10.

TABLE 10

| Example | Structure | Spectral data |
|---|---|---|
| 76 | 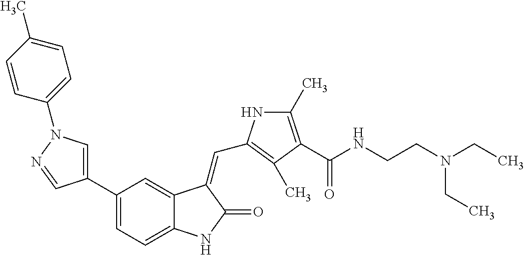 [SMILES: CCN(CC)CCNC(=O)c1c(C)[nH]c(/C=C2\C(=O)Nc3ccc(-c4cnn(-c5ccc(C)cc5)c4)cc32)c1C] | 300 MHz ¹H-NMR (DMSO-d₆, δ) 13.65 (s, 1H), 10.95 (s, 1H), 8.87 (s, 1H), 8.20 (s, 1H), 8.12 (s, 1H), 7.78- 7.71 (m, 3H), 7.47-7.42 (m, 2H), 7.33-7.31 (m, 2H), 6.91-6.88 (m, 1H), 3.31-3.24 (m, 4H), 2.66- 2.24 (m, 10H), 2.25 (s, 3H), 0.99- 0.95 (m, 6H) |
| 77 | 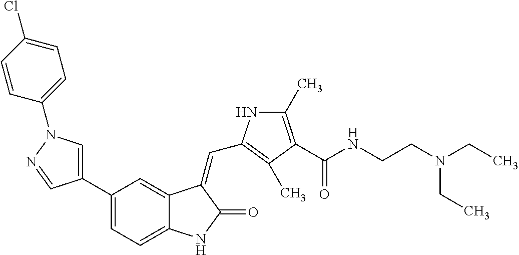 [SMILES: CCN(CC)CCNC(=O)c1c(C)[nH]c(/C=C2\C(=O)Nc3ccc(-c4cnn(-c5ccc(Cl)cc5)c4)cc32)c1C] | 300 MHz ¹H-NMR (DMSO-d₆, δ) 13.64 (s, 1H), 10.96 (s, 1H), 8.96 (s, 1H), 8.26 (s, 1H), 8.13 (s, 1H), 7.94- 9.93 (m, 2H), 7.71 (s, 1H), 7.60- 7.56 (m, 2H), 7.48-7.45 (m, 2H), 6.92-6.89 (m, 1H), 3.41-3.25 (m, 4H), 2.54-2.46 (m, 10H), 1.04- 0.97 (m, 6H) |
| 78 | 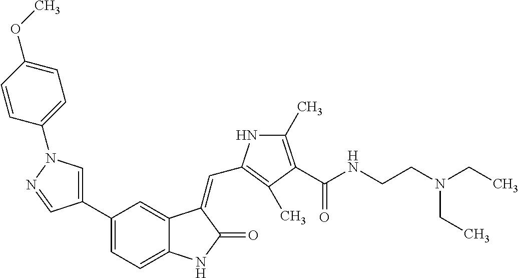 [SMILES: CCN(CC)CCNC(=O)c1c(C)[nH]c(/C=C2\C(=O)Nc3ccc(-c4cnn(-c5ccc(OC)cc5)c4)cc32)c1C] | 300 MHz ¹H-NMR (DMSO-d₆, δ) 13.66 (s, 1H), 10.95 (s, 1H), 8.81 (s, 1H), 8.18-8.12 (m, 2H), 7.80- 7.77 (m, 2H), 7.62 (s, 1H), 7.48- 7.46 (m, 2H), 7.09-7.06 (m, 2H), 6.91-6.88 (m, 1H), 3.80 (s, 3H), 3.34-3.31 (m, 4H), 2.53-2.44 (m, 10H), 0.98-0.96 m, 6H) |
| 79 | 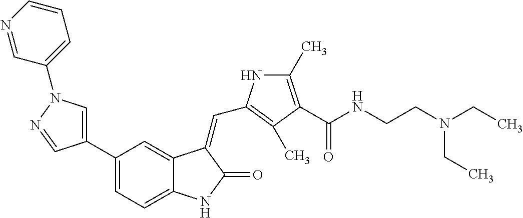 [SMILES: CCN(CC)CCNC(=O)c1c(C)[nH]c(/C=C2\C(=O)Nc3ccc(-c4cnn(-c5cccnc5)c4)cc32)c1C] | LCMS m/z 524.63 (M + H) |
| 80 | 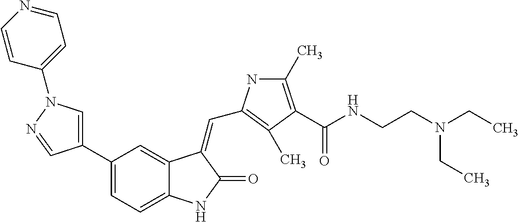 [SMILES: CCN(CC)CCNC(=O)c1c(C)[nH]c(/C=C2\C(=O)Nc3ccc(-c4cnn(-c5ccncc5)c4)cc32)c1C] | 300 MHz ¹H-NMR (DMSO-d₆, δ) 13.64 (s, 1H), 11.00 (s, 1H), 9.12 (s, 1H), 8.68-8.66 (m, 2H), 8.37 (s, 1H), 8.15 (s, 1H), 7.90-7.88 (m, 2H) 7.72 (s, 1H), 7.50-7.43 (m, 2H), 6.94-6.91 (m, 1H), 3.31-3.25 (m, 4H), 2.54-2.37 (m, 10H), 0.99-0.95 (m, 6H). |
| 81 | 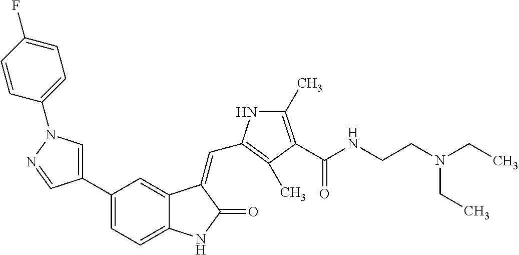 [SMILES: CCN(CC)CCNC(=O)c1c(C)[nH]c(/C=C2\C(=O)Nc3ccc(-c4cnn(-c5ccc(F)cc5)c4)cc32)c1C] | 300 MHz ¹H-NMR (DMSO-d₆, δ) 13.65 (s, 1H), 10.96 (s, 1H), 8.91 (s, 1H), 8.23 (s, 1H), 8.13 (s, 1H), 7.92- 7.89 (m, 2H), 7.71 (s, 1H), 7.50- 7.38 (m, 4H), 6.92-6.89 (m, 1H), 3.34-3.30 (m, 4H), 2.53-2.45 (m, 10H), 0.98-0.96 (m, 6H) |
| 82 | 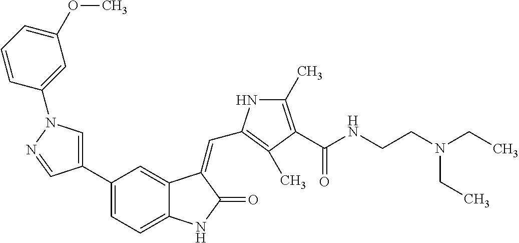 [SMILES: CCN(CC)CCNC(=O)c1c(C)[nH]c(/C=C2\C(=O)Nc3ccc(-c4cnn(-c5cccc(OC)c5)c4)cc32)c1C] | LCMS m/z 553.37 (M + H) |
| 83 | 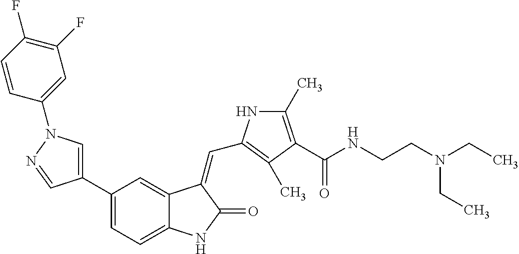 [SMILES: CCN(CC)CCNC(=O)c1c(C)[nH]c(/C=C2\C(=O)Nc3ccc(-c4cnn(-c5ccc(F)c(F)c5)c4)cc32)c1C] | LCMS m/z 559.33 (M + H) |
| 84 | 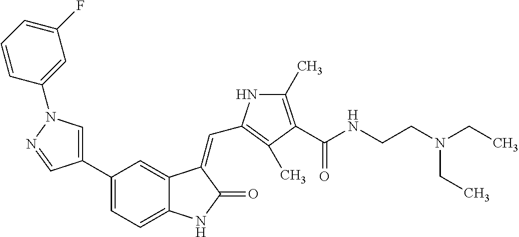 [SMILES: CCN(CC)CCNC(=O)c1c(C)[nH]c(/C=C2\C(=O)Nc3ccc(-c4cnn(-c5cccc(F)c5)c4)cc32)c1C] | LCMS m/z 541.33 (M + H) |
| 85 | 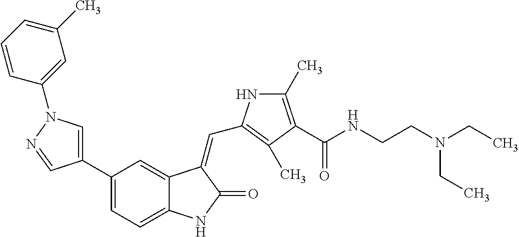 [SMILES: CCN(CC)CCNC(=O)c1c(C)[nH]c(/C=C2\C(=O)Nc3ccc(-c4cnn(-c5cccc(C)c5)c4)cc32)c1C] | LCMS m/z 537.36 (M + H) |
| 86 | 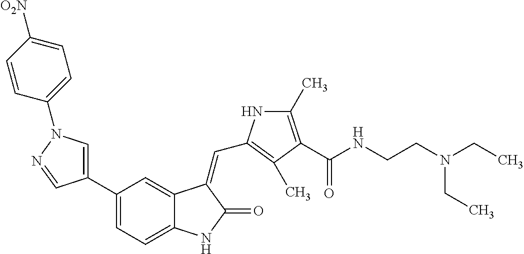 [SMILES: CCN(CC)CCNC(=O)c1c(C)[nH]c(/C=C2\C(=O)Nc3ccc(-c4cnn(-c5ccc([N+](=O)[O-])cc5)c4)cc32)c1C] | LCMS m/z 568.36 (M + H) |
| 87 | 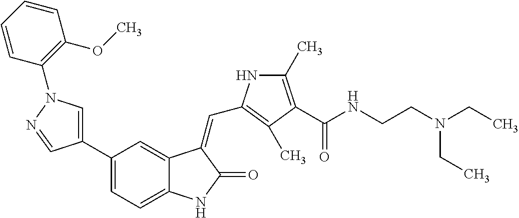 [SMILES: CCN(CC)CCNC(=O)c1c(C)[nH]c(/C=C2\C(=O)Nc3ccc(-c4cnn(-c5ccccc5OC)c4)cc32)c1C] | LCMS m/z 553.40 (M + H) |

### Example 88

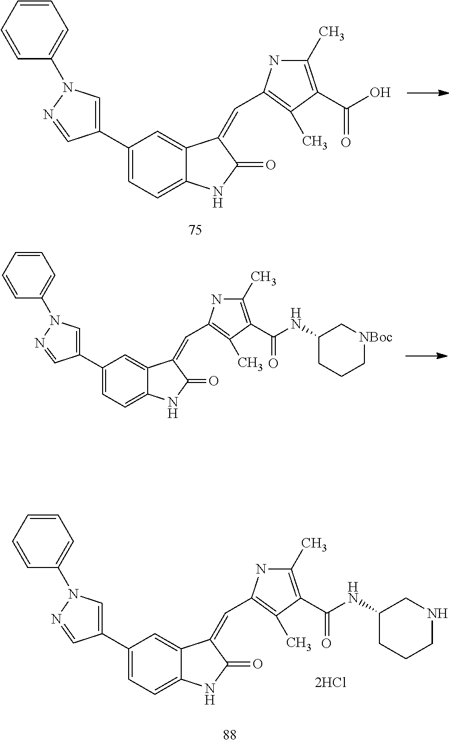 [SMILES: Cc1[nH]c(/C=C2\C(=O)Nc3ccc(-c4cnn(-c5ccccc5)c4)cc32)c(C)c1C(=O)O ; Cc1[nH]c(/C=C2\C(=O)Nc3ccc(-c4cnn(-c5ccccc5)c4)cc32)c(C)c1C(=O)N[C@H]1CCCN(C(=O)OC(C)(C)C)C1 ; Cc1[nH]c(/C=C2\C(=O)Nc3ccc(-c4cnn(-c5ccccc5)c4)cc32)c(C)c1C(=O)N[C@H]1CCCNC1]

Step 1

To a solution of (Z)-2,4-dimethyl-5-((2-oxo-5-(1-phenyl-1H-pyrazol-4-yl) indolin-3-ylidene)methyl)-1H-pyrrole-3-carboxylic acid (20 mg, 0.047 mmol) 75 in DMF (1 ml) were added WSCI (14 mg, 0.071 mmol), HOBt (10 mg, 0.071 mmol), Et₃N (19 uL, 0.14 mmol) and (S)-1-Boc-3-aminopiperidine (14 mg, 0.071 mmol). The mixture was stirred overnight at room temperature, and poured into water. The mixture was extracted with EtOAc, and washed with saturated aqueous NH₄Cl, water, and brine. The organic layer was dried over Na₂SO₄ and concentrated to afford tert-butyl (S,Z)-3-(2,4-dimethyl-5-((2-oxo-5-(1-phenyl-1H-pyrazol-4-yl)indolin-3-ylidene)methyl)-1H-pyrrole-3-carboxamido)piperidine-1-carboxylate (30 mg).

MS m/z 607.40 (M+H).

Step 2

To a solution of tert-butyl (S,Z)-3-(2,4-dimethyl-5-((2-oxo-5-(1-phenyl-1H-pyrazol-4-yl)indolin-3-ylidene)methyl)-1H-pyrrole-3-carboxamido)piperidine-1-carboxylate (30 mg) in CHCl₃ (2 ml) was added 4NHCl/dioxane (1 ml), and the reaction mixture was stirred for 30 min. The solvent was evaporated to give (S,Z)-2,4-dimethyl-5-((2-oxo-5-(1-phenyl-1H-pyrazol-4-yl)indolin-3-ylidene)methyl)-N-(piperidin-3-yl)-1H-pyrrole-3-carboxamide hydrochloride 88 (25 mg).

¹H NMR (400 MHz, DMSO-d₆) δ 13.70 (s, 1H), 10.98 (s, 1H), 9.29 (m, 1H), 9.05 (m, 1H), 8.94 (s, 1H), 8.54 (br, 1H), 8.24 (s, 1H), 8.16 (s, 1H), 7.90 (d, 2H, J=7.3 Hz), 7.85 (d, 1H, J=8.0 Hz), 7.75 (s, 1H), 7.55-7.49 (m, 3H), 7.33 (m, 1H), 6.92 (d, 1H, J=8.0 Hz), 4.17 (m, 1H), 3.18 (m, 1H), 2.90-2.70 (m, 3H), 2.47 (s, 3H), 2.46 (s, 3H), 1.98-1.50 (m, 4H).

Reactions and treatments were carried out in the same manner as in Example 88 using the corresponding starting material compounds, thereby giving the compounds of Examples 89 to 101 shown in Table 11.

TABLE 11

| Example | Structure | Spectral data |
|---|---|---|
| 89 | 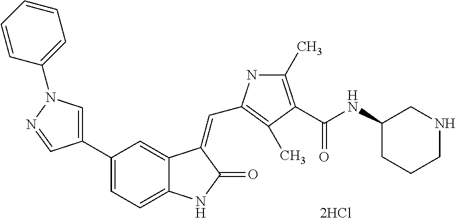 [SMILES: Cc1[nH]c(/C=C2\C(=O)Nc3ccc(-c4cnn(-c5ccccc5)c4)cc32)c(C)c1C(=O)N[C@@H]1CCCNC1 ; "2HCl"] | 400 MHz ¹H-NMR (DMSO-d₆, δ) 13.70 (s, 1H), 10.98 (s, 1H), 9.05 (m, 1H), 8.94 (s, 1H), 8.84 (m, 1H), 8.42 (br, 1H), 8.24 (s, 1H), 8.16 (s, 1H), 7.90 (d, 2H, J = 7.3 Hz), 7.81 (d, 1H, J = 8.0 Hz), 7.75 (s, 1H), 7.55-7.49 (m, 3H), 7.33 (m, 1H), 6.92 (d, 1H, J = 8.0 Hz), 4.17 (m, 1H), 3.18 (m, 1H), 2.90- 2.70 (m, 3H), 2.47 (s, 3H), 2.46 (s, 3H), 1.98-1.50 (m, 4H). |
| 90 | 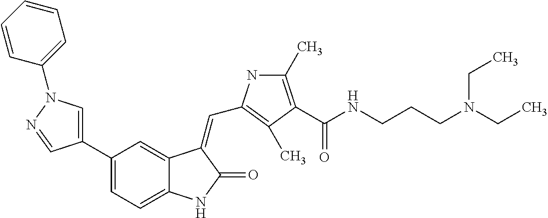 [SMILES: CCN(CC)CCCNC(=O)c1c(C)[nH]c(/C=C2\C(=O)Nc3ccc(-c4cnn(-c5ccccc5)c4)cc32)c1C] | 400 MHz ¹H-NMR (DMSO-d₆, δ) 13.71 (s, 1H), 11.00 (s, 1H), 8.99 (s, 1H), 8.31 (s, 1H), 8.21 (s, 1H), 7.96 (m, 2H), 7.79 (s, 1H), 7.73 (t, 1H, J = 6.1 Hz), 7.63-7.55 (m, 3H), 7.39 (m, 1H), 6.98 (d, 1H, J = 8.0 Hz), 3.32 (q, 2H, J = 6.1 Hz), 2.54- 2.48 (m, 12H), 1.69 (m, 2H), 1.02 (t, 6H, J = 7.5 Hz) |
| 91 | 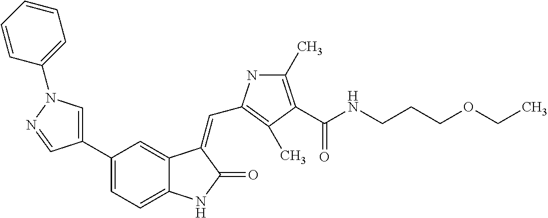 [SMILES: CCOCCCNC(=O)c1c(C)[nH]c(/C=C2\C(=O)Nc3ccc(-c4cnn(-c5ccccc5)c4)cc32)c1C] | 400 MHz ¹H-NMR (DMSO-d₆, δ) 13.64 (s, 1H), 10.93 (s, 1H), 8.92 (s, 1H), 8.23 (s, 1H), 8.13 (s, 1H), 7.88 (d, 2H, J = 7.3 Hz), 7.71 (s, 1H), 7.62-7.48 (m, 4H), 7.26 (t, 1H, J = 7.3 Hz), 6.91 (d, 1H, J = 7.9 Hz), 3.46-3.24 (m, 6H), 2.45 (s, 3H), 2.43 (s, 3H), 1.74 (m, 2H), 1.11 (t, 3H, J = 7.5 Hz) |
| 92 | 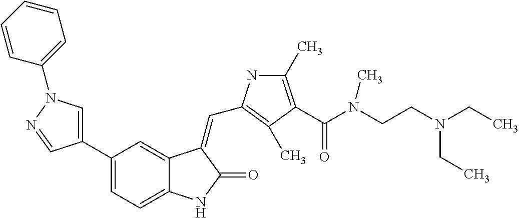 [SMILES: CCN(CC)CCN(C)C(=O)c1c(C)[nH]c(/C=C2\C(=O)Nc3ccc(-c4cnn(-c5ccccc5)c4)cc32)c1C] | 400 MHz ¹H-NMR (DMSO-d₆, δ) 13.55 (s, 1H), 10.91 (s, 1H), 8.91 (s, 1H), 8.22 (s, 1H), 8.11 (s, 1H), 7.88 (d, 2H, J = 7.3 Hz), 7.68 (s, 1H), 7.55-7.48 (m, 3H), 7.32 (t, 1H, J = 7.3 Hz), 6.91 (d, 1H, J = 7.9 Hz), 3.55-3.26 (m, 2H), 2.67- 2.35 (m, 6H), 2.30 (s, 3H), 2.29 (s, 3H), 1.03-0.73 (m, 6H) |
| 93 | 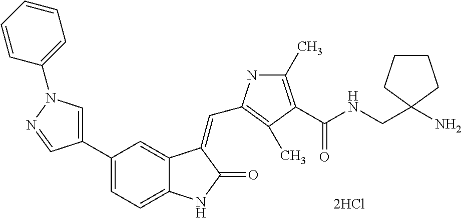 [SMILES: Cc1[nH]c(/C=C2\C(=O)Nc3ccc(-c4cnn(-c5ccccc5)c4)cc32)c(C)c1C(=O)NCC1(N)CCCC1] | 400 MHz ¹H-NMR (DMSO-d₆, δ) 13.73 (s, 1H), 10.97 (s, 1H), 8.93 (s, 1H), 8.24 (s, 1H), 8.15 (s, 1H), 7.96- 7.85 (m, 6H), 7.74 (s, 1H), 7.55- 7.48 (m, 3H), 7.32 (t, 1H, J = 7.3 Hz), 6.92 (d, 1H, J = 8.0 Hz), 3.47 (d, 2H, J = 6.1 Hz), 1.92-1.58 (m, 8H) |
| 94 | 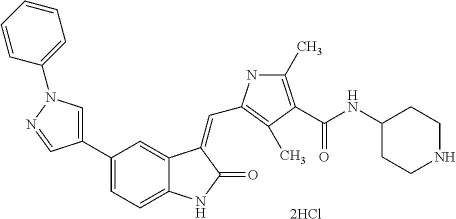 [SMILES: Cc1[nH]c(/C=C2\C(=O)Nc3ccc(-c4cnn(-c5ccccc5)c4)cc32)c(C)c1C(=O)NC1CCNCC1 ; "2HCl"] | 400 MHz ¹H-NMR (DMSO-d₆, δ) 13.59 (s, 1H), 10.88 (s, 1H), 8.85 (s, 1H), 8.62 (br, 1H), 8.39 (br, 1H), 8.16 (s, 1H), 8.06 (s, 1H), 7.81 (d, 2H, J = 8.0 Hz), 7.77 (d, 1H, J = 7.3 Hz), 7.65 (s, 1H), 7.48-7.40 (m, 3H), 7.20 (t, 1H, J = 7.3 Hz), 6.84 (d, 1H, J = 8.0 Hz), 3.94 (m, 1H), 3.20 (m, 2H), 2.94 (m, 2H), 2.37 (s, 3H), 2.35 (s, 3H), 1.93 (m, 2H), 1.63 (m, 2H) |
| 95 | 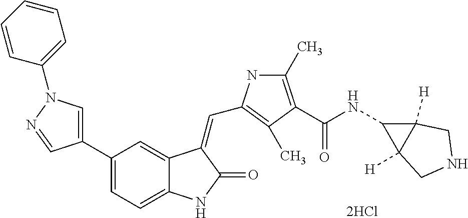 | 400 MHz ¹H-NMR (DMSO-d₆, δ) 13.59 (s, 1H), 10.88 (s, 1H), 9.36 (br, 1H), 8.85 (s, 1H), 8.78 (br, 1H), 8.16 (s, 1H), 8.07 (s, 1H), 7.81 (d, 2H, J = 8.0 Hz), 7.77 (d, 1H, J = 4.3 Hz), 7.48-7.40 (m, 3H), 7.25 (t, 1H, J = 7.3 Hz), 6.84 (d, 1H, J = 8.0 Hz), 3.65-3.20 (m, 4H), 2.88 (m, 1H), 2.36 (s, 3H), 2.34 (s, 3H), 1.88 (br, 2H) |
| 96 | 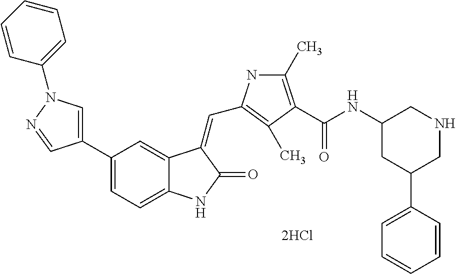 [SMILES: Cc1[nH]c(/C=C2\C(=O)Nc3ccc(-c4cnn(-c5ccccc5)c4)cc32)c(C)c1C(=O)NC1CNCC(c2ccccc2)C1 ; "2HCl"] | LCMS m/z 583.39 (M + H) |
| 97 | 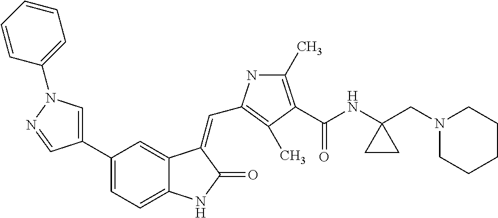 [SMILES: Cc1[nH]c(/C=C2\C(=O)Nc3ccc(-c4cnn(-c5ccccc5)c4)cc32)c(C)c1C(=O)NC1(CN2CCCCC2)CC1] | 400 MHz ¹H-NMR (DMSO-d₆, δ) 13.59 (s, 1H), 10.93 (s, 1H), 8.93 (s, 1H), 8.24 (s, 1H), 8.13 (s, 1H), 7.90 (d, 2H, J = Hz), 7.71 (s, 1H), 7.55-7.48 (m, 3H), 7.33 (t, 1H, J = 7.3 Hz), 6.92 (d, 1H, J = 8.0 Hz), 2.55-2.35 (m, 6H), 2.48 (s, 3H), 2.45 (s, 3H), 1.60-1.35 (m, 6H), 0.88-0.55 (m, 4H). |
| 98 | 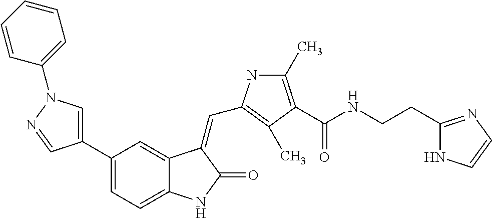 [SMILES: Cc1[nH]c(/C=C2\C(=O)Nc3ccc(-c4cnn(-c5ccccc5)c4)cc32)c(C)c1C(=O)NCCc1ncc[nH]1] | LCMS m/z 518.28 (M + H) |
| 99 | 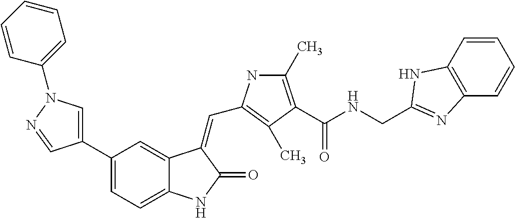 [SMILES: Cc1[nH]c(/C=C2\C(=O)Nc3ccc(-c4cnn(-c5ccccc5)c4)cc32)c(C)c1C(=O)NCc1nc2ccccc2[nH]1] | LCMS m/z 554.29 (M + H) |
| 100 | 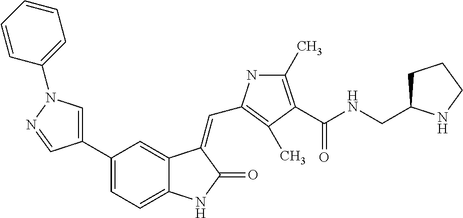 [SMILES: Cc1[nH]c(/C=C2\C(=O)Nc3ccc(-c4cnn(-c5ccccc5)c4)cc32)c(C)c1C(=O)NC[C@H]1CCCN1] | LCMS m/z 507.28 (M + H) |
| 101 | 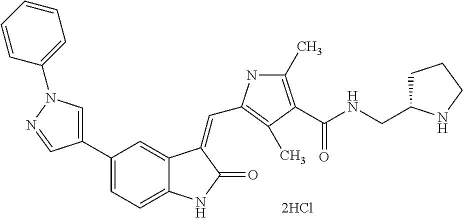 [SMILES: Cc1[nH]c(/C=C2\C(=O)Nc3ccc(-c4cnn(-c5ccccc5)c4)cc32)c(C)c1C(=O)NC[C@@H]1CCCN1 ; "2HCl"] | LCMS m/z 507.28 (M + H) |

### Examples 102 to 128

Reactions and treatments were carried out in the same manner as in Example 1 using the corresponding starting material compounds, thereby giving the compounds of Examples 102 to 128 shown in Table 12.

TABLE 12

| Example | Structure | Spectral data |
|---|---|---|
| 102 | 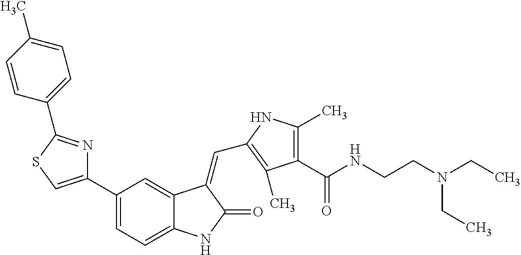 [SMILES: CCN(CC)CCNC(=O)c1c(C)[nH]c(/C=C2\C(=O)Nc3ccc(-c4csc(-c5ccc(C)cc5)n4)cc32)c1C] | LCMS m/z 554.5 (M + H) |
| 103 | 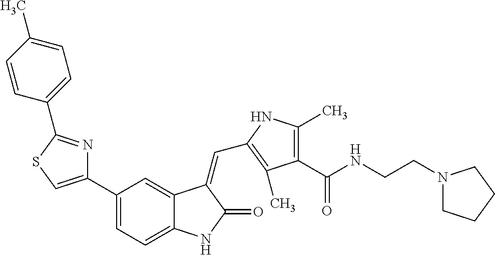 [SMILES: Cc1ccc(-c2nc(-c3ccc4c(c3)/C(=C/c3[nH]c(C)c(C(=O)NCCN5CCCC5)c3C)C(=O)N4)cs2)cc1] | LCMS m/z 552.4 (M + H) |
| 104 | 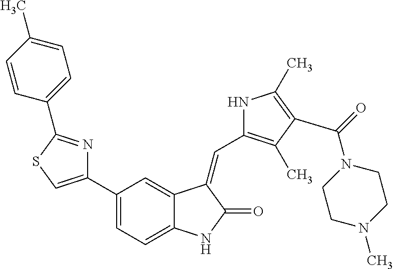 [SMILES: Cc1ccc(-c2nc(-c3ccc4c(c3)/C(=C/c3[nH]c(C)c(C(=O)N5CCN(C)CC5)c3C)C(=O)N4)cs2)cc1] | LCMS m/z 538.5 (M + H) |
| 105 | 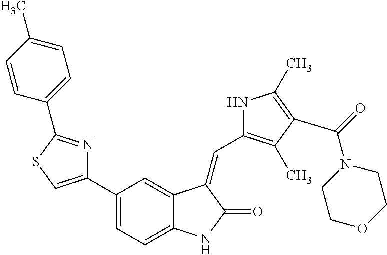 [SMILES: Cc1ccc(-c2nc(-c3ccc4c(c3)/C(=C/c3[nH]c(C)c(C(=O)N5CCOCC5)c3C)C(=O)N4)cs2)cc1] | LCMS m/z 525.7 (M + H) |
| 106 | 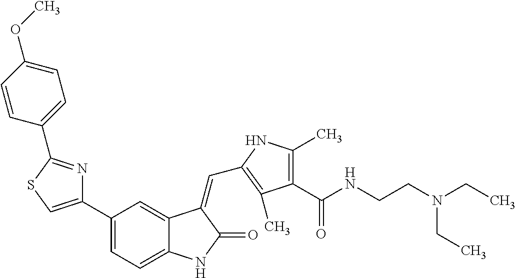 [SMILES: CCN(CC)CCNC(=O)c1c(C)[nH]c(/C=C2\C(=O)Nc3ccc(-c4csc(-c5ccc(OC)cc5)n4)cc32)c1C] | LCMS m/z 570.5 (M + H) |
| 107 | 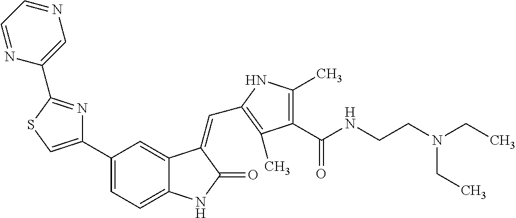 [SMILES: CCN(CC)CCNC(=O)c1c(C)[nH]c(/C=C2\C(=O)Nc3ccc(-c4csc(-c5cnccn5)n4)cc32)c1C] | LCMS m/z 542.4 (M + H) |
| 108 | 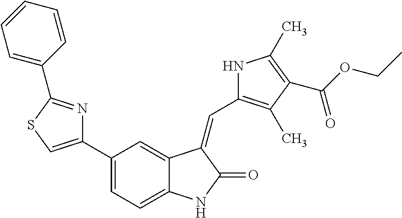 [SMILES: CCOC(=O)c1c(C)[nH]c(/C=C2\C(=O)Nc3ccc(-c4csc(-c5ccccc5)n4)cc32)c1C] | LCMS m/z 470.4 (M + H) |
| 109 | 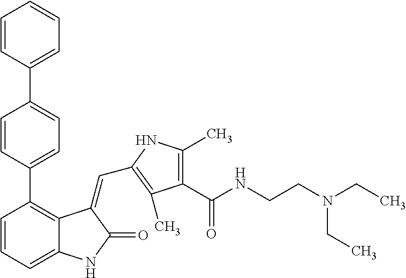 [SMILES: CCN(CC)CCNC(=O)c1c(C)[nH]c(/C=C2\C(=O)Nc3cccc(-c4ccc(-c5ccccc5)cc4)c32)c1C] | LCMS m/z 534 (M + H) |
| 110 | 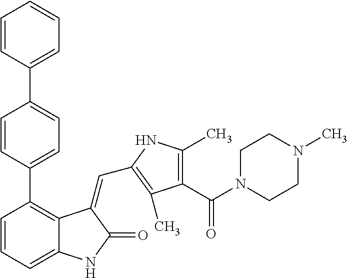 [SMILES: Cc1[nH]c(/C=C2\C(=O)Nc3cccc(-c4ccc(-c5ccccc5)cc4)c32)c(C)c1C(=O)N1CCN(C)CC1] | LCMS m/z 518 (M + H) |
| 111 | 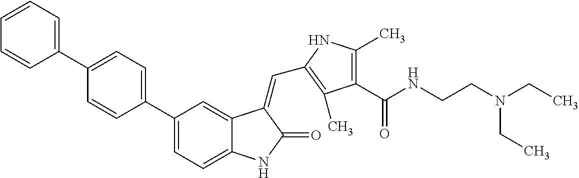 [SMILES: CCN(CC)CCNC(=O)c1c(C)[nH]c(/C=C2\C(=O)Nc3ccc(-c4ccc(-c5ccccc5)cc4)cc32)c1C] | LCMS m/z 533.4 (M + H) |
| 112 | 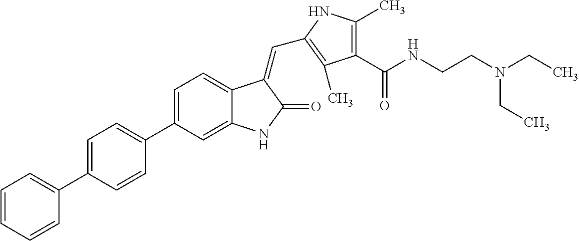 [SMILES: CCN(CC)CCNC(=O)c1c(C)[nH]c(/C=C2\C(=O)Nc3cc(-c4ccc(-c5ccccc5)cc4)ccc32)c1C] | LCMS m/z 267 (M + 2H)/2 |
| 113 | 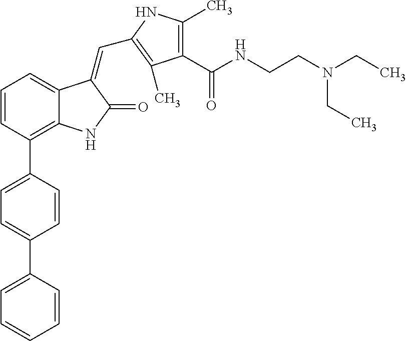 [SMILES: CCN(CC)CCNC(=O)c1c(C)[nH]c(/C=C2\C(=O)Nc3c2cccc3-c2ccc(-c3ccccc3)cc2)c1C] | LCMS m/z 533.4 (M + H) |
| 114 | 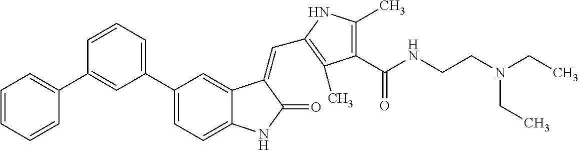 [SMILES: CCN(CC)CCNC(=O)c1c(C)[nH]c(/C=C2\C(=O)Nc3ccc(-c4cccc(-c5ccccc5)c4)cc32)c1C] | LCMS m/z 533.4 (M + H) |
| 115 | 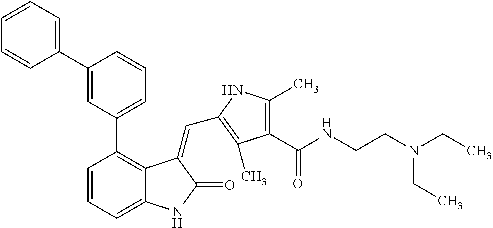 [SMILES: CCN(CC)CCNC(=O)c1c(C)[nH]c(/C=C2\C(=O)Nc3cccc(-c4cccc(-c5ccccc5)c4)c32)c1C] | LCMS m/z 533.4 (M + H) |
| 116 | 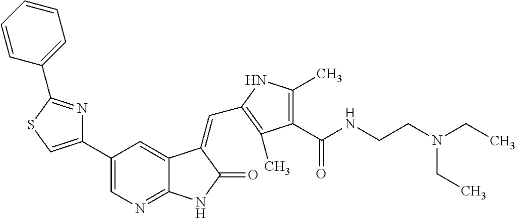 [SMILES: CCN(CC)CCNC(=O)c1c(C)[nH]c(/C=C2\C(=O)Nc3ncc(-c4csc(-c5ccccc5)n4)cc32)c1C] | LCMS m/z 541.3 (M + H) |
| 117 | 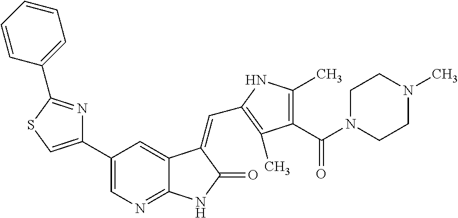 [SMILES: Cc1[nH]c(/C=C2\C(=O)Nc3ncc(-c4csc(-c5ccccc5)n4)cc32)c(C)c1C(=O)N1CCN(C)CC1] | LCMS m/z 525.3 (M + H) |
| 118 | 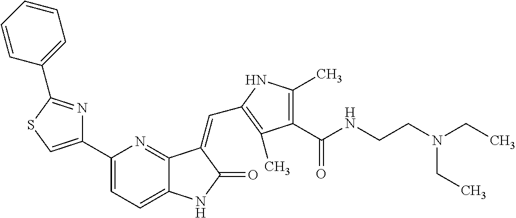 [SMILES: CCN(CC)CCNC(=O)c1c(C)[nH]c(/C=C2\C(=O)Nc3ccc(-c4csc(-c5ccccc5)n4)nc32)c1C] | LCMS m/z 541.3 (M + H) |
| 119 | 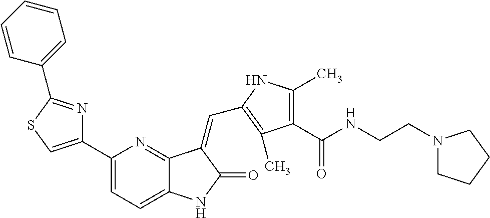 [SMILES: Cc1[nH]c(/C=C2\C(=O)Nc3ccc(-c4csc(-c5ccccc5)n4)nc32)c(C)c1C(=O)NCCN1CCCC1] | LCMS m/z 539.3 (M + H) |
| 120 | 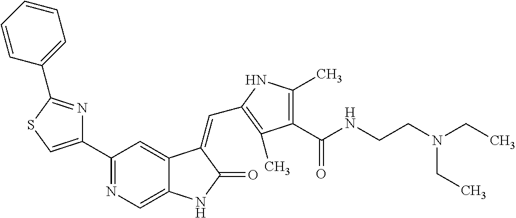 [SMILES: CCN(CC)CCNC(=O)c1c(C)[nH]c(/C=C2\C(=O)Nc3cnc(-c4csc(-c5ccccc5)n4)cc32)c1C] | LCMS m/z 541.3 (M + H) |
| 121 | 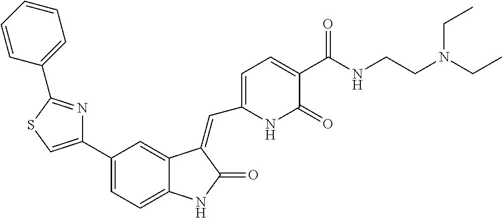 [SMILES: CCN(CC)CCNC(=O)c1ccc(/C=C2\C(=O)Nc3ccc(-c4csc(-c5ccccc5)n4)cc32)[nH]c1=O] | LCMS m/z 540.3 (M + H) |
| 122 | 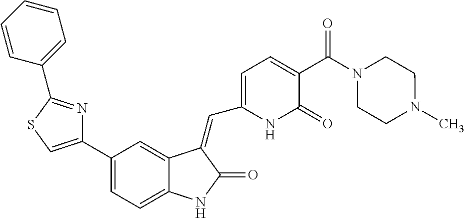 [SMILES: CN1CCN(C(=O)c2ccc(/C=C3\C(=O)Nc4ccc(-c5csc(-c6ccccc6)n5)cc43)[nH]c2=O)CC1] | LCMS m/z 524 (M + H) |
| 123 | 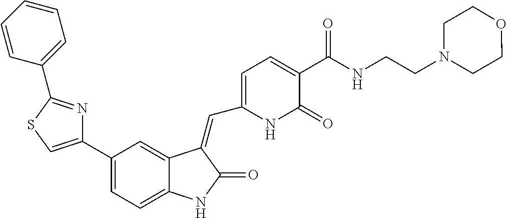 [SMILES: O=C1Nc2ccc(-c3csc(-c4ccccc4)n3)cc2/C1=C/c1ccc(C(=O)NCCN2CCOCC2)c(=O)[nH]1] | LCMS m/z 554 (M + H) |
| 124 | 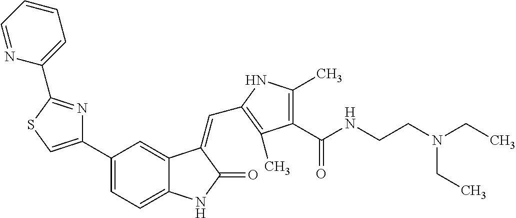 [SMILES: CCN(CC)CCNC(=O)c1c(C)[nH]c(/C=C2\C(=O)Nc3ccc(-c4csc(-c5ccccn5)n4)cc32)c1C] | LCMS m/z 541.5 (M + H) |
| 125 | 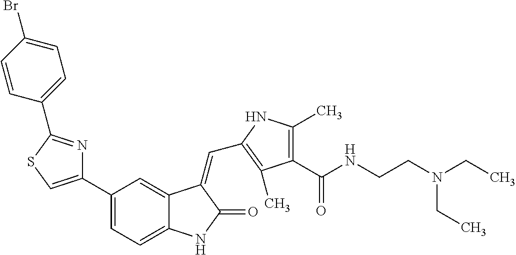 [SMILES: CCN(CC)CCNC(=O)c1c(C)[nH]c(/C=C2\C(=O)Nc3ccc(-c4csc(-c5ccc(Br)cc5)n4)cc32)c1C] | LCMS m/z 618.5 (M + H) |
| 126 | 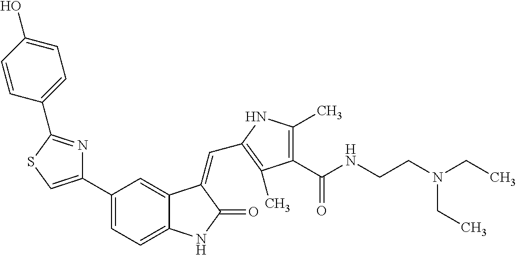 [SMILES: CCN(CC)CCNC(=O)c1c(C)[nH]c(/C=C2\C(=O)Nc3ccc(-c4csc(-c5ccc(O)cc5)n4)cc32)c1C] | LCMS m/z 556.5 (M + H) |
| 127 | 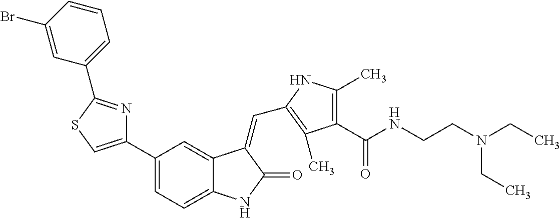 [SMILES: CCN(CC)CCNC(=O)c1c(C)[nH]c(/C=C2\C(=O)Nc3ccc(-c4csc(-c5cccc(Br)c5)n4)cc32)c1C] | LCMS m/z 618.4 (M + H) |
| 128 | 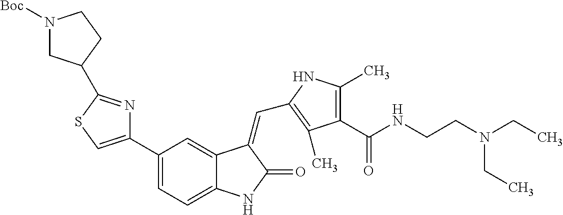 [SMILES: CCN(CC)CCNC(=O)c1c(C)[nH]c(/C=C2\C(=O)Nc3ccc(-c4csc(C5CCN(C(=O)OC(C)(C)C)C5)n4)cc32)c1C] | LCMS m/z 633.6 (M + H) |

### Example 129

### Preparation of Compound 129

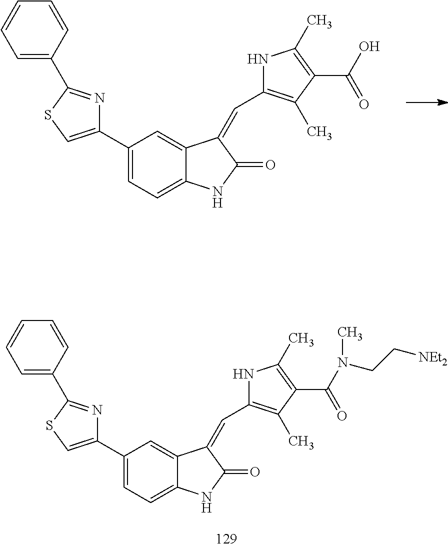

To a suspension of (Z)—N-(2-(diethylamino)ethyl)-N,2,4-trimethyl-5-((2-oxo-5-(2-phenylthiazol-4-yl)indolin-3-ylidene)methyl)-1H-pyrrole-3-carboxamide (Z)-2,4-dimethyl-5-((2-oxo-5-(2-phenylthiazol-4-yl)indolin-3-ylidene)methyl)-1H-pyrrole-3-carboxylate (30 mg, 0067 mmol) in tetrahydrofuran (1 mL) were added WSC (14.3 mg), HOBt (10 mg), and N,N-diethyl-N′-methylethane-1,2-diamine (22 μL) at rt. After stirring overnight, the suspension was filtered through a filtrate paper. The residual solid was washed with H2O and ethyl acetate, then dried over in vacuo to obtain product 129 (33 mg). m/z 554.5 [M+1]

### Examples 130 to 131

Reactions and treatments were carried out in the same manner as in Example 129 using the corresponding starting material compounds, thereby giving the compounds of Examples 130 to 131 shown in Table 13.

TABLE 13

| Example | Structure | Spectral data |
|---|---|---|
| 130 | 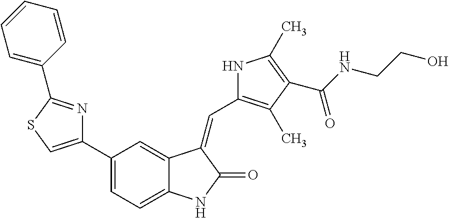 [SMILES: Cc1[nH]c(/C=C2\C(=O)Nc3ccc(-c4csc(-c5ccccc5)n4)cc32)c(C)c1C(=O)NCCO] | LCMS m/z 485.6 (M + H) |
| 131 | 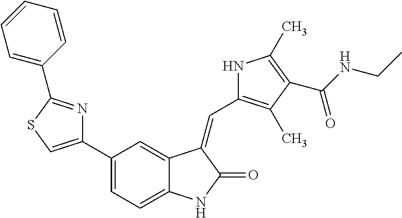 [SMILES: CCNC(=O)c1c(C)[nH]c(/C=C2\C(=O)Nc3ccc(-c4csc(-c5ccccc5)n4)cc32)c1C] | LCMS m/z 469 (M + H) |

### Example 132

### Preparation of Compound 132

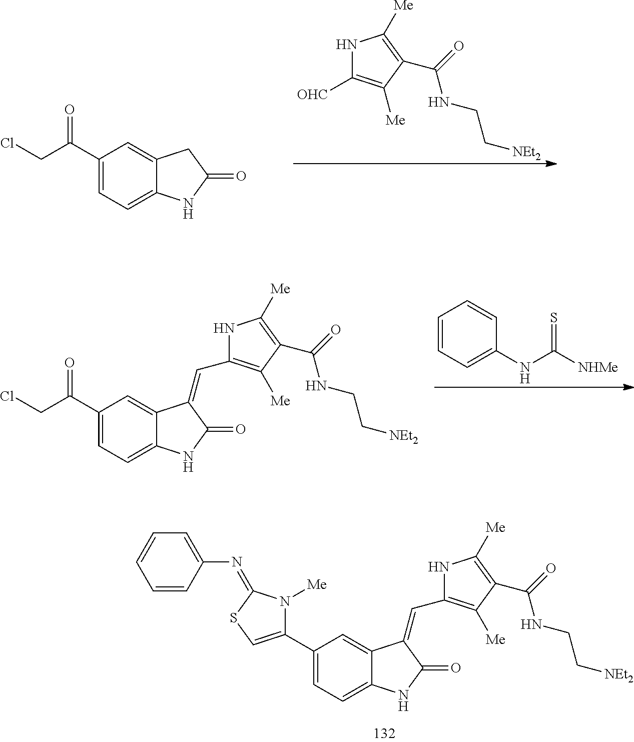

Step 1

To a suspension of 5-(2-chloroacetyl)indolin-2-one (58 mg, 0.2 mmol) in EtOH (3 mL) was added N-(2-(diethylamino)ethyl)-5-formyl-2,4-dimethyl-1H-pyrrole-3-carboxamide (28 mg, 0.2 mmol) and piperidine (a drop). The resulting mixture was heated for 2 hrs at 80° C. for 16 h before cooling, and then concentrated in vacuo. The reaction mixture was concentrated. The residue was purified by prep-HPLC to obtain product (Z)-5-((5-(2-chloroacetyl)-2-oxoindolin-3-ylidene)methyl)-N-(2-(diethylamino)ethyl)-2,4-dimethyl-1H-pyrrole-3-carboxamide (30 mg).

Step 2

(Z)-5-((5-(2-chloroacetyl)-2-oxoindolin-3-ylidene)methyl)-N-(2-(diethylamino)ethyl)-2,4-dimethyl-1H-pyrrole-3-carboxamide (30 mg, 0.066 mmol) and 1-methyl-3-phenylthiourea (12 mg, 0.072 mmol) were dissolved in DMF (0.5 mL) and heated to 130 C for 1 h. After cooling to rt, reaction mixture was poured into water (2 ml) and neutralized by sat.NaHCO₃. Precipitate was filtered and purified by SiO₂ column to get the compound 132 (31 mg).

### Example 133

### Preparation of Compound 133

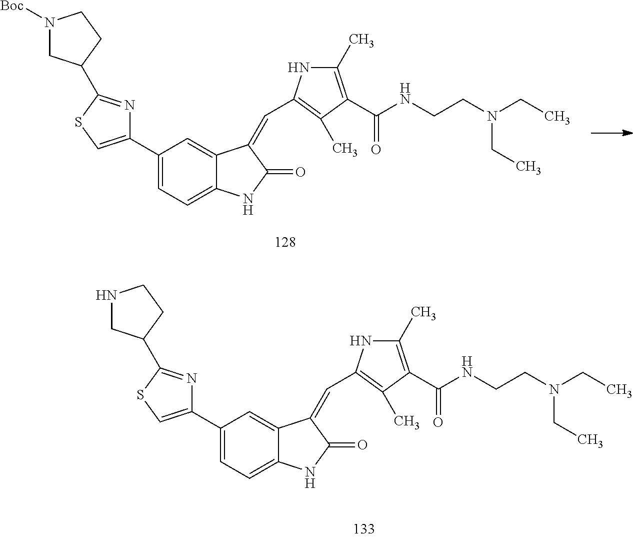 [SMILES: CCN(CC)CCNC(=O)c1c(C)[nH]c(/C=C2\C(=O)Nc3ccc(-c4csc(C5CCN(C(=O)OC(C)(C)C)C5)n4)cc32)c1C ; CCN(CC)CCNC(=O)c1c(C)[nH]c(/C=C2\C(=O)Nc3ccc(-c4csc(C5CCNC5)n4)cc32)c1C]

Step 1

Compound 128 (229 mg, 0.36 mmol) was dissolved in trifluoroacetic acid (20 mL). After 1 h, the mixture was concentrated in vacuo. The residual solid was suspended in ethyl acetate (2 mL), and the mixture was filtered through a filtrate paper. The residual brown solid was dried in vacuo to get the compound 133 (205 mg). m/z 533.5 [M+1]

### Example 134 and 135

### Preparation of Compound 134 and 135

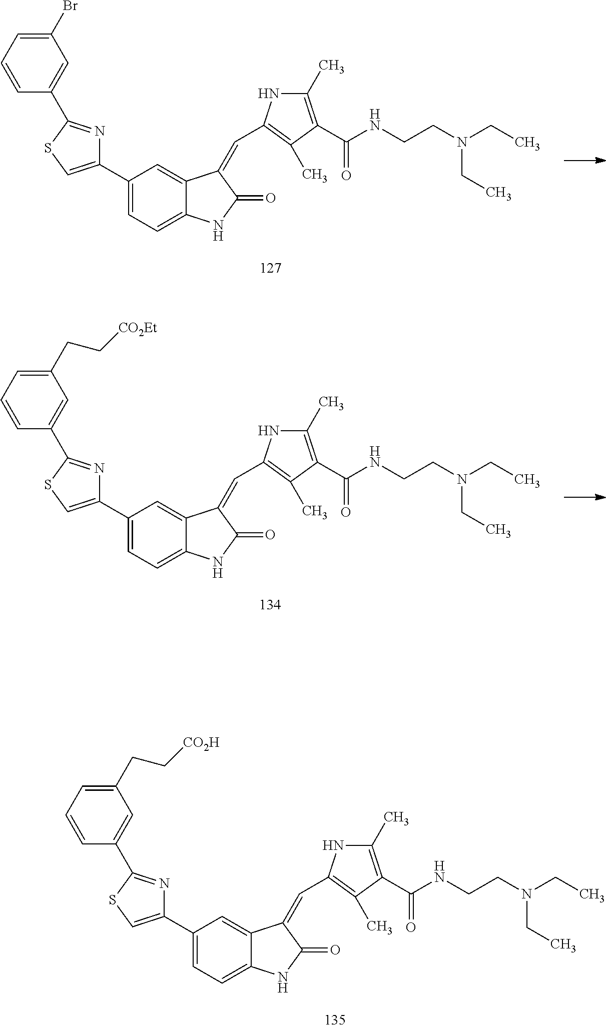 [SMILES: CCN(CC)CCNC(=O)c1c(C)[nH]c(/C=C2\C(=O)Nc3ccc(-c4csc(-c5cccc(Br)c5)n4)cc32)c1C ; CCOC(=O)CCc1cccc(-c2nc(-c3ccc4c(c3)/C(=C/c3[nH]c(C)c(C(=O)NCCN(CC)CC)c3C)C(=O)N4)cs2)c1 ; CCN(CC)CCNC(=O)c1c(C)[nH]c(/C=C2\C(=O)Nc3ccc(-c4csc(-c5cccc(CCC(=O)O)c5)n4)cc32)c1C]

Step 1

A mixture of compound 127 (2.8 mg, 0.045 mmol), bis(tri-t-butylphosphine)palladium(0) (6.7 mg, 0.013 mmol), Zn powder (3.5 mg, 0.054 mmol), and 2-(ethoxycarbonyl)ethylzinc bromide (0.5 M in ether, 0.45 mL, 0.23 mmol) in tetrahydrofurane (1 mL) was heated at 70 C. After 2 h, the mixture was concentrated in vacuo. The residual solid was chromatographed on silica gel to get the compound 134 (67 mg). m/z 640.6 [M+1]

Step 2

To a solution of compound 134 (67 mg, 0.045 mmol) in tetrahydrofurane (1 mL) was added 5 N aq. NaOH (1 mL). After stirring for 2 h at 80 C, 5 N aq. HCl was added to the reaction mixture. The reaction mixture was neutralized with sat. NaHCO₃, then extracted with EtOH/CHCl₃ four times. The organic extracts were concentrated in vacuo. The residual solid was suspended in hexane/ethyl acetate=1/1, then filtered to get the compound 135 (30 mg). m/z 612.5 [M+1]

### Example 136 and 137

### Preparation of Compound 136 and 137

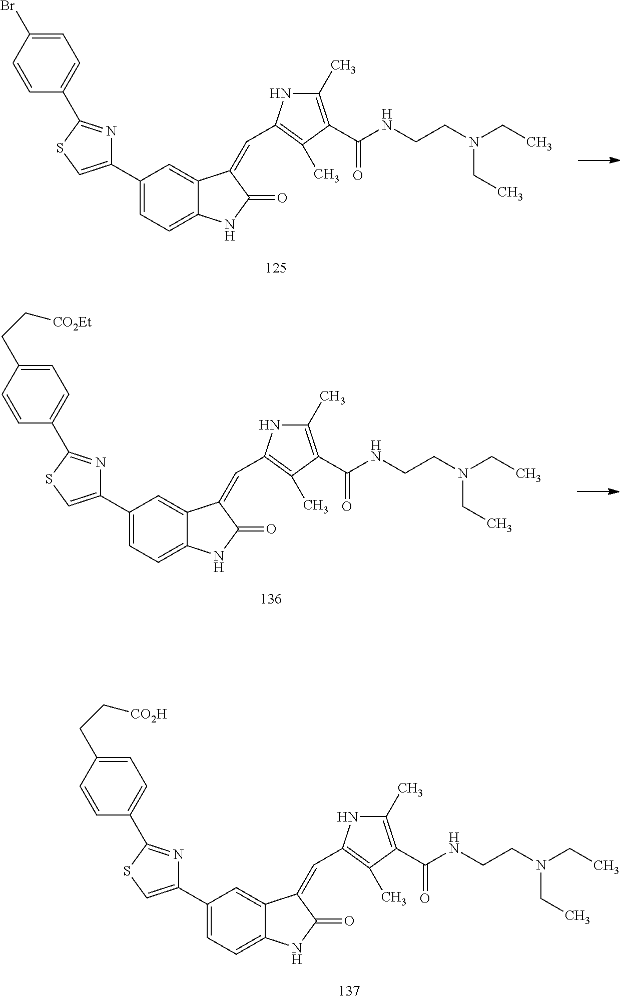 [SMILES: CCN(CC)CCNC(=O)c1c(C)[nH]c(/C=C2\C(=O)Nc3ccc(-c4csc(-c5ccc(Br)cc5)n4)cc32)c1C ; CCOC(=O)CCc1ccc(-c2nc(-c3ccc4c(c3)/C(=C/c3[nH]c(C)c(C(=O)NCCN(CC)CC)c3C)C(=O)N4)cs2)cc1 ; CCN(CC)CCNC(=O)c1c(C)[nH]c(/C=C2\C(=O)Nc3ccc(-c4csc(-c5ccc(CCC(=O)O)cc5)n4)cc32)c1C]

Step 1

Reactions and treatments were carried out in the same manner as in Example 134 using the corresponding starting material compound 125, thereby giving the compound 136. m/z 640.5 [M+1]

Step 2

Reactions and treatments were carried out in the same manner as in Example 135 using the corresponding starting material compound 136, thereby giving the compound 137. m/z 612.5 [M+1]

### Example 138

### Preparation of Compound 138

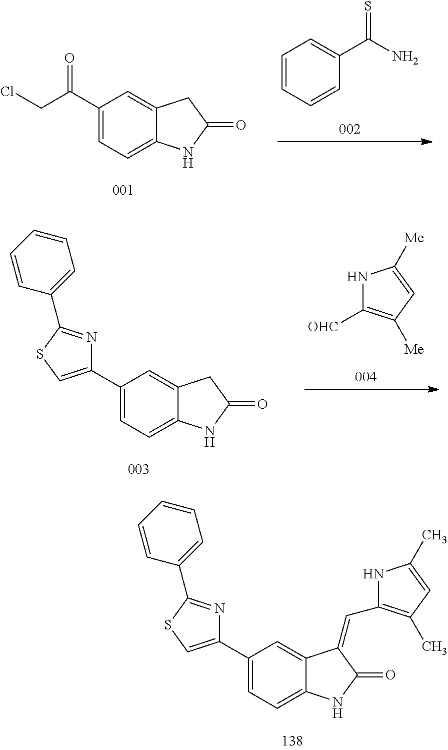 [SMILES: O=C1Cc2cc(C(=O)CCl)ccc2N1 ; NC(=S)c1ccccc1 ; O=C1Cc2cc(-c3csc(-c4ccccc4)n3)ccc2N1 ; Cc1cc(C)c(C=O)[nH]1 ; Cc1cc(C)c(/C=C2\C(=O)Nc3ccc(-c4csc(-c5ccccc5)n4)cc32)[nH]1]

Step 1

A suspension of 5-chloroacetyloxindole 001 (838 mg, 4 mmol) and thiobenzamide 002 (550 mg, 4 mmol) in DMF (8 mL) was heated at 70° C. for 16 h and then cooled down to room temperature. At 0° C., while stirring, Na₂CO₃ aq (1N, 8 mL) was added drop wise to the reaction mixture. The mixture was stirred at room temperature for 20 min, filtrated, and washed with H₂O (5 mL×2). The cake was put into a flask and EtOH (5 mL) was added. The mixture was stirred at room temperature for 30 min, filtrated, and washed with EtOH (2 mL×2). The collected solid was dried down under vacuum to yield the compound 003 as a light brown solid (1.0 g, 85%).

Step 2

To a suspension of 003 (58 mg, 0.2 mmol) in EtOH (3 mL) was added 004 (28 mg, 0.2 mmol) and piperidine (a drop). The resulting mixture was heated for 2 hrs at 80° C. before cooling, and then concentrated in vacuo. The reaction mixture was concentrated. The residue was purified by prep-HPLC to obtain product 138 (20 mg). m/z 398 [M+1]

### Example 139

### Preparation of Compound 139

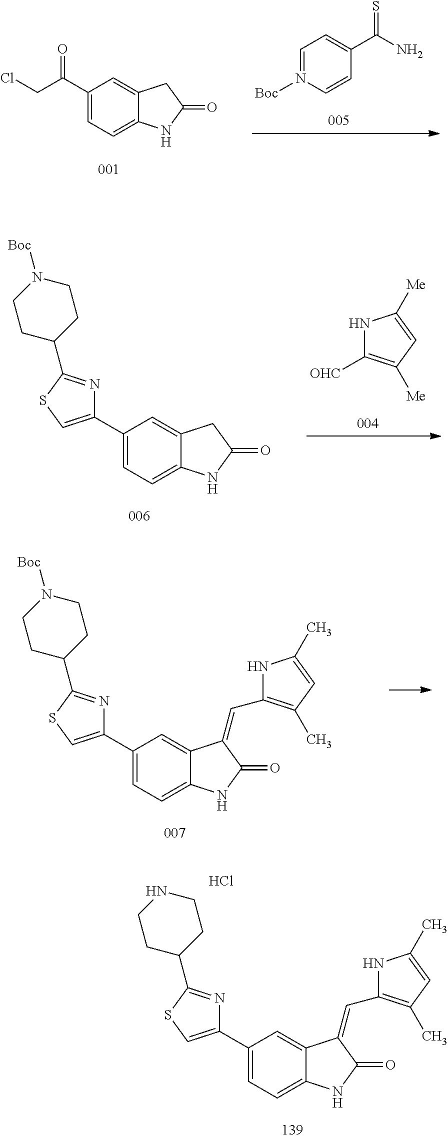

Step 1

A suspension of 5-chloroacetyloxindole 001 (419 mg, 2 mmol) and thiamide 005 (489 mg, 2 mmol) in DMF (10 mL) was heated at 80° C. for 16 h and then cooled down to room temperature. The mixture was concentrated, and the residue was partitioned in EtOAc and 1N NaHCO₃ aq. The organic layer was washed with H₂O, and brine, dried over Na₂SO₄, and concentrated in vacuo. The residue was purified by prep-HPLC to obtain product 006.

Step 2

To a suspension of 006 (80 mg, 0.2 mmol) in EtOH (3 mL) was added 004 (28 mg, 0.2 mmol) and piperidine (0.1 mL). The resulting mixture was heated for 2 hrs at 80° C. before cooling, and then concentrated in vacuo. The reaction mixture was concentrated. The residue was partitioned in EtOAc and H₂O, and the combined organic layers were washed with H₂O and brine, dried over Na₂SO₄, and concentrated in vacuo. The residue was purified by prep-HPLC to obtain product 007.

Step 3

To a solution of 007 (65 mg, 0.13 mmol) in MeOH (5 mL) was added HCl (4N in dioxane, 1 mL). The resulting mixture was stirred at room temperature for overnight. The reaction mixture was concentrated. The residue was purified by prep-HPLC to obtain product 139 (37 mg). m/z 405 [M+1]

### Example 140

### Preparation of Compound 140

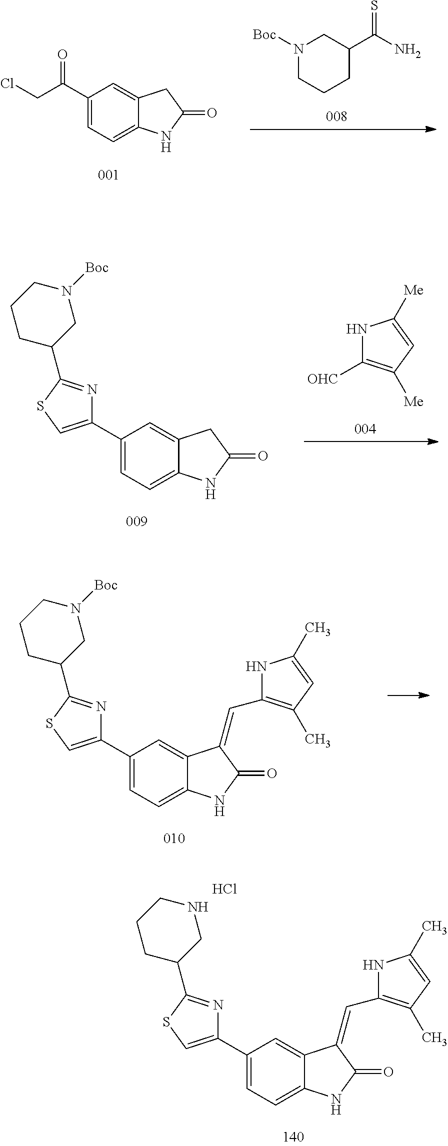

Step 1

A suspension of 5-chloroacetyloxindole 001 (419 mg, 2 mmol) and thiamide 008 (489 mg, 2 mmol) in DMF (10 mL) was heated at 80° C. for 16 h and then cooled down to room temperature. The mixture was concentrated, and the residue was partitioned in EtOAc and 1N NaHCO₃ aq. The organic layer was washed with H₂O, and brine, dried over Na₂SO₄, and concentrated in vacuo. The residue was purified by prep-HPLC to obtain product 009.

Step 2

To a suspension of 009 (80 mg, 0.2 mmol) in EtOH (3 mL) was added 004 (2.8 mg, 0.2 mmol) and piperidine (0.1 mL). The resulting mixture was heated for 2 hrs at 80° C. before cooling, and then concentrated in vacuo. The reaction mixture was concentrated. The residue was partitioned in EtOAc and H₂O, and the combined organic layers were washed with H₂O and brine, dried over Na₂SO₄, and concentrated in vacuo. The residue was purified by prep-HPLC to obtain product 010.

Step 3

To a solution of 010 (70 mg, 0.14 mmol) in MeOH (5 mL) was added HCl (4N in dioxane, 1 mL). The resulting mixture was stirred at room temperature for overnight. The reaction mixture was concentrated. The residue was purified by prep-HPLC to obtain product 140 (21 mg). m/z 405 [M+1]

### Example 141

### Preparation of Compound 141

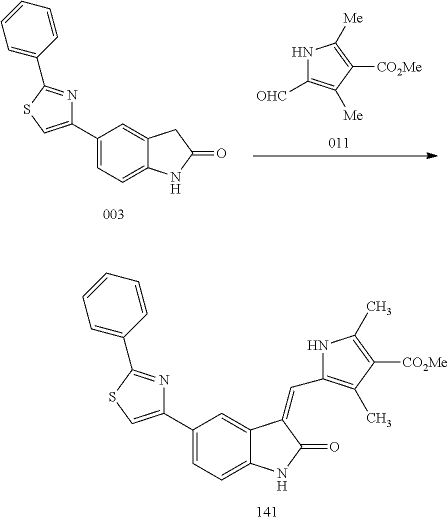 [SMILES: O=C1Cc2cc(-c3csc(-c4ccccc4)n3)ccc2N1 ; COC(=O)c1c(C)[nH]c(C=O)c1C ; COC(=O)c1c(C)[nH]c(/C=C2\C(=O)Nc3ccc(-c4csc(-c5ccccc5)n4)cc32)c1C]

Step 1

To a suspension of 003 (58 mg, 0.2 mmol) in EtOH (3 mL) was added 011 (40 mg, 0.22 mmol) and piperidine (one drop). The resulting mixture was heated for 16 hrs at 80° C. before cooling, and then concentrated in vacuo. The reaction mixture was concentrated. The residue was purified by prep-HPLC to obtain product 141 (81 mg). m/z 456 [M+1]

### Example 142

### Preparation of Compound 142

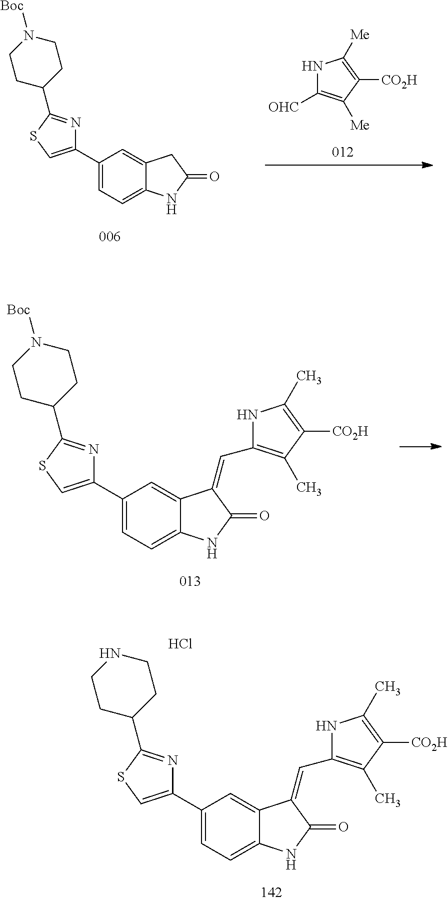 [SMILES: CC(C)(C)OC(=O)N1CCC(c2nc(-c3ccc4c(c3)CC(=O)N4)cs2)CC1 ; Cc1[nH]c(C=O)c(C)c1C(=O)O ; Cc1[nH]c(/C=C2\C(=O)Nc3ccc(-c4csc(C5CCN(C(=O)OC(C)(C)C)CC5)n4)cc32)c(C)c1C(=O)O ; Cl ; Cc1[nH]c(/C=C2\C(=O)Nc3ccc(-c4csc(C5CCNCC5)n4)cc32)c(C)c1C(=O)O]

Step 1

To a suspension of 006 (80 mg, 0.2 mmol) in EtOH (3 mL) was added 012 (34 mg, 0.2 mmol) and piperidine (0.1 mL). The resulting mixture was heated for 2 hrs at 80° C. for before cooling, and then concentrated in vacuo. The reaction mixture was concentrated. The residue was partitioned in EtOAc and H₂O, adjusted pH to ˜5, and the combined organic layers were washed with H₂O and brine, dried over Na₂SO₄, and concentrated in vacuo. The residue was purified by prep-HPLC to obtain product 013.

Step 2

To a solution of 013 (35 mg, 0.064 mmol) in MeOH (5 mL) was added HCl (4N in dioxane, 1 mL). The resulting mixture was stirred at room temperature for overnight. The reaction mixture was concentrated. The residue was purified by prep-HPLC to obtain product 142 (19 mg). m/z 449 [M+1]

### Example 143

### Preparation of Compound 143

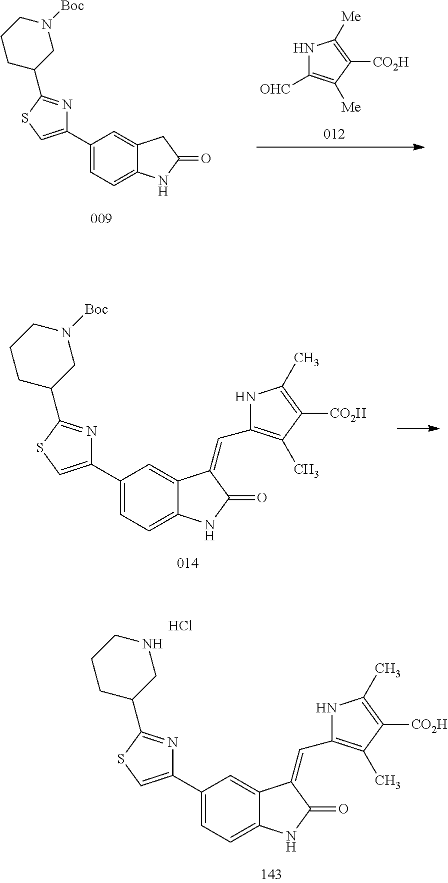 [SMILES: CC(C)(C)OC(=O)N1CCCC(c2nc(-c3ccc4c(c3)CC(=O)N4)cs2)C1 ; Cc1[nH]c(C=O)c(C)c1C(=O)O ; Cc1[nH]c(/C=C2\C(=O)Nc3ccc(-c4csc(C5CCCN(C(=O)OC(C)(C)C)C5)n4)cc32)c(C)c1C(=O)O ; Cl ; Cc1[nH]c(/C=C2\C(=O)Nc3ccc(-c4csc(C5CCCNC5)n4)cc32)c(C)c1C(=O)O]

Step 1

To a suspension of 009 (80 mg, 0.2 mmol) in EtOH (3 mL) was added 012 (34 mg, 0.2 mmol) and piperidine (0.1 mL). The resulting mixture was heated for 2 hrs at 80° C. for before cooling, and then concentrated in vacuo. The reaction mixture was concentrated. The residue was partitioned in EtOAc and H₂O, adjusted pH to ˜5, and the combined organic layers were washed with H₂O and brine, dried over Na₂SO₄, and concentrated in vacuo. The residue was purified by prep-HPLC to obtain product 014.

Step 2

To a solution of 014 (31 mg, 0.06 mmol) in MeOH (5 mL) was added HCl (4N in dioxane, 1 mL). The resulting mixture was stirred at room temperature for overnight. The reaction mixture was concentrated. The residue was purified by prep-HPLC to obtain product 143 (15 mg). m/z 449 [M+1]

### Example 144

### Preparation of Compound 144

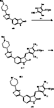 [SMILES: CC(C)(C)OC(=O)N1CCC(c2nc(-c3ccc4c(c3)CC(=O)N4)cs2)CC1 ; COC(=O)c1c(C)[nH]c(C=O)c1C ; COC(=O)c1c(C)[nH]c(/C=C2\C(=O)Nc3ccc(-c4csc(C5CCN(C(=O)OC(C)(C)C)CC5)n4)cc32)c1C ; Cl ; COC(=O)c1c(C)[nH]c(/C=C2\C(=O)Nc3ccc(-c4csc(C5CCNCC5)n4)cc32)c1C]

Step 1

To a suspension of 006 (80 mg, 0.2 mmol) in EtOH (3 mL) was added 011 (40 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 015.

Step 2

To a solution of 015 (56 mg, 0.1 mmol) in MeOH (5 mL) was added HCl (4N in dioxane, 1 mL). The resulting mixture was stirred at rt for overnight. The reaction mixture was concentrated. The residue was purified by prep-HPLC to obtain product 144 (45 mg). m/z 463 [M+1]

### Example 145

### Preparation of Compound 145

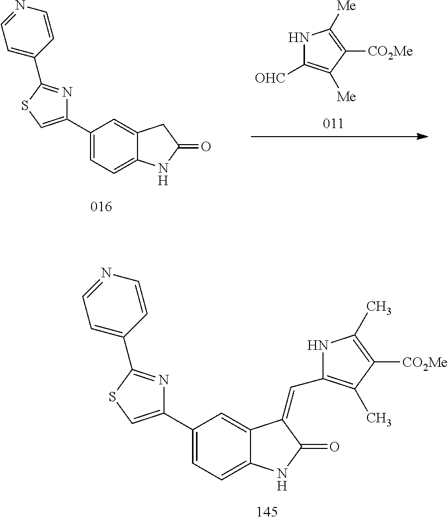 [SMILES: O=C1Cc2cc(-c3csc(-c4ccncc4)n3)ccc2N1 ; COC(=O)c1c(C)[nH]c(C=O)c1C ; COC(=O)c1c(C)[nH]c(/C=C2\C(=O)Nc3ccc(-c4csc(-c5ccncc5)n4)cc32)c1C]

Step 1

To a suspension of 016 (80 mg, 0.2 mmol) in EtOH (3 mL) was added 011 (40 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 145 (55 mg). m/z 457 [M+1]

### Example 146

### Preparation of Compound 146

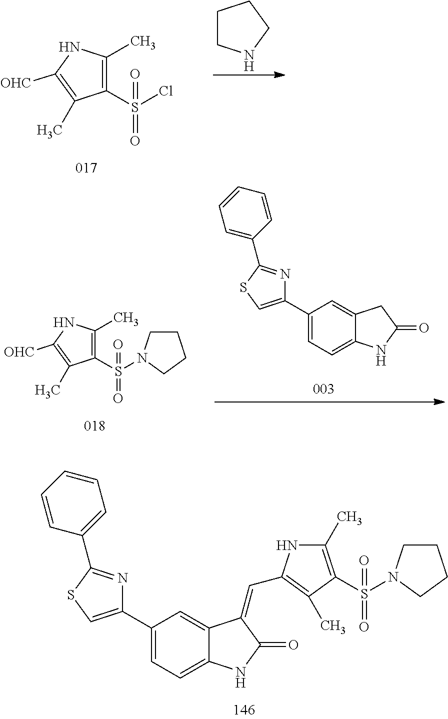 [SMILES: Cc1[nH]c(C=O)c(C)c1S(=O)(=O)Cl ; C1CCNC1 ; Cc1[nH]c(C=O)c(C)c1S(=O)(=O)N1CCCC1 ; O=C1Cc2cc(-c3csc(-c4ccccc4)n3)ccc2N1 ; Cc1[nH]c(/C=C2\C(=O)Nc3ccc(-c4csc(-c5ccccc5)n4)cc32)c(C)c1S(=O)(=O)N1CCCC1]

Step 1

To a solution of 017 (66 mg, 0.3 mmol) in dichloromethane (2 mL) at 0 C was added pyrrolidine (50 ul) and then Et3N (200 ul). The reaction mixture was stirred for 2 h before quenching with aq NH4Cl then regular aqueous work-up. The residue was purified by prep-HPLC to obtain product 018.

Step 2

To a suspension of 003 (58 mg, 0.2 mmol) in EtOH (3 mL) was added 018 (54 mg, 0.21 mmol) and piperidine (a drop). The resulting mixture was heated for 16 hrs at 80° C. for before cooling, and then concentrated in vacuo. The reaction mixture was concentrated. The residue was purified by prep-HPLC to obtain product 146 (85 mg). m/z 531 [M+1]

### Example 147

### Preparation of Compound 147

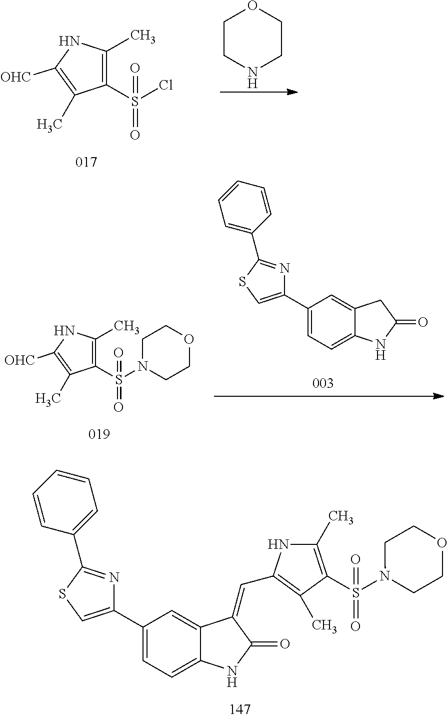 [SMILES: Cc1[nH]c(C=O)c(C)c1S(=O)(=O)Cl ; C1COCCN1 ; Cc1[nH]c(C=O)c(C)c1S(=O)(=O)N1CCOCC1 ; O=C1Cc2cc(-c3csc(-c4ccccc4)n3)ccc2N1 ; Cc1[nH]c(/C=C2\C(=O)Nc3ccc(-c4csc(-c5ccccc5)n4)cc32)c(C)c1S(=O)(=O)N1CCOCC1]

Step 1

To a solution of 017 (66 mg, 0.3 mmol) in dichloromethane (2 mL) at 0 C was added morphline (50 ul) and then Et₃N (200 ul). The reaction mixture was stirred for 2 h before quenching with aq NH₄Cl then regular aqueous work-up. The residue was purified by prep-HPLC to obtain product 019.

Step 2

To a suspension of 003 (58 mg, 0.2 mmol) in EtOH (3 mL) was added 019 (59 mg, 0.21 mmol) and piperidine (a drop). The resulting mixture was heated for 16 hrs at 80° C. for before cooling, and then concentrated in vacuo. The reaction mixture was concentrated. The residue was purified by prep-HPLC to obtain product 147 (98 mg). m/z 547 [M+1]

### Example 148

### Preparation of Compound 148

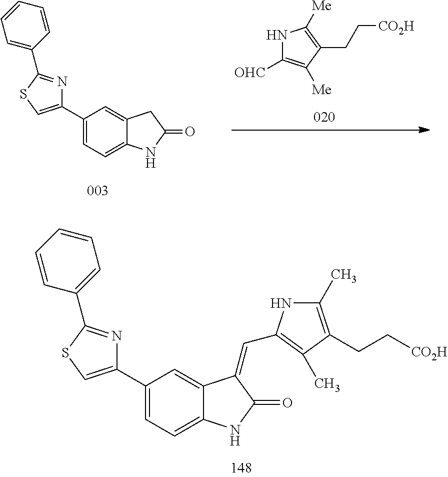 [SMILES: O=C1Cc2cc(-c3csc(-c4ccccc4)n3)ccc2N1 ; Cc1[nH]c(C=O)c(C)c1CCC(=O)O ; Cc1[nH]c(/C=C2\C(=O)Nc3ccc(-c4csc(-c5ccccc5)n4)cc32)c(C)c1CCC(=O)O]

Step 1

To a suspension of 003 (58 mg, 0.2 mmol) in EtOH (3 mL) was added 020 (42 mg, 0.22 mmol) and piperidine (one drop). The resulting mixture was heated for 16 hrs at 80° C. for before cooling, and then concentrated in vacuo. The reaction mixture was concentrated and neutralized to pH=4-5. The residue was purified by prep-HPLC to obtain product 148 (20 mg). m/z 470 [M+1]

### Example 149

### Preparation of Compound 149

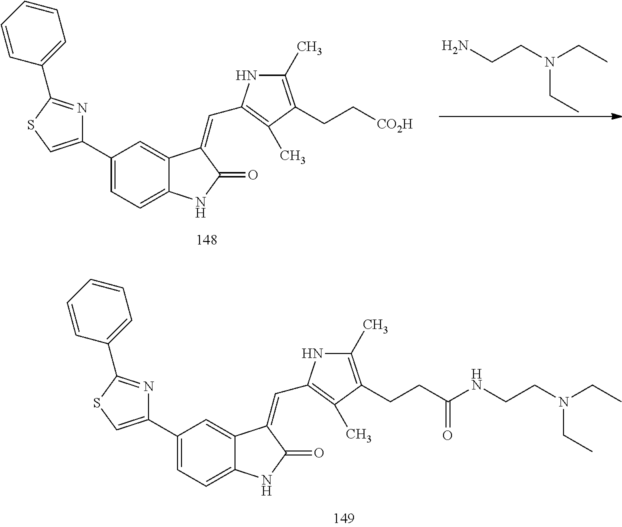

Step 1

To a solution of 148 (41 mg, 0.088 mmol) in DMF (1.5 mL) was added HATU (67 mg), diisopropylethylamine (500 uL), and diethylethylenediamine (25 mg). The mixture was stirred at room temperature for 16 hours then concentrated in vacuo. The residue was added CH₂Cl₂ and washed with H₂O. The organic layer was dried over Na₂SO₄ and concentrated in vacuo. The residue was purified by prep-HPLC to obtain product 149 (40 mg). m/z 568 [M+1]

### Example 150

### Preparation of Compound 150

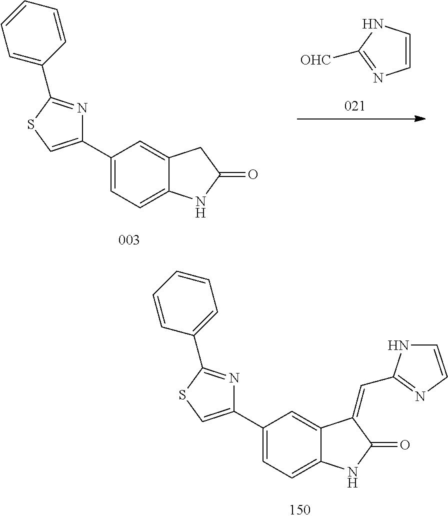 [SMILES: O=C1Cc2cc(-c3csc(-c4ccccc4)n3)ccc2N1 ; O=Cc1ncc[nH]1 ; O=C1Nc2ccc(-c3csc(-c4ccccc4)n3)cc2/C1=C/c1ncc[nH]1]

Step 1

To a suspension of 003 (58 mg, 0.2 mmol) in EtOH (3 mL) was added 021 (21 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 150 (42 mg). m/z 371 [M+1]

### Example 151

### Preparation of Compound 151

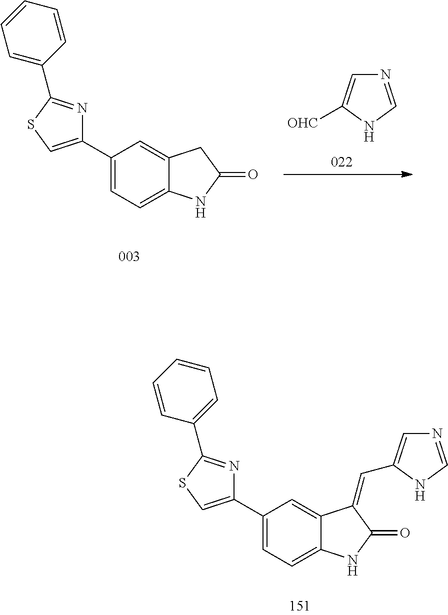 [SMILES: O=C1Cc2cc(-c3csc(-c4ccccc4)n3)ccc2N1 ; O=Cc1cnc[nH]1 ; O=C1Nc2ccc(-c3csc(-c4ccccc4)n3)cc2/C1=C/c1cnc[nH]1]

Step 1

To a suspension of 003 (58 mg, 0.2 mmol) in EtOH (3 mL) was added 022 (21 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 151 (45 mg). m/z 371 [M+1]

### Example 152

### Preparation of Compound 152

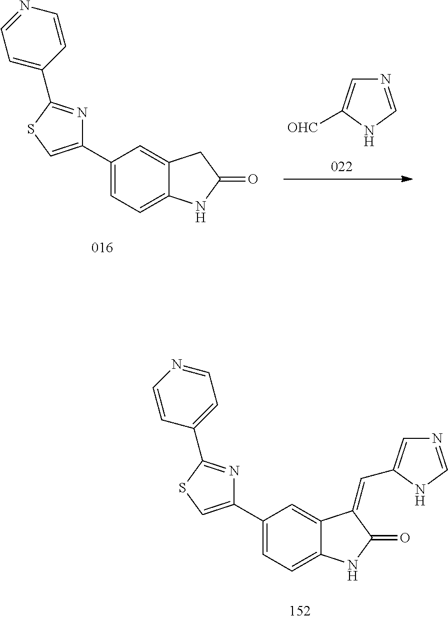 [SMILES: O=C1Cc2cc(-c3csc(-c4ccncc4)n3)ccc2N1 ; O=Cc1cnc[nH]1 ; O=C1Nc2ccc(-c3csc(-c4ccncc4)n3)cc2/C1=C/c1cnc[nH]1]

Step 1

To a suspension of 016 (59 mg, 0.2 mmol) in EtOH (3 mL) was added 022 (21 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 152 (38 mg). m/z 372 [M+1]

### Example 153

### Preparation of Compound 153

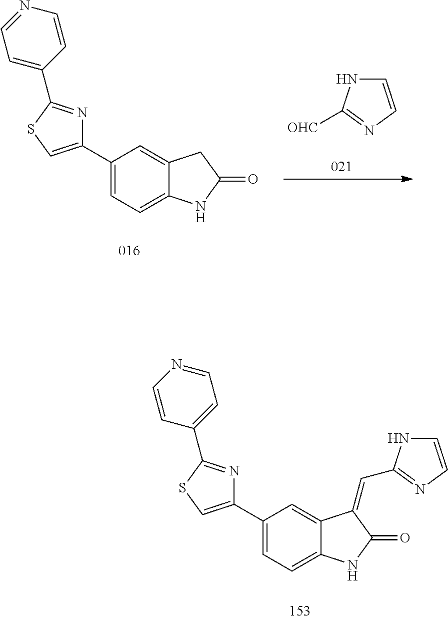 [SMILES: O=C1Cc2cc(-c3csc(-c4ccncc4)n3)ccc2N1 ; O=Cc1ncc[nH]1 ; O=C1Nc2ccc(-c3csc(-c4ccncc4)n3)cc2/C1=C/c1ncc[nH]1]

Step 1

To a suspension of 016 (59 mg, 0.2 mmol) in EtOH (3 mL) was added 021 (21 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 153 (23 mg). m/z 372 [M+1]

### Example 154

### Preparation of Compound 154

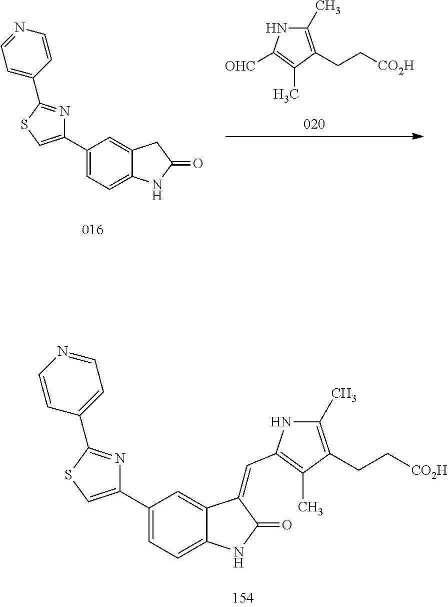 [SMILES: O=C1Cc2cc(-c3csc(-c4ccncc4)n3)ccc2N1 ; Cc1[nH]c(C=O)c(C)c1CCC(=O)O ; Cc1[nH]c(/C=C2\C(=O)Nc3ccc(-c4csc(-c5ccncc5)n4)cc32)c(C)c1CCC(=O)O]

Step 1

To a suspension of 016 (59 mg, 0.2 mmol) in EtOH (3 mL) was added 020 (42 mg, 0.22 mmol) and piperidine (one drop). The resulting mixture was heated for 16 hrs at 80° C. for before cooling, and then concentrated in vacuo. The reaction mixture was concentrated and neutralized to pH=4-5. The residue was purified by prep-HPLC to obtain product 154 (25 mg). m/z 471 [M+1]

### Example 155

### Preparation of Compound 155

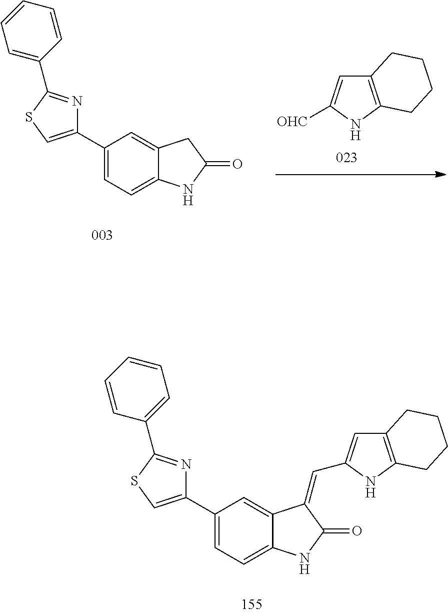 [SMILES: O=C1Cc2cc(-c3csc(-c4ccccc4)n3)ccc2N1 ; O=Cc1cc2c([nH]1)CCCC2 ; O=C1Nc2ccc(-c3csc(-c4ccccc4)n3)cc2/C1=C/c1cc2c([nH]1)CCCC2]

Step 1

To a suspension of 003 (58 mg, 0.2 mmol) in EtOH (3 mL) was added 023 (33 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 155 (20 mg). m/z 424 [M+1]

### Example 156

### Preparation of Compound 156

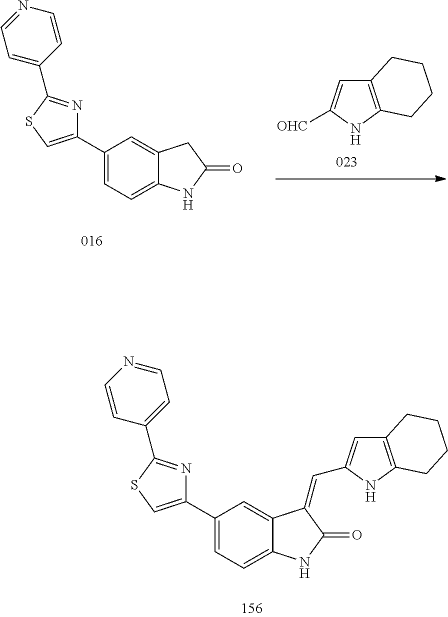 [SMILES: O=C1Cc2cc(-c3csc(-c4ccncc4)n3)ccc2N1 ; O=Cc1cc2c([nH]1)CCCC2 ; O=C1Nc2ccc(-c3csc(-c4ccncc4)n3)cc2/C1=C/c1cc2c([nH]1)CCCC2]

Step 1

To a suspension of 016 (59 mg, 0.2 mmol) in EtOH (3 mL) was added 023 (33 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 156 (65 mg). m/z 425 [M+l]

### Example 157

### Preparation of Compound 157

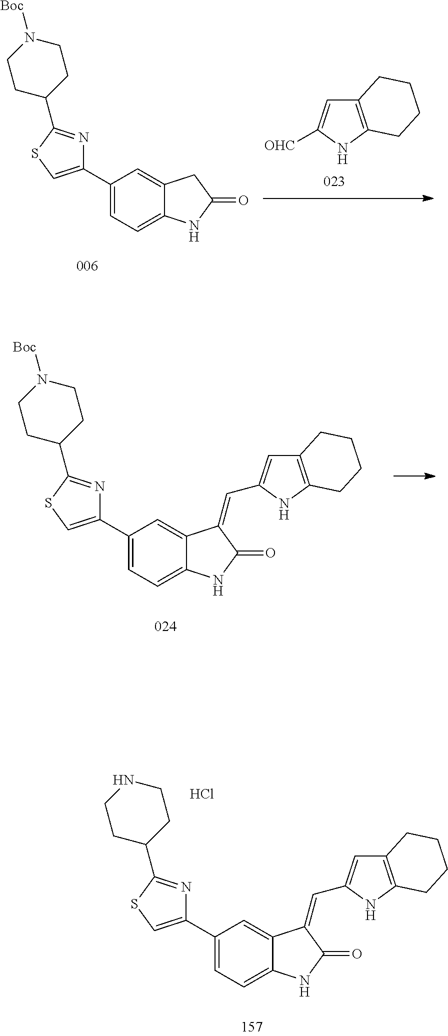 [SMILES: CC(C)(C)OC(=O)N1CCC(c2nc(-c3ccc4c(c3)CC(=O)N4)cs2)CC1 ; O=Cc1cc2c([nH]1)CCCC2 ; CC(C)(C)OC(=O)N1CCC(c2nc(-c3ccc4c(c3)/C(=C/c3cc5c([nH]3)CCCC5)C(=O)N4)cs2)CC1 ; O=C1Nc2ccc(-c3csc(C4CCNCC4)n3)cc2/C1=C/c1cc2c([nH]1)CCCC2 ; Cl]

Step 1

To a suspension of 006 (80 mg, 0.2 mmol) in EtOH (3 mL) was added 023 (33 mg, 0.22 mmol) and piperidine (0.1 mL). The resulting mixture was heated for 2 hrs at 80° C. for before cooling, and then concentrated in vacuo. The reaction mixture was concentrated. The residue was partitioned in EtOAc and H₂O, and the combined organic layers were washed with H₂O and brine, dried over Na₂SO₄, and concentrated in vacuo. The residue was purified by prep-HPLC to obtain product 024.

Step 2

To a solution of 024 (53 mg, 0.13 mmol) in MeOH (5 mL) was added HCl (4N in dioxane, 1 mL). The resulting mixture was stirred at room temperature for overnight. The reaction mixture was concentrated. The residue was purified by prep-HPLC to obtain product 157 (13 mg). m/z 431 [M+1]

### Example 158

### Preparation of Compound 158

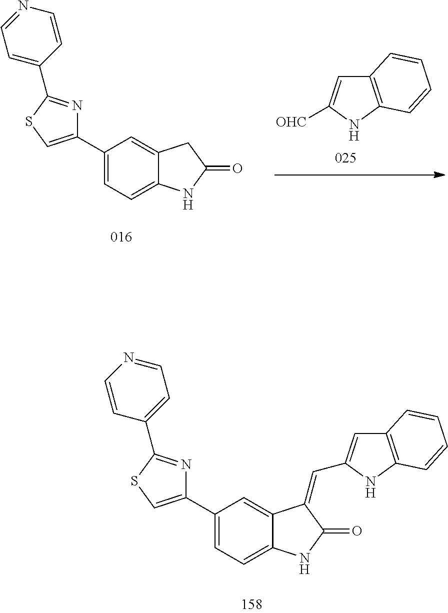 [SMILES: O=C1Cc2cc(-c3csc(-c4ccncc4)n3)ccc2N1 ; O=Cc1cc2ccccc2[nH]1 ; O=C1Nc2ccc(-c3csc(-c4ccncc4)n3)cc2/C1=C/c1cc2ccccc2[nH]1]

Step 1

To a suspension of 016 (59 mg, 0.2 mmol) in EtOH (3 mL) was added 025 (31 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 158 (48 mg). m/z 421 [M+1]

### Example 159

### Preparation of Compound 159

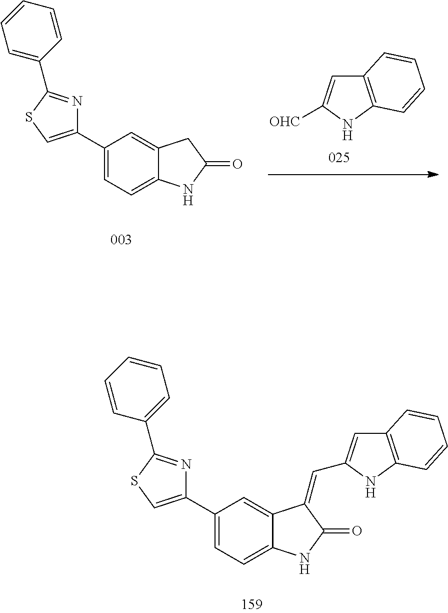 [SMILES: O=C1Cc2cc(-c3csc(-c4ccccc4)n3)ccc2N1 ; O=Cc1cc2ccccc2[nH]1 ; O=C1Nc2ccc(-c3csc(-c4ccccc4)n3)cc2/C1=C/c1cc2ccccc2[nH]1]

Step 1

To a suspension of 003 (58 mg, 0.2 mmol) in EtOH (3 mL) was added 025 (31 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 159 (26 mg). m/z 420 [M+1]

### Example 160

### Preparation of Compound 160

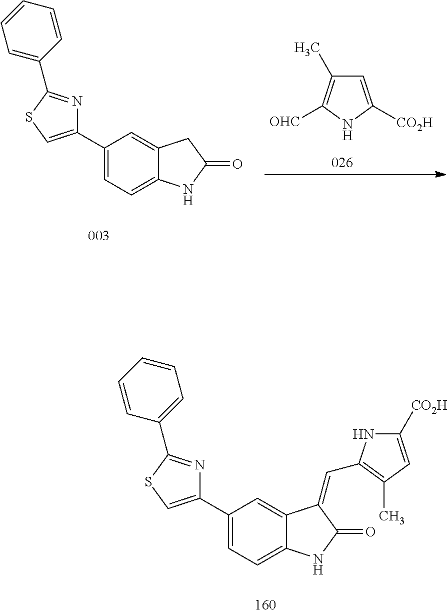 [SMILES: O=C1Cc2cc(-c3csc(-c4ccccc4)n3)ccc2N1 ; Cc1cc(C(=O)O)[nH]c1C=O ; Cc1cc(C(=O)O)[nH]c1/C=C1\C(=O)Nc2ccc(-c3csc(-c4ccccc4)n3)cc21]

Step 1

To a suspension of 003 (58 mg, 0.2 mmol) in EtOH (3 mL) was added 026 (33 mg, 0.22 mmol) and piperidine (one drop). The resulting mixture was heated for 16 hrs at 80° C. for before cooling, and then concentrated in vacuo. The reaction mixture was adjusted pH to ˜5 and concentrated. The residue was purified by prep-HPLC to obtain product 160 (35 mg). m/z 428 [M+1]

### Example 161

### Preparation of Compound 161

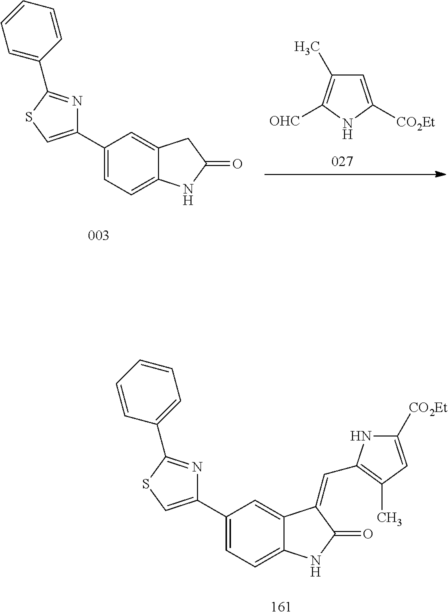 [SMILES: O=C1Cc2cc(-c3csc(-c4ccccc4)n3)ccc2N1 ; CCOC(=O)c1cc(C)c(C=O)[nH]1 ; CCOC(=O)c1cc(C)c(/C=C2\C(=O)Nc3ccc(-c4csc(-c5ccccc5)n4)cc32)[nH]1]

Step 1

To a suspension of 003 (58 mg, 0.2 mmol) in EtOH (3 mL) was added 027 (41 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 161 (41 mg). m/z 456 [M+1]

### Example 162

### Preparation of Compound 162

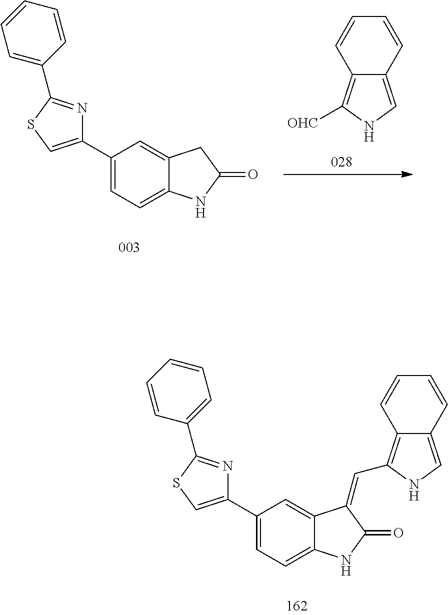 [SMILES: O=C1Cc2cc(-c3csc(-c4ccccc4)n3)ccc2N1 ; O=Cc1[nH]cc2ccccc12 ; O=C1Nc2ccc(-c3csc(-c4ccccc4)n3)cc2/C1=C/c1[nH]cc2ccccc12]

Step 1

To a suspension of 003 (58 mg, 0.2 mmol) in EtOH (3 mL) was added 028 (31 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 162 (35 mg). m/z 420 [M+1]

### Example 163

### Preparation of Compound 163

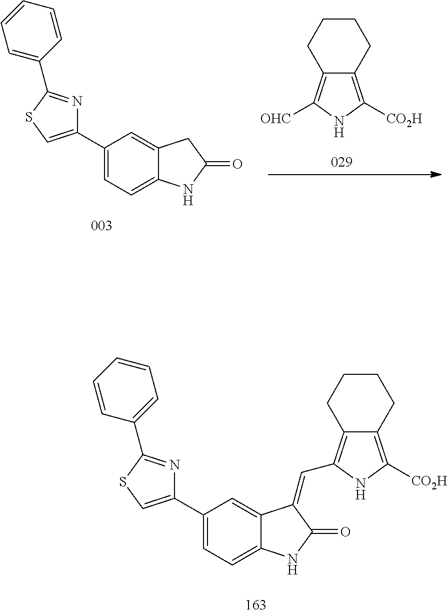 [SMILES: O=C1Cc2cc(-c3csc(-c4ccccc4)n3)ccc2N1 ; O=Cc1[nH]c(C(=O)O)c2c1CCCC2 ; O=C1Nc2ccc(-c3csc(-c4ccccc4)n3)cc2/C1=C/c1[nH]c(C(=O)O)c2c1CCCC2]

Step 1

To a suspension of 003 (58 mg, 0.2 mmol) in EtOH (3 mL) was added 029 (42 mg, 0.22 mmol) and piperidine (one drop). The resulting mixture was heated for 16 hrs at 80° C. for before cooling, and then concentrated in vacuo. The reaction mixture was adjusted pH=5 and concentrated. The residue was purified by prep-HPLC to obtain product 163 (35 mg). m/z 468 [M+1]

### Example 164

### Preparation of Compound 164

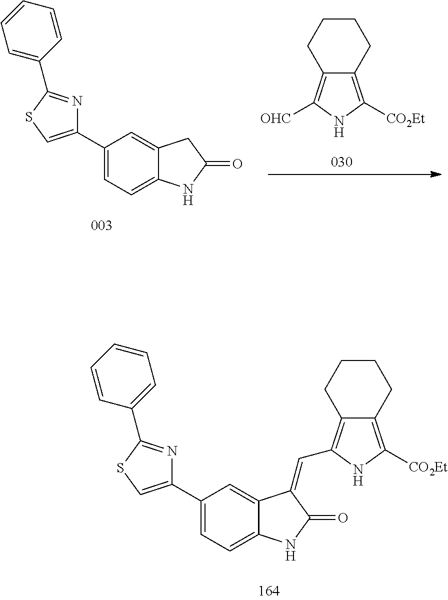 [SMILES: O=C1Cc2cc(-c3csc(-c4ccccc4)n3)ccc2N1 ; CCOC(=O)c1[nH]c(C=O)c2c1CCCC2 ; CCOC(=O)c1[nH]c(/C=C2\C(=O)Nc3ccc(-c4csc(-c5ccccc5)n4)cc32)c2c1CCCC2]

Step 1

To a suspension of 003 (58 mg, 0.2 mmol) in EtOH (3 mL) was added 030 (46 mg, 0.22 mmol) and piperidine (one drop). The resulting mixture was heated for 16 hrs at 80° C. for before cooling, and then concentrated in vacuo. The reaction mixture was concentrated. The residue was purified by prep-HPLC to obtain product 164 (40 mg). m/z 496 [M+1]

### Example 165

### Preparation of Compound 165

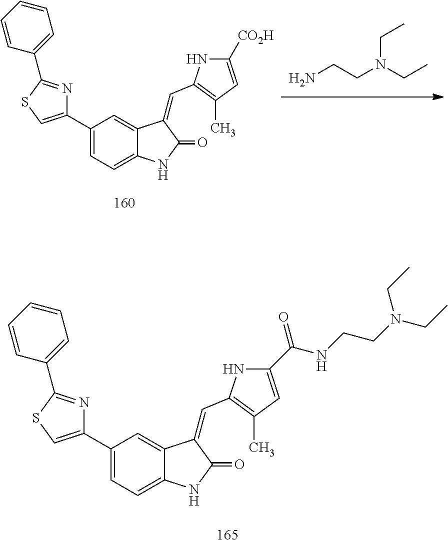

Step 1

To a solution of compound 160 (43 mg, 0.1 mmol) in DMF (2 mL) was added HATU (69 mg), diisopropylethylamine (500 uL), and diethylethylenediamine (25 mg). The mixture was stirred at room temperature for 16 hours then concentrated in vacuo. The residue was added CH₂Cl₂ and washed with H₂O. The organic layer was dried over Na₂SO₄ and concentrated in vacuo. The residue was purified by prep-HPLC to obtain product 165 (35 mg). m/z 526 [M+1]

### Example 166

### Preparation of Compound 166

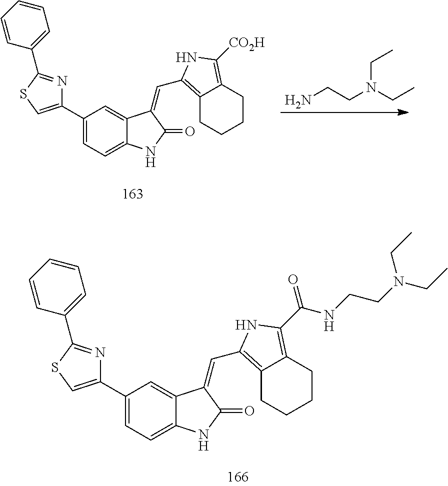

Step 1

To a solution of compound 163 (47 mg, 0.1 mmol) in DMF (2 mL) was added HATU (69 mg), diisopropylethylamine (500 uL), and diethylethylenediamine (25 mg). The mixture was stirred at room temperature for 16 hours then concentrated in vacuo. The residue was added CH₂Cl₂ and washed with H₂O. The organic layer was dried over Na₂SO₄ and concentrated in vacuo. The residue was purified by prep-HPLC to obtain product 166 (37 mg). m/z 566 [M+1]

### Example 167

### Preparation of Compound 167

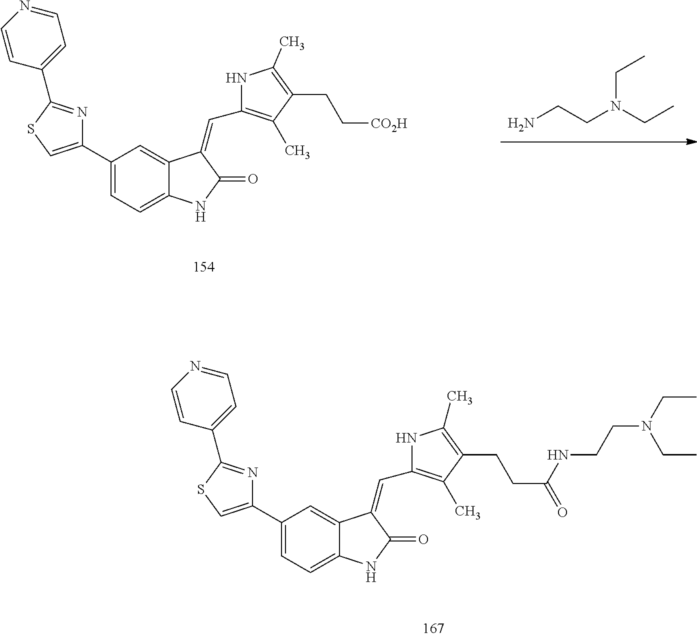

Step 1

To a solution of compound 154 (47 mg, 0.1 mmol) in DMF (2 mL) was added HATU (69 mg), diisopropylethylamine (500 uL), diethylethylenediamine (25 mg). The mixture was stirred at room temperature for 16 hours then concentrated in vacuo. The residue was added CH₂Cl₂ and washed with H₂O. The organic layer was dried over Na₂SO₄ and concentrated in vacuo. The residue was purified by prep-HPLC to obtain product 167 (18 mg). m/z 569 [M+1]

### Example 168

### Preparation of Compound 168

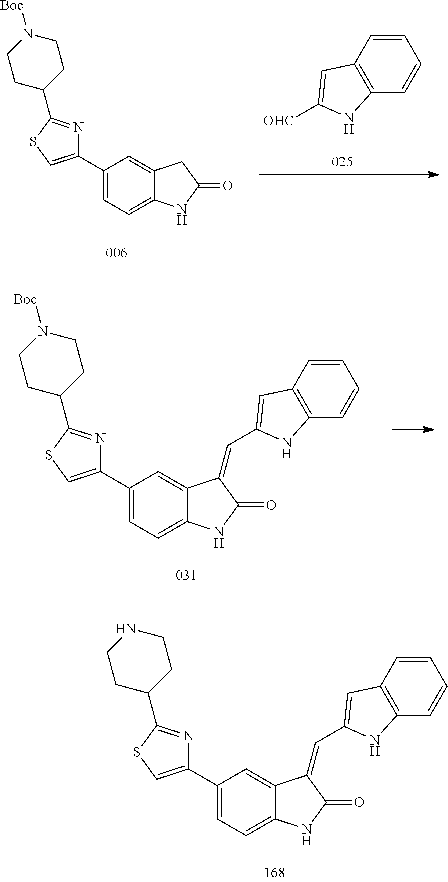 [SMILES: CC(C)(C)OC(=O)N1CCC(c2nc(-c3ccc4c(c3)CC(=O)N4)cs2)CC1 ; O=Cc1cc2ccccc2[nH]1 ; CC(C)(C)OC(=O)N1CCC(c2nc(-c3ccc4c(c3)/C(=C/c3cc5ccccc5[nH]3)C(=O)N4)cs2)CC1 ; O=C1Nc2ccc(-c3csc(C4CCNCC4)n3)cc2/C1=C/c1cc2ccccc2[nH]1]

Step 1

To a suspension of 006 (80 mg, 0.2 mmol) in EtOH (3 mL) was added 025 (32 mg, 0.22 mmol) and piperidine (0.1 mL). The resulting mixture was heated for 2 hrs at 80° C. for before cooling, and then concentrated in vacuo. The reaction mixture was concentrated. The residue was partitioned in EtOAc and H₂O, and the combined organic layers were washed with H₂O and brine, dried over Na₂SO₄, and concentrated in vacuo. The residue was purified by prep-HPLC to obtain product 031.

Step 2

To a solution of 031 (25 mg, 0.05 mmol) in MeOH (5 mL) was added HCl (4N in dioxane, 1 mL). The resulting mixture was stirred at rt for overnight and concentrated. The residue was purified by prep-HPLC to obtain product 168 (18 mg). m/z 427 [M+1]

### Example 169

### Preparation of Compound 169

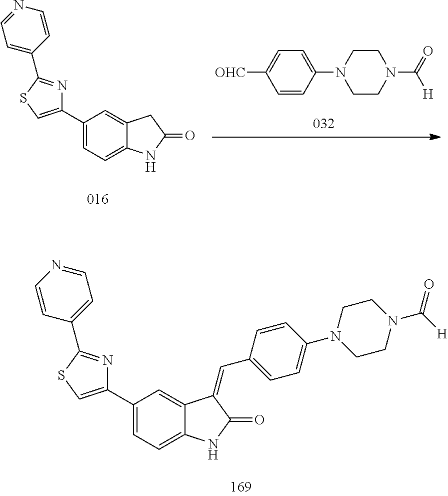 [SMILES: O=C1Cc2cc(-c3csc(-c4ccncc4)n3)ccc2N1 ; O=Cc1ccc(N2CCN(C=O)CC2)cc1 ; O=CN1CCN(c2ccc(/C=C3\C(=O)Nc4ccc(-c5csc(-c6ccncc6)n5)cc43)cc2)CC1]

Step 1

To a suspension of 016 (59 mg, 0.2 mmol) in EtOH (3 mL) was added 032 (47 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 169 (43 mg). m/z 494 [M+1]

### Example 170

### Preparation of Compound 170

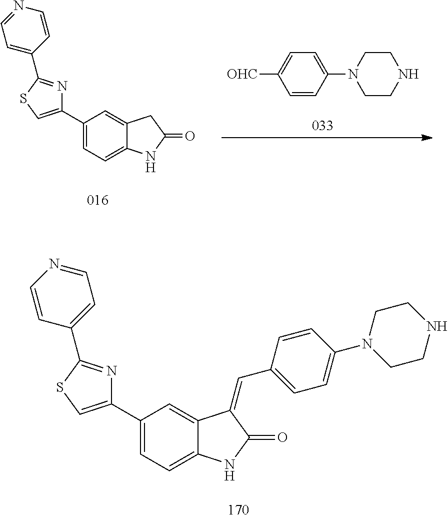 [SMILES: O=C1Cc2cc(-c3csc(-c4ccncc4)n3)ccc2N1 ; O=Cc1ccc(N2CCNCC2)cc1 ; O=C1Nc2ccc(-c3csc(-c4ccncc4)n3)cc2/C1=C/c1ccc(N2CCNCC2)cc1]

Step 1

To a suspension of 016 (59 mg, 0.2 mmol) in EtOH (3 mL) was added 033 (42 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 170 (25 mg). m/z 466 [M+1]

### Example 171

### Preparation of Compound 171

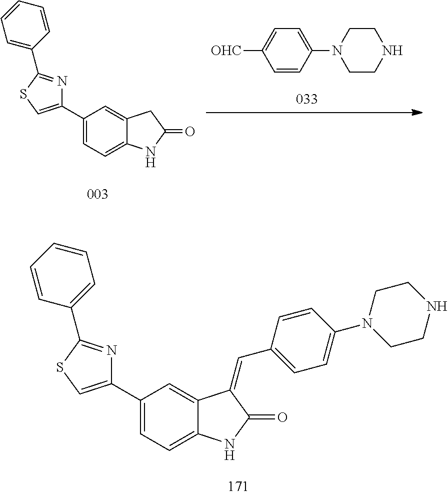 [SMILES: O=C1Cc2cc(-c3csc(-c4ccccc4)n3)ccc2N1 ; O=Cc1ccc(N2CCNCC2)cc1 ; O=C1Nc2ccc(-c3csc(-c4ccccc4)n3)cc2/C1=C/c1ccc(N2CCNCC2)cc1]

Step 1

To a suspension of 003 (58 mg, 0.2 mmol) in EtOH (3 mL) was added 033 (42 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 171 (26 mg). m/z 465 [M+1]

### Example 172

### Preparation of Compound 172

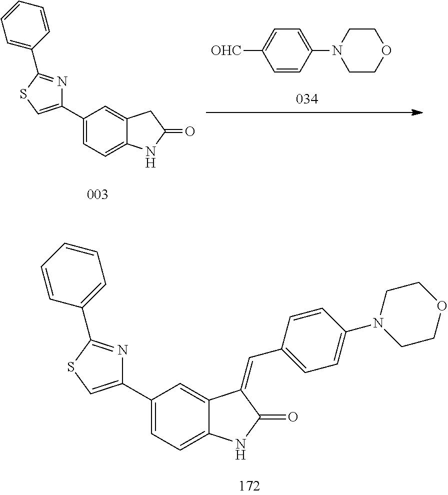 [SMILES: O=C1Cc2cc(-c3csc(-c4ccccc4)n3)ccc2N1 ; O=Cc1ccc(N2CCOCC2)cc1 ; O=C1Nc2ccc(-c3csc(-c4ccccc4)n3)cc2/C1=C/c1ccc(N2CCOCC2)cc1]

Step 1

To a suspension of 003 (58 mg, 0.2 mmol) in EtOH (3 mL) was added 034 (42 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 172 (42 mg). m/z 466 [M+1]

### Example 173

### Preparation of Compound 173

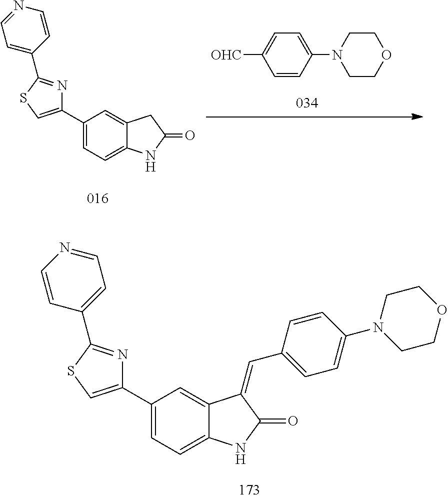 [SMILES: O=C1Cc2cc(-c3csc(-c4ccncc4)n3)ccc2N1 ; O=Cc1ccc(N2CCOCC2)cc1 ; O=C1Nc2ccc(-c3csc(-c4ccncc4)n3)cc2/C1=C/c1ccc(N2CCOCC2)cc1]

Step 1

To a suspension of 016 (59 mg, 0.2 mmol) in EtOH (3 mL) was added 034 (42 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 173 (39 mg). m/z 467 [M+1]

### Example 174

### Preparation of Compound 174

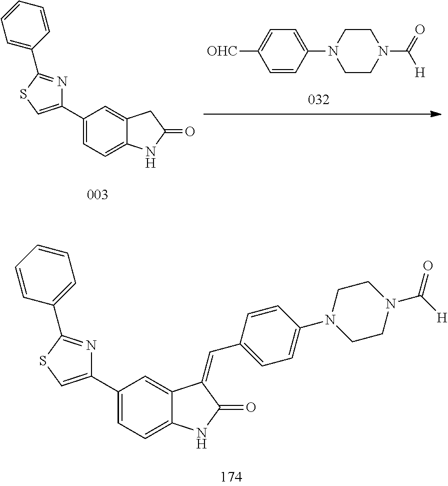 [SMILES: O=C1Cc2cc(-c3csc(-c4ccccc4)n3)ccc2N1 ; O=Cc1ccc(N2CCN(C=O)CC2)cc1 ; O=CN1CCN(c2ccc(/C=C3\C(=O)Nc4ccc(-c5csc(-c6ccccc6)n5)cc43)cc2)CC1]

Step 1

To a suspension of 003 (59 mg, 0.2 mmol) in EtOH (3 mL) was added 032 (47 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 174 (37 mg). m/z 494 [M+l]

### Example 175

### Preparation of Compound 175

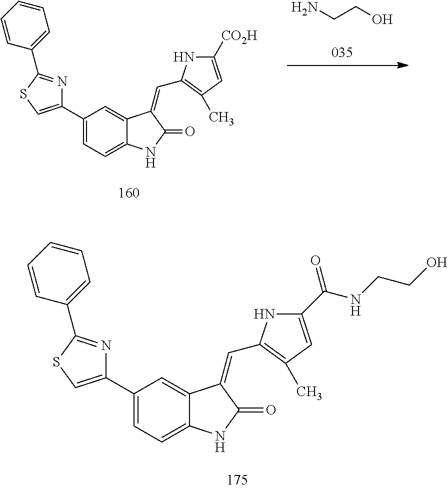

Step 1

To a solution of 160 (64 mg, 0.15 mmol) in DMF (2 mL) was added EDCI (58 mg, 0.3 mmol), HOBT (41 mg, 0.3 mmol), diisopropylethylamine (78 uL, 0.45 mmol), and amine 035 (68 uL). The mixture was stirred at room temperature for 24 hours then was added CH₂Cl₂ (20 mL) and washed with H₂O. The organic layer was dried over Na₂SO₄ and concentrated in vacuo. The residue was purified by prep-HPLC to obtain product 175 (19 mg). m/z 471 [M+1]

### Example 176

### Preparation of Compound 176

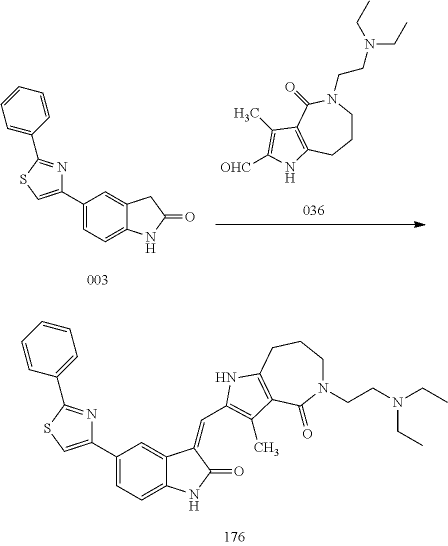 [SMILES: O=C1Cc2cc(-c3csc(-c4ccccc4)n3)ccc2N1 ; CCN(CC)CCN1CCCc2[nH]c(C=O)c(C)c2C1=O ; CCN(CC)CCN1CCCc2[nH]c(/C=C3\C(=O)Nc4ccc(-c5csc(-c6ccccc6)n5)cc43)c(C)c2C1=O]

Step 1

To a suspension of 003 (58 mg, 0.2 mmol) in EtOH (3 mL) was added 036 (65 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 176 (25 mg). m/z 566 [M+1]

### Example 177

### Preparation of Compound 177

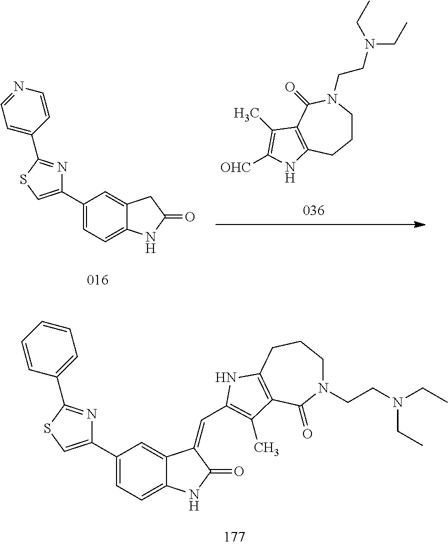 [SMILES: O=C1Cc2cc(-c3csc(-c4ccncc4)n3)ccc2N1 ; CCN(CC)CCN1CCCc2[nH]c(C=O)c(C)c2C1=O ; CCN(CC)CCN1CCCc2[nH]c(/C=C3\C(=O)Nc4ccc(-c5csc(-c6ccccc6)n5)cc43)c(C)c2C1=O]

Step 1

To a suspension of 016 (30 mg, 0.2 mmol) in EtOH (3 mL) was added 036 (38 mg, 0.22 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 177 (11 mg). m/z 567 [M+1]

### Example 178

### Preparation of Compound 178

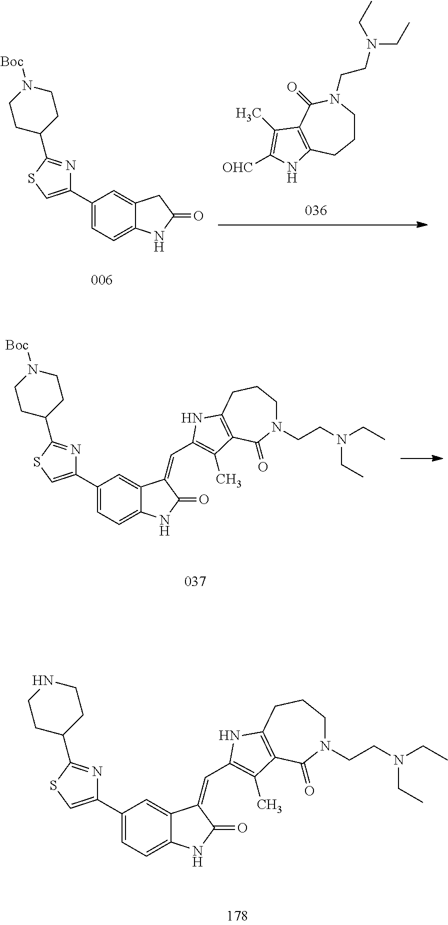 [SMILES: CC(C)(C)OC(=O)N1CCC(c2nc(-c3ccc4c(c3)CC(=O)N4)cs2)CC1 ; CCN(CC)CCN1CCCc2[nH]c(C=O)c(C)c2C1=O ; CCN(CC)CCN1CCCc2[nH]c(/C=C3\C(=O)Nc4ccc(-c5csc(C6CCN(C(=O)OC(C)(C)C)CC6)n5)cc43)c(C)c2C1=O ; CCN(CC)CCN1CCCc2[nH]c(/C=C3\C(=O)Nc4ccc(-c5csc(C6CCNCC6)n5)cc43)c(C)c2C1=O]

Step 1

To a suspension of 006 (40 mg, 0.1 mmol) in EtOH (3 mL) was added 036 (40 mg, 0.13 mmol) and piperidine (0.1 mL). The resulting mixture was heated for 16 hrs at 80° C. for before cooling, and then concentrated in vacuo. The reaction mixture was concentrated. The residue was purified by prep-HPLC to obtain product 037.

Step 2

To a solution of 037 (34 mg, 0.1 mmol) in MeOH (5 mL) was added HCl (4 N in dioxane, 1 mL). The resulting mixture was stirred at room temperature for overnight. The reaction mixture was concentrated. The residue was purified by prep-HPLC to obtain product 178 (28 mg). m/z 573 [M+1]

### Examples 179 to 287

Reactions and treatments were carried out in the same manner as in Example 1 using the corresponding starting material compounds, thereby giving the compounds of Examples 179 to 287 shown in Table 18.

TABLE 18

| Example | Structure |
|---|---|
| 179 | 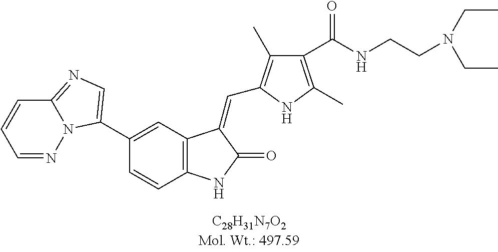 [SMILES: CCN(CC)CCNC(=O)c1c(C)[nH]c(/C=C2\C(=O)Nc3ccc(-c4cnc5cccnn45)cc32)c1C] |
| 180 | 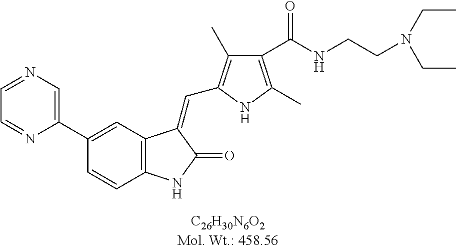 [SMILES: CCN(CC)CCNC(=O)c1c(C)[nH]c(/C=C2\C(=O)Nc3ccc(-c4cnccn4)cc32)c1C] |
| 181 | 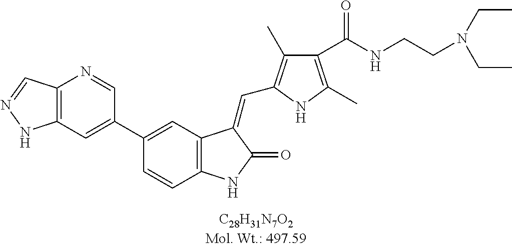 [SMILES: CCN(CC)CCNC(=O)c1c(C)[nH]c(/C=C2\C(=O)Nc3ccc(-c4cnc5cn[nH]c5c4)cc32)c1C] |
| 182 | 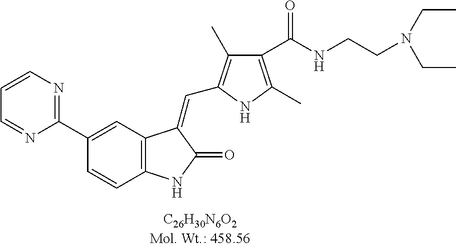 [SMILES: CCN(CC)CCNC(=O)c1c(C)[nH]c(/C=C2\C(=O)Nc3ccc(-c4ncccn4)cc32)c1C] |
| 183 | 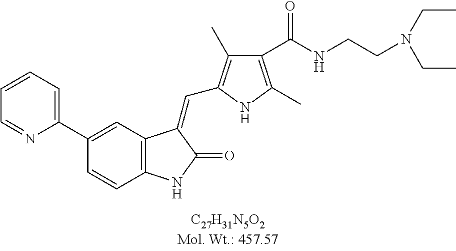 [SMILES: CCN(CC)CCNC(=O)c1c(C)[nH]c(/C=C2\C(=O)Nc3ccc(-c4ccccn4)cc32)c1C] |
| 184 | 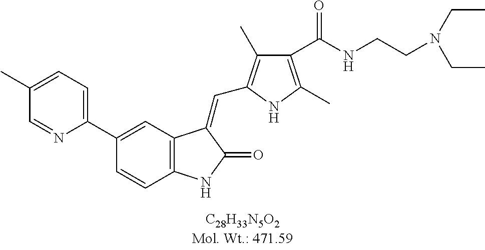 [SMILES: CCN(CC)CCNC(=O)c1c(C)[nH]c(/C=C2\C(=O)Nc3ccc(-c4ccc(C)cn4)cc32)c1C] |
| 185 | 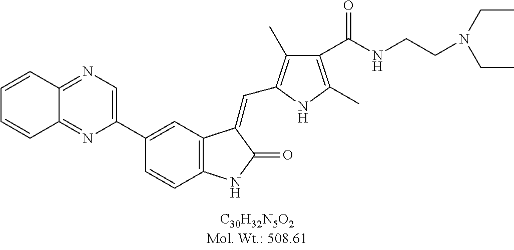 [SMILES: CCN(CC)CCNC(=O)c1c(C)[nH]c(/C=C2\C(=O)Nc3ccc(-c4cnc5ccccc5n4)cc32)c1C] |
| 186 | 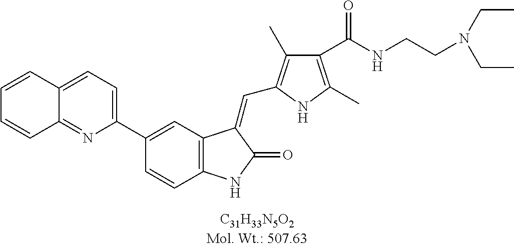 [SMILES: CCN(CC)CCNC(=O)c1c(C)[nH]c(/C=C2\C(=O)Nc3ccc(-c4ccc5ccccc5n4)cc32)c1C] |
| 187 | 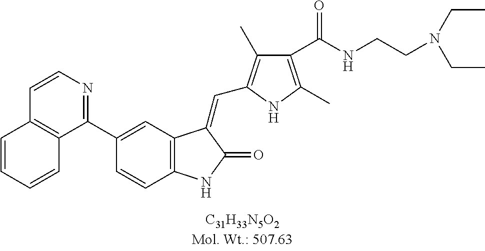 [SMILES: CCN(CC)CCNC(=O)c1c(C)[nH]c(/C=C2\C(=O)Nc3ccc(-c4nccc5ccccc45)cc32)c1C] |
| 188 | 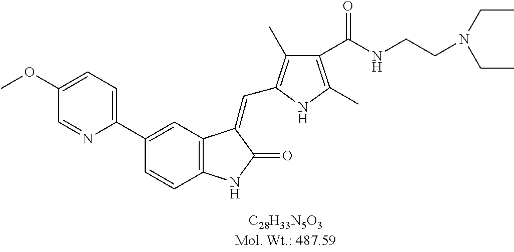 [SMILES: CCN(CC)CCNC(=O)c1c(C)[nH]c(/C=C2\C(=O)Nc3ccc(-c4ccc(OC)cn4)cc32)c1C] |
| 189 | 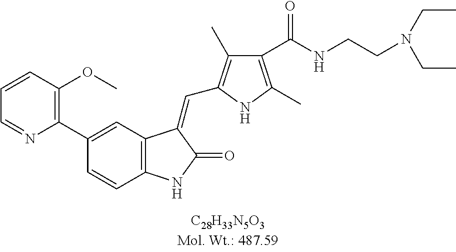 [SMILES: CCN(CC)CCNC(=O)c1c(C)[nH]c(/C=C2\C(=O)Nc3ccc(-c4ncccc4OC)cc32)c1C] |
| 190 | 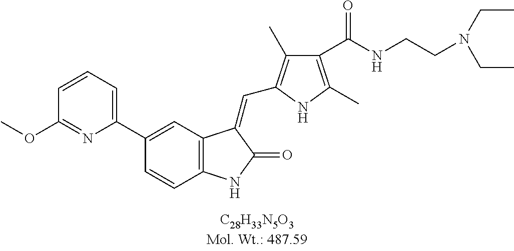 [SMILES: CCN(CC)CCNC(=O)c1c(C)[nH]c(/C=C2\C(=O)Nc3ccc(-c4cccc(OC)n4)cc32)c1C] |
| 191 | 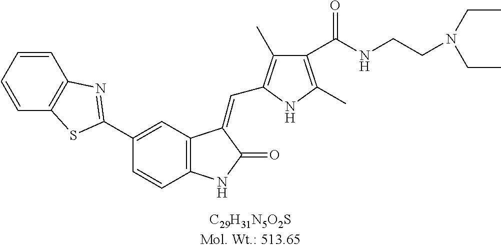 [SMILES: CCN(CC)CCNC(=O)c1c(C)[nH]c(/C=C2\C(=O)Nc3ccc(-c4nc5ccccc5s4)cc32)c1C] |
| 192 | 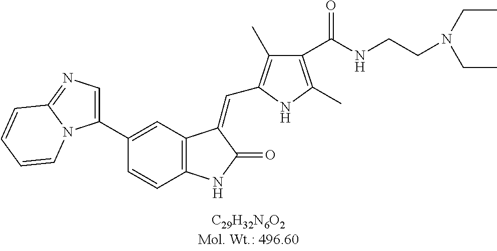 [SMILES: CCN(CC)CCNC(=O)c1c(C)[nH]c(/C=C2\C(=O)Nc3ccc(-c4cnc5ccccn45)cc32)c1C] |
| 193 | 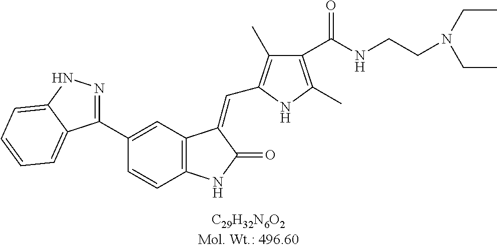 [SMILES: CCN(CC)CCNC(=O)c1c(C)[nH]c(/C=C2\C(=O)Nc3ccc(-c4n[nH]c5ccccc45)cc32)c1C] |
| 194 | 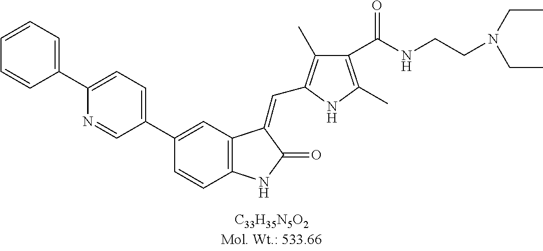 [SMILES: CCN(CC)CCNC(=O)c1c(C)[nH]c(/C=C2\C(=O)Nc3ccc(-c4ccc(-c5ccccc5)nc4)cc32)c1C] |
| 195 | 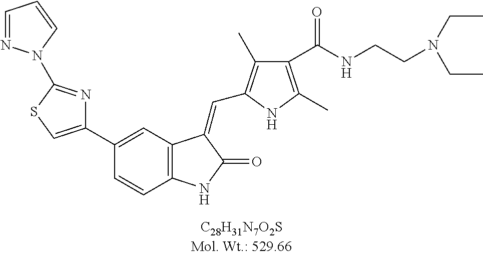 [SMILES: CCN(CC)CCNC(=O)c1c(C)[nH]c(/C=C2\C(=O)Nc3ccc(-c4csc(-n5cccn5)n4)cc32)c1C] |
| 196 | 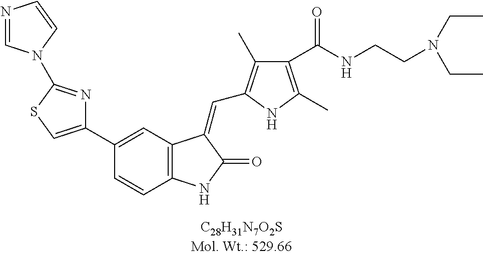 [SMILES: CCN(CC)CCNC(=O)c1c(C)[nH]c(/C=C2\C(=O)Nc3ccc(-c4csc(-n5ccnc5)n4)cc32)c1C] |
| 197 | 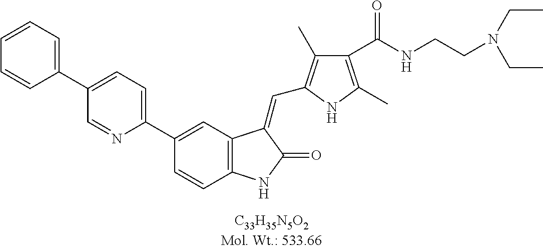 [SMILES: CCN(CC)CCNC(=O)c1c(C)[nH]c(/C=C2\C(=O)Nc3ccc(-c4ccc(-c5ccccc5)cn4)cc32)c1C] |
| 198 | 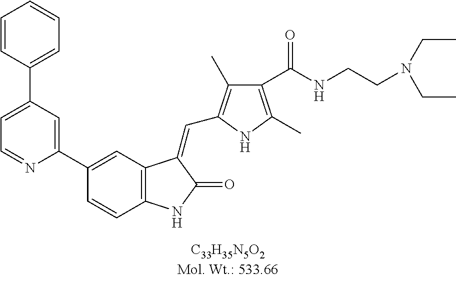 [SMILES: CCN(CC)CCNC(=O)c1c(C)[nH]c(/C=C2\C(=O)Nc3ccc(-c4cc(-c5ccccc5)ccn4)cc32)c1C] |
| 199 | 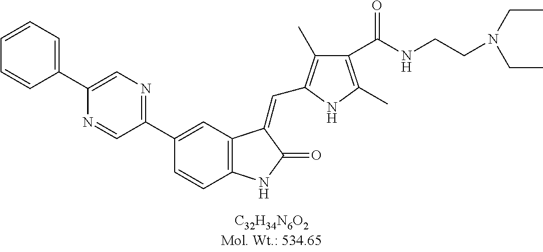 [SMILES: CCN(CC)CCNC(=O)c1c(C)[nH]c(/C=C2\C(=O)Nc3ccc(-c4cnc(-c5ccccc5)cn4)cc32)c1C] |
| 200 | 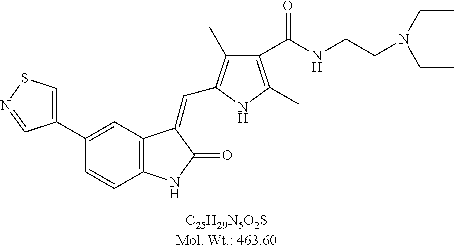 [SMILES: CCN(CC)CCNC(=O)c1c(C)[nH]c(/C=C2\C(=O)Nc3ccc(-c4cnsc4)cc32)c1C] |
| 201 | 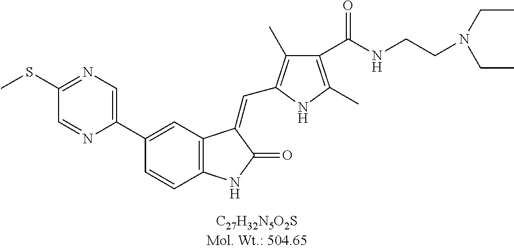 [SMILES: CCN(CC)CCNC(=O)c1c(C)[nH]c(/C=C2\C(=O)Nc3ccc(-c4cnc(SC)cn4)cc32)c1C] |
| 202 | 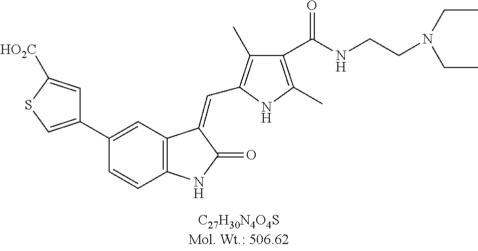 [SMILES: CCN(CC)CCNC(=O)c1c(C)[nH]c(/C=C2\C(=O)Nc3ccc(-c4csc(C(=O)O)c4)cc32)c1C] |
| 203 | 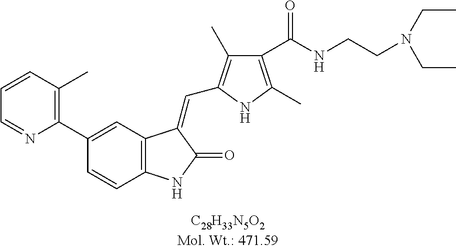 [SMILES: CCN(CC)CCNC(=O)c1c(C)[nH]c(/C=C2\C(=O)Nc3ccc(-c4ncccc4C)cc32)c1C] |
| 204 | 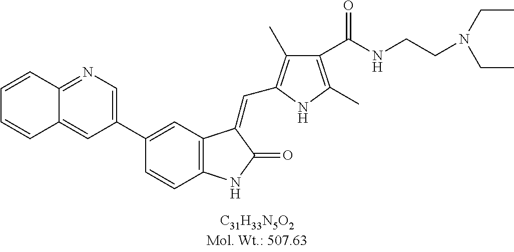 [SMILES: CCN(CC)CCNC(=O)c1c(C)[nH]c(/C=C2\C(=O)Nc3ccc(-c4cnc5ccccc5c4)cc32)c1C] |
| 205 | 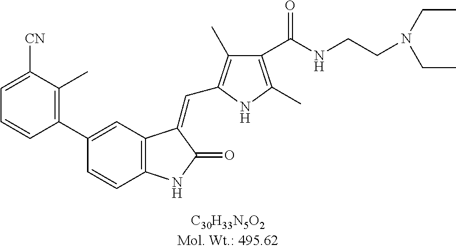 [SMILES: CCN(CC)CCNC(=O)c1c(C)[nH]c(/C=C2\C(=O)Nc3ccc(-c4cccc(C#N)c4C)cc32)c1C] |
| 206 | 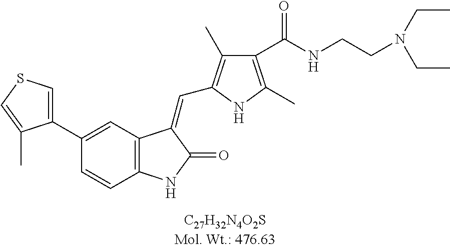 [SMILES: CCN(CC)CCNC(=O)c1c(C)[nH]c(/C=C2\C(=O)Nc3ccc(-c4cscc4C)cc32)c1C] |
| 207 | 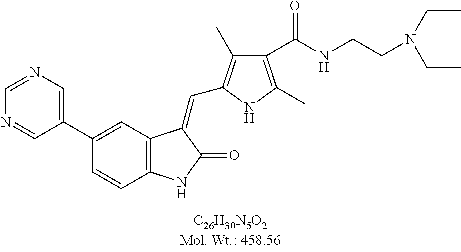 [SMILES: CCN(CC)CCNC(=O)c1c(C)[nH]c(/C=C2\C(=O)Nc3ccc(-c4cncnc4)cc32)c1C] |
| 208 | 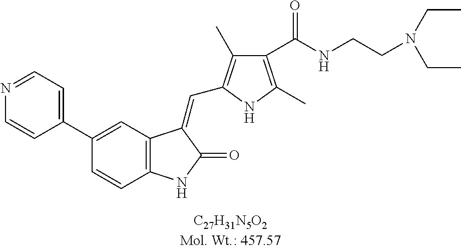 [SMILES: CCN(CC)CCNC(=O)c1c(C)[nH]c(/C=C2\C(=O)Nc3ccc(-c4ccncc4)cc32)c1C] |
| 209 | 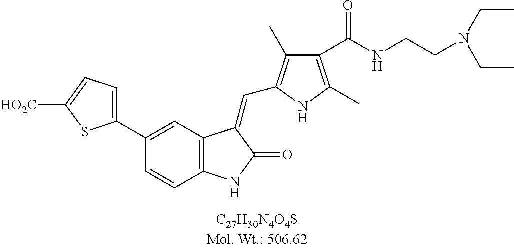 [SMILES: CCN(CC)CCNC(=O)c1c(C)[nH]c(/C=C2\C(=O)Nc3ccc(-c4ccc(C(=O)O)s4)cc32)c1C] |
| 210 | 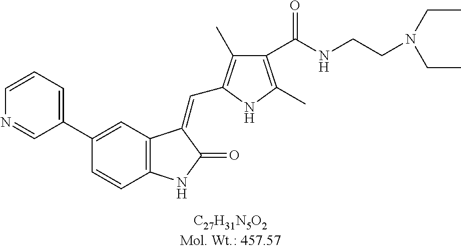 [SMILES: CCN(CC)CCNC(=O)c1c(C)[nH]c(/C=C2\C(=O)Nc3ccc(-c4cccnc4)cc32)c1C] |
| 211 | 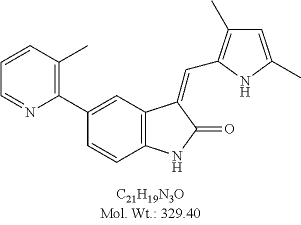 [SMILES: Cc1cc(C)c(/C=C2\C(=O)Nc3ccc(-c4ncccc4C)cc32)[nH]1] |
| 212 | 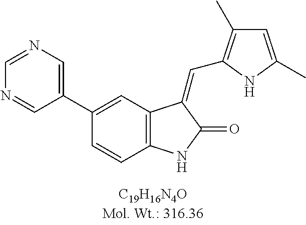 [SMILES: Cc1cc(C)c(/C=C2\C(=O)Nc3ccc(-c4cncnc4)cc32)[nH]1] |
| 213 | 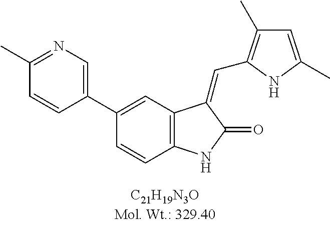 |
| 214 | 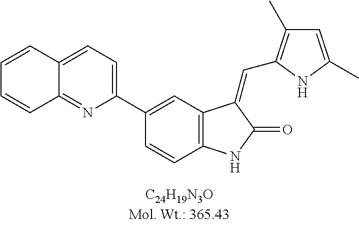 [SMILES: Cc1cc(C)c(/C=C2\C(=O)Nc3ccc(-c4ccc5ccccc5n4)cc32)[nH]1] |
| 215 | 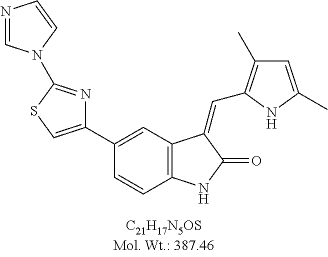 [SMILES: Cc1cc(C)c(/C=C2\C(=O)Nc3ccc(-c4csc(-n5ccnc5)n4)cc32)[nH]1] |
| 216 | 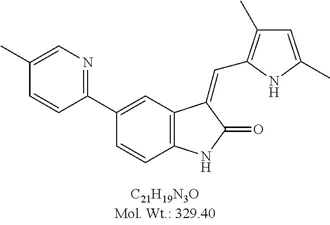 |
| 217 | 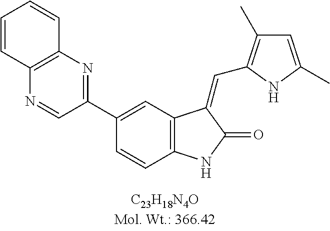 [SMILES: Cc1cc(C)c(/C=C2\C(=O)Nc3ccc(-c4cnc5ccccc5n4)cc32)[nH]1] |
| 218 | 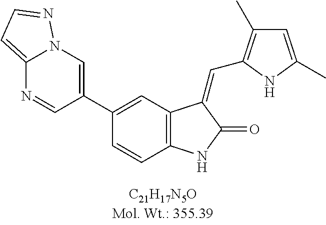 [SMILES: Cc1cc(C)c(/C=C2\C(=O)Nc3ccc(-c4cnc5ccnn5c4)cc32)[nH]1] |
| 219 | 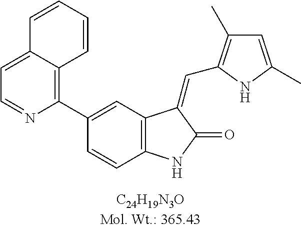 [SMILES: Cc1cc(C)c(/C=C2\C(=O)Nc3ccc(-c4nccc5ccccc45)cc32)[nH]1] |
| 220 | 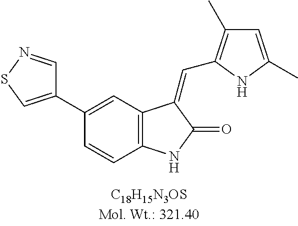 [SMILES: Cc1cc(C)c(/C=C2\C(=O)Nc3ccc(-c4cnsc4)cc32)[nH]1] |
| 221 | 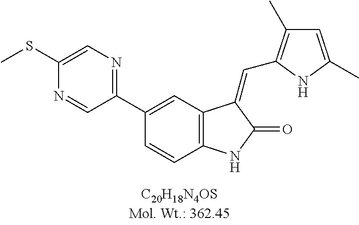 |
| 222 | 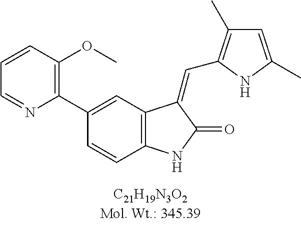 [SMILES: COc1cccnc1-c1ccc2c(c1)/C(=C/c1[nH]c(C)cc1C)C(=O)N2] |
| 223 | 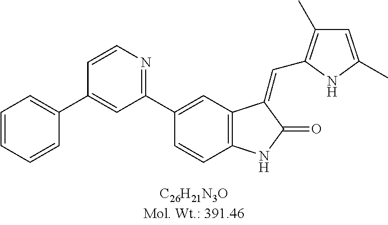 [SMILES: Cc1cc(C)c(/C=C2\C(=O)Nc3ccc(-c4cc(-c5ccccc5)ccn4)cc32)[nH]1] |
| 224 | 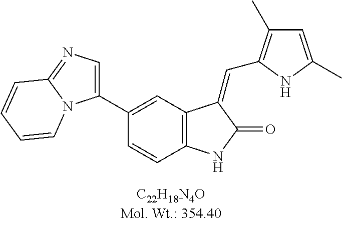 [SMILES: Cc1cc(C)c(/C=C2\C(=O)Nc3ccc(-c4cnc5ccccn45)cc32)[nH]1] |
| 225 | 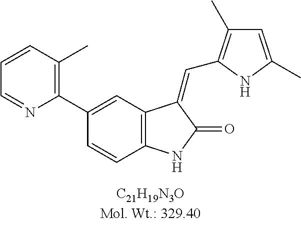 [SMILES: Cc1cc(C)c(/C=C2\C(=O)Nc3ccc(-c4ncccc4C)cc32)[nH]1] |
| 226 | 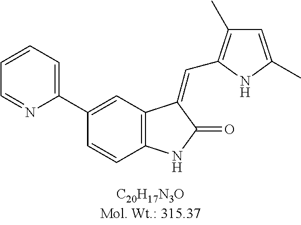 [SMILES: Cc1cc(C)c(/C=C2\C(=O)Nc3ccc(-c4ccccn4)cc32)[nH]1] |
| 227 | 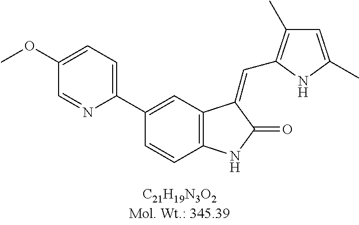 |
| 228 | 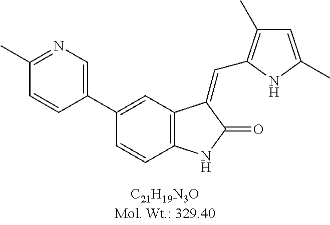 |
| 229 | 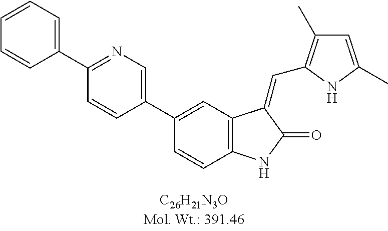 [SMILES: Cc1cc(C)c(/C=C2\C(=O)Nc3ccc(-c4ccc(-c5ccccc5)nc4)cc32)[nH]1] |
| 230 | 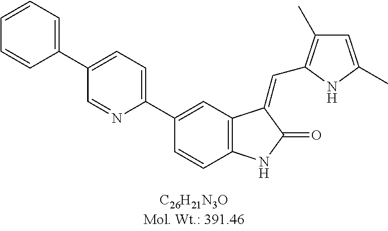 [SMILES: Cc1cc(C)c(/C=C2\C(=O)Nc3ccc(-c4ccc(-c5ccccc5)cn4)cc32)[nH]1] |
| 231 | 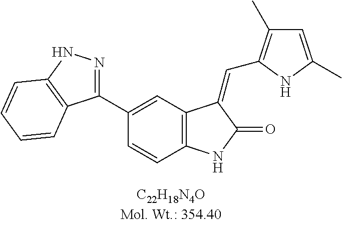 [SMILES: Cc1cc(C)c(/C=C2\C(=O)Nc3ccc(-c4n[nH]c5ccccc45)cc32)[nH]1] |
| 232 | 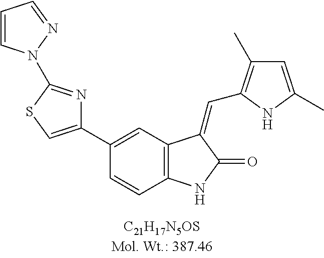 [SMILES: Cc1cc(C)c(/C=C2\C(=O)Nc3ccc(-c4csc(-n5cccn5)n4)cc32)[nH]1] |
| 233 | 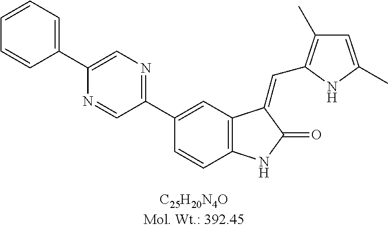 [SMILES: Cc1cc(C)c(/C=C2\C(=O)Nc3ccc(-c4cnc(-c5ccccc5)cn4)cc32)[nH]1] |
| 234 | 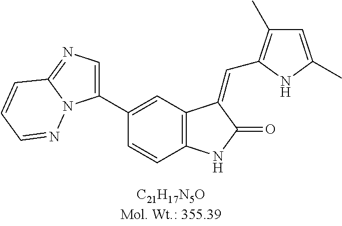 [SMILES: Cc1cc(C)c(/C=C2\C(=O)Nc3ccc(-c4cnc5cccnn45)cc32)[nH]1] |
| 235 | 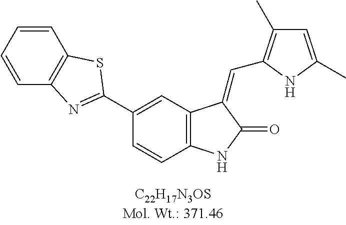 [SMILES: Cc1cc(C)c(/C=C2\C(=O)Nc3ccc(-c4nc5ccccc5s4)cc32)[nH]1] |
| 236 | 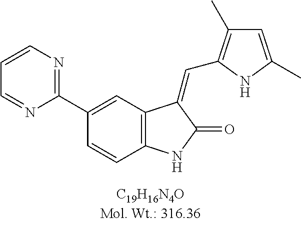 [SMILES: Cc1cc(C)c(/C=C2\C(=O)Nc3ccc(-c4ncccn4)cc32)[nH]1] |
| 237 | 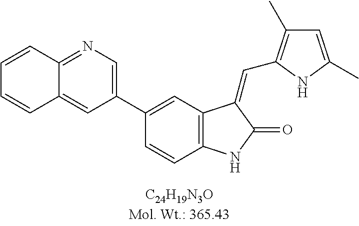 [SMILES: Cc1cc(C)c(/C=C2\C(=O)Nc3ccc(-c4cnc5ccccc5c4)cc32)[nH]1] |
| 238 | 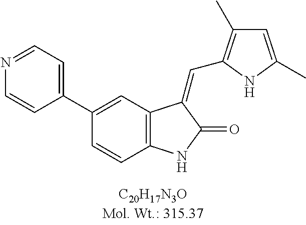 [SMILES: Cc1cc(C)c(/C=C2\C(=O)Nc3ccc(-c4ccncc4)cc32)[nH]1] |
| 239 | 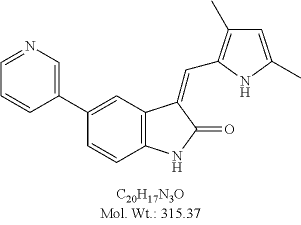 [SMILES: Cc1cc(C)c(/C=C2\C(=O)Nc3ccc(-c4cccnc4)cc32)[nH]1] |
| 240 | 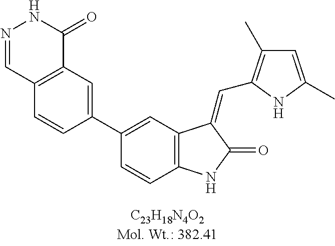 [SMILES: Cc1cc(C)c(/C=C2\C(=O)Nc3ccc(-c4ccc5cn[nH]c(=O)c5c4)cc32)[nH]1] |
| 241 | 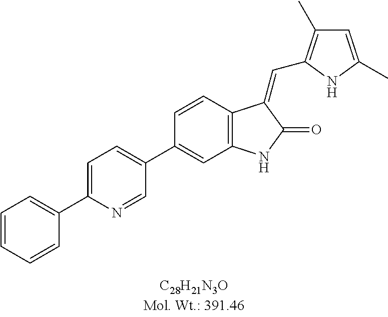 [SMILES: Cc1cc(C)c(/C=C2\C(=O)Nc3cc(-c4ccc(-c5ccccc5)nc4)ccc32)[nH]1] |
| 242 | 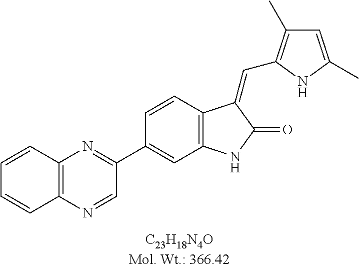 [SMILES: Cc1cc(C)c(/C=C2\C(=O)Nc3cc(-c4cnc5ccccc5n4)ccc32)[nH]1] |
| 243 | 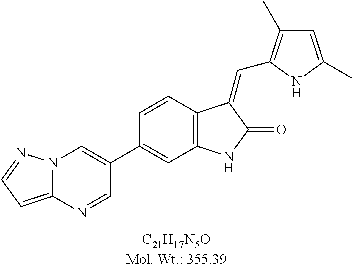 [SMILES: Cc1cc(C)c(/C=C2\C(=O)Nc3cc(-c4cnc5ccnn5c4)ccc32)[nH]1] |
| 244 | 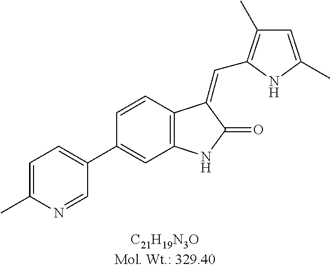 |
| 245 | 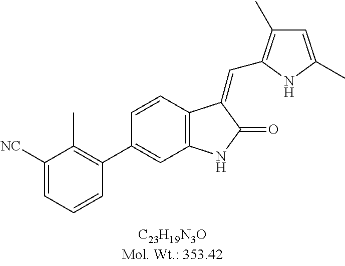 [SMILES: Cc1cc(C)c(/C=C2\C(=O)Nc3cc(-c4cccc(C#N)c4C)ccc32)[nH]1] |
| 246 | 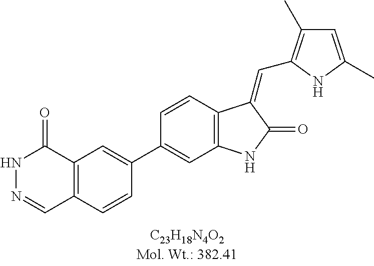 [SMILES: Cc1cc(C)c(/C=C2\C(=O)Nc3cc(-c4ccc5cn[nH]c(=O)c5c4)ccc32)[nH]1] |
| 247 | 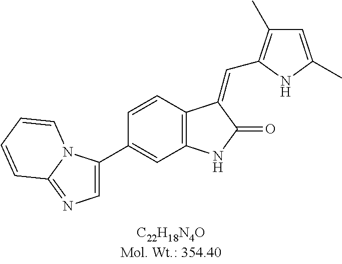 [SMILES: Cc1cc(C)c(/C=C2\C(=O)Nc3cc(-c4cnc5ccccn45)ccc32)[nH]1] |
| 248 | 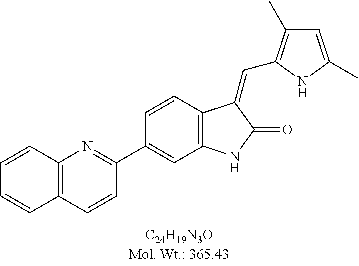 [SMILES: Cc1cc(C)c(/C=C2\C(=O)Nc3cc(-c4ccc5ccccc5n4)ccc32)[nH]1] |
| 249 | 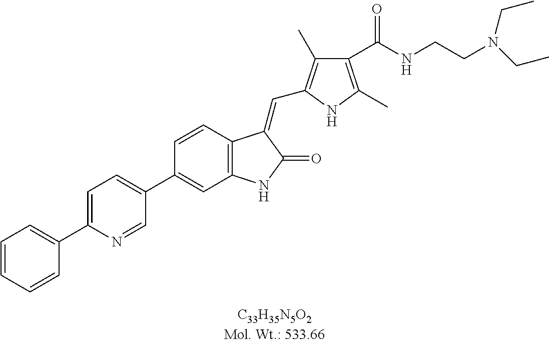 [SMILES: CCN(CC)CCNC(=O)c1c(C)[nH]c(/C=C2\C(=O)Nc3cc(-c4ccc(-c5ccccc5)nc4)ccc32)c1C] |
| 250 | 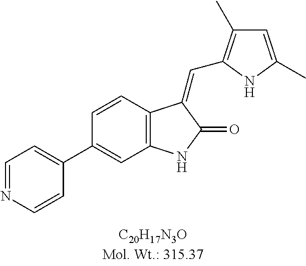 [SMILES: Cc1cc(C)c(/C=C2\C(=O)Nc3cc(-c4ccncc4)ccc32)[nH]1] |
| 251 | 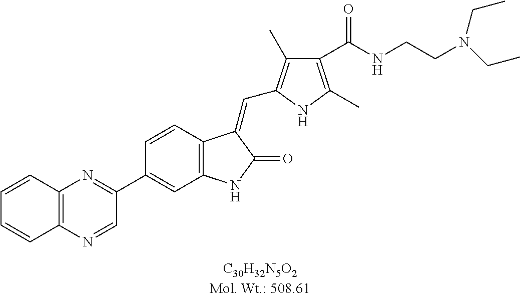 [SMILES: CCN(CC)CCNC(=O)c1c(C)[nH]c(/C=C2\C(=O)Nc3cc(-c4cnc5ccccc5n4)ccc32)c1C] |
| 252 | 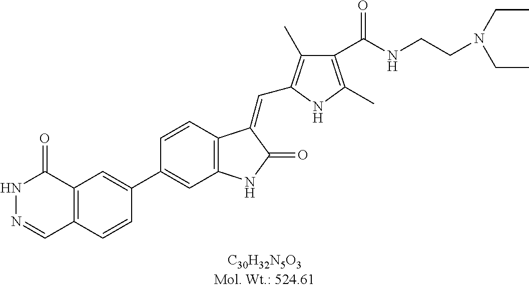 [SMILES: CCN(CC)CCNC(=O)c1c(C)[nH]c(/C=C2\C(=O)Nc3cc(-c4ccc5cn[nH]c(=O)c5c4)ccc32)c1C] |
| 253 | 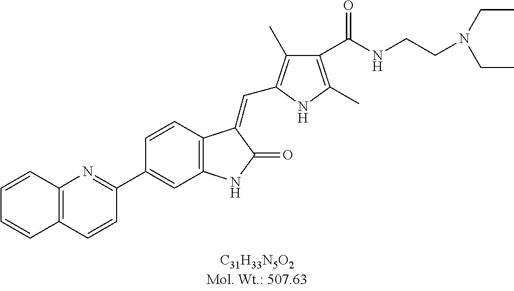 [SMILES: CCN(CC)CCNC(=O)c1c(C)[nH]c(/C=C2\C(=O)Nc3cc(-c4ccc5ccccc5n4)ccc32)c1C] |
| 254 | 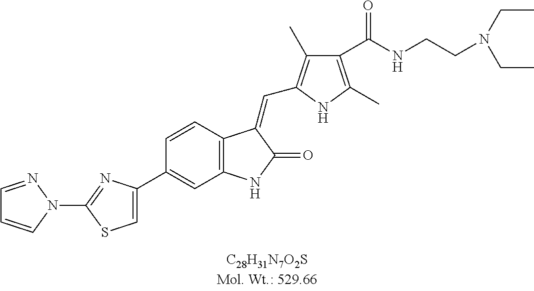 [SMILES: CCN(CC)CCNC(=O)c1c(C)[nH]c(/C=C2\C(=O)Nc3cc(-c4csc(-n5cccn5)n4)ccc32)c1C] |
| 255 | 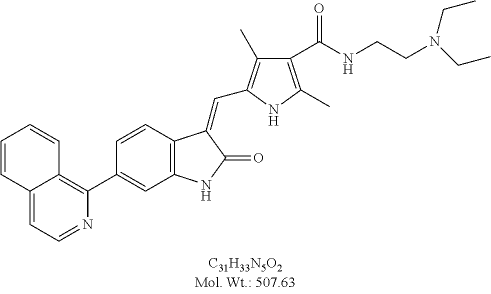 [SMILES: CCN(CC)CCNC(=O)c1c(C)[nH]c(/C=C2\C(=O)Nc3cc(-c4nccc5ccccc45)ccc32)c1C] |
| 256 | 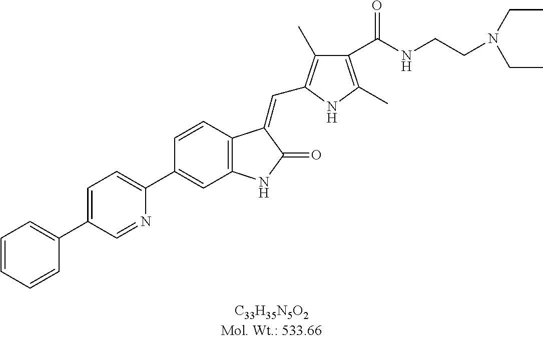 [SMILES: CCN(CC)CCNC(=O)c1c(C)[nH]c(/C=C2\C(=O)Nc3cc(-c4ccc(-c5ccccc5)cn4)ccc32)c1C] |
| 257 | 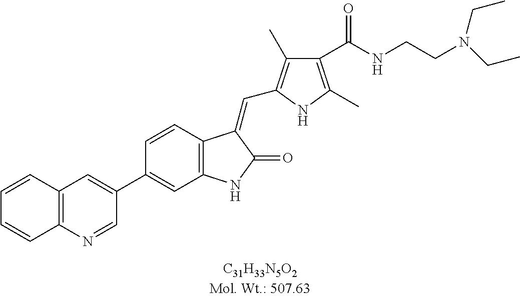 [SMILES: CCN(CC)CCNC(=O)c1c(C)[nH]c(/C=C2\C(=O)Nc3cc(-c4cnc5ccccc5c4)ccc32)c1C] |
| 258 | 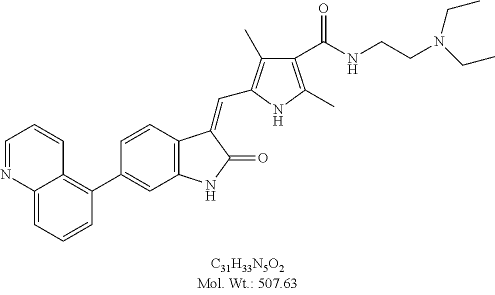 [SMILES: CCN(CC)CCNC(=O)c1c(C)[nH]c(/C=C2\C(=O)Nc3cc(-c4cccc5ncccc45)ccc32)c1C] |
| 259 | 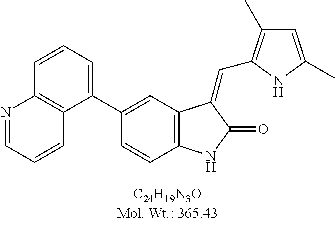 [SMILES: Cc1cc(C)c(/C=C2\C(=O)Nc3ccc(-c4cccc5ncccc45)cc32)[nH]1] |
| 260 | 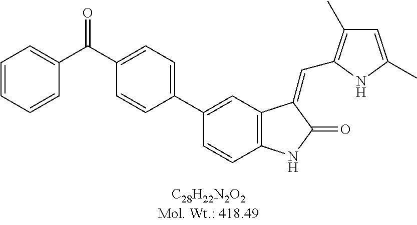 [SMILES: Cc1cc(C)c(/C=C2\C(=O)Nc3ccc(-c4ccc(C(=O)c5ccccc5)cc4)cc32)[nH]1] |
| 261 | 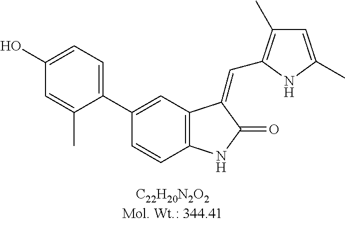 [SMILES: Cc1cc(C)c(/C=C2\C(=O)Nc3ccc(-c4ccc(O)cc4C)cc32)[nH]1] |
| 262 | 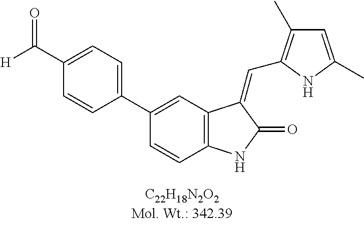 [SMILES: Cc1cc(C)c(/C=C2\C(=O)Nc3ccc(-c4ccc(C=O)cc4)cc32)[nH]1] |
| 263 | 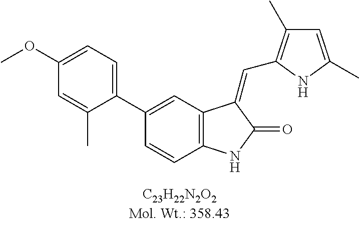 |
| 264 | 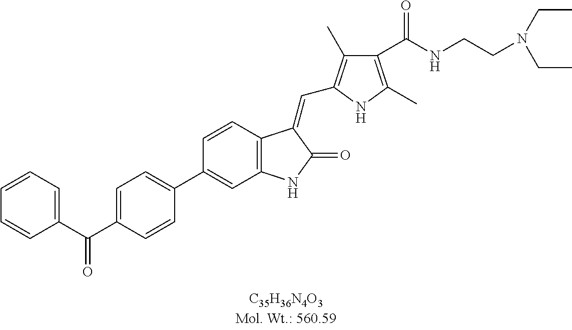 [SMILES: CCN(CC)CCNC(=O)c1c(C)[nH]c(/C=C2\C(=O)Nc3cc(-c4ccc(C(=O)c5ccccc5)cc4)ccc32)c1C] |
| 265 | 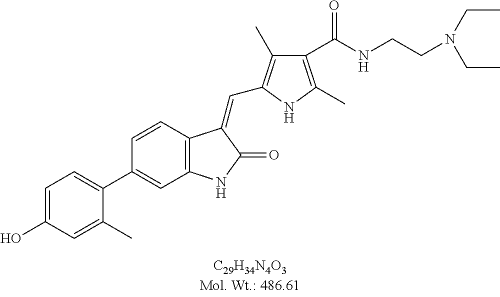 [SMILES: CCN(CC)CCNC(=O)c1c(C)[nH]c(/C=C2\C(=O)Nc3cc(-c4ccc(O)cc4C)ccc32)c1C] |
| 266 | 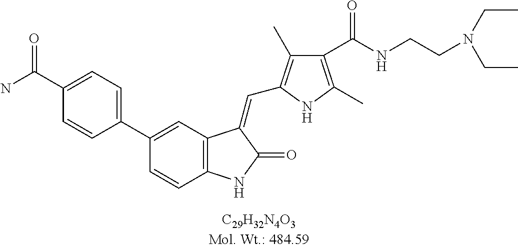 [SMILES: CCN(CC)CCNC(=O)c1c(C)[nH]c(/C=C2\C(=O)Nc3ccc(-c4ccc(C(N)=O)cc4)cc32)c1C] |
| 267 | 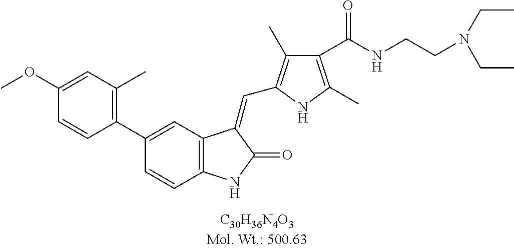 [SMILES: CCN(CC)CCNC(=O)c1c(C)[nH]c(/C=C2\C(=O)Nc3ccc(-c4ccc(OC)cc4C)cc32)c1C] |
| 268 | 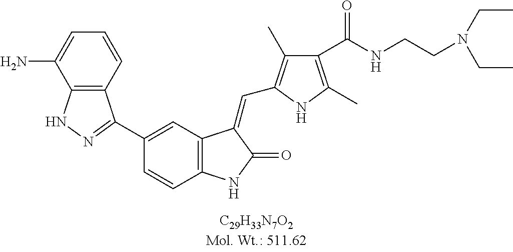 [SMILES: CCN(CC)CCNC(=O)c1c(C)[nH]c(/C=C2\C(=O)Nc3ccc(-c4n[nH]c5c(N)cccc45)cc32)c1C] |
| 269 | 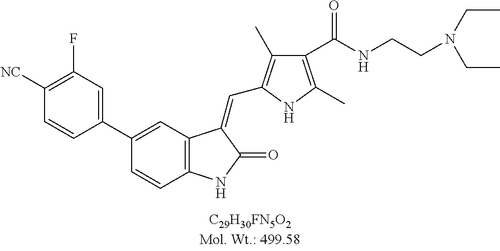 [SMILES: CCN(CC)CCNC(=O)c1c(C)[nH]c(/C=C2\C(=O)Nc3ccc(-c4ccc(C#N)c(F)c4)cc32)c1C] |
| 270 | 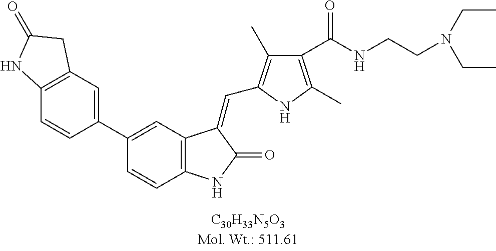 [SMILES: CCN(CC)CCNC(=O)c1c(C)[nH]c(/C=C2\C(=O)Nc3ccc(-c4ccc5c(c4)CC(=O)N5)cc32)c1C] |
| 271 | 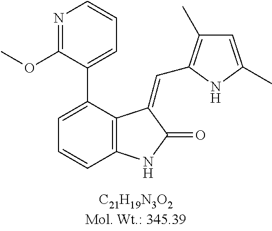 |
| 272 | 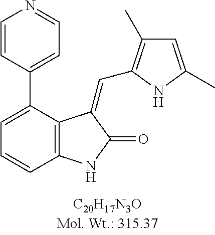 [SMILES: Cc1cc(C)c(/C=C2\C(=O)Nc3cccc(-c4ccncc4)c32)[nH]1] |
| 273 | 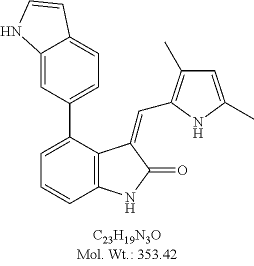 [SMILES: Cc1cc(C)c(/C=C2\C(=O)Nc3cccc(-c4ccc5cc[nH]c5c4)c32)[nH]1] |
| 274 | 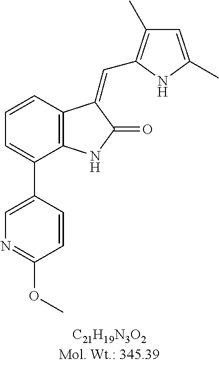 [SMILES: COc1ccc(-c2cccc3c2NC(=O)/C3=C\c2[nH]c(C)cc2C)cn1] |
| 275 | 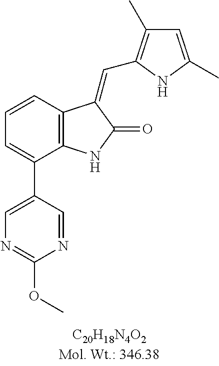 [SMILES: COc1ncc(-c2cccc3c2NC(=O)/C3=C\c2[nH]c(C)cc2C)cn1] |
| 276 | 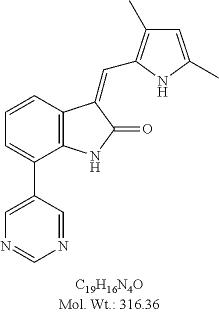 [SMILES: Cc1cc(C)c(/C=C2\C(=O)Nc3c2cccc3-c2cncnc2)[nH]1] |
| 277 | 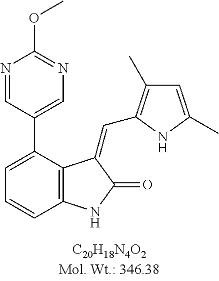 [SMILES: COc1ncc(-c2cccc3c2/C(=C/c2[nH]c(C)cc2C)C(=O)N3)cn1] |
| 278 | 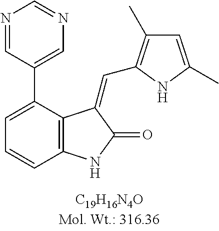 [SMILES: Cc1cc(C)c(/C=C2\C(=O)Nc3cccc(-c4cncnc4)c32)[nH]1] |
| 279 | 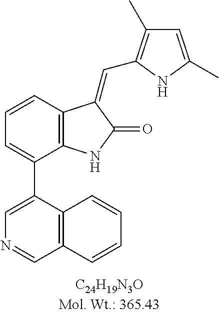 [SMILES: Cc1cc(C)c(/C=C2\C(=O)Nc3c2cccc3-c2cncc3ccccc23)[nH]1] |
| 280 | 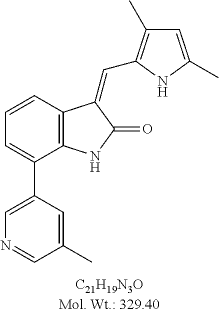 [SMILES: Cc1cncc(-c2cccc3c2NC(=O)/C3=C\c2[nH]c(C)cc2C)c1] |
| 281 | 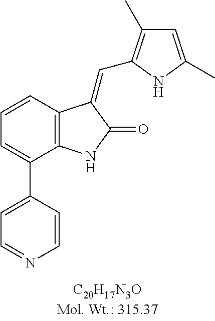 [SMILES: Cc1cc(C)c(/C=C2\C(=O)Nc3c2cccc3-c2ccncc2)[nH]1] |
| 282 | 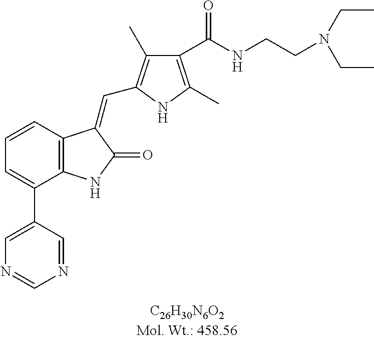 [SMILES: CCN(CC)CCNC(=O)c1c(C)[nH]c(/C=C2\C(=O)Nc3c2cccc3-c2cncnc2)c1C] |
| 283 | 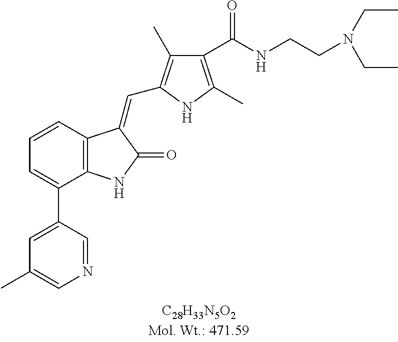 [SMILES: CCN(CC)CCNC(=O)c1c(C)[nH]c(/C=C2\C(=O)Nc3c2cccc3-c2cncc(C)c2)c1C] |
| 284 | 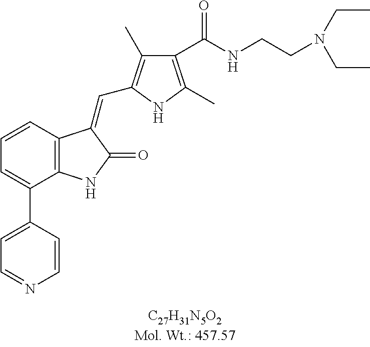 [SMILES: CCN(CC)CCNC(=O)c1c(C)[nH]c(/C=C2\C(=O)Nc3c2cccc3-c2ccncc2)c1C] |
| 285 | 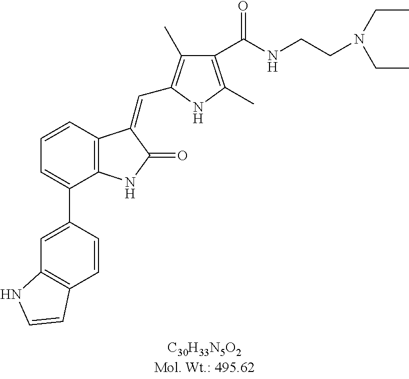 [SMILES: CCN(CC)CCNC(=O)c1c(C)[nH]c(/C=C2\C(=O)Nc3c2cccc3-c2ccc3cc[nH]c3c2)c1C] |
| 286 | 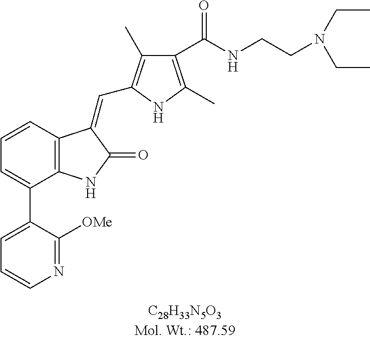 [SMILES: CCN(CC)CCNC(=O)c1c(C)[nH]c(/C=C2\C(=O)Nc3c2cccc3-c2cccnc2OC)c1C] |
| 287 | 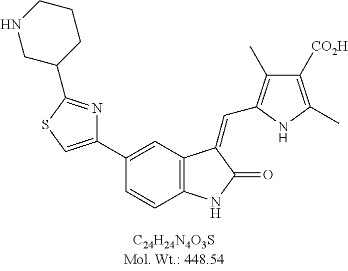 [SMILES: Cc1[nH]c(/C=C2\C(=O)Nc3ccc(-c4csc(C5CCCNC5)n4)cc32)c(C)c1C(=O)O] |

Biological Assays

### Test Example 1

### Identification of Compounds that Inhibit Kinases

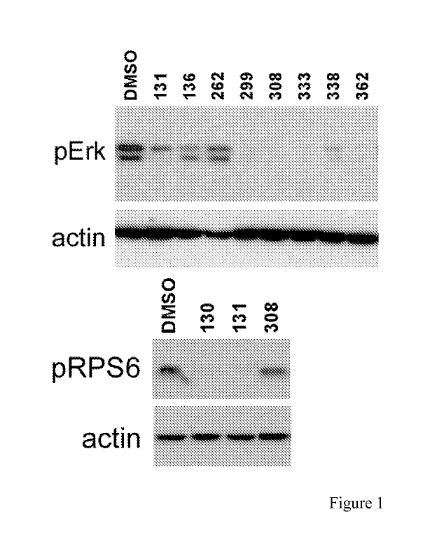

The ability of the Compounds was evaluated for its ability to inhibit certain oncogenic kinases. Cells were treated with each of the compounds for 6 hours. Western blot analysis was performed to determine levels of the phosphorylated forms of ERK and RPS6. It was found that incubation of cells with compounds of the present invention blocked phosphorylation of ERK and RPS6 (FIG. 1).

### Test Example 2

### Identification of Compounds that Target Bulk Cancer Cells

Bulk cells (FaDu, A549, and ACHN cell lines) were plated at 5,000 cells per well in black-walled clear-bottom 96 well plates in 100 uL per well of complete media (10% DMEM+penicillin/streptomycin+plasmocin) and allowed to grow for 24 hours. Tubes of 3× drug (60 uM in 500 uL complete media) were prepared from 5 mM stock solutions in DMSO. Compounds were serially diluted in deep-welled 96 well plates in complete media, giving 3× drug concentrations of 60, 30, 15, 7.5, 3.75, 1.875, 0.938, and 0.469 uM. To treat cells, 50 uL of drug from the dilution plate was removed and added to the cell plates. Drugs were tested in triplicate at final concentrations of 20, 10, 5, 2.5, 1.25, 0.625, 0.313, and 0.156 uM. Cells were harvested 72 hours after drug addition using Cell Titer-Glo (Promega) and luminescence measured on a plate reader. The results are shown in Table 14.

TABLE 14

| | FaDu | A549 | ACHN |
|---|---|---|---|
| Compound | IC50 (uM) | IC50 (uM) | IC50 (uM) |
| 1 | 4.74 | | 7.60 |
| 2 | 14.06 | 48.90 | |
| 3 | 25.42 | | |
| 4 | 6.25 | 5.35 | 2.28 |
| 6 | 3.62 | 6.91 | 3.40 |
| 7 | 1.80 | | |
| 8 | 4.74 | 26.34 | 11.61 |
| 9 | 3.75 | 1.96 | 3.20 |
| 10 | 5.41 | 5.41 | 15.16 |
| 11 | 21.11 | 10.14 | 12.82 |
| 12 | 23.94 | | |
| 13 | 6.76 | | |
| 14 | 4.44 | | |
| 16 | 2.84 | 29.20 | 3.54 |
| 17 | 9.73 | | 12.20 |
| 18 | 12.10 | | |
| 19 | 7.35 | | |
| 22 | 3.01 | 2.94 | 6.57 |
| 23 | 4.55 | | |
| 25 | 17.29 | | |
| 26 | 14.30 | | |
| 27 | 2.51 | | |
| 28 | 5.79 | | |
| 31 | 3.67 | 18.90 | |
| 32 | 0.51 | 1.72 | 1.24 |
| 33 | 2.53 | 2.53 | 1.64 |
| 37 | 3.55 | 7.49 | 2.97 |
| 41 | 5.34 | | 20.60 |
| 42 | 1.89 | 16.03 | |
| 43 | 6.47 | | |
| 44 | 5.54 | | |
| 45 | 3.32 | 29.63 | 4.80 |
| 46 | 23.24 | | |
| 47 | 4.22 | 16.01 | 10.40 |
| 48 | 6.74 | 4.88 | 4.73 |
| 49 | 6.58 | | 7.16 |
| 50 | 6.16 | | |
| 51 | 1.18 | 4.74 | 1.64 |
| 52 | 2.15 | 48.90 | |
| 53 | 8.23 | | |
| 54 | 23.70 | | |
| 56 | 3.09 | 4.30 | 3.66 |
| 103 | 4.22 | 5.99 | 2.32 |
| 104 | 7.87 | 8.55 | 4.25 |
| 105 | 3.05 | | |
| 109 | 1.11 | 5.09 | 1.60 |
| 110 | 7.56 | 21.50 | 14.90 |
| 117 | 39.21 | | |
| 118 | 1.37 | 1.74 | 1.3675 |
| 119 | 1.73 | 1.43 | 1.52 |
| 120 | 11.2 | | |
| 121 | 3.10 | 3.64 | 3.31 |
| 124 | 46.90 | | |
| 125 | 3.68 | | |
| 126 | 0.70 | 3.06 | 1.38 |
| 127 | 24.80 | | |
| 129 | 18.28 | 9.94 | 11.26 |
| 132 | 5.35 | | |
| 133 | 2.42 | 0.72 | 6.75 |
| 135 | 1.77 | | 1.27 |
| 138 | 7.87 | | 5.31 |
| 139 | 1.62 | 1.29 | 1.56 |
| 140 | 1.58 | 1.34 | 1.64 |
| 141 | 21.76 | 16.32 | 1.16 |
| 144 | 0.69 | 0.70 | 0.52 |
| 145 | 18.62 | 9.61 | 12.60 |
| 146 | 5.48 | 2.86 | 12.62 |
| 147 | 11.36 | 7.28 | 10.67 |
| 149 | 3.85 | 1.11 | 2.48 |
| 150 | 7.57 | 17.25 | 1.44 |
| 151 | 6.14 | 11.15 | 8.74 |
| 152 | 1.66 | 9.19 | 3.61 |
| 153 | 3.42 | 14.75 | 0.29 |
| 154 | | | 14.59 |
| 155 | 20.25 | | 34.61 |
| 156 | | | 37.82 |
| 157 | 21.12 | 9.39 | 18.81 |
| 159 | 49.26 | | |
| 161 | 13.52 | 40.03 | 0.39 |
| 163 | | | 24.92 |
| 164 | | | 0.83 |
| 165 | 11.37 | 4.00 | 14.91 |
| 166 | | | 9.91 |
| 167 | 27.98 | 10.07 | 7.42 |
| 168 | 5.16 | 5.55 | 4.12 |
| 170 | 25.18 | 8.73 | |
| 171 | 2.59 | 5.79 | |
| 172 | 35.47 | | |
| 173 | 17.51 | | |
| 176 | 5.31 | 18.03 | |
| 178 | 8.67 | 7.06 | |
| 179 | 1.74 | 3.66 | 3.64 |
| 180 | 6.62 | 10.55 | 10.07 |
| 181 | 1.49 | 3.24 | 2.74 |
| 182 | 6.02 | 15.43 | 3.50 |
| 183 | 2.56 | 7.23 | 2.26 |
| 184 | 3.05 | 6.51 | 2.92 |
| 185 | 2.66 | 8.13 | 5.73 |
| 186 | 0.92 | 1.80 | 2.74 |
| 187 | 2.13 | 3.18 | 5.05 |
| 188 | 7.01 | 10.81 | 6.44 |
| 189 | 2.63 | 7.48 | 7.14 |
| 190 | 1.76 | 2.79 | 1.96 |
| 191 | 4.93 | 3.73 | 9.53 |
| 192 | 4.63 | 5.86 | 4.65 |
| 193 | 2.85 | 3.02 | 4.31 |
| 194 | 4.92 | 2.30 | 7.41 |
| 195 | 4.68 | 3.71 | 7.88 |
| 196 | 5.57 | 6.16 | 9.57 |
| 197 | 2.72 | 2.02 | 5.38 |
| 198 | 2.58 | 1.50 | 2.41 |
| 199 | 17.47 | 16.93 | 25.82 |
| 200 | 3.17 | 3.79 | 4.58 |
| 201 | 5.96 | 6.06 | 12.32 |
| 202 | | 7.79 | 7.74 |
| 203 | 7.67 | 4.14 | 4.15 |
| 204 | 4.59 | 3.85 | 6.39 |
| 205 | 4.27 | 2.80 | 5.23 |
| 206 | 3.74 | 2.01 | 2.68 |
| 207 | 4.55 | 7.33 | 3.66 |
| 208 | 6.81 | 7.07 | 11.59 |
| 209 | 1.21 | 2.20 | 2.04 |
| 210 | 2.82 | 0.84 | 1.31 |
| 211 | | 22.45 | 7.95 |
| 212 | | 20.79 | 12.82 |
| 213 | | 25.81 | 24.95 |
| 214 | | | 7.44 |
| 215 | 14.51 | 9.31 | 8.84 |
| 216 | 37.31 | | |
| 217 | 25.82 | 7.89 | 10.57 |
| 219 | 23.51 | 18.36 | |
| 220 | 26.66 | | 42.81 |
| 221 | | 35.83 | 0.16 |
| 222 | 33.22 | | |
| 223 | 22.38 | 13.80 | |
| 224 | 11.36 | 16.59 | 25.22 |
| 227 | | 0.07 | |
| 228 | | | 22.39 |
| 231 | 4.43 | 6.45 | 8.73 |
| 232 | 20.82 | 9.76 | 9.00 |
| 234 | 5.23 | 12.51 | |
| 235 | 26.81 | 16.15 | 14.69 |
| 236 | 26.14 | | 25.27 |
| 239 | 15.88 | | 37.21 |
| 240 | 2.55 | 1.40 | 1.12 |
| 242 | | 18.22 | |
| 244 | | | 22.01 |
| 247 | 19.40 | | 20.55 |
| 248 | 14.03 | | |
| 249 | | 14.84 | |
| 250 | 18.43 | | |
| 251 | 7.45 | 7.07 | 10.88 |
| 252 | 23.94 | | |
| 253 | 16.65 | 16.35 | |
| 254 | 5.30 | 5.80 | 2.60 |
| 255 | 9.12 | 7.14 | 6.51 |
| 256 | 19.82 | | |
| 257 | 12.88 | 15.78 | |
| 258 | 4.83 | 2.88 | 2.66 |
| 259 | 13.42 | 11.06 | 13.24 |
| 260 | 22.36 | | 21.77 |
| 261 | 14.72 | 12.54 | 14.89 |
| 262 | 26.77 | 29.24 | |
| 263 | 19.77 | | 24.80 |
| 264 | 4.46 | 3.65 | 4.11 |
| 265 | 2.14 | 2.76 | 2.53 |
| 266 | 12.12 | 10.62 | 2.81 |
| 267 | 2.31 | 1.74 | 2.26 |
| 268 | 7.09 | 11.63 | 12.02 |
| 269 | 13.98 | 14.51 | 13.33 |
| 270 | 25.38 | 19.66 | 18.94 |
| 271 | 22.59 | | |
| 272 | 19.58 | 13.11 | 19.75 |
| 273 | 16.35 | 16.65 | 20.39 |
| 274 | 28.44 | 24.39 | 28.26 |
| 275 | 20.79 | 17.70 | 21.94 |
| 276 | 28.10 | 25.15 | 26.90 |
| 277 | 35.32 | 21.08 | 28.10 |
| 278 | 39.73 | 16.29 | 16.80 |
| 279 | 25.78 | 19.71 | 20.97 |
| 280 | 26.43 | 21.15 | 15.96 |
| 281 | | 23.53 | |
| 282 | 36.31 | 21.46 | 16.43 |
| 283 | 6.23 | 3.36 | 1.68 |
| 284 | 6.74 | 6.85 | 6.39 |
| 285 | 2.77 | 2.91 | 3.00 |
| 286 | 3.81 | 5.32 | 3.64 |

### Test Example 3

### Identification of Compounds that Target Cancer Stem Cells

Cancer Stem Cell (CSC) cultures were initiated from heterogeneous cancer cell lines. Cancer stem cells (CSCs; FaDu, A549, and ACHN cell lines) that have grown for a minimum of 1 passage in complete CSC media (DMEM/F12 media supplemented with Gibco B-27, 20 ng/mL EGF, 10 ng/mL basic FGF, and 0.4% BSA) were dissociated in 2 mL accutase, washed in CSC media, filtered through a 40 um cell strainer, and counted. CSCs were plated at 1,000 cells per well in black-walled clear-bottom 96 well plates, which had been coated in 0.5% agar, in 100 uL per well of CSC media and allowed to grow for 72 hours. Tubes of 3× drug (60 uM in 500 uL CSC media) were prepared from 5 mM stock solutions in DMSO. Compounds were serially diluted in deep-welled 96 well plates in CSC media, giving 3× drug concentrations of 60, 30, 15, 7.5, 3.75, 1.875, 0.938, and 0.469 uM. To treat cells, 50 uL of drug from the dilution plate was removed and added to the cell plates. Drugs were tested in triplicate at final concentrations of 20, 10, 5, 2.5, 1.25, 0.625, 0.313, and 0.156 uM. Cells were harvested 72 hours after drug addition using Cell Titer-Glo (Promega) and luminescence measured on a plate reader. The results are shown in Table 15.

TABLE 15

| | FaDu CSC | A549 CSC | ACHN CSC |
|---|---|---|---|
| | IC50 (uM) | IC50 (uM) | IC50 (uM) |
| 1 | 1.20 | | |
| 2 | 0.23 | | |
| 3 | 1.16 | | |
| 4 | 0.60 | 4.83 | |
| 5 | 14.78 | | |
| 6 | 0.31 | 4.49 | |
| 7 | 0.60 | 3.73 | 0.19 |
| 8 | 1.03 | 1.22 | 0.39 |
| 9 | 1.26 | 1.57 | 0.28 |
| 10 | 2.99 | 4.19 | 0.50 |
| 11 | 2.47 | 11.03 | 0.23 |
| 13 | 37.24 | | |
| 14 | 1.64 | | |
| 15 | 30.52 | | |
| 16 | 1.31 | 4.90 | |
| 17 | 41.51 | | |
| 18 | 9.35 | | |
| 19 | 27.58 | 0.35 | |
| 20 | 2.38 | | |
| 22 | 3.46 | 5.54 | 1.17 |
| 23 | 11.48 | | |
| 24 | 48.22 | | |
| 25 | 4.96 | | |
| 26 | 40.82 | | |
| 27 | 0.83 | | |
| 28 | 1.31 | | |
| 30 | 8.94 | | |
| 31 | 0.24 | 11.70 | |
| 32 | 0.05 | 2.53 | 0.13 |
| 33 | 1.65 | 11.70 | 0.22 |
| 34 | 18.18 | | |
| 36 | 41.90 | | |
| 37 | 8.09 | | 1.65 |
| 38 | 1.60 | | 0.50 |
| 42 | 1.30 | | |
| 43 | 18.84 | | |
| 44 | 13.23 | | |
| 45 | 1.00 | | |
| 46 | 15.56 | | |
| 47 | 2.57 | | |
| 48 | 2.90 | | |
| 49 | 3.00 | | |
| 50 | 2.80 | | |
| 51 | 0.27 | 4.28 | |
| 52 | 0.23 | | |
| 53 | 21.51 | | |
| 56 | 1.41 | 8.25 | 0.25 |
| 102 | 3.40 | 10.51 | 0.69 |
| 103 | 0.31 | 12.20 | 0.14 |
| 104 | 1.98 | 0.45 | |
| 108 | 20.16 | | |
| 109 | 0.28 | 4.32 | 0.33 |
| 110 | 1.21 | 5.76 | |
| 116 | 1.60 | | |
| 118 | 0.11 | 0.62 | 0.97 |
| 119 | 0.17 | 2.47 | 0.40 |
| 120 | 4.75 | | |
| 121 | 6.15 | | |
| 122 | 23.15 | | |
| 123 | 33.67 | | |
| 125 | 1.33 | | |
| 126 | 1.85 | 6.99 | 0.50 |
| 127 | 7.82 | | |
| 129 | 3.60 | 10.51 | 0.69 |
| 132 | 2.17 | | |
| 133 | | 32.50 | |
| 134 | 2.34 | | |
| 135 | 4.88 | 4.21 | |
| 136 | 2.52 | | |
| 137 | 4.88 | 4.21 | |
| 138 | 6.49 | | |
| 139 | 0.43 | 0.88 | 0.09 |
| 140 | 0.48 | 1.70 | 0.05 |
| 142 | 5.62 | | |
| 144 | 0.22 | 2.48 | |
| 145 | 6.74 | 4.80 | 6.16 |
| 146 | 6.56 | | 5.05 |
| 147 | 0.21 | 3.73 | 0.56 |
| 148 | | 1.65 | |
| 149 | 3.53 | 8.74 | 4.23 |
| 150 | 2.50 | 2.31 | 2.91 |
| 151 | 6.32 | 0.90 | |
| 152 | 22.06 | 7.18 | 28.90 |
| 153 | | 2.60 | 9.21 |
| 155 | 4.09 | 1.42 | 2.52 |
| 156 | | 0.80 | |
| 157 | 11.95 | 22.11 | 2.68 |
| 158 | 3.07 | | |
| 161 | | 13.17 | |
| 162 | 5.04 | | 4.08 |
| 163 | 4.49 | | |
| 164 | 2.91 | | 1.60 |
| 165 | 3.13 | 26.66 | 4.04 |
| 167 | 4.57 | 35.71 | 1.24 |
| 168 | 0.76 | 2.04 | 0.10 |
| 178 | | | 11.87 |
| 179 | 0.06 | 6.64 | 2.73 |
| 180 | 0.23 | 13.22 | 2.65 |
| 181 | 0.16 | 26.40 | 10.82 |
| 182 | 0.09 | 18.04 | 1.15 |
| 183 | 0.18 | 5.45 | 1.06 |
| 184 | 0.22 | 3.38 | 1.16 |
| 185 | 0.04 | 42.33 | 5.59 |
| 186 | | 1.51 | 0.33 |
| 187 | 0.03 | 13.86 | 0.71 |
| 188 | 0.16 | 8.89 | 1.08 |
| 189 | 0.07 | 6.84 | 1.41 |
| 190 | 0.19 | 2.24 | 0.24 |
| 191 | 5.97 | 4.13 | 0.98 |
| 192 | 17.62 | 17.22 | 3.44 |
| 193 | 10.17 | 5.65 | 1.31 |
| 194 | 3.96 | 6.45 | 0.63 |
| 195 | 5.11 | 6.64 | 0.62 |
| 196 | 21.72 | 18.83 | 2.99 |
| 197 | 4.81 | 3.79 | 0.65 |
| 198 | 9.67 | 2.00 | 0.90 |
| 199 | 39.07 | 43.57 | |
| 200 | 3.42 | 6.66 | 0.64 |
| 201 | | 30.03 | |
| 202 | 14.13 | 14.11 | 1.28 |
| 203 | 19.98 | 13.12 | 1.88 |
| 204 | 5.47 | 6.83 | 0.62 |
| 205 | 4.73 | 4.48 | 0.57 |
| 206 | 9.10 | 3.94 | 0.48 |
| 207 | 10.07 | 17.98 | 0.55 |
| 208 | 16.18 | 9.85 | 2.14 |
| 209 | 4.30 | 8.82 | 2.28 |
| 210 | 4.31 | 3.90 | 1.06 |
| 211 | 41.13 | | 0.01 |
| 213 | | | 0.01 |
| 215 | 37.35 | 35.00 | 2.72 |
| 216 | | | 0.05 |
| 217 | 0.09 | | |
| 220 | 43.39 | | 17.14 |
| 221 | | | 18.31 |
| 223 | | 35.71 | |
| 224 | 17.01 | 20.24 | |
| 225 | 47.37 | | 0.01 |
| 226 | | | 0.02 |
| 228 | | 25.43 | 3.42 |
| 231 | 5.33 | 7.28 | 3.10 |
| 232 | 27.16 | 16.18 | 0.00 |
| 233 | | | 6.80 |
| 234 | 1.55 | 8.66 | |
| 236 | 25.24 | 28.74 | |
| 238 | | | 6.95 |
| 239 | 8.22 | | 3.48 |
| 240 | 2.96 | 3.00 | 0.26 |
| 241 | 18.15 | | |
| 242 | 8.80 | 5.83 | 2.54 |
| 243 | 14.93 | | |
| 244 | 3.61 | 23.67 | 4.55 |
| 245 | 39.07 | | |
| 249 | 1.12 | 3.59 | 0.21 |
| 250 | | 0.98 | |
| 251 | 2.24 | 42.57 | 6.97 |
| 253 | 5.15 | 6.59 | 1.36 |
| 254 | 0.78 | 2.93 | 0.17 |
| 255 | 24.42 | | 1.91 |
| 256 | 1.18 | 4.93 | 2.88 |
| 257 | 2.83 | 11.63 | 1.07 |
| 258 | 2.64 | 8.61 | 0.42 |
| 259 | 13.13 | 40.00 | |
| 261 | 14.43 | 36.80 | 21.07 |
| 264 | 1.46 | 3.16 | 1.63 |
| 265 | 1.54 | 7.55 | 2.17 |
| 266 | 7.51 | 5.84 | 4.34 |
| 267 | 1.40 | 4.61 | 1.42 |
| 268 | 28.42 | | 30.85 |
| 269 | 3.48 | 11.98 | 1.71 |
| 270 | 15.48 | 29.18 | 20.82 |
| 272 | 12.24 | 30.28 | 16.24 |
| 273 | | 20.19 | 18.94 |
| 274 | | 43.39 | |
| 275 | | 34.69 | 17.50 |
| 280 | | 32.01 | 32.03 |
| 282 | 6.73 | 27.54 | 10.91 |
| 283 | 2.20 | 2.23 | 1.97 |
| 284 | 4.41 | 12.55 | 4.99 |
| 285 | 2.06 | 3.90 | 1.86 |
| 286 | 1.92 | 6.87 | 2.71 |

### Test Example 4

### Right Open Reading Frame Kinase 2 (RIOK2) Inhibitor Screening System Adopting the Autophosphorylation Activity of the Human RIOK2 Protein as an Indicator

A protein in which glutathione S-transferase is fused to N-terminal of a full-length human RIOK2 protein was used as a recombinant human RIOK2 protein. The protein was prepared using a baculovirus-Sf21 cell expression system from CarnaBio, Inc. A kinase reaction solution was prepared by mixing 25 μg/mL of the recombinant human RIOK2 protein and 100 μM of ATP in 20 μl of kinase reaction buffer (50 mM Tris-HCl, 10 mM MgCl₂, 10 mM MnCl₂, 1 mM DTT, 0.01% Brij, 1×PhosSTOP (manufactured by Roche Applied Science)), and was incubated at a temperature of 30° C. for 1 hour. After adding 30 μl of 40 mM EDTA solution to this kinase reaction solution, the kinase reaction solution was transferred to a MaxiSorp 96 well plate (manufactured by Nunc). Thereafter, phosphorylated human RIOK2 protein was allowed to adsorb onto the wells by leaving the plate in a refrigerator overnight.

The following day, the supernatant was removed, and the wells were washed three times with ELISA buffer (20 mM Tris-HCl, 150 mM NaCl, 0.2% Tween-20, 0.1% BSA). Thereafter, 100 μl of Blocking-One P (product name; manufactured by Nacalai Tesque, Inc.) was applied to the wells, and a blocking treatment was performed for 1 hour at room temperature. After washing the wells using a similar method as given above, an anti-phospho Thr antibody (manufactured by Cell Signaling) solution diluted to 1/4000 with ELISA buffer was applied to the wells, and incubation was performed for 30 minutes at room temperature. After further washing the wells, a secondary antibody, anti-rabbit IgG conjugated with horseradish peroxidase (manufactured by Jackson ImmunoResearch; 1/4000 ELISA buffer-diluted solution), was applied to the wells, and incubation was performed for 30 minutes at room temperature. After washing the wells once again, 50 μl of tetramethyl benzidine (TMB) aqueous solution (manufactured by Nacalai Tesque, Inc.) was applied to the wells, and a coloring reaction was performed for 30 minutes at room temperature. Thereafter, the coloring reaction was stopped by adding 50 μl of 2M H₂SO₄ to the wells. An absorptiometric level of 450 nm was measured in each well using ARVO SX (manufactured by Wallac). As a result, autophosphorylation activity was at least four times higher compared to when ATP was not added.

Additionally, the following method showed that the kinase reaction is blocked by staurosporine, which is a typical kinase inhibitor. That is, first staurosporine of a predetermined concentration (manufactured by Wako Pure Chemical Industries, Ltd.) was applied to a kinase reaction buffer together with a full-length human RIOK2 protein, and then reacted under incubation conditions at 30° C. for 1 hour. Upon measuring kinase activity (phosphorylation activity) of RIOK2 in the reaction solution thereafter, it was found that the RIOK2 kinase activity was suppressed depending on the concentration of staurosporine. As a result, it was found that the RIOK2 kinase activity measurement system is a technique that can be used in searching for RIOK2 inhibitors. The results are shown in Table 16.

TABLE 16

| | Inhibitory activity | Inhibitory activity |
|---|---|---|
| Example | at 1 μM (%) | at 10 μM (%) |
| 1 | — | 14 |
| 2 | — | 19 |
| 3 | — | 65 |
| 4 | 45 | 94 |
| 16 | 55 | 76 |
| 19 | 32 | 56 |
| 22 | 55 | 100 |
| 32 | 36 | 86 |
| 33 | 53 | 91 |
| 47 | 32 | 68 |
| 57 | 39 | 104 |
| 58 | 56 | 116 |
| 59 | 34 | 106 |
| 60 | 9 | 68 |
| 61 | 29 | 137 |
| 62 | 51 | 98 |
| 63 | 14 | 93 |
| 65 | 18 | 87 |
| 66 | −6 | 44 |
| 68 | 47 | 95 |
| 69 | 40 | 109 |
| 70 | 42 | 112 |
| 71 | 4 | 99 |
| 72 | 41 | 99 |
| 73 | 18 | 111 |
| 74 | 26 | 71 |
| 79 | 42 | 82 |
| 88 | 1.4 | 122 |
| 89 | 22 | 130 |
| 90 | 20 | 96 |
| 91 | −7 | 43 |
| 92 | 3 | 113 |
| 93 | 7 | 65 |
| 94 | 17 | 140 |
| 95 | 22 | 134 |
| 96 | −13 | 69 |
| 97 | 6 | 107 |
| 98 | −2 | 25 |
| 99 | −11 | 79 |
| 100 | 9 | 126 |
| 101 | 13 | 94 |

These RIOK2 inhibitors inhibit cancer stem cell growth by inhibiting a function of a RIOK2 protein.

### Test Example 5

### Kinase Inhibition

HeLa cells were treated with 5 uM of the indicated compound for 6 hours. Following compound incubation, cells were washed twice with ice-cold PBS and lysed in lysis buffer (50 mM HEPES, pH 7.5, 1% Nonidet P-40, 150 mM NaCl, 1 mM EDTA, 1× protease inhibitor cocktail (Promega)). 30 micrograms of soluble protein was separated by SDS-PAGE and transferred to PVDF membranes. Primary antibodies against p-AMPK Thr172, p-RPS6 Ser235/236, p-RPS6 Ser240/244, and Tubulin (Cell Signaling Technology) were used. The antigen-antibody complexes were visualized by enhanced chemiluminescence (BioRad). The results are shown below in Tables 17A-17E.

TABLE 17A

| | | p-RPS6 | |
|---|---|---|---|
| | p-AMPK | Ser235/236 | p-RPS6 Ser240/244 |
| | Relative Intensity | Relative Intensity | Relative Intensity |
| Compound | (%) | (%) | (%) |
| DMSO | 100 | 100 | 100 |
| 8 | 150 | 33 | 73 |
| 9 | 144 | 2 | 32 |
| 10 | 122 | 2 | 32 |
| 15 | 150 | 5 | 56 |
| 109 | 8 | 2 | 23 |
| 110 | 105 | 2 | 36 |
| 118 | 35 | 5 | 42 |

TABLE 17B

| | | p-RPS6 | |
|---|---|---|---|
| | p-AMPK | Ser235/236 | p-RPS6 Ser240/244 |
| | Relative Intensity | Relative Intensity | Relative Intensity |
| Compound | (%) | (%) | (%) |
| DMSO | 100 | 100 | 100 |
| 119 | 41 | 13 | 51 |
| 36 | 118 | 4 | 44 |
| 27 | 21 | 5 | 41 |
| 29 | 108 | 36 | 68 |
| 37 | 151 | 4 | 43 |
| 38 | 103 | 2 | 21 |
| 51 | 12 | 21 | 64 |
| 33 | 52 | 82 | 92 |
| 56 | 144 | 19 | 64 |
| 126 | 47 | 79 | 88 |
| 139 | 47 | 76 | 76 |

TABLE 17C

| | | p-RPS6 | |
|---|---|---|---|
| | p-AMPK | Ser235/236 | p-RPS6 Ser240/244 |
| | Relative Intensity | Relative Intensity | Relative Intensity |
| Compound | (%) | (%) | (%) |
| DMSO | 100 | 100 | 100 |
| 140 | 69 | 6 | 34 |
| 144 | 79 | 70 | 83 |
| 193 | 22 | 4 | 26 |
| 197 | 118 | 61 | 82 |
| 210 | 8 | 1 | 31 |
| 240 | 39 | 1 | 24 |

TABLE 17D

| | | p-RPS6 | |
|---|---|---|---|
| | p-AMPK | Ser235/236 | p-RPS6 Ser240/244 |
| | Relative Intensity | Relative Intensity | Relative Intensity |
| Compound | (%) | (%) | (%) |
| DMSO | 100 | 100 | 100 |
| 271 | 105 | 67 | 67 |
| 272 | 75 | 50 | 47 |
| 273 | 27 | 36 | 35 |
| 274 | 91 | 63 | 59 |
| 275 | 96 | 57 | 54 |
| 276 | 113 | 51 | 50 |
| 277 | 145 | 77 | 75 |
| 278 | 198 | 44 | 43 |
| 279 | 108 | 86 | 83 |
| 280 | 103 | 74 | 75 |
| 281 | 83 | 68 | 65 |

TABLE 17E

| | | p-RPS6 | |
|---|---|---|---|
| | p-AMPK | Ser235/236 | p-RPS6 Ser240/244 |
| | Relative Intensity | Relative Intensity | Relative Intensity |
| Compound | (%) | (%) | (%) |
| DMSO | 100 | 100 | 100 |
| 282 | 60 | 71 | 72 |
| 283 | 50 | 47 | 59 |
| 284 | 56 | 60 | 65 |
| 285 | 39 | 29 | 44 |
| 286 | 31 | 48 | 54 |

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound of Formula I, 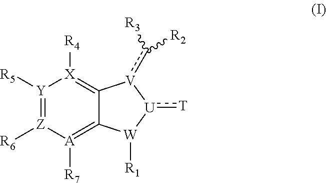
or an enantiomer, diastereomer, tautomer, or pharmaceutically acceptable salt or solvate thereof, wherein:
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₂ is monocyclic or bicyclic heterocycle or substituted heterocycle, aryl or substituted aryl;
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, S(O)₂NRₐR_{b};
R₄, R₅, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
T is O, S or Rₐ;
U and V are each independently a carbon;
W is N;
X, Y, Z, and A are each independently a carbon or N, with the proviso that the ring in which X, Y, Z, and A exist is aromatic;
with the provision that
one of R₄, R₅, R₆, and R₇ is substituted heterocycle or substituted aryl, and
R₄, R₅, R₆, or R₇ is absent if X, Y, Z, or A, respectively, is a heteroatom;
wherein
substituted heterocycle and substituted aryl in R₄, R₅, R₆, and R₇ is the following group: 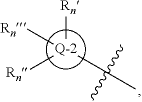
wherein:
Q-2 is heterocycle or aryl selected from the group consisting of pyrrole, furan, thiophene, imidazole, pyrazole, oxazole, isoxazole, thiazole, isothiazole, triazole, thiadiazole, oxadiazole, pyrrolidine, piperidine, azepane, tetrahydrofuran, oxane, oxepane, indole, indolinone, indazole, benzothiazole, quinoline, quinazoline, quinoxaline, imidazopyridine, imidazopyridazine, pyrazolopyridine, pyrazolopyrimidine, and phthalazinone;
Rₙ′, Rₙ″ and Rₙ′″ are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, pyrrolidinyl, piperidinyl, azepanyl, tetrahydrofuranyl, oxanyl, oxepanyl, pyranyl, phenyl, thiophenyl, pyrazinyl, pyrimidinyl, pyridazinyl, or pyridyl (said piperidinyl, pyranyl, phenyl, thiophenyl, pyrazinyl, pyrimidinyl, pyridazinyl, and pyridyl are optionally substituted with halogen, cyano, nitro, alkyl or substituted alkyl, ORₐ, NR_{b}R_{c}, C(═O)ORₑ, C(═O)Rₐ, or C(═O)NR_{b}R_{c} (wherein Rₐ is hydrogen, or alkyl or substituted alkyl, R_{b} and R_{c} are independently hydrogen, or alkyl or substituted alkyl, and Rₑ is alkyl or substituted alkyl (substituted alkyl is optionally substituted with one or more substituent(s) selected from the group consisting of hydroxy, amino, nitro, cyano, halogen, alkoxy, alkylcarbonyl, alkoxycarbonyl, aminocarbonyl, aryl, cycloalkyl, and heterocycle));
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are each independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

2. The compound of claim 1, wherein T is O or S.

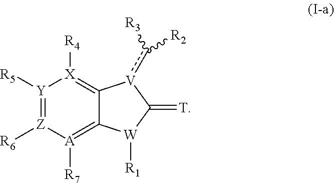

3. The compound of claim 2, wherein the compound is selected from the group consisting of:
(i) a compound wherein T is O, 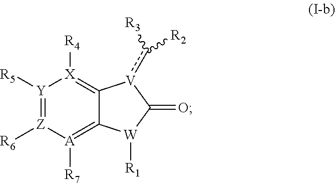
(ii) a compound wherein V is carbon, 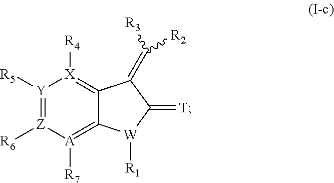
(iii) a compound wherein W is N, 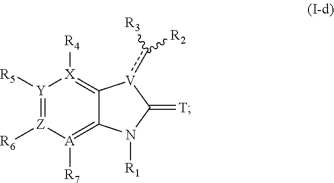
(iv) a compound wherein T is O and W is N, 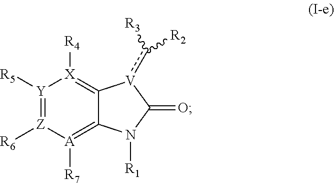
(v) a compound wherein T is O and V is carbon, 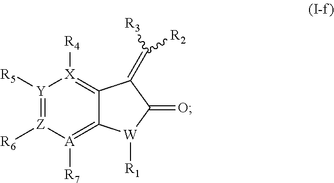
(vi) a compound wherein U is carbon, V is carbon, W is N, and T is O,

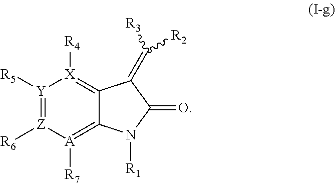

4-10. (canceled)

11. The compound of claim 1, wherein R₂ is 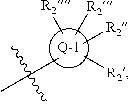
wherein
Q-1 is heterocycle or aryl selected from the group consisting of pyrrole, furan, thiophene, pyridine, pyrimidine, pyrazine, pyridazine, imidazole, indole, pyrrolopyridinone, pyridone, pyrrolidine, pyridinone, piperidine, and pyrroloazepinone; and
R₂′, R₂″, R₂′″, and R₂″″ are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ.

12-19. (canceled)

20. The compound of claim 11, wherein R₂′, R₂″, R₂′″, and R₂″″ are independently absent, hydrogen, alkyl, substituted alkyl, substituted heterocycle, substituted aryl, C(═O)ORₑ, or C(═O)NR_{b}R_{c},
wherein
R_{b} and R_{c} are independently hydrogen, alkyl, substituted alkyl, substituted heterocycle, or said R_{b} and R_{c} together with the N to which they are bonded optionally form a heterocycle or substituted heterocycle, and
Rₑ is hydrogen.

21. The compound of claim 20, wherein one of R₂′, R₂″, R₂′″, and R₂″″ is C(═O)NR_{b}R_{c},
wherein
R_{b} is hydrogen, and
R_{c} is alkyl substituted with NR_{bn}R_{cn} (wherein R_{bn} and R_{cn} are alkyl, or said R_{bn} and R_{cn} together with the N to which they are bonded optionally form a substituted heterocycle (wherein said heterocycle is piperidine, or morpholine)), or R_{b} and R_{c} together with the N to which they are bonded optionally form a substituted heterocycle (wherein said heterocycle is piperidine, or morpholine), and
two of R₂′, R₂″, R₂′″, and R₂″″ are independently alkyl, and the other is hydrogen.

22. The compound of claim 21, wherein one of R₂′, R₂″, R₂′″, and R₂″″ is C(═O)NR_{b}R_{c},
wherein
NR_{b}R_{c} is 2-(di-ethyl amino)ethyl, amino, 2-pyrrolidino ethyl amino, 4-methyl piperazinyl, or morpholino.

23. The compound of claim 11, wherein Q-1 is pyrrole, one of R₂′, R₂″, R₂′″, and R₂″″ is absent, two of R₂′, R₂″, R₂′″, and R₂″″ are alkyl (e.g., methyl), and one of R₂′, R₂″, R₂′″, and R₂″″ is C(═O)NR_{b}R_{c}.

24. The compound of claim 23, wherein
R_{b} is hydrogen, and
R_{c} is alkyl substituted with NR_{bn}R_{cn}, wherein R_{bn} and R_{cn} are alkyl, or said R_{bn} and R_{cn} together with the N to which they are bonded optionally form a substituted heterocycle, and wherein said heterocycle is piperidine, or morpholine.

25. The compound of claim 24, wherein NR_{b}R_{c} is 2-(di-ethyl amino)ethyl, amino, or 2-pyrrolidino ethyl amino.

26. The compound of claim 23, wherein R_{b} and R_{c} together with the N to which they are bonded optionally form a heterocycle or substituted heterocycle.

27. The compound of claim 26, wherein NR_{b}R_{c} is 4-methyl piperazinyl, or morpholino.

28-44. (canceled)

45. The compound of claim 1, wherein Q-2 is selected from the group consisting of the following group:

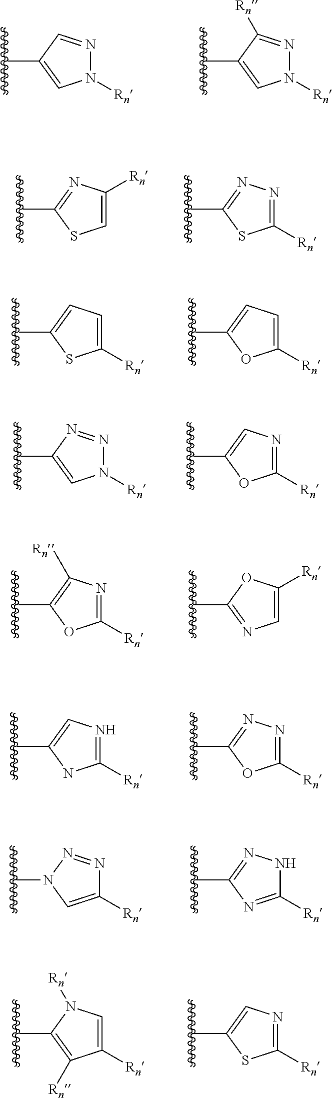

46. The compound of claim 45, wherein Q-2 is selected from the group consisting of the following group:

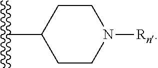

47. The compound of claim 1, wherein the compound is selected from the group consisting of:

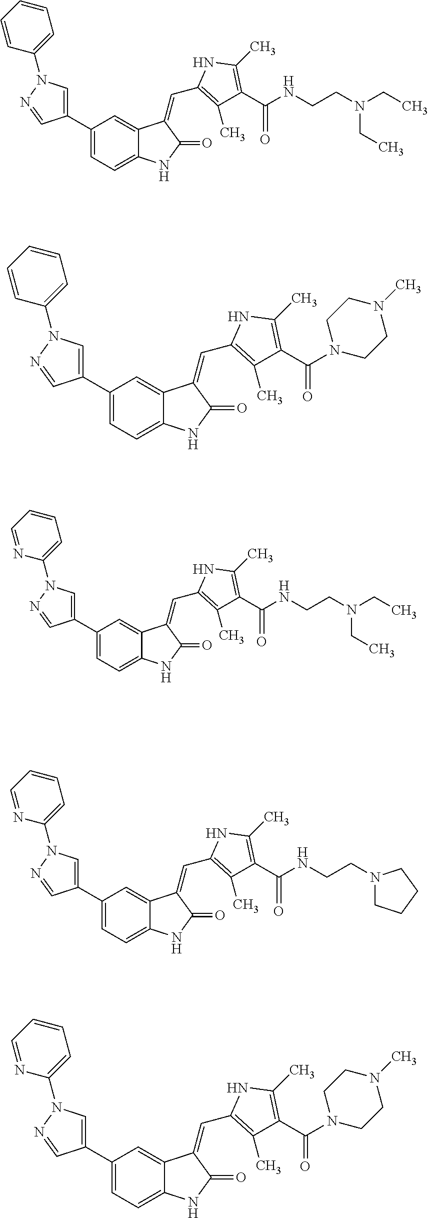 [SMILES: CCN(CC)CCNC(=O)c1c(C)[nH]c(/C=C2\C(=O)Nc3ccc(-c4cnn(-c5ccccc5)c4)cc32)c1C ; Cc1[nH]c(/C=C2\C(=O)Nc3ccc(-c4cnn(-c5ccccc5)c4)cc32)c(C)c1C(=O)N1CCN(C)CC1 ; CCN(CC)CCNC(=O)c1c(C)[nH]c(/C=C2\C(=O)Nc3ccc(-c4cnn(-c5ccccn5)c4)cc32)c1C ; Cc1[nH]c(/C=C2\C(=O)Nc3ccc(-c4cnn(-c5ccccn5)c4)cc32)c(C)c1C(=O)NCCN1CCCC1 ; Cc1[nH]c(/C=C2\C(=O)Nc3ccc(-c4cnn(-c5ccccn5)c4)cc32)c(C)c1C(=O)N1CCN(C)CC1]

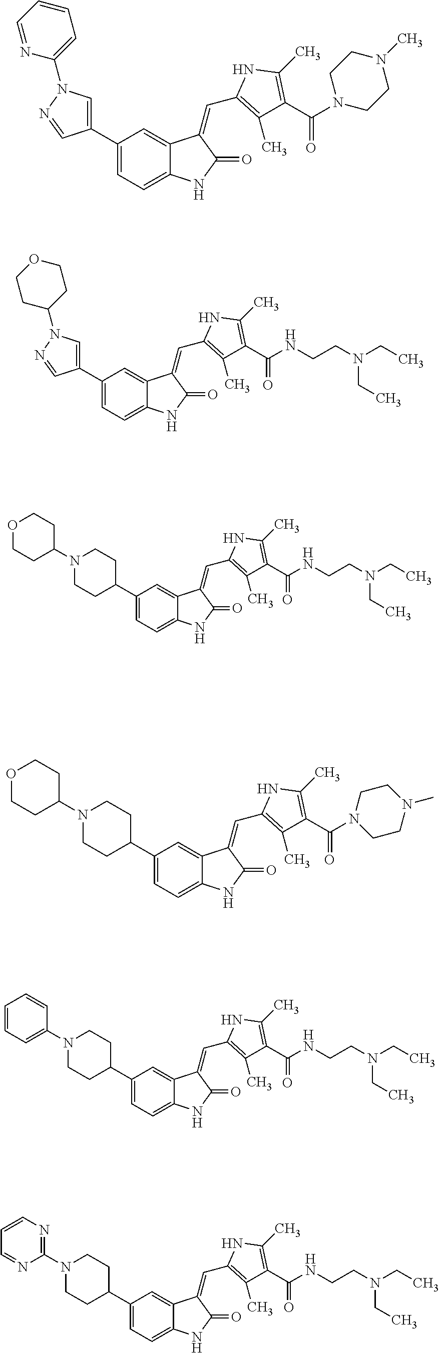 [SMILES: Cc1[nH]c(/C=C2\C(=O)Nc3ccc(-c4cnn(-c5ccccn5)c4)cc32)c(C)c1C(=O)N1CCN(C)CC1 ; CCN(CC)CCNC(=O)c1c(C)[nH]c(/C=C2\C(=O)Nc3ccc(-c4cnn(C5CCOCC5)c4)cc32)c1C ; CCN(CC)CCNC(=O)c1c(C)[nH]c(/C=C2\C(=O)Nc3ccc(C4CCN(C5CCOCC5)CC4)cc32)c1C ; Cc1[nH]c(/C=C2\C(=O)Nc3ccc(C4CCN(C5CCOCC5)CC4)cc32)c(C)c1C(=O)N1CCN(C)CC1 ; CCN(CC)CCNC(=O)c1c(C)[nH]c(/C=C2\C(=O)Nc3ccc(C4CCN(c5ccccc5)CC4)cc32)c1C ; CCN(CC)CCNC(=O)c1c(C)[nH]c(/C=C2\C(=O)Nc3ccc(C4CCN(c5ncccn5)CC4)cc32)c1C]

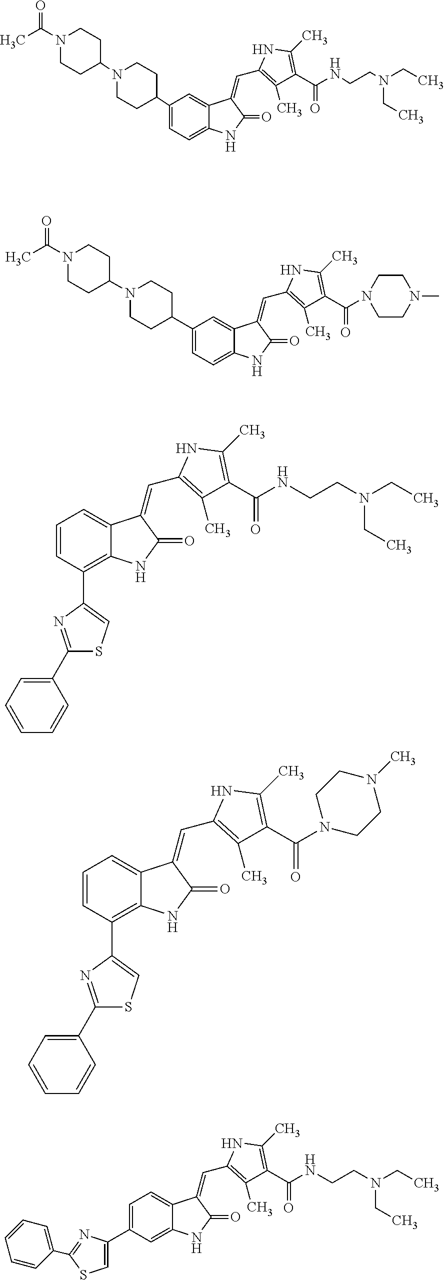

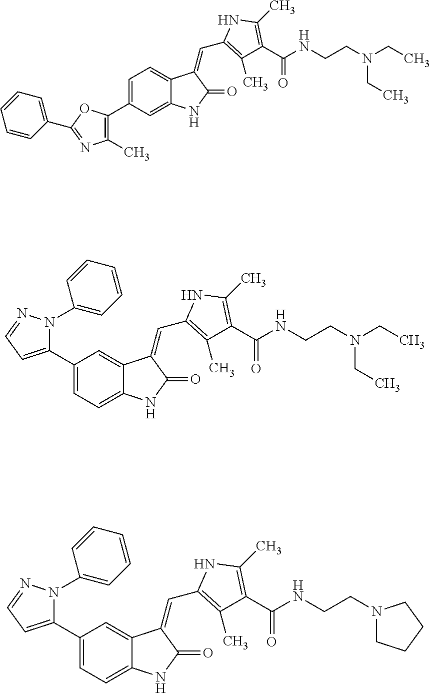 [SMILES: CCN(CC)CCNC(=O)c1c(C)[nH]c(/C=C2\C(=O)Nc3cc(-c4oc(-c5ccccc5)nc4C)ccc32)c1C ; CCN(CC)CCNC(=O)c1c(C)[nH]c(/C=C2\C(=O)Nc3ccc(-c4ccnn4-c4ccccc4)cc32)c1C ; Cc1[nH]c(/C=C2\C(=O)Nc3ccc(-c4ccnn4-c4ccccc4)cc32)c(C)c1C(=O)NCCN1CCCC1]

48. The compound of claim 1, wherein the compound is a compound of Formula II: 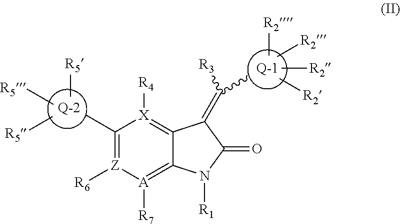
or an enantiomer, diastereomer, tautomer, or pharmaceutically acceptable salt or solvate thereof, wherein
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, S(O)₂NRₐR_{b};
R₄, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Z, and A are each independently carbon or N, with the proviso that the ring in which X, Z, and A exist is aromatic;
Q-1 and Q-2 is independently is heterocycle, or aryl;
R₂′, R₂″, R₂′″, and R₂″″ are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R₅′, R₅″ and R₅′″ are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
wherein
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

49-50. (canceled)

51. The compound of claim 48, wherein the compound is selected from the group consisting of:
(i) a compound having the formula 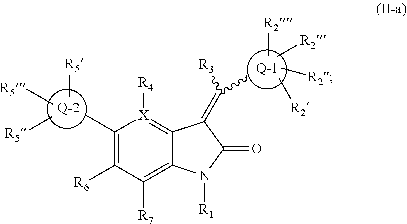
(ii) a compound of the formula, 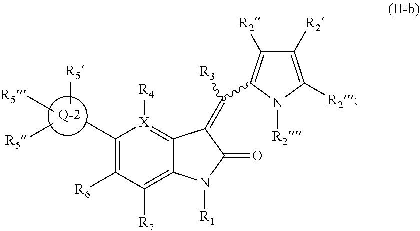
(iii) a compound of the formula 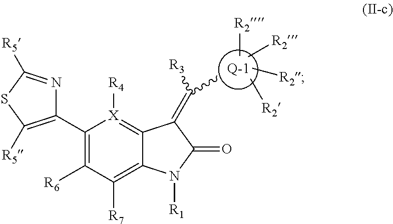
(iv) a compound of the formula of 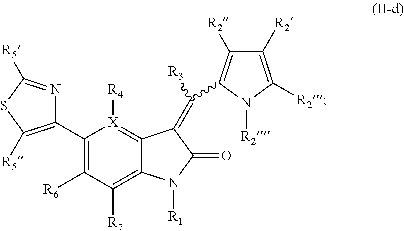
(v) a compound of formula (II-e), 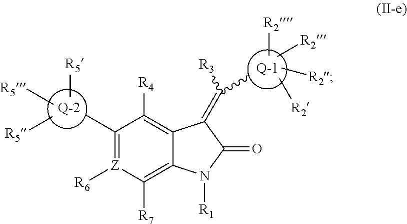
(vi) a compound of the formula of 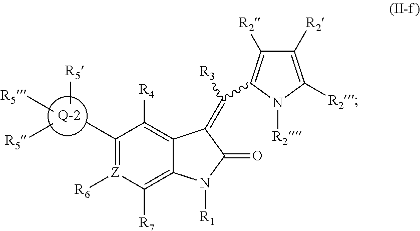
(vii) a compound of the formula 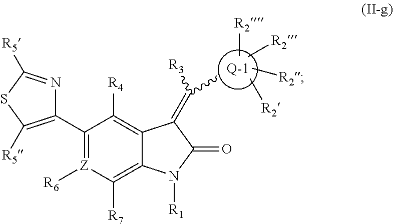
(viii) a compound of the formula of 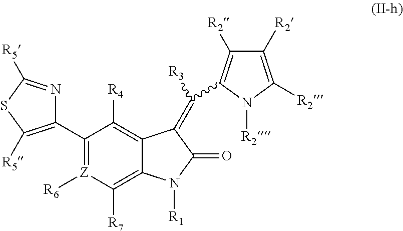
(ix) a compound of the formula of 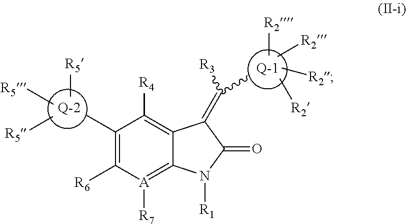
(x) a compound of the formula of 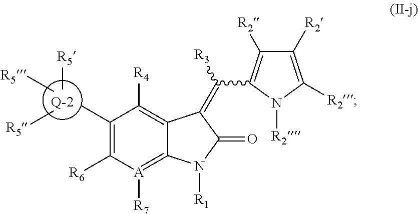
(xi) a compound of the formula of 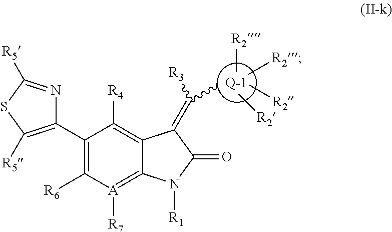
(xii) a compound of the formula of 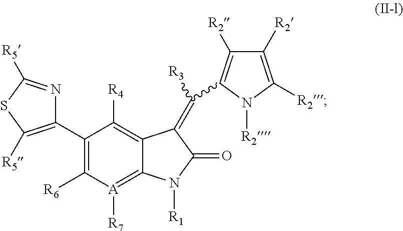
(xiii) a compound of the formula of 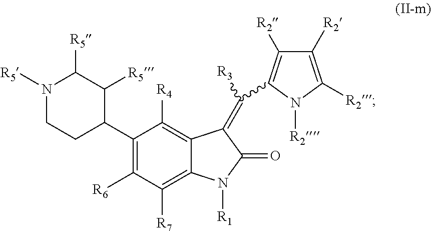
(xiii) a compound of the formula of 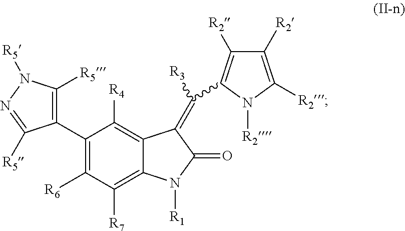
and
(xiv) a compound of the formula of

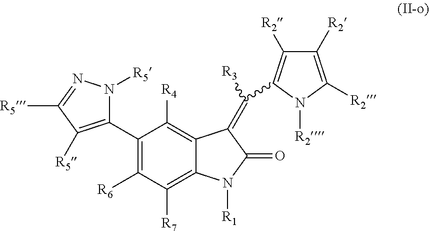

52-81. (canceled)

82. The compound of claim 1, wherein the compound is a compound of Formula III, 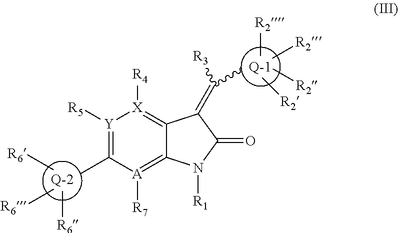
or an enantiomer, diastereomer, tautomer, or pharmaceutically acceptable salt or solvate thereof, wherein:
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Y, and A are each independently carbon or N, with the proviso that the ring in which X, Y, and A exist is aromatic;
Q-1 and Q-2 are each independently is heterocycle or aryl;
R₂′, R₂″, R₂′″ and R₂″″ are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
R₆′, R₆″ and R₆′″ are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are each independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

83-84. (canceled)

85. The compound of claim 82, wherein the compound is selected from the group consisting of:
(i) a compound having the formula of 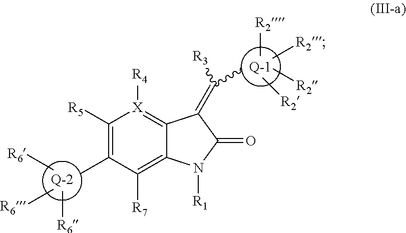
(ii) a compound of the formula of 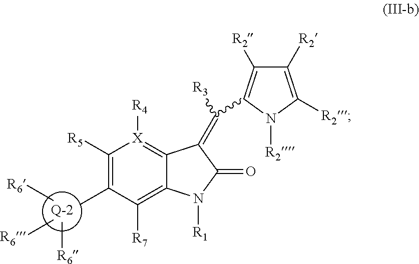
wherein
(iii) a compound of the formula of 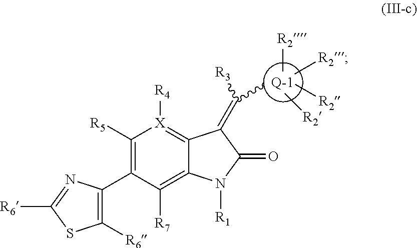
(iv) a compound of the formula of 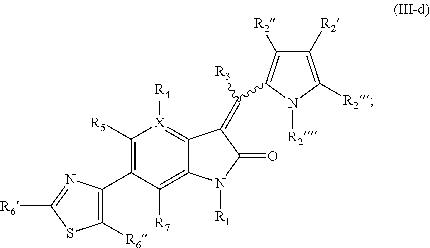
(v) a compound of the formula of 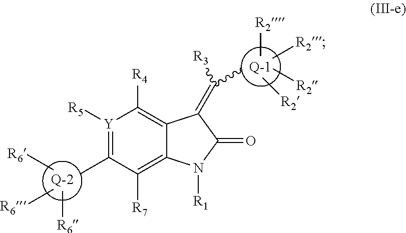
(vi) a compound of the formula of 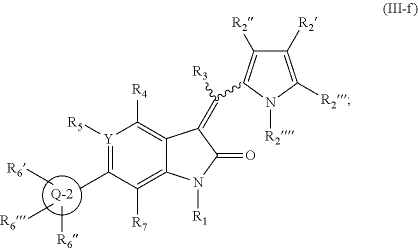
(vii) a compound of the formula of 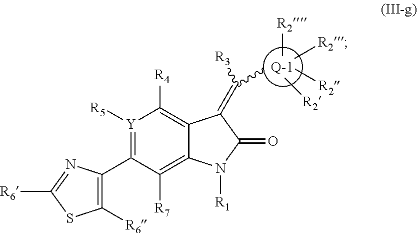
(viii) a compound of the formula of 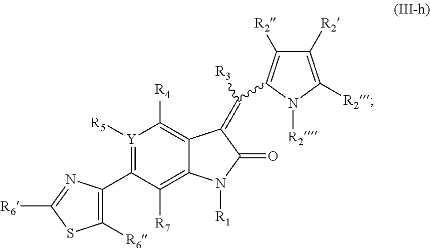
(ix) a compound of the formula of 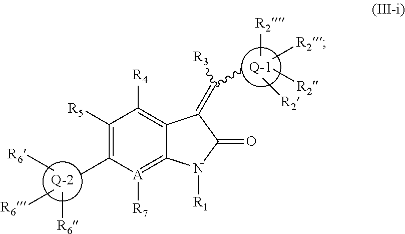
(x) a compound of the formula of 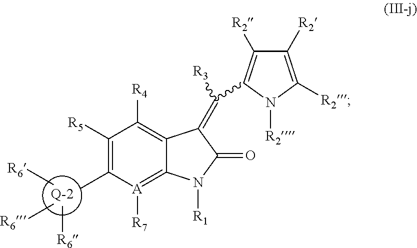
(xi) a compound of the formula of 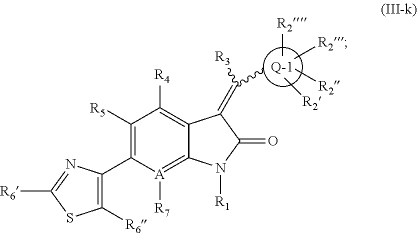
and
(xii) a compound of the formula of

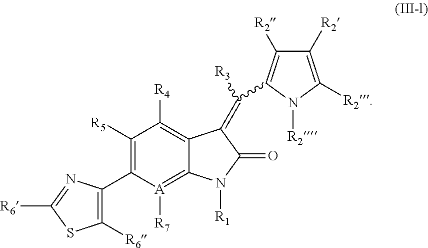

86-112. (canceled)

113. The compound of claim 1, wherein the compound is a compound of Formula IV, 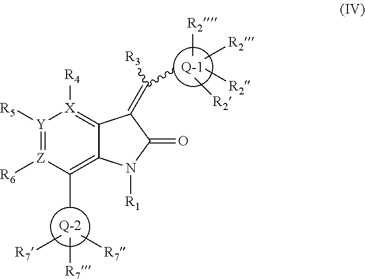
or an enantiomer, diastereomer, tautomer, or pharmaceutically acceptable salt or solvate thereof, wherein:
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₄, R₅, and R₆ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
X, Y, and Z are each independently a carbon or N, with the proviso that the ring in which X, Y, and Z exist is aromatic;
Q-1 and Q-2 are each independently is heterocycle or aryl;
R₂′, R₂″, R₂′″ and R₂″″ are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, or ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
R₇′, R₇″ and R₇′″ are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are each independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

114-115. (canceled)

116. The compound of claim 113, wherein the compound is selected from the group consisting of
(i) a compound having the formula: 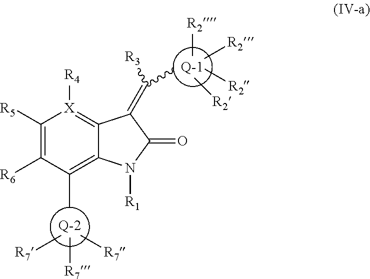
(ii) a compound having the formula: 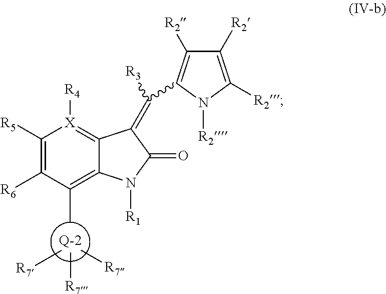
(iii) a compound of the formula of 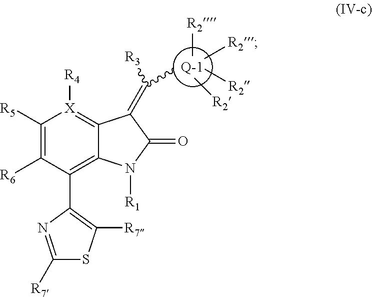
(iv) a compound of the formula of 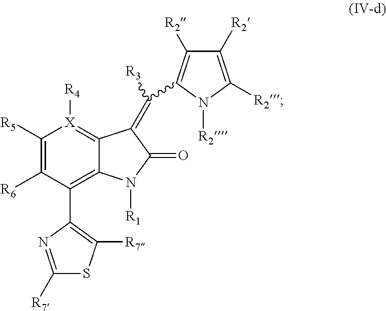
(v) a compound of the formula of 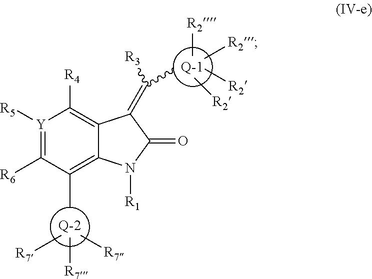
(vi) a compound of the formula of 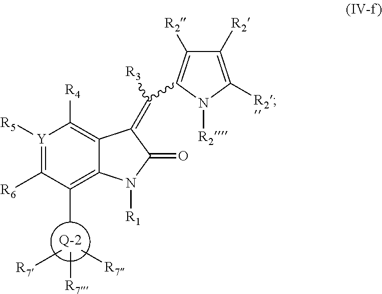
(vii) a compound of the formula of 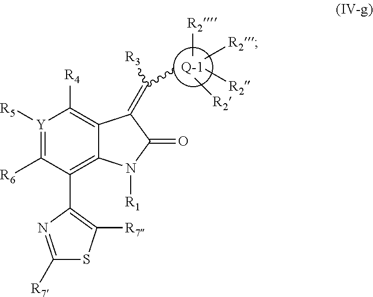
(viii) a compound of the formula of 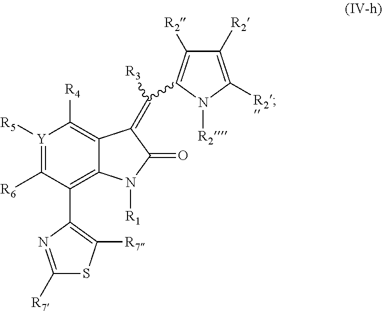
(ix) a compound of the formula of 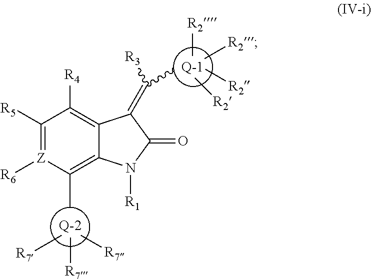
(x) a compound of the formula: 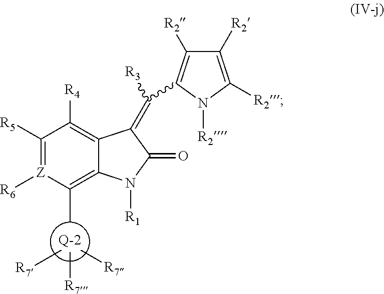
(xi) a compound of the formula: 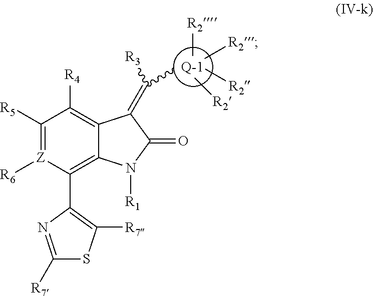
(xii) a compound of the formula:

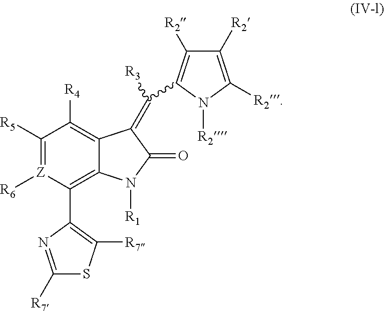

117-143. (canceled)

144. The compound of claim 1, wherein the compound is a compound of Formula V, 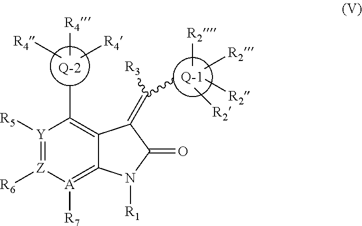
or an enantiomer, diastereomer, tautomer, or pharmaceutically acceptable salt or solvate thereof, wherein:
R₁ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)₂Rₑ, S(═O)₂ORₑ, C(═O)OR_{d}, C(═O)Rₐ, or C(═O)NR_{b}R_{c};
R₃ is hydrogen, alkyl or substituted alkyl, cycloalkyl or substituted cycloalkyl, heterocycle or substituted heterocycle, aryl or substituted aryl, halogen, —ORₐ, —C(O)Rₐ, —C(O)ORₐ, —NRₐR_{b}, or S(O)₂NRₐR_{b};
R₅, R₆, and R₇ are each independently hydrogen, halogen, cyano, nitro, trihalomethyl, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
Y, Z and A are each independently carbon or N, with the proviso that the ring in which Y, Z and A exist is aromatic;
Q-1 and Q-2 are each independently is heterocycle or aryl;
R₂′, R₂″, R₂′″ and R₂″″ are each independently absent, hydrogen, halogen, cyano, nitro, trihalomethyl, OCF₃, alkyl or substituted alkyl, cycloalkyl or substituted cycloalkyl, heterocycle or substituted heterocycle, aryl or substituted aryl, ORₐ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ;
R₄′, R₄″ and R₄′″ are each independently hydrogen, halogen, cyano, nitro, trihalomethyl, OCF₃, alkyl or substituted alkyl, ORₐ, SRₐ, C(═O)Rₐ, C(═O)ORₐ, NH₂, S(O)₂NH₂, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are each independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

145-146. (canceled)

147. The compound of claim 144, wherein the compound is selected from the group consisting of
(i) a compound having the formula of 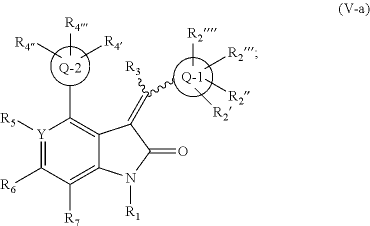
(ii) a compound of the formula of 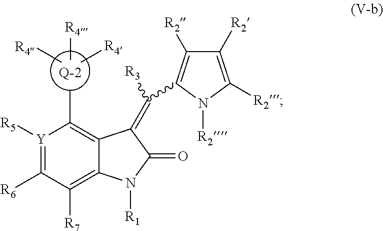
(iii) a compound of the formula of 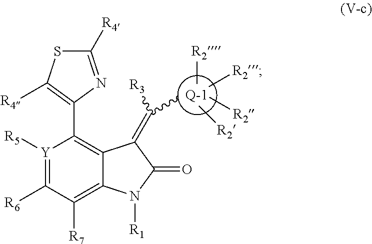
(iv) a compound of the formula of 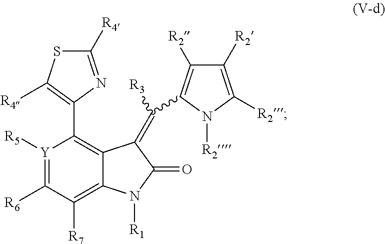
(v) a compound of the formula of 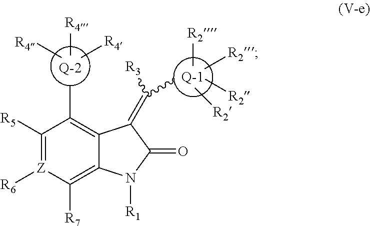
(vi) a compound of the formula of 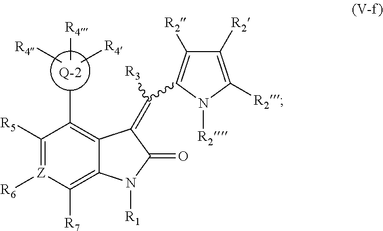
(vii) a compound of the formula of 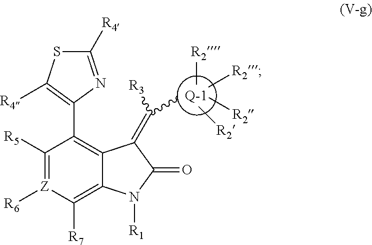
(viii) a compound of the formula of 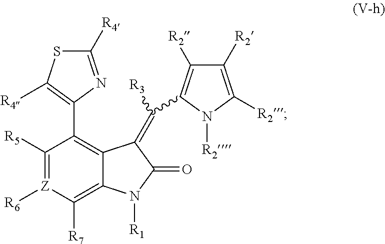
(ix) a compound of the formula of 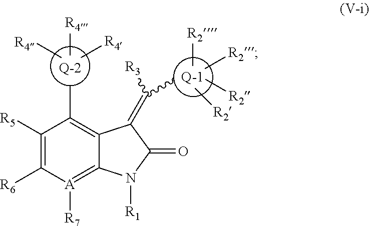
(x) a compound of the formula of 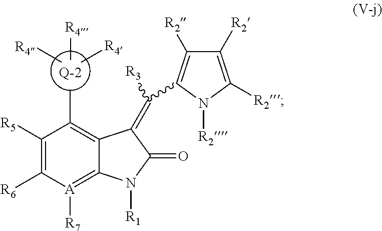
(xi) a compound of the formula of 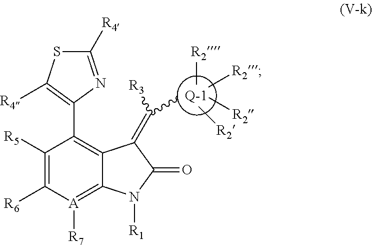
(xii) a compound of the formula of

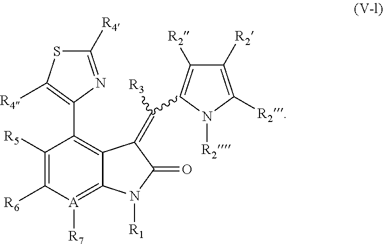

148-173. (canceled)

174. A pharmaceutical composition comprising a compound of claim 1, or a pharmaceutically acceptable salt, ester or pro-drug thereof, and a pharmaceutically acceptable excipient, carrier, or diluent.

175. (canceled)

176. A method of treating, preventing or ameliorating a protein kinase related disorder in a mammal, comprising administering to the mammal in need thereof a therapeutically effective amount of a pharmaceutical composition comprising a compound of claim 1.

177-185. (canceled)
